(19) United States
(12) Patent Application Publication
(10) Pub. No.: US 2023/0346777 A1
(43) Pub. Date: Nov. 2, 2023

(54) USE OF SGC ACTIVATORS FOR THE TREATMENT OF OPHTHALMOLOGIC DISEASES

(71) Applicant: Bayer Aktiengesellschaft, Leverkusen (DE)

(72) Inventors: William Ernst SCHUBERT, Wuppertal (DE); Khaled NASSAR, Haan (DE); Peter SANDNER, Wuppertal (DE); Carsten TERJUNG, Bochum (DE); Elia DUH, Lutherville, MD (US); Hongkwan CHO, Baltimore, MD (US); Zhenhua XU, Ellicott City, MD (US); Lijuan WU, Baltimore, MD (US); Lingli ZHOU, Baltimore, MD (US)

(21) Appl. No.: 18/134,875

(22) Filed: Apr. 14, 2023

Related U.S. Application Data
(63) Continuation of application No. 17/667,418, filed on Feb. 8, 2022.
Application No. 17/667,418 is a continuation of application No. PCT/EP2021/084991, filed on Dec. 9, 2021.
(60) Provisional application No. 63/123,787, filed on Dec. 10, 2020.

(51) Int. Cl. A61K 31/496; A61P 27/02; A61K 45/06

(52) U.S. Cl. CPC A61K 31/496; A61P 27/02; A61K 45/06

(57) ABSTRACT

The invention relates to substituted pyrazolo piperidine carboxylic acids, their salts and their use for preparing medicaments for the treatment and/or prophylaxis of diseases, in particular of ophthalmologic diseases, including non-proliferative diabetic retinopathy (NPDR), diabetic macular edema (DME), retinal ganglion cell/photoreceptor neurodegeneration and cataract.

This application is a U.S. continuation patent application of U.S. application Ser. No. 17/667,418, filed Feb. 8, 2022, which is a U.S. continuation application of International PCT Patent Application No. PCT/EP2021/084991, filed Dec. 9, 2021, which is incorporated herein by reference in its entirety, which claims benefit of priority to U.S. Provisional Patent Application No. 63/123,787, filed Dec. 10, 2020.

The present invention relates to soluble guanylate cyclase (sGC) activators for use in the treatment and/or prophylaxis of ophthalmologic diseases, including non-proliferative diabetic retinopathy (NPDR), diabetic macular edema (DME), retinal ganglion cell/photoreceptor neurodegeneration and cataract, especially wherein the soluble guanylate cyclase (sGC) activators are compounds of formula (I)

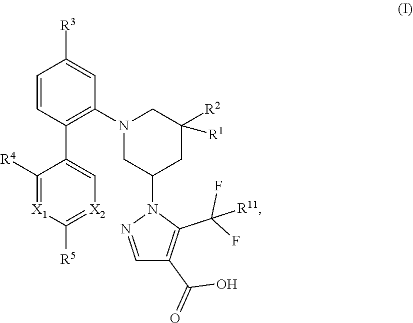

in which
R¹ represents hydrogen or halogen,
R² represents hydrogen or halogen,
R³ represents chloro or trifluoromethyl,
R⁴ represents hydrogen, C₁-C₄-alkyl,
R⁵ represents a group of the formula

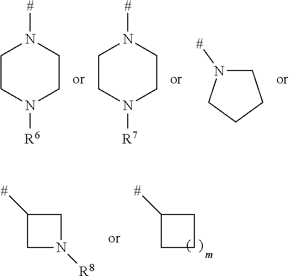

where #is the point of attachment to the aromatic or heteroaromatic 6 ring system; wherein m is 0-4
R⁶ represents
   C₁-C₆-alkyl, optionally substituted by one or more substituent independently selected from the group consisting of methyl, trifluoromethoxy, nitril, amido,
   C₂-C₆-halogenoalkyl, optionally substituted by 1 to 5 fluoro substituents,
   C₃-C₆-cycloalkyl,
   C₃-C₆-cycloalkyl-methyl, optionally substituted by 1 to 5 fluoro substituents or a trifluoromethyl group,
   C₁-C₆-alkylcarbonyl, optionally substituted by 1 to 3 fluoro substituents,
   C₃-C₆-cycloalkyl-carbonyl, optionally substituted by 1 to 3 fluoro substituents or
   (C₁-C₆)-alkoxy-carbonyl, optionally substituted with methoxy, trifluoromethoxy, C₃-C₆-cycloalkyl,
   (C₃-C₆)-cycloalkoxy-carbonyl,
   mono-(C₁-C₄)-alkylaminocarbonyl,
   (C₁-C₄)-alkylsulfonyl or
   oxetanyl,
   spiro[2.2]pentan-2-ylmethyl or [(3-fluoro-1-bicyclo[1.1.1]pentanyl)methyl,
R⁷ represents C₁-C₄-alkylcarbonyl, optionally substituted by a C₃-C₆-cycloalkyl group,
R⁸ represents C₂-C₄-alkyl, C₂-C₄-halogenoalkyl substituted by 1 to 6 fluoro substituents,
R¹¹ represents hydrogen or fluoro substituent
X₁ represents nitrogen or carbon or C—F
X₂ represents nitrogen or carbon
and the salts thereof, the solvates thereof and the solvates of the salts thereof.

The present invention also relates to soluble guanylate cyclase (sGC) activators for use in the treatment and/or prophylaxis of ophthalmologic diseases, including non-proliferative diabetic retinopathy (NPDR), diabetic macular edema (DME), retinal ganglion cell/photoreceptor neurodegeneration and cataract, especially wherein the soluble guanylate cyclase (sGC) activators are compounds of formula (I-A)

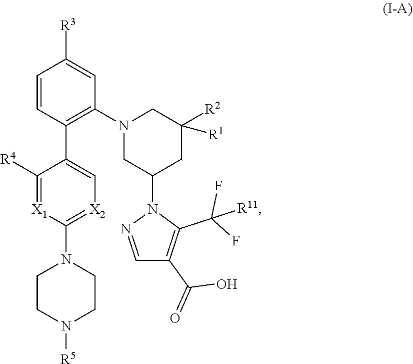

in which
R¹ represents hydrogen or halogen,
R² represents hydrogen or halogen,
R³ represents chloro or trifluoromethyl,
R⁴ represents hydrogen or C₁-C₄-alkyl
R⁵ represents optionally substituted C₁-C₆-alkyl
R¹¹ represents hydrogen or fluoro substituent
X₁ represents nitrogen or carbon
X₂ represents nitrogen or carbon
and the salts thereof, the solvates thereof and the solvates of the salts thereof.

The term “substituted” means that one or more hydrogen atoms on the designated atom or group are replaced with a selection from the indicated group, provided that the designated atom's normal valence under the existing circumstances is not exceeded. Combinations of substituents and/or variables are permissible.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

In the context of the present invention, unless specified otherwise, the substituents are defined as follows: The term “halogen” or “halogeno” like in combinations e.g. in halogenoalkyl means a fluorine, chlorine, bromine or iodine atom, particularly a fluorine, chlorine or bromine atom, even more particularly fluorine or chlorine.

The term “C₁-C₄-alkyl”, “C₁-C₅-alkyl” and “C₁-C₆-alkyl” means a linear or branched, saturated, monovalent hydrocarbon group having 1, 2, 3, or 4 carbon atoms, 1, 2, 3, 4 or 5 carbon atoms, and 1, 2, 3, 4, 5 or 6 carbon atoms, e.g. a methyl, ethyl, propyl, isopropyl, n-butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-halogenoalkyl”, “C₂-C₆-halogenoalkyl”, “C₁-C₄-halogenoalkyl”, “C₂-C₄-halogenoalkyl”, “C₁-C₃-halogenoalkyl” and “C₁-C₂-halogenoalkyl” represents a linear or branched, saturated, monovalent hydrocarbon group in which the term “alkyl” is as defined supra, and in which one or more of the hydrogen atoms are replaced, identically or differently, with a halogen atom. Particularly, said halogen atom is a fluorine atom. Said C₁-C₆-halogenoalkyl group is, for example fluoromethyl, difluoromethyl, trifluoromethyl, 2-fluoroethyl, 2,2-difluoroethyl, 2,2,2-trifluoroethyl, pentafluoroethyl, 3,3,3-tri-fluoropropan-1-yl, 1,1,1-trifluoropropan-2-yl, 1,3-difluoropropan-2-yl, 3-fluoropropan-1-yl, 1,1,1-trifluoro-butan-2-yl, and 3,3,3-trifluoro-1-methyl-propan-1-yl.

The term “C₁-C₄-halogenoalkoxy” and “C₁-C₃-halogenoalkoxy” represents a linear or branched, saturated, monovalent C₁-C₄-alkoxy or C₁-C₃-alkoxy group (where alkoxy represents a straight-chain or branched, saturated, monovalent alkoxy radical having 1 to 4 or 1 to 3 carbon atoms, by way of example and with preference methoxy, ethoxy, n-propoxy, isopropoxy), in which one or more of the hydrogen atoms is replaced, identically or differently, with a halogen atom. Particularly, said halogen atom is a fluorine atom.

Said C₁-C₃-halogenoalkoxy group is, for example, fluoromethoxy, difluoromethoxy, trifluoromethoxy, 2,2,2-trifluoroethoxy or pentafluoroethoxy.

The term “(C₁-C₄)-alkylcarbonyl” represents a straight-chain or branched alkyl radical having 1 to 4 carbon atoms which is attached via a carbonyl group [—C(═O)—] to the remainder of the molecule. The following may be mentioned by way of example and by way of preference: acetyl, propionyl, n-butyryl, isobutyryl, t-butyryl, n-pentanoyl and pivaloyl.

The term “mono-(C₁-C₄)-alkylaminocarbonyl” represents an amino group which is bound to the remainder of the molecule via a carbonyl group [—C(═O)—] and which has one straight-chain or branched alkyl substituent having 1, 2, 3 or 4 carbon atoms, such as: methylaminocarbonyl, ethylaminocarbonyl, n-propylaminocarbonyl, isopropylaminocarbonyl, n-butylaminocarbonyl, and tert-butylaminocarbonyl, for example.

The term “(C₁-C₄)-alkylsulfonyl” represents a linear or branched, saturated, monovalent group of formula (C₁-C₄-alkyl)-S(═O)—, in which the term “C₁-C₄-alkyl” is as defined supra, e.g. a methylsulfonyl, ethylsulfonyl, propylsulfonyl, isopropylsulfonyl, butylsulfonyl, sec-butylsulfonyl, isobutylsulfonyl, tert-butylsulfonyl group.

The term “(C₁-C₄)-alkoxy-carbonyl” represents a straight-chain or branched alkoxy group having 1, 2, 3 or 4 carbon atoms which is bound to the rest of the molecule via a carbonyl group [—C(═O)—], such as: methoxycarbonyl, ethoxycarbonyl, n-propoxycarbonyl, isopropoxycarbonyl, n-butoxycarbonyl, and tert-butoxycarbonyl, for example.

The term “(C₃-C₆)-cycloalkoxy-carbonyl” represents a saturated, monovalent, monocyclic hydrocarbon ring which contains 3, 4, 5 or 6 carbon atoms. Said C₃-C₆-cycloalkoxy group is for example a cyclopropyloxy, cyclobutyloxy, cyclopentyloxy or cyclohexyloxy group which is bound to the rest of the molecule via a carbonyl group [—C(═O)—], such as: cyclopropyloxycarbonyl, cyclobutyloxycarbonyl, cyclopentyloxycarbonyl, cyclohexyloxycarbonyl, for example.

The term “C₃-C₆-cycloalkyl” means a saturated, monovalent, monocyclic hydrocarbon ring which contains 3, 4, 5 or 6 carbon atoms. Said C₃-C₆-cycloalkyl group is for example a cyclopropyl, cyclobutyl, cyclopentyl or cyclohexyl group.

Compounds according to the invention are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The inventive compounds may, depending on their structure, exist in different stereoisomeric forms, i.e. in the form of configurational isomers or else, if appropriate, of conformational isomers (enantiomers and/or diastereomers, including those in the case of rotamers and atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

The present invention includes all possible tautomers of the compounds of the present invention as single tautomers, or as any mixture of said tautomers, in any ratio.

In the context of the present invention, the term “enantiomerically pure” is understood to mean that the compound in question with respect to the absolute configuration of the chiral centre is present in an enantiomeric excess of more than 95%, preferably more than 97%. The enantiomeric excess (ee value) is calculated in this case by evaluation of the corresponding HPLC chromatogram on a chiral phase with the aid of the formula below:

ee=[E^{A} (area %)−E^{B} (area %)]×100%/[E^{A} (area %)+E^{B} (area %)]

(E^{A}: enantiomer in excess, E^{B}: enantiomer in deficiency)

The present invention also encompasses all suitable isotopic variants of the compounds according to the invention. An isotopic variant of an inventive compound is understood here as meaning a compound in which at least one atom within the inventive compound has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴, S³⁵, S³⁶, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the inventive compounds may therefore in some cases also constitute a preferred embodiment of the present invention. Isotopic variants of the compounds according to the invention can be prepared by the processes known to those skilled in the art, for example by the methods described further below and the procedures described in the working examples, by using corresponding isotopic modifications of the respective reagents and/or starting compounds.

Preferred salts in the context of the present invention are physiologically acceptable salts of the compounds according to the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

The present invention includes all possible salts of the compounds according to the invention as single salts, or as any mixture of said salts, in any ratio.

Solvates in the context of the invention are described as those forms of the inventive compounds which form a complex in the solid or liquid state by coordination with solvent molecules. The compounds according to the invention may contain polar solvents, in particular water, methanol or ethanol for example, as structural element of the crystal lattice of the compounds. Hydrates are a specific form of the solvates in which the coordination is with water. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds according to the invention can exist as N-oxides, which are defined in that at least one nitrogen of the compounds of the present invention is oxidised in a known manner. The present invention includes all such possible N-oxides.

The present invention additionally also encompasses prodrugs of the inventive compounds. The term “prodrugs” encompasses compounds which for their part may be biologically active or inactive but are converted during their residence time in the body into compounds according to the invention (for example by metabolism or hydrolysis).

In the formulae of the group which may represent R⁵, the end point of the line marked by #in each case does not represent a carbon atom or a CH₂ group, but is part of the bond to the atom to which R⁵ is attached.

The present invention preferably relates to soluble guanylate cyclase (sGC) activators for use in the treatment and/or prophylaxis of ophthalmologic diseases, including non-proliferative diabetic retinopathy (NPDR), diabetic macular edema (DME), retinal ganglion cell/photoreceptor neurodegeneration and cataract, especially wherein the soluble guanylate cyclase (sGC) activators are compounds selected from the group consisting of

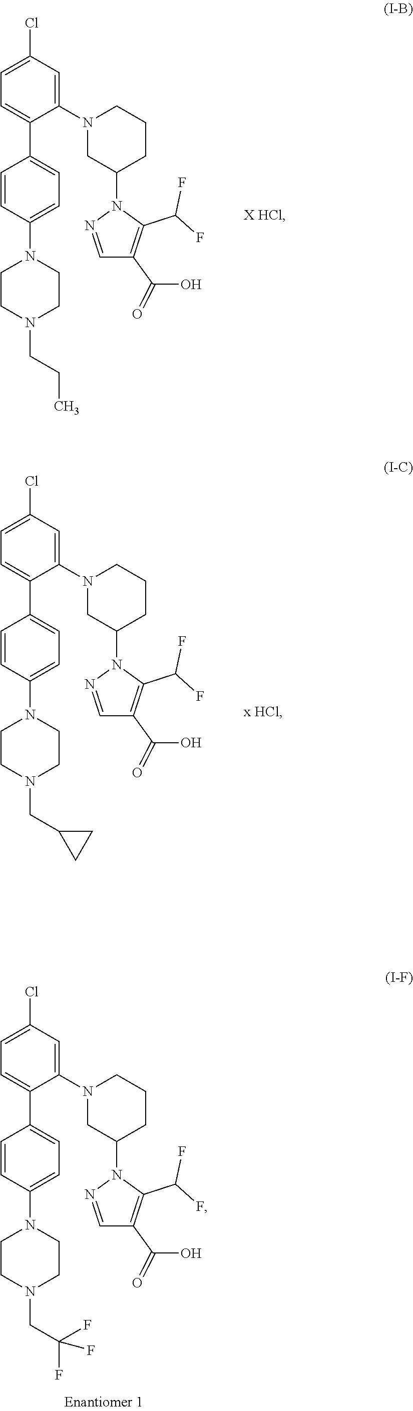 [SMILES: CCCN1CCN(c2ccc(-c3ccc(Cl)cc3N3CCCC(n4ncc(C(=O)O)c4C(F)F)C3)cc2)CC1 ; O=C(O)c1cnn(C2CCCN(c3cc(Cl)ccc3-c3ccc(N4CCN(CC5CC5)CC4)cc3)C2)c1C(F)F ; O=C(O)c1cnn(C2CCCN(c3cc(Cl)ccc3-c3ccc(N4CCN(CC(F)(F)F)CC4)cc3)C2)c1C(F)F]

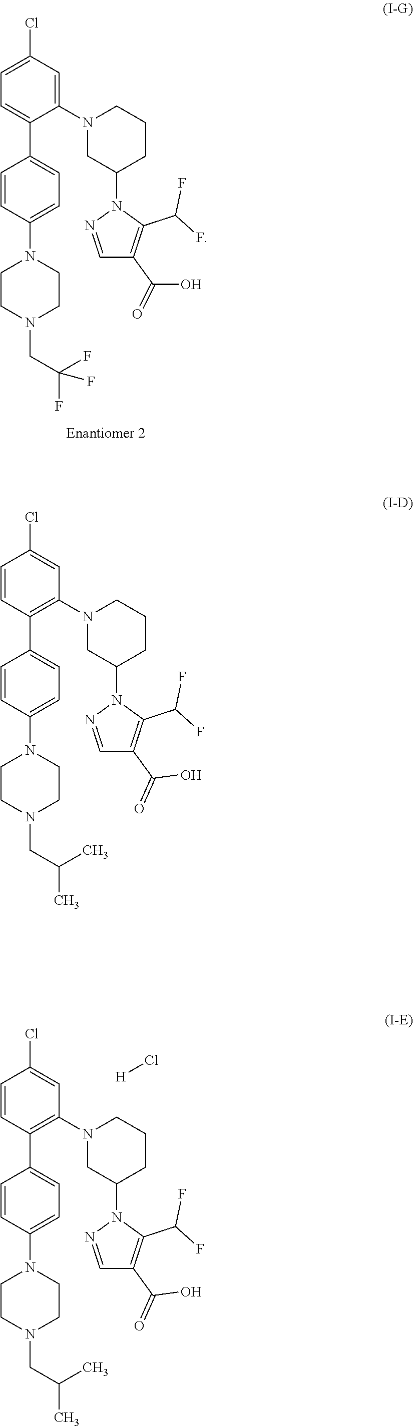

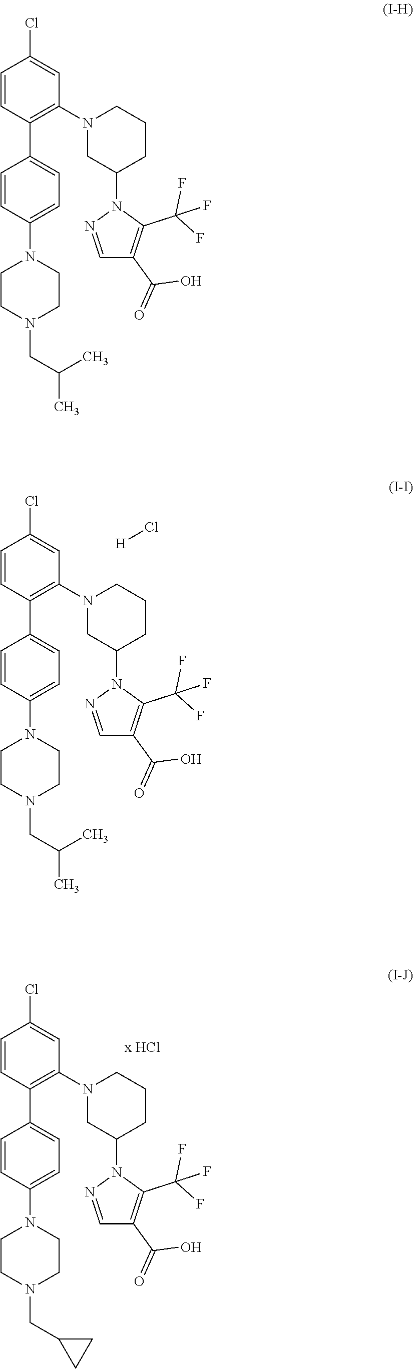

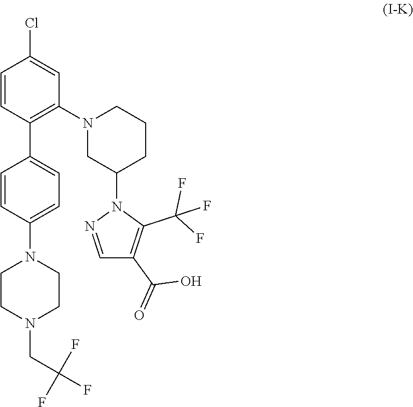 [SMILES: O=C(O)c1cnn(C2CCCN(c3cc(Cl)ccc3-c3ccc(N4CCN(CC(F)(F)F)CC4)cc3)C2)c1C(F)(F)F]

and the salts thereof, the solvates thereof and the solvates of the salts thereof.

The present invention preferably also relates to soluble guanylate cyclase (sGC) activators for use in the treatment and/or prophylaxis of ophthalmologic diseases, including non-proliferative diabetic retinopathy (NPDR), diabetic macular edema (DME), retinal ganglion cell/photoreceptor neurodegeneration and cataract, especially wherein the soluble guanylate cyclase (sGC) activators are compounds selected from the group consisting of

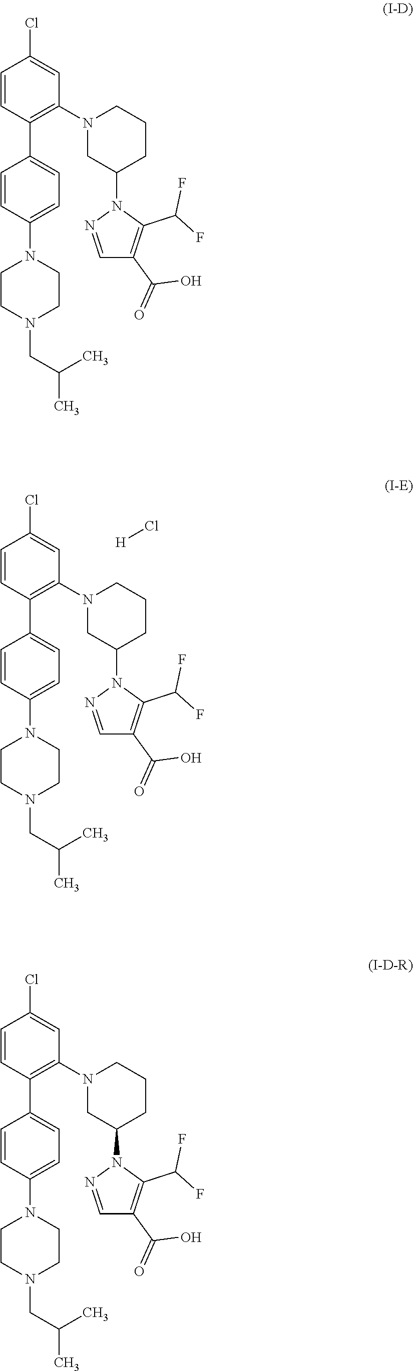

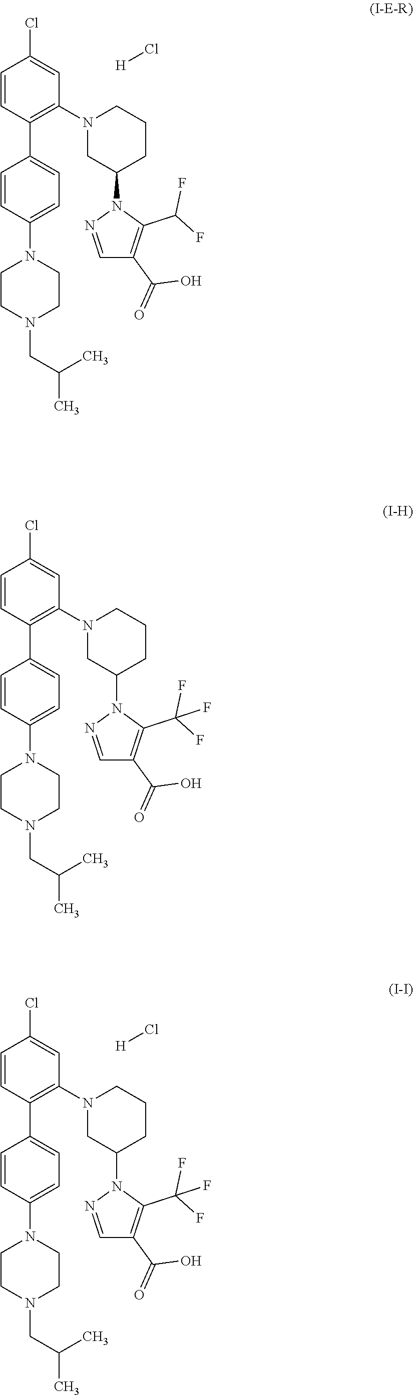

The present invention preferably also relates to soluble guanylate cyclase (sGC) activators for use in the treatment and/or prophylaxis of ophthalmologic diseases, including non-proliferative diabetic retinopathy (NPDR), diabetic macular edema (DME), retinal ganglion cell/photoreceptor neurodegeneration and cataract, especially wherein the soluble guanylate cyclase (sGC) activators are compounds selected from the group consisting of

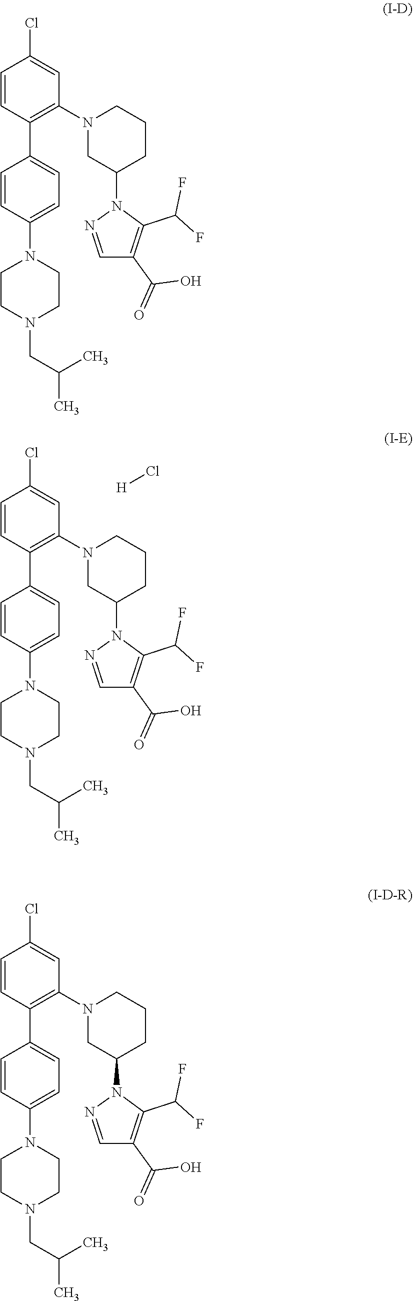 [SMILES: CC(C)CN1CCN(c2ccc(-c3ccc(Cl)cc3N3CCCC(n4ncc(C(=O)O)c4C(F)F)C3)cc2)CC1 ; CC(C)CN1CCN(c2ccc(-c3ccc(Cl)cc3N3CCCC(n4ncc(C(=O)O)c4C(F)F)C3)cc2)CC1 ; Cl ; CC(C)CN1CCN(c2ccc(-c3ccc(Cl)cc3N3CCC[C@@H](n4ncc(C(=O)O)c4C(F)F)C3)cc2)CC1]

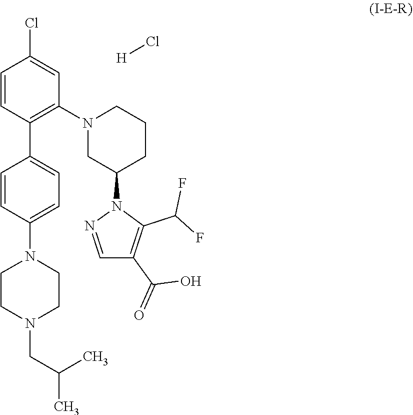 [SMILES: CC(C)CN1CCN(c2ccc(-c3ccc(Cl)cc3N3CCC[C@@H](n4ncc(C(=O)O)c4C(F)F)C3)cc2)CC1 ; Cl]

The present invention preferably also relates to soluble guanylate cyclase (sGC) activators for use in the oral treatment and/or prophylaxis of non-proliferative diabetic retinopathy (NPDR), especially wherein the soluble guanylate cyclase (sGC) activators are compounds selected from the group consisting of

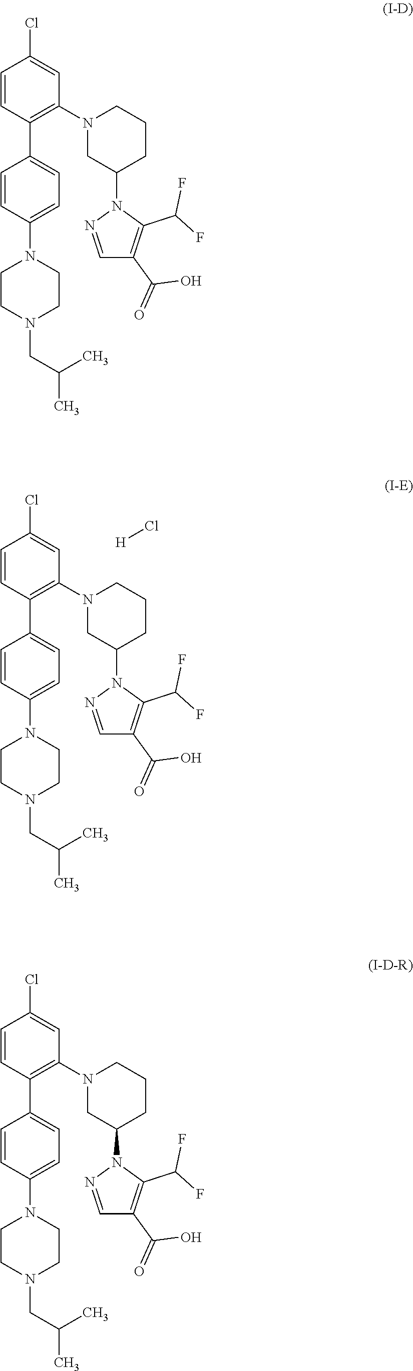

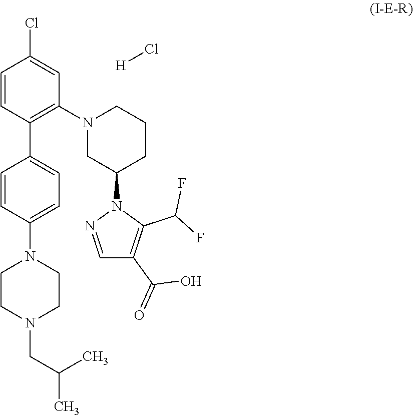

Preference is also given to compounds of the formula (I-D)

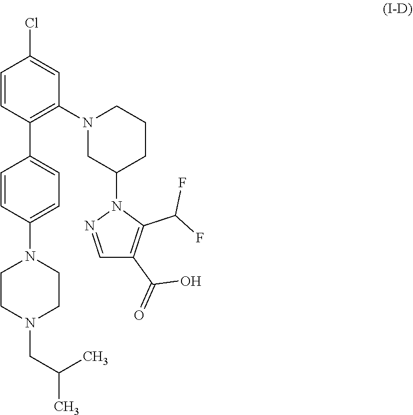 [SMILES: CC(C)CN1CCN(c2ccc(-c3ccc(Cl)cc3N3CCCC(n4ncc(C(=O)O)c4C(F)F)C3)cc2)CC1]

and the salts thereof, the solvates thereof and the solvates of the salts thereof and all potential enantiomeric forms.

Preference is also given to compounds of the formula (I-D-R)

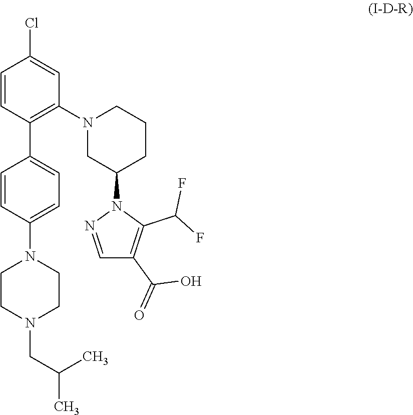 [SMILES: CC(C)CN1CCN(c2ccc(-c3ccc(Cl)cc3N3CCC[C@@H](n4ncc(C(=O)O)c4C(F)F)C3)cc2)CC1]

and the salts thereof, the solvates thereof and the solvates of the salts thereof.

Especially preference is given to compounds of formula (I-E)

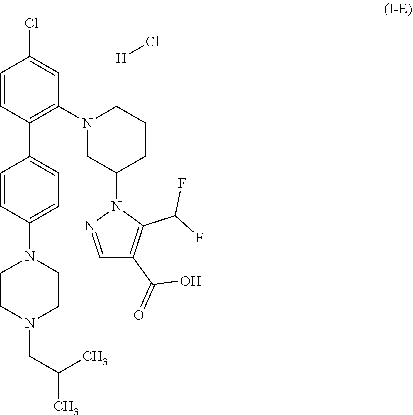 [SMILES: CC(C)CN1CCN(c2ccc(-c3ccc(Cl)cc3N3CCCC(n4ncc(C(=O)O)c4C(F)F)C3)cc2)CC1 ; Cl]

and all potential enantiomeric forms.

Especially preference is given to compounds of formula (I-E-R) in form of its R enantiomer

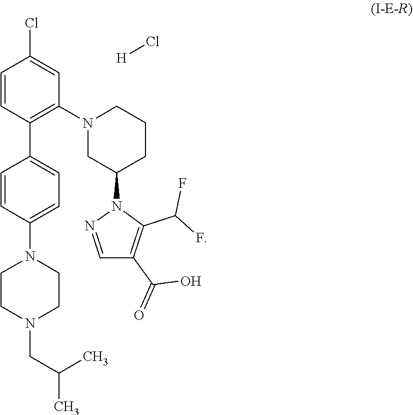 [SMILES: CC(C)CN1CCN(c2ccc(-c3ccc(Cl)cc3N3CCC[C@@H](n4ncc(C(=O)O)c4C(F)F)C3)cc2)CC1 ; Cl]

Especially preference is given to compound of formula

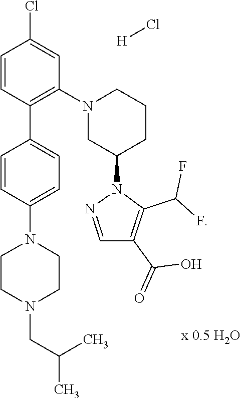 [SMILES: CC(C)CN1CCN(c2ccc(-c3ccc(Cl)cc3N3CCC[C@@H](n4ncc(C(=O)O)c4C(F)F)C3)cc2)CC1 ; Cl]

Especially preference is given to compound of formula

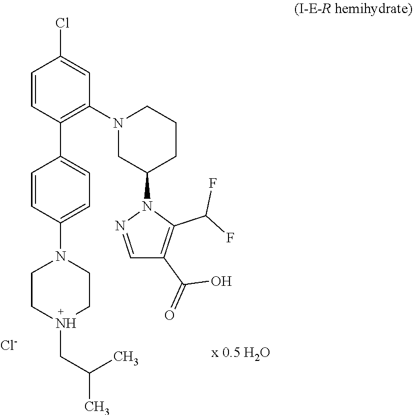 [SMILES: [Cl-] ; CC(C)CN1CCN(c2ccc(-c3ccc(Cl)cc3N3CCC[C@@H](n4ncc(C(=O)O)c4C(F)F)C3)cc2)CC1]

The invention further provides a process for preparing compounds of the formula (I), or salts thereof, solvates thereof or solvates of the salts thereof, wherein
in a first step [B] the compounds of the formula (III)

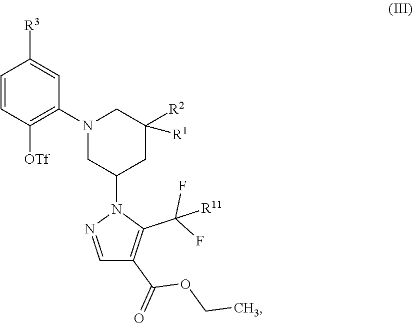

in which R¹, R², R³ and R¹¹ are defined as above,
are reacted with compounds of the formula (IV)

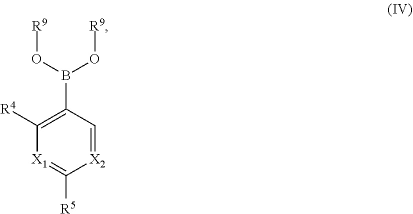

in which R⁴, R⁵, and X₁ and X₂ are defined as above,
and
in which R⁹ represents hydrogen, methyl, or both R⁹ form via the adjacent oxygen atoms a 4,4,5,5-tetramethyl-1,3,2-dioxaborolane
in the presence of a palladium source, a suitable ligand and a base to provide compounds of the formula (II)

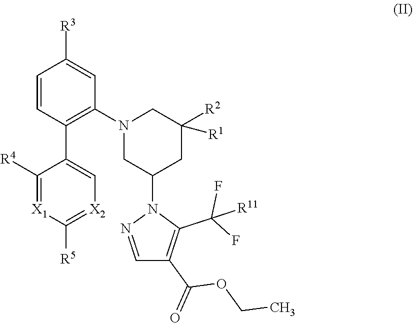

in which R¹, R², R³, R⁴, R⁵, R¹¹ and X₁ and X₂ are defined as above
and
in a second step [A]
compounds of formula (II) are reacted with a base to provide compounds of the formula (I),

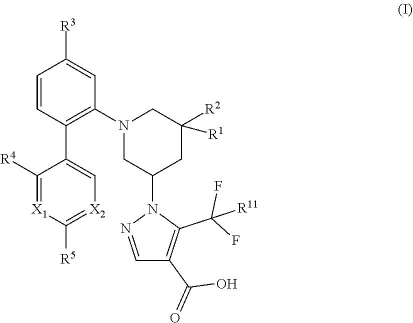

in which R¹, R², R³, R⁴, R⁵, R¹¹ and X₁ and X₂ are defined as above.
optionally compounds of formula (I) are transferred in a third step [A]* into the corresponding salts of formula (Ia)

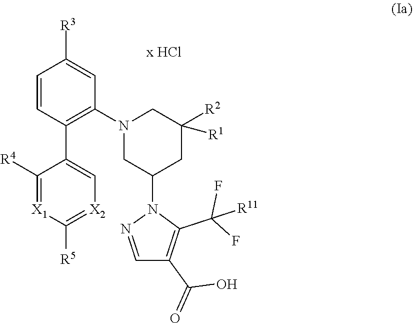

in the presence of a suitable acid in a suitable solvent
in which R¹, R², R³, R⁴, R⁵, R¹¹ and X₁ and X₂ are defined as above.

Reaction [A]* (Salt Formation)

The reaction [A]* is generally carried out in inert solvents in the presence of an acid preferably in a temperature range from 0° C. to 60° C. at atmospheric pressure.

Suitable acids for the salt formation are generally sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, optionally with addition of water. Preference is given to hydrogen chloride hydrogen bromide, toluenesulfonic acid, methanesulfonic acid or sulfuric acid.

Suitable inert solvents for the salt formation are, for example, ethers such as diethyl ether, dioxane, tetrahydrofuran, glycol dimethyl ether or diethylene glycol dimethyl ether, or other solvents such as acetone, ethyl acetate, ethanol, n-propanol, isopropanol, acetonitrile, dimethyl sulphoxide, N,N-dimethylformamide, N,N-dimethylacetamide, N,N′-dimethylpropyleneurea (DMPU) or N-methylpyrrolidone (NMP). It is also possible to use mixtures of the solvents mentioned. Preference is given to diethyl ether, dioxane, tetrahydrofuran or mixtures of these solvents.

Reaction [A] (Ester Hydrolyses)

The hydrolysis of the ester group in compounds of formula II is carried out by customary methods, by treating the esters in inert solvents with acids or bases, where in the latter variant the salts initially formed are converted into the free carboxylic acids by treatment with acid. In the case of the tert-butyl esters, the ester hydrolysis is preferably effected with acids.

Suitable inert solvents for these reactions are water or the organic solvents customary for ester cleavage. These preferably include alcohols such as methanol, ethanol, n-propanol, isopropanol, n-butanol or tert-butanol, ethers such as diethyl ether, tetrahydrofuran, 1,4-dioxane or 1,2-dimethoxyethane, or other solvents such as dichloromethane, acetone, methyl ethyl ketone, N,N-dimethylformamide or dimethyl sulphoxide. It is equally possible to use mixtures of these solvents. In the case of a basic ester hydrolysis, preference is given to using mixtures of water with dioxane, tetrahydrofuran, methanol, ethanol and/or dimethylformamide or mixtures of tetrahydrofuran and methanol or ethanol. In the case of the reaction with trifluoroacetic acid, preference is given to using dichloromethane, and in the case of the reaction with hydrogen chloride preference is given to using tetrahydrofuran, diethyl ether, dioxane or water.

Suitable bases are the customary inorganic bases. These especially include alkali metal or alkaline earth metal hydroxides, for example lithium hydroxide, sodium hydroxide, potassium hydroxide or barium hydroxide, or alkali metal or alkaline earth metal carbonates, such as sodium carbonate, potassium carbonate or calcium carbonate. Preference is given to lithium hydroxide, sodium hydroxide or potassium hydroxide.

Suitable acids for the ester hydrolysis are generally sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, optionally with addition of water. Preference is given to hydrogen chloride or trifluoroacetic acid in the case of the tert-butyl esters and to hydrochloric acid in the case of the methyl esters.

The ester hydrolysis is generally carried out within a temperature range from −20° C. to +120° C., preferably at 0° C. to +80° C.

The compounds of the formula (II)

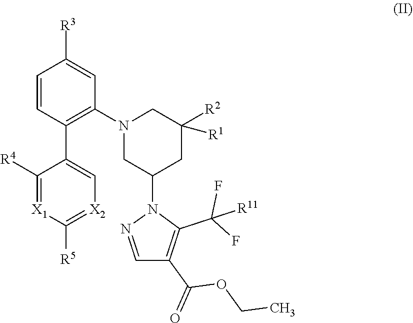

in which R¹, R², R³, R⁴, R⁵, R¹¹ and X₁ and X₂ are defined as above are novel.

The compounds of the formula (II) can be synthesized from the corresponding starting compounds of formula (III) by

[B] reacting the compounds of the formula (III)

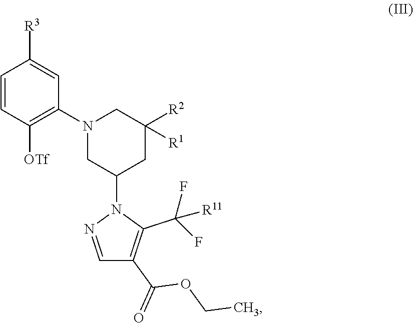

in which R¹, R², R³ and R¹¹ are defined as above,

in the presence of a suitable palladium catalyst, base and a suitable solvent

with compounds of the formula (IV)

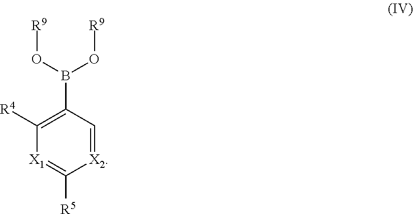

in which R⁴, R⁵, R⁹ and X₁ and X₂ are defined as above,

in the presence of a palladium source, a suitable ligand and a base to provide compounds of the formula (II).

Reaction [B] (Suzuki Coupling)

The reaction [B] is generally carried out in the presence of a suitable palladium catalyst and a suitable base in inert solvents, preferably at temperature range from room temperature up to reflux of the solvents at atmospheric pressure.

Inert solvents for reaction step [B] are for example alcohols like methanol, ethanol, n-propanol, isopropanol, n-butanol or tert.-butanol, ether like diethylether, dioxane, tetrahydrofuran, glycoldimethylether or di-ethylenglycoldimethylether, hydrocarbons like benzene, xylol, toluene, hexane, cyclohexane or petroleum oil, or other solvents like dimethylformamide (DMF), dimethylsulfoxide (DMSO), N,N′-dimethylpropylene urea (DMPU), N-methylpyrrolidone (NMP), pyridine, acetonitrile or also water. It is also possible to utilize mixtures of the aforementioned solvents. Preferred is a mixture of dimethylformamide/water and toluene/ethanol.

Suitable bases for reaction steps are the customary inorganic bases. These especially include alkali metal or alkaline earth metal hydroxides, for example lithium hydroxide, sodium hydroxide, potassium hydroxide or barium hydroxide alkali metal hydrogencarbonates like sodium or potassium hydrogencarbonate, or alkali metal or alkaline earth metal carbonates such as lithium, sodium, potassium, calcium or cesium carbonate, or alkali hydrogenphosphates like disodium or dipotassium hydrogenphosphate. Preferably used bases are sodium or potassium carbonate.

Examples of suitable palladium catalysts for reaction steps [“Suzuki-coupling” ] are e.g. palladium on charcoal, palladium(II)-acetate, tetrakis-(triphenylphosphine)-palladium(0), bis-(triphenylphosphine)-palladium(II)-chloride, bis-(acetonitrile)-palladium(II)-chloride and [1,1′-bis(diphenylphosphino)ferrocene]dichloropalladium(II)-dichlormethane-complex [cf. e.g. Hassan J. et al., Chem. Rev. 102, 1359-1469 (2002)].

The reaction steps are generally carried out within a temperature range from +20° C. to +150° C., preferably at +50° C. to +100° C.

The compounds of the formula (IV)

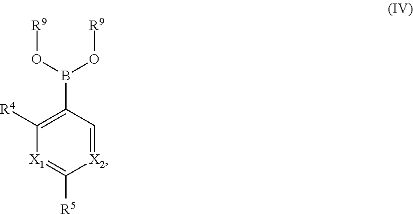

in which R⁴, R⁵, R⁹ and X₁ and X₂ are defined as above

and

in which
R⁵ represents a group of the formula

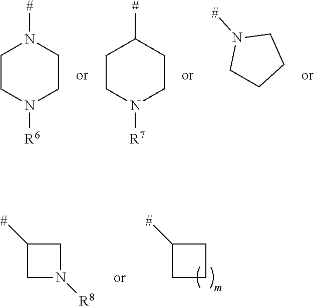

where #is the point of attachment to the aromatic or heteroaromatic 6 ring system; wherein m is 0-4
R⁶ represents C₁-C₆-alkyl, optionally substituted by one or more substituent independently selected from the group consisting of methyl, trifluoromethoxy, nitril, amido,
   C₂-C₆-halogenoalkyl, substituted by 1 to 5 fluoro substituents,
   C₃-C₆-cycloalkyl,
   C₃-C₆-cycloalkyl-methyl, optionally substituted by 1 to 5 fluoro substituents or a trifluoromethyl group,
   C₁-C₆-alkylcarbonyl, optionally substituted by 1 to 3 fluoro substituents,
   C₃-C₆-cycloalkyl-carbonyl, optionally substituted by 1 to 3 fluoro substituents,
   (C₁-C₆)-alkoxy-carbonyl, optionally substituted with methoxy, trifluoromethoxy, C₃-C₆-cycloalkyl,
   (C₃-C₆)-cycloalkoxy-carbonyl,
   mono-(C₁-C₄)-alkylaminocarbonyl,
   (C₁-C₄)-alkylsulfonyl oxetanyl,
   spiro[2.2]pentan-2-ylmethyl or [(3-fluoro-1-bicyclo[1.1.1]pentanyl)methyl,
R⁷ represents C₁-C₄-alkylcarbonyl, optionally substituted by a C₃-C₆-cycloalkyl group,
R⁸ represents C₂-C₄-alkyl, C₂-C₄-halogenoalkyl substituted by 1 to 6 fluoro substituents,
are novel.

The compounds of the formula (IVb)

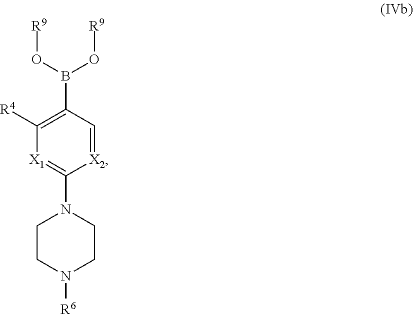

in which R⁴, R⁶, R⁹ and X₁ and X₂ are defined as above are novel and

can be prepared

[C] by reacting compounds of the formula (IVa)

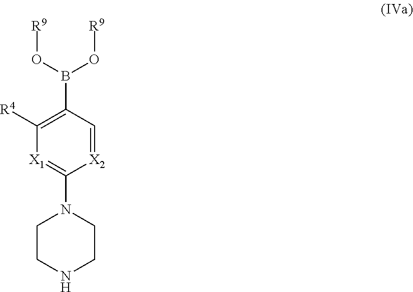

in which R⁴, R⁹ and X₁ and X₂ are defined as above

with compounds of formula (XV)

R^{6a}—CHO (XV)

in which
R^{6a} represents C₁-C₅-alkyl, optionally substituted by one or more substituent independently selected from the group consisting of methyl, trifluoromethoxy, nitril, amido,
   C₂-C₅-halogenoalkyl, substituted by 1 to 5 fluoro substituents,
   C₃-C₆-cycloalkyl, optionally substituted by 1 to 5 fluoro substituents or a trifluoromethyl group,
   spiro[2.2]butan-2-ylmethyl or [(3-fluoro-1-bicyclo[1.1.1]butanyl)methyl,
in the presence of a reducing agent, a base and a suitable solvent
or alternatively

[D] by reacting compounds of the formula (IVa)

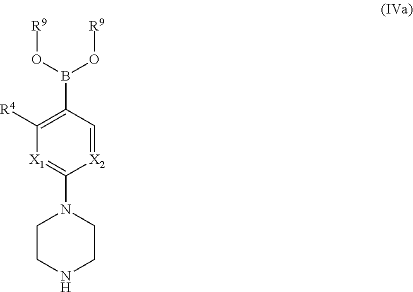

in which R⁴, R⁹ and X₁ and X₂ are defined as above

with compounds of formula (XVI)

R⁶—X (XVI)

in which R⁶ is as defined above and X is Br, OTs, OTf

in the presence of a base and a suitable solvent.

or alternatively

[F] first by reacting compounds of the formula (IVa)

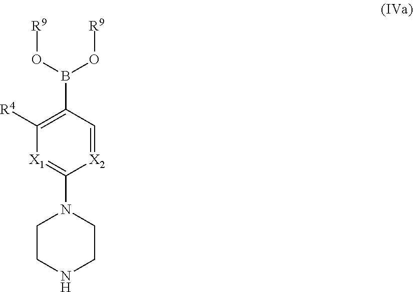

in which R⁴, R⁹ and X₁ and X₂ are defined as above

with compounds of formula (XVII)

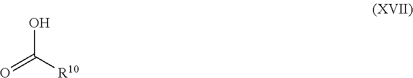

in which
R¹⁰ represents C₁-C₅-alkyl, optionally substituted by one or more substituent independently selected from the group consisting of methyl, trifluoromethoxy, nitril, amido,
   C₂-C₅-halogenoalkyl, substituted by 1 to 5 fluoro substituents,
   C₃-C₆-cycloalkyl, optionally substituted by 1 to 5 fluoro substituents or a trifluoromethyl group,
   spiro[2.2]butan-2-ylmethyl or [(3-fluoro-1-bicyclo[1.1.1]butanyl)methyl,
in the presence of a base, and a suitable solvent
to provide compounds of formula (IVc)

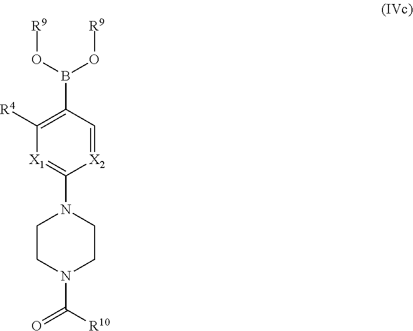

in which R⁴, R⁹, R¹⁰ and X₁ and X₂ are as defined above and

[E] by further reacting compounds of the formula (IVc)

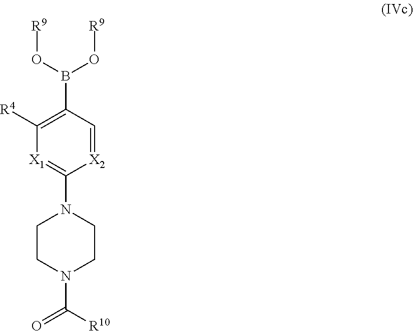

in which R⁴, R⁹, R¹⁰ and X₁ and X₂ are as defined above

in the presence of a reducing agent and a suitable solvent

to provide compounds of formula (IVd)

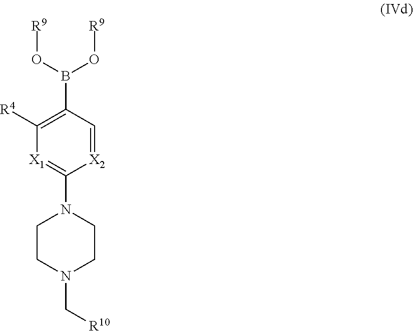

in which R⁴, R⁹, R¹⁰ and X₁ and X₂ are as defined above

Compounds of formula (IVc) will also be utilized in reaction [B] (Suzuki coupling) mentioned above.

Reaction [C] (Reductive Amination)

The reaction [C] is generally carried out in inert solvents in the presence of a reducing agent, if appropriate in the presence of a base and or a dehydrating agent, preferably in a temperature range from 0° C. to 60° C. at atmospheric pressure.

Suitable reducing agents for reductive aminations are alkali metal borohydrides customary for such purposes such as sodium borohydride, sodium cyanoborohydride or sodium triacetoxyborohydride; preference is given to using sodium triacetoxyborohydride.

The addition of an acid, such as acetic acid in particular, and/or of a dehydrating agent, for example molecular sieve or trimethyl orthoformate or triethyl orthoformate, may be advantageous in these reactions.

Bases are, for example organic bases such as trialkylamines, for example triethylamine, N-methylmorpholine, N-methylpiperidine, 4-dimethylaminopyridine or diisopropylethylamin, or pyridine.

Bases, such as N,N-diisopropylethylamine and triethylamine in particular, may be advantageous in these reactions.

Suitable solvents for these reactions are especially alcohols such as methanol, ethanol, n-propanol or isopropanol, ethers such as diisopropyl ether, methyl tert-butyl ether, tetrahydrofuran, 1,4-dioxane or 1,2-dimethoxyethane, polar aprotic solvents such as acetonitrile or N,N-dimethylformamide (DMF) or mixtures of such solvents; preference is given to using tetrahydrofuran.

The reactions are generally conducted within a temperature range of 0° C. to +60° C.

The aldehydes of formula (XV) are commercial available or can be synthesized from known starting materials by known processes.

The starting material of formula (IVa) is either commercial available, known or available by known processes.

Reaction [D] (Alkylation)

The reaction [D] is generally carried out in a temperature range of from 0° C. to +120° C., preferably at from +20° C. to +80° C., if appropriate in a microwave. The reaction can be carried out at atmospheric, elevated or reduced pressure (for example from 0.5 to 5 bar).

Suitable inert solvents for the alkylations are, for example, halogenated hydrocarbons such as dichloromethane, trichloromethane, carbon tetrachloride, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as acetone, methyl ethyl ketone, ethyl acetate, acetonitrile, N,N-dimethylformamide, N,N-dimethylacetamide, dimethyl sulphoxide, N,N′-dimethylpropyleneurea (DMPU), N-methylpyrrolidone (NMP) or pyridine. It is also possible to use mixtures of the solvents mentioned. Preference is given to using dimethylformamide, dimethyl sulphoxide or tetrahydrofuran.

Suitable bases for the alkylations are the customary inorganic or organic bases. These preferably include alkali metal hydroxides, for example lithium hydroxide, sodium hydroxide or potassium hydroxide, alkali metal or alkaline metal carbonates such as lithium carbonate, sodium carbonate, potassium carbonate, calcium carbonate or caesium carbonate, if appropriate with addition of an alkali metal iodide, for example sodium iodide or potassium iodide, alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 1,5-diazabicyclo[4.3.0]non-5-ene (DBN), 4-(N,N-dimethylamino)pyridine (DMAP), 1,8-diazabicyclo[5.4.0]undec-7-ene (DBU) or 1,4-diazabicyclo[2.2.2]octane (DABCO®). Preference is given to using potassium carbonate, caesium carbonate or N,N-diisopropylethylamine.

The alkylating agents of formula ((XVI) are known, commercial available or obtainable by known methods.

The starting material of formula (IVa) is either commercial available, known or available by known processes.

Reaction [E] (Reduction)

The reaction [E] is generally carried out in inert solvents, preferably in a temperature range from 0° C. to +65° C., preferably at from 0° C. to +40° C., if appropriate in a microwave. The reaction can be carried out at atmospheric, elevated or reduced pressure (for example from 0.5 to 5 bar).

Suitable inert solvents for the reductions are, for example, halogenated hydrocarbons such as dichloromethane, trichloromethane, carbon tetrachloride, trichloroethylene or chlorobenzene, ethers such as diethyl ether, dioxane, tetrahydrofuran, hydrocarbons such as benzene, toluene, xylene, hexane, cyclohexane or mineral oil fractions. It is also possible to use mixtures of the solvents mentioned. Preference is given to using tetrahydrofuran.

Suitable reducing agents for the amide reductions in process steps are, for example lithium aluminium hydride or borane tetrahydrofuran complex. Preference is given to using borane tetrahydrofuran complex.

The starting material of formula (IVc) is either commercial available, known or available by known processes or reaction [F].

Reaction [F] (Amide Formation)

The reaction [F] is generally carried out in inert solvents, in presence of a condensing agent preferably in a temperature of from −20° C. to +100° C., preferably at from 0° C. to +60° C. The reaction can be performed at atmospheric, elevated or at reduced pressure (for example from 0.5 to 5 bar). In general, the reaction is carried out at atmospheric pressure.

Inert solvents for the amide formation are, for example, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, halogenated hydrocarbons such as dichloromethane, trichloromethane, carbon tetrachloride, 1,2-dichloroethane, trichloroethylene or chlorobenzene, or other solvents such as acetone, ethyl acetate, acetonitrile, pyridine, dimethyl sulphoxide, N,N-dimethylformamide, N,N-dimethylacetamide, N,N′-dimethylpropyleneurea (DMPU) or N-methyl-pyrrolidone (NMP). It is also possible to use mixtures of the solvents mentioned. Preference is given to dichloromethane, tetrahydrofuran, dimethylformamide or mixtures of these solvents.

Suitable condensing agents for the amide formation are, for example, carbodiimides such as N,N′-diethyl-, N,N′-dipropyl-, N,N′-diisopropyl-, N,N′-dicyclohexylcarbodiimide (DCC) or N-(3-dimethylaminopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium 3-sulphate or 2-tert-butyl-5-methylisoxazolium perchlorate, acylamino compounds such as 2-ethoxy-1-ethoxycarbonyl-1,2-dihydro-quinoline, or isobutyl chloroformate, propanephosphonic anhydride (T3P), 1-chloro-N,N,2-trimethylpropl-ene-1-amine, diethyl cyanophosphonate, bis-(2-oxo-3-oxazolidinyl)phosphoryl chloride, benzotriazol-1-yl-oxytris(dimethylamino)phosphonium hexafluorophosphate, benzotriazol-1-yloxytris(pyrrolidino)-phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzo-triazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), if appropriate in combination with further auxiliaries such as 1-hydroxybenzotriazole (HOBt) or N-hydroxysuccinimide (HOSu), and also as bases alkali metal carbonates, for example sodium carbonate or potassium carbonate or sodium bicarbonate or potassium bicarbonate, or organic bases such as trialkylamines, for example triethylamine, N-methylmorpholine, N-methylpiperidine or N,N-diisopropylethylamine. Preference is given to using TBTU in combination with N-methylmorpholine, 1-chloro-N,N,2-trimethylprop-1-ene-1amine or HATU in combination with N,N-diisopropylethylamine.

Alternatively, the carboxylic acids can also initially be converted into the corresponding carbonyl chloride and this can then be reacted directly or in a separate reaction with an amine to give the compounds according to the invention. The formation of carbonyl chlorides from carboxylic acids is carried out by methods known to the person skilled in the art, for example by treatment with thionyl chloride, sulphuryl chloride or oxalyl chloride in the presence of a suitable base, for example in the presence of pyridine, and also optionally with addition of dimethylformamide, optionally in a suitable inert solvent.

The starting material of formula (IVc) is either commercially available, known or available by known processes or reaction [F].

The acylating agent of formula (XVII) is either commercially available, known or available by known processes.

Compounds of the formula (IVf)

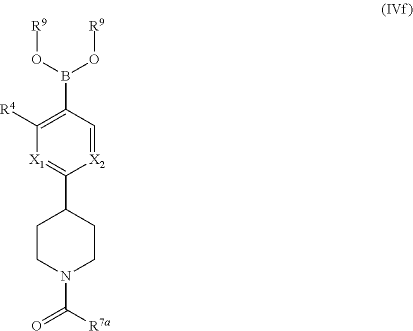

in which R⁴, R⁹ and X₁ and X₂ are defined as above are novel.

in which R^{7a} represents C₁-C₂-alkyl, cyclopropyl

They can be obtained by

[G] reacting compounds of formula (IVe)

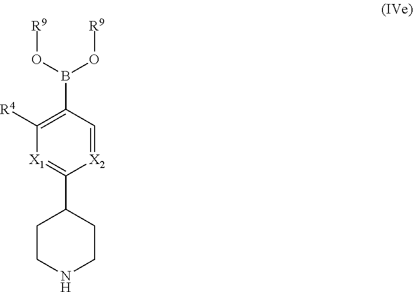

in which R⁴, R⁹ and X₁ and X₂ are defined as above

with compounds of formula (XVIII)

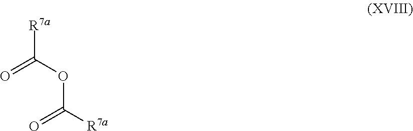

in which R^{7a} represents C₁-C₂-alkyl, cyclopropyl

in the presence of a base, a suitable solvent.

Reaction [G] (Acylation)

The reaction [G] is generally carried out in inert solvents, in presence of a base and a dehydrating agent preferably in a temperature range of from 0° C. to +100° C., preferably at from 0° C. to +40° C., if appropriate in a microwave. The reaction can be carried out at atmospheric, elevated or reduced pressure (for example from 0.5 to 5 bar).

Suitable inert solvents for the acylation are, for example, halogenated hydrocarbons such as dichloromethane, trichloromethane, carbon tetrachloride, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as acetone, methyl ethyl ketone, ethyl acetate, acetonitrile, N,N-dimethylformamide, N,N-dimethylacetamide, dimethyl sulphoxide, N,N′-dimethylpropyleneurea (DMPU), N-methylpyrrolidone (NMP) or pyridine. It is also possible to use mixtures of the solvents mentioned. Preference is given to using dimethylformamide or dichloromethane.

Suitable bases for the alkylations are the customary inorganic or organic bases. These preferably include alkali metal hydroxides, for example lithium hydroxide, sodium hydroxide or potassium hydroxide, alkali metal or alkaline metal carbonates such as lithium carbonate, sodium carbonate, potassium carbonate, calcium carbonate or caesium carbonate, if appropriate with addition of an alkali metal iodide, for example sodium iodide or potassium iodide, alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 1,5-diazabicyclo[4.3.0]non-5-ene (DBN), 4-(N,N-dimethylamino)pyridine (DMAP), 1,8-diazabicyclo[5.4.0]undec-7-ene (DBU) or 1,4-diazabicyclo[2.2.2]octane (DABCO®). Preference is given to using pyridine, triethylamine or N,N-diisopropylethylamine.

Compounds of the formula (IVe)

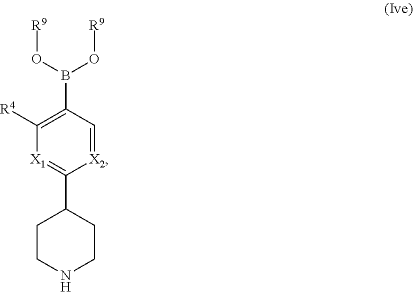

in which R⁴, R⁹ and X₁ and X₂ are defined as above

are known, commercial available or obtainable by known processes.

Compounds of the formula (XVIII)

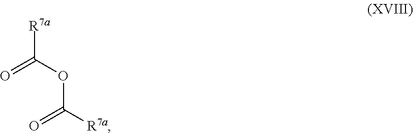

in which R^{7a} is defined as above

are known, commercial available or obtainable by known processes.

Compounds of the formula (IVi)

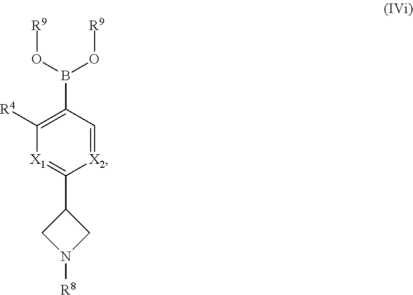

in which R⁴, R⁸, R⁹ and X₁ and X₂ are defined as above are novel and can be obtained by

[I] first reacting compounds of formula (IVg)

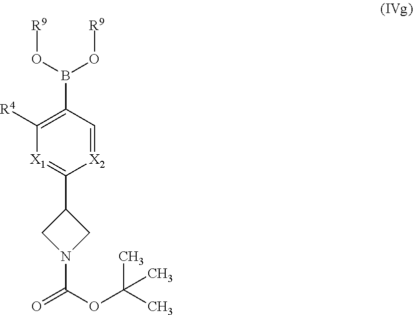

in which R⁴, R⁹ and X₁ and X₂ are defined as above are

with an acid in a suitable solvent

to obtain compounds of formula (IVh)

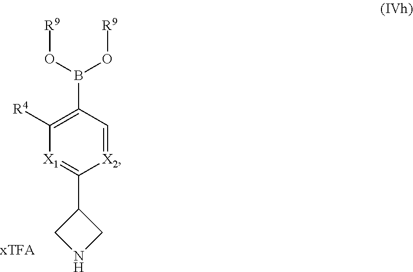

in which R⁴, R⁹ and X₁ and X₂ are defined as above

and

[H] secondly reacting compounds of formula (IVh)

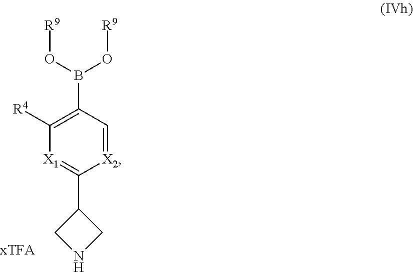

in which R⁴, R⁹ and X₁ and X₂ are defined as above

with compounds of formula ((XVIII)

X—R⁸ (XIX)

in which X is 1, OTf

and in which R⁸ is as defined above

in the presence of a base and a suitable solvent

to obtain compounds of formula (IVi)

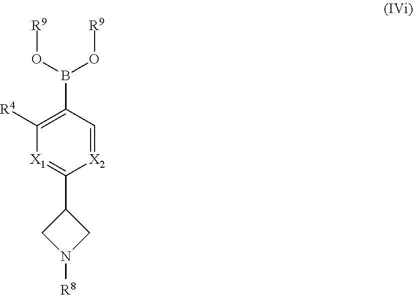

in which R⁴, R⁸, R⁹ and X₁ and X₂ are defined as above.

Reaction [H] (Alkylation)

The reaction [H] is generally carried out in a temperature range of from 0° C. to +120° C., preferably at from +20° C. to +80° C., if appropriate in a microwave. The reaction can be carried out at atmospheric, elevated or reduced pressure (for example from 0.5 to 5 bar).

Suitable inert solvents for the alkylations are, for example, halogenated hydrocarbons such as dichloromethane, trichloromethane, carbon tetrachloride, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as acetone, methyl ethyl ketone, ethyl acetate, acetonitrile, N,N-dimethylformamide, N,N-dimethylacetamide, dimethyl sulphoxide, N,N′-dimethylpropyleneurea (DMPU), N-methylpyrrolidone is (NMP) or pyridine. It is also possible to use mixtures of the solvents mentioned. Preference is given to using dimethylformamide, dimethyl sulphoxide or tetrahydrofuran.

Suitable bases for the alkylations are the customary inorganic or organic bases. These preferably include alkali metal hydroxides, for example lithium hydroxide, sodium hydroxide or potassium hydroxide, alkali metal or alkaline metal carbonates such as lithium carbonate, sodium carbonate, potassium carbonate, calcium carbonate or caesium carbonate, if appropriate with addition of an alkali metal iodide, for example sodium iodide or potassium iodide, alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 1,5-diazabicyclo[4.3.0]non-5-ene (DBN), 4-(N,N-dimethylamino)pyridine (DMAP), 1,8-diazabicyclo[5.4.0]undec-7-ene (DBU) or 1,4-diazabicyclo[2.2.2]octane (DABCO®). Preference is given to using potassium carbonate, caesium carbonate or N,N-diisopropylethylamine.

Reaction [I] (Deprotection)

The reaction [I] is generally carried out in inert solvents in the presence of a suitable acid, preferably in a temperature range from 0° C. to 60° C. at atmospheric pressure.

Acids are, for example organic or inorganic acids such as sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, optionally with addition of water. Preference is given to hydrogen chloride or trifluoroacetic acid.

Suitable solvents for these reactions are especially alcohols such as methanol, ethanol, n-propanol or isopropanol, ethers such as diisopropyl ether, methyl tert-butyl ether, tetrahydrofuran, 1,4-dioxane or 1,2-dimethoxyethane, polar aprotic solvents such as acetonitrile or N,N-dimethylformamide (DMF) or mixtures of such solvents; preference is given to using tetrahydrofuran.

The reactions are generally conducted within a temperature range of 0° C. to +60° C.

The alkylating agents of formula ((XVI) are known, commercially available or obtainable by known methods.

The starting material of formula (IVa) is either commercially available, known or available by known processes.

The compounds of the formula (IVg) are known, commercially available or obtainable form known starting materials by known processes.

The compounds of formula (XIX) are known, commercially available or obtainable form known starting materials by known processes.

The compounds of formula (III)

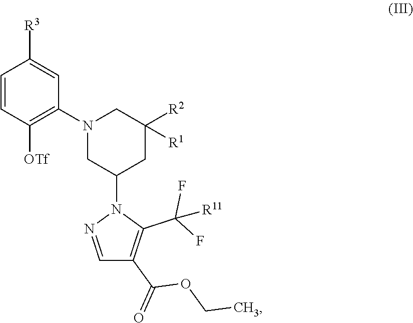

in which R¹, R², R³ and R¹¹ are defined as above are novel and can be prepared

[J] by reacting compounds of the formula (V)

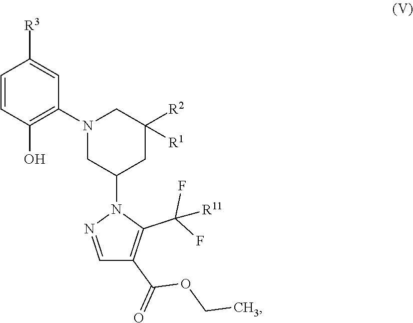

in which R¹, R², R³ and R¹¹ are as defined above,

with triflic acid anhydride in the presence of base and an inert solvent.

Reaction [J] (Triflatization)

The reaction [J] is generally carried out in inert solvents, preferably in a temperature range from room temperature up to reflux of the solvents at atmospheric pressure.

Bases are, for example, organic bases like alkali amines or pyridines or inorganic bases such as sodium hydroxide, lithium hydroxide or potassium hydroxide, or alkali metal carbonates such as caesium carbonate, sodium carbonate or potassium carbonate, or alkoxides such as potassium tert-butoxide or sodium tert-butoxide, or pyridines such as pyridine or 2,6-lutidine, or alkali amines such as triethylamine or N,N-diisopropylethylamine; preference is given to triethylamine.

Inert solvents are, for example, ethers such as diethyl ether, methyl tert-butyl ether, 1,2-dimethoxyethane, dioxane or tetrahydrofuran, or other solvents such as dichloromethane, dimethylformamide, dimethylacetamide, acetonitrile or pyridine, or mixtures of solvents; preference is given to dichloromethane.

The compounds of the formula (V) are novel

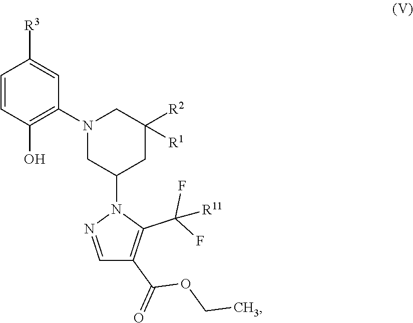

in which R¹, R², R³ and R¹¹ are defined as above.

The compounds of the formula (V) can be prepared

[K] by reacting compounds of the formula (VI)

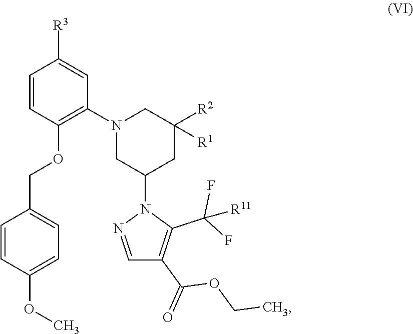

in which R¹, R², R³ and R¹¹ are as defined above,

with an acid optionally in an inert solvent.

Reaction [K] (Acidic Deprotection)

The reaction [K] is generally carried out in inert solvents or without solvent, preferably in a temperature range from 0° C. up to reflux of the solvents at atmospheric pressure.

Inert solvents are, for example, halogenated hydrocarbons such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethoxy ethane, N-methyl-pyrrolidone, dimethylacetamide, acetonitrile, acetone or pyridine, or mixtures of solvents; preference is given to dichloromethane or dioxane.

Suitable acids for the acidic deprotection are generally sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, optionally with addition of water. Preference is given to hydrogen chloride or trifluoroacetic acid.

Compounds of the formula (VI)

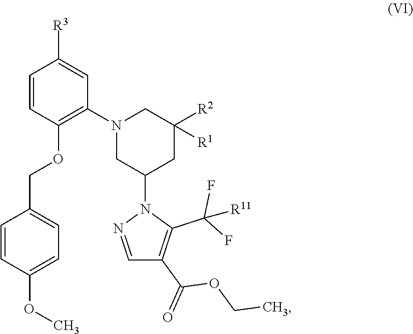

in which R¹, R², R³ and R¹¹ are as defined above are novel.

The compounds of the formula (VI) can be prepared

[L] by reacting compounds of the formula (VII)

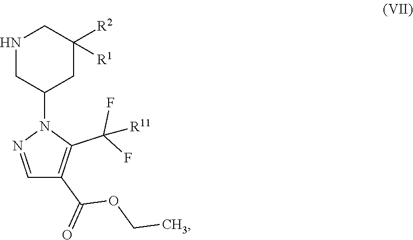

in which R¹, R² and R¹¹ are as defined above,

with compounds of the formula (VIII)

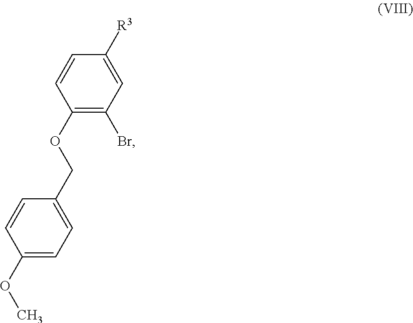

in which R³ is as defined above,

in the presence of a palladium source, a suitable ligand and a base.

Reaction [L] (Buchwald Hartwig Coupling)

The reaction [L] is generally carried out in the presence of a palladium source, a suitable ligand and a base in inert solvents, preferably in a temperature range from room temperature up to reflux of the solvents at atmospheric pressure.

The palladium source and a suitable ligand are, for example, palladium on charcoal, palladium(II)-acetate, tris(dibenzylideneacetone)palladium(0), tetrakis-(triphenylphosphine)-palladium(0), bis-(triphenylphosphine)-palladium(II) chloride, bis-(acetonitrile)-palladium(II) chloride, [1,1′-bis(diphenylphosphino)ferrocene]dichloro palladium (II) and corresponding dichloromethan-complex, optionally in conjunction with additional phosphane ligands like for example 2,2′-Bis(diphenylphosphino)-1,1′-binaphthyl (BINAP), (2-dicyclohexylphosphino-2′,4′,6′-triisopropyl-1,1′-biphenyl)[2-(2′-amino-1,1′-biphenyl)]palladium(II) methanesulfonate (XPhos-Pd-G3, CAS-No: 445085-55-1), (2-biphenyl)di-tert.-butylphosphine, dicyclohexyl[2′,4′,6′-tris(1-methylethyl)biphenyl-2-yl]phosphane (XPhos, CAS-No: CAS-No: 564483-18-7), Bis(2-phenylphosphinophenyl)ether (DPEphos), or 4,5-bis(diphenyl-phosphino)-9,9-dimethylxanthene (Xantphos: CAS-No: 161265-03-8) [cf e.g. Hassan J. et al., Chem. Rev. 2002, 102, 1359-1469], 2-(dicyclohexylphosphine)-3,6-dimethoxy-2′,4′,6′-triisopropyl-1,1′-biphenyl (BrettPhos, CAS-No: 1070663-78-3), 2-dicyclohexylphosphino-2′,6′-dimethoxybiphenyl (SPhos, CAS-No: 657408-07-6), 2-dicyclohexylphosphino-2′,6′-diisopropoxybiphenyl (RuPhos, CAS-No: 787618-22-8), 2-(di-tert-butylphosphino)-3-methoxy-6-methyl-2′,4′,6′-tri-i-propyl-1,1′-biphenyl (RockPhos) and 2-di-tert-butylphosphino-2′,4′,6′-triisopropylbiphenyl (tert-ButylXPhos). It is also possible to use corresponding precatalysts such as chloro-[2-(dicyclohexylphosphine)-3,6-dimethoxy-2′,4′,6′-triisopropyl-1,1′-biphenyl][2-(2-aminoethyl)-phenyl]palladium(II) (BrettPhos precatalysts) [cf e.g. S. L. Buchwald et al., Chem. Sci. 2013, 4, 916] optionally be used in conjunction with additional phosphine ligands such as 2-(dicyclohexylphosphine)-3,6-dimethoxy-2′,4′,6′-triisopropyl-1,1′-biphenyl (BrettPhos).

Preference is given to 2,2′-Bis(diphenylphosphino)-1,1′-binaphthyl (BINAP), tris(dibenzylideneacetone)palladium(0), or in combination with 4,5-bis(diphenylphosphino)-9,9-dimethyl-xanthen (Xantphos) or dicyclohexyl[2′,4′,6′-tris(1-methylethyl)biphenyl-2-yl]phosphane (XPhos).

Bases are, for example, suitable inorganic or organic bases like e.g. alkali or earth alkali metal carbonates such as lithium, sodium, potassium, calcium or cesium carbonate, or sodium bicarbonate or potassium bicarbonate, alkali metal hydrogencarbonates such as sodium hydrogencarbonate or potassium hydrogencarbonate, alkali metal or earth alkali hydroxides such as sodium, barium or potassium hydroxide; alkali metal or earth alkali phosphates like potassium phosphate; alkali metal alcoholates like sodium or potassium tert.-butylate and sodium methanolate, alkali metal phenolates like sodium phenolate, potassium acetate, amides like sodium amide, lithium-, sodium- or potassium-bis(trimethylsilyl)amide or lithium-diisopropylamide or organic amines like 1,5-diazabicyclo[4.3.0]non-5-ene (DBN), 1,8-diazabicyclo[5.4.0]undec-7-en (DBU). Preference is given to caesium carbonate, sodium carbonate, potassium carbonate or sodium hydrogencarbonate.

Inert solvents are, for example, ethers such as dioxane, diethyl ether, tetrahydrofuran, 2-methyl-tetrahydrofuran, di-n-butylether, cyclopentylmethylether, glycoldimethylether or diethyleneglycoldimethyl-ether, alcohols like tert.-butanol or amylalcohols or dimethylformamide, dimethylacetamide, dimethyl sulphoxide, N-methylpyrrolidone, toluene or acetonitrile, or mixtures of the solvents; preference is given to tert.-butanol, 1,4-dioxane and toluene.

The compounds of the formula (VIII) are known or can be synthesized from the corresponding, commercial available starting compounds by known processes.

The compounds of the formula (VII)

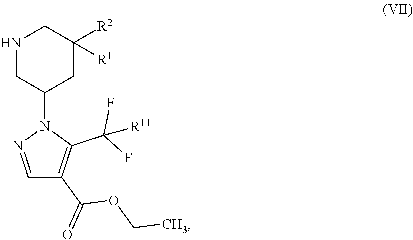

in which R¹, R² and R¹¹ are as defined above are novel.

The compounds of the formula (VII) can be prepared

[M] by reacting compounds of the formula (IX)

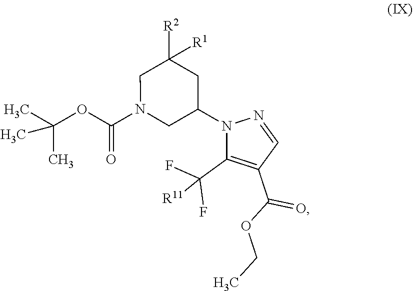

in which R¹, R² and R¹¹ are as defined above,

with an acid in an inert solvent.

Reaction [M] (Debocylation)

The reaction [M] is generally carried out in inert solvents in the presence of a suitable acid, preferably in a temperature range from 0° C. to 60° C. at atmospheric pressure.

Acids are for example organic or inorganic acids such as sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, optionally with addition of water. Preference is given to hydrogen chloride or trifluoroacetic acid

Inert solvents are alcohols such as methanol, ethanol or isopropanol, ethers such as diethyl ether, diisopropyl ether, methyl tert-butyl ether, tetrahydrofuran or 1,4-dioxane, dichloromethane, polar aprotic solvents such as acetonitrile or N,N-dimethylformamide (DMF) or mixtures of such solvents; preference is given to using 1,4-dioxane.

The compounds of the formula (IX)

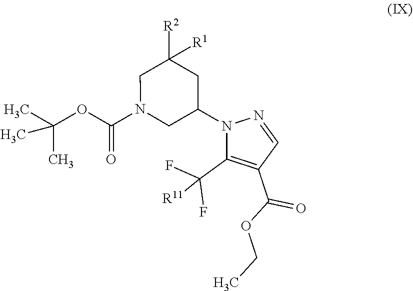

in which R¹, R² and R¹¹ are as defined above, are novel.

The compounds of the formula (IX) can be prepared

[N] by reacting compounds of the formula (X)

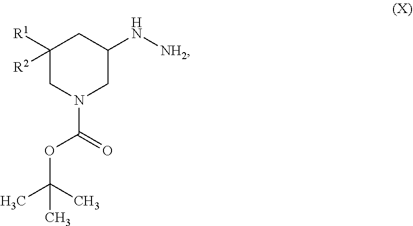

in which R¹ and R² are as defined above,

with compounds of the formula (XI)

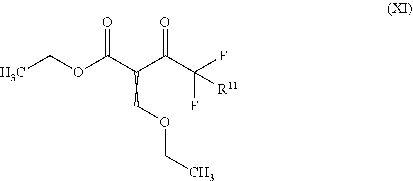

in a solvent.

Reaction [N] (Pyrazole Formation)

The reaction [L] is generally carried out in a solvent at temperatures from room temperature to reflux.

Suitable solvents are alcohols such as methanol, ethanol or isopropanol, ethers such as diethyl ether, diisopropyl ether, methyl tert-butyl ether, tetrahydrofuran or 1,4-dioxane, dichloromethane, polar aprotic solvents such as acetonitrile or N,N-dimethylformamide (DMF) or mixtures of such solvents; preference is given to using ethanol.

The compound of the formula (XI) are known or can be synthesized from the corresponding starting compounds by known processes.

The compounds of the formula (X)

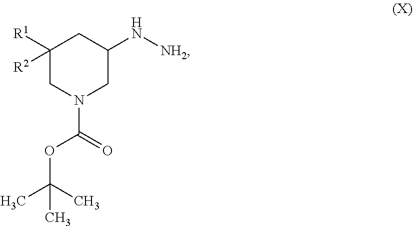

in which R¹ and R² are as defined above are novel.

The compounds of the formula (X) can be prepared

[O] by reacting compounds of the formula (XII)

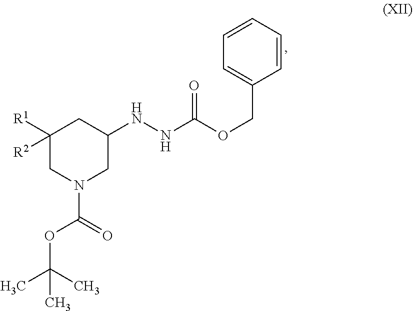

in which R¹ and R² are as defined above

with palladium on charcoal in the presence of hydrogen in a suitable solvent.

Reaction [O] (Z Deprotection)

The reaction [O] is generally carried out in the presence of palladium on charcoal in a suitable solvent at from room temperature to reflux, preferable at 1 bar.

Suitable solvents are alcohols such as methanol, ethanol or isopropanol, ethers such as diethyl ether, diisopropyl ether, methyl tert-butyl ether, tetrahydrofuran or 1,4-dioxane, dichloromethane, polar solvents such as acetonitrile, N,N-dimethylformamide (DMF), NMP, acetic acid or water or mixtures of such solvents; preference is given to ethanol/acetic acid.

The compounds of the formula (XII)

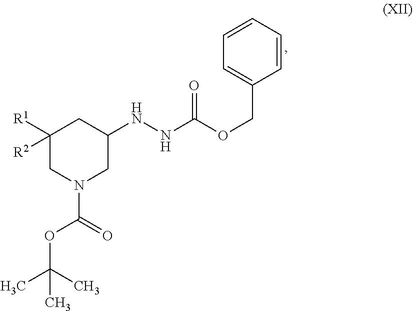

in which R¹ and R² are as defined above are novel.

The compounds of the formula (XII) can be prepared

[P] by reacting compounds of the formula (XIII)

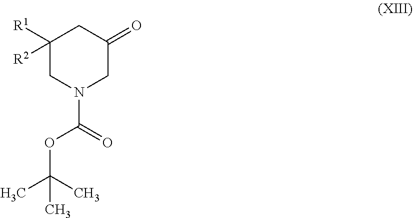

in which R¹ and R² are as defined above

with a compound of the formula (XIV)

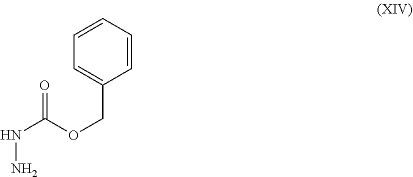 [SMILES: NNC(=O)OCc1ccccc1]

in the presence of a reducing agent and a suitable solvent.

The compound of the formula (XIV) is known and commercially available or can be synthesized from the corresponding starting compounds by known processes.

The compound of the formula (XIII) is known and commercially available or can be synthesized from the corresponding starting compounds by known processes.

The preparation of the starting compounds and of the compounds of the formula (I) can be illustrated by the synthesis schemes 1 to 4 which follow.

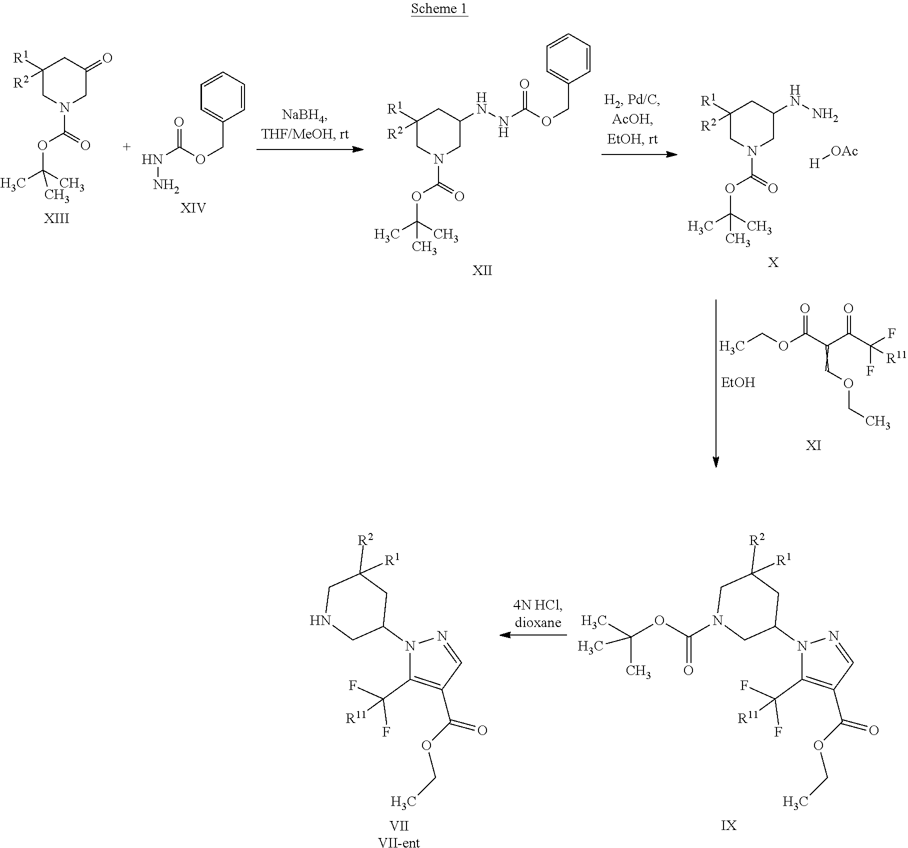

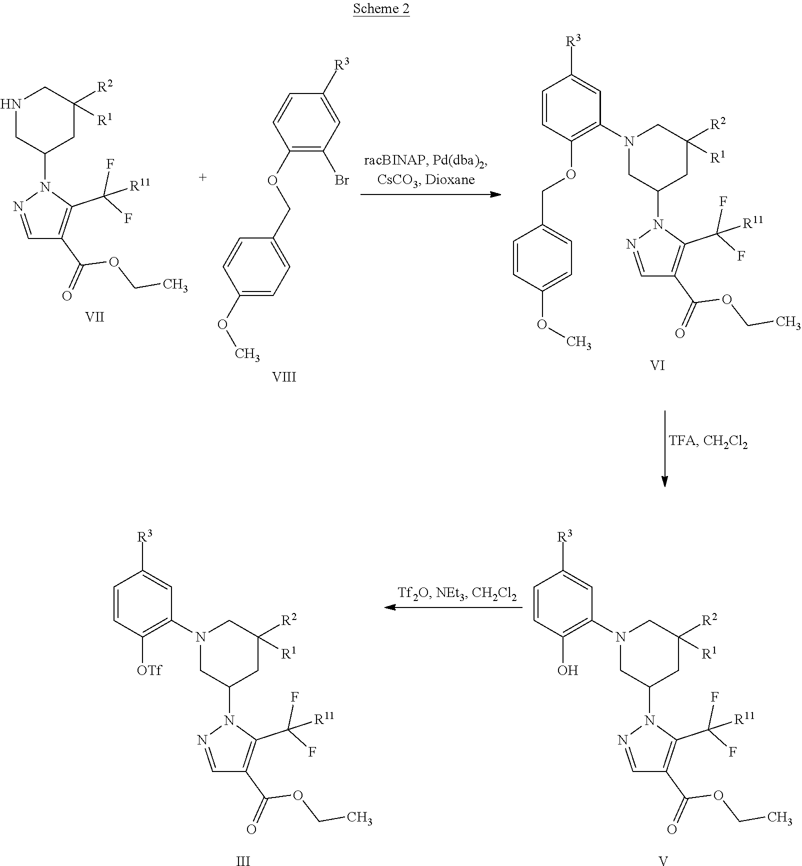

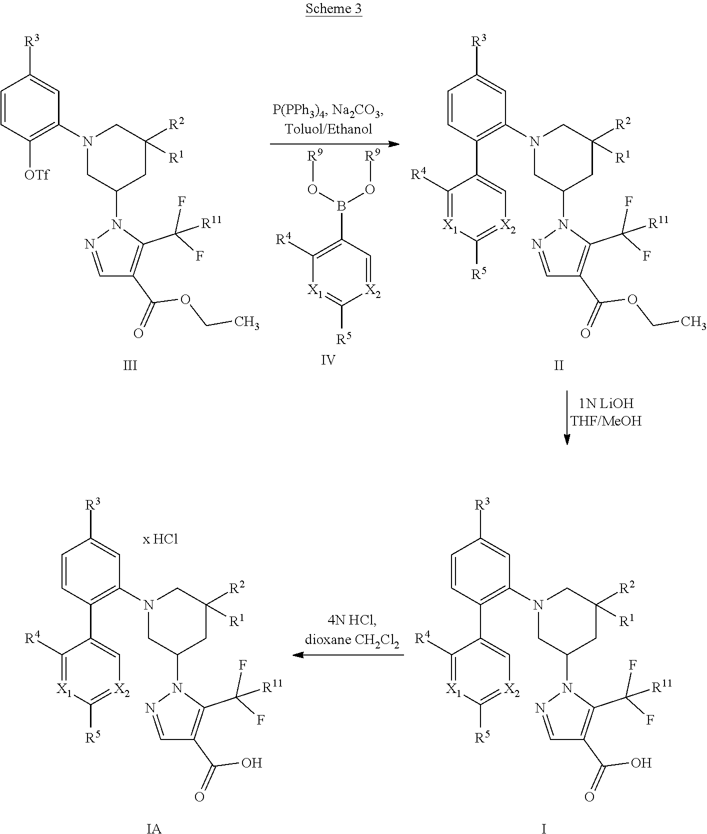

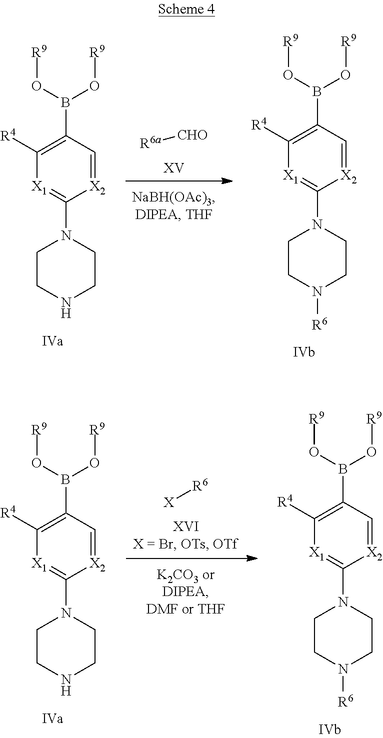

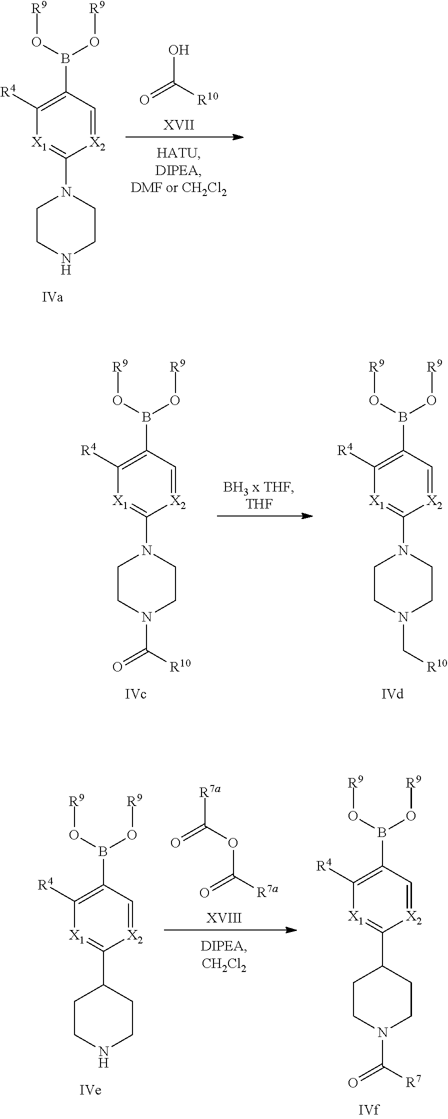

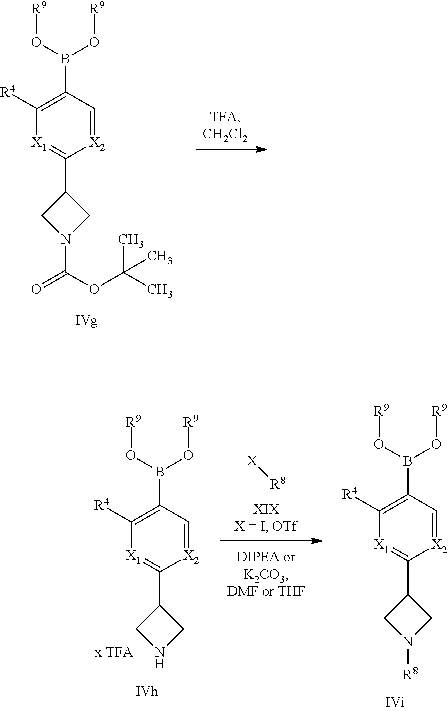

The compounds of the invention have valuable pharmacological properties and can be used for prevention and treatment of diseases in humans and animals.

The compounds according to the invention are potent activators of soluble guanylate cyclase. They lead to vasorelaxation, inhibition of platelet aggregation and lowering of blood pressure and increase of coronary blood flow. These effects are mediated via direct haem-independent activation of soluble guanylate cyclase and an increase of intracellular cGMP.

In addition, the compounds according to the invention have advantageous pharmacokinetic properties, in particular with respect to their bioavailability and/or duration of action after intravenous or oral administration.

The compounds according to the invention have an unforeseeable useful pharmacological activity spectrum and good pharmacokinetic behavior, in particular a sufficient exposure of such a compound in the blood above the minimal effective concentration within a given dosing interval after oral administration. Such a profile results in an improved peak-to-trough ratio (quotient of maximum to minimum concentration) within a given dosing interval, which has the advantage that the compound can be administered less frequently and at a significantly lower dose to achieve an effect. They are compounds that activate soluble guanylate cyclase.

Diabetic retinopathy (DR) is the most frequent cause of new cases of blindness among adults aged 20-74 years in developed countries. Indeed, crude global prevalence of blindness and vision impairment decreased markedly between 1990 and 2015 for the major causes, except for diabetic retinopathy, which increased.

In general, DR advances starting from mild non-proliferative abnormalities, characterized by increased numbers of microaneurysms that may wax and wane. With increasing severity, there is increased vascular permeability and occlusion and progression from non-proliferative diabetic retinopathy (mild, moderate and severe NPDR) to proliferative diabetic retinopathy (PDR) (Solomon et al. 2017). The damage to the retinal neurovascular unit has an important role in disease pathogenesis. The retinal neurovascular unit comprises vascular cells (endothelial cells and pericytes) as well as non-vascular cells including neurons, macroglia and microglia. The close associations between these cell populations allow critical information about blood flow and metabolic activity to be integrated to maintain normal retinal function. Although the interactions between these cell populations in the diabetic retina are not fully understood, there is substantial evidence that there are distinct alterations in the health and function of these cell types that impact on the development of DR. The degeneration of capillaries (acellular capillaries) comprised of apoptotic vascular endothelial cells and pericytes as well as basement membrane thickening results in neurovascular unit damage, which is a hall mark of NPDR (Metea et al. 2007; Gardner and Davila 2017).

Currently, DR drug-treatment options include controlling of blood sugar and/or treatment with anti-VEGF antibodies (anti-VEGF Abs). Both options have several limitations, which are discussed in the following.

Two landmark clinical trials, the Diabetes Control and Complications Trial (DCCT) and the United Kingdom Prospective Diabetes Study (UKPDS) have demonstrated the beneficial effects of intensive glycemic control in patients with type 1 and type 2 diabetes mellitus (DM), respectively, with the decrease in the incidence and progression of DR. Nevertheless, a disadvantage of tight glycemic control was an early initial worsening in DR status, reported in the DCCT, while hypoglycemic episodes were also common (Chatziralli 2018).

Recently, few clinical programs with limited sample size have been run for anti-VEGF Abs with observed delayed disease progression (Gross et al. 2018). However, anti-VEGF treatment has some limitations.

Only one eye can be treated at one time. However, both eyes can require treatment at the same time. The therapy is delivered to the eye following intra-ocular injection with the risk of ocular hemorrhage, retinal injury and infection. Further adverse effects include eye pain, cataract, vitreous detachment, floaters, and ocular hypertension. In addition, repeated injections at regular intervals are required. For a prevention-setting as present in NPDR (prevention of progression from NPDR to PDR or DME as vision-threatening events) the benefit-risk assessment is therefore at least borderline.

Although a significant number of publications support the use of anti-VEGF Abs as treatment option for the proliferative stage of the disease PDR, (Zhao et al. 2018, Sivaprasad et al. 2017, Gross et al. 2018), limited knowledge is present regarding the early stage NPDR (Gross et al. 2018). There is no non-invasive treatment to halt progression of disease from NPDR to PDR and restore vision loss (Zhao et al. 2018, Bolinger et al. 2016).

Therefore, there is a high need for an oral treatment that allows for simultaneous treatment of both eyes without the risk of repeated intraocular injection of both eyes for prevention of NPDR progression to more advanced stages. Especially there is a need for such treatment which can reverse disease progression and vision loss.

Soluble guanylate cyclase (sGC) plays a key role in a variety of physiological processes, such as vasodilatation, aggregation of thrombocytes, proliferation of smooth muscle cells and neuronal signaling. The enzyme converts GTP to the second messenger cGMP. Diabetes is characterized by increased levels of reactive oxygen species (ROS) that destroy the biological activity of nitric oxide (NO) and limit cGMP formation. Diabetic rats show oxidative stress and NO/sGC signaling deregulation in the retina (Schaefer et al. 2003). Diabetic mice with disrupted NO/sGC signaling show more severe DR compared with diabetic wild-type mice (Li et al. 2010).

With the discovery of BAY 58-2667 (Cinaciguat), a new chemical matter has found which is able to activate heme-free apo sGC. This class is defined as NO-independent and heme-independent sGC activator. Common characteristics of these substances are that in combination with NO they only have an additive effect on enzyme activation, and that the activation of the oxidized or heme-free enzyme is markedly higher than that of the heme-containing enzyme (Evgenov O V et al. 2006; Stasch J P et al. 2002; Stasch J P et al. 2006). Spectroscopic studies show that cinaciguat displaces the oxidized heme group in the beta1 subunit which, as a result of the weakening of the iron-histidine bond, is attached only weakly to the sGC. It has also been shown that the characteristic sGC heme binding motif Tyr-x-Ser-x-Arg is absolutely essential both for the interaction of the negatively charged propionic acids of the heme group and for the action of Cinaciguat. Therefore, it is assumed that the binding site of Cinaciguat at the sGC is identical to the binding site of the heme group in the beta1 subunit. (Stasch J P et al. 2006). More recently other classes of sGC activators have been discovered which are different in pharmacokinetics but also in organ distribution which might have an impact on their treatment potential.

WO 2012/139888 and WO 2012/076466 disclose activators of sGC, their synthesis as well their use in the treatment of cardiovascular and renal diseases. In a large list of different potential indications DR is mentioned. The documents do not disclose the use in the treatment of diabetic macular edema (DME), retinal ganglion cell/photoreceptor neurodegeneration nor cataract.

WO 2012/058132 discloses substituted pyrazolo pyridine carboxylic acids as sGC activators. In contrast to the compounds according to the present invention these compounds do have a heteroaromatic pyridine moiety linking the pyrazole carboxylic acid to the rest of the molecular structure. Furthermore the pyridine nitrogen has another position than the piperidine nitrogen of the compounds according to the present invention. However there is no disclosure about these compounds being suitable for use in the treatment and/or prophylaxis of ophthalmologic diseases, including non-proliferative diabetic retinopathy (NPDR), diabetic macular edema (DME), retinal ganglion cell/photoreceptor neurodegeneration and cataract treatment.

It is therefore an object of the present invention to provide novel sGC activator compounds for the treatment and/or prophylaxis of diseases, in of ophthalmologic diseases, including non-proliferative diabetic retinopathy (NPDR), diabetic macular edema (DME), retinal ganglion cell/photoreceptor neurodegeneration and cataract, in humans and animals, which compounds have a wide therapeutic window and, in addition, a good pharmacokinetic behavior as well as beneficial physico chemical properties (e.g. solubility).

Surprisingly, it has now been found that certain substituted pyrazolo piperidine carboxylic acids as well as their corresponding salts represent highly potent sGC activators with good pharmacokinetic behavior as well as beneficial physico chemical properties (e.g. solubility).

As discussed above, PDR and NPDR describe different medical conditions. It was not considered that early stages of DR such as NPDR can be subjected to treatment as they are mostly asymptomatic. Therefore a person skilled in the art trying to find a treatment for DR would have considered treating advanced stages such as PDR. Surprisingly the compounds of the invention lead to a reversal of disease progression, shown by in vivo experiment ED001-2020 (“STZ model experiments”).

As mentioned earlier, the retinal neurovascular unit comprises vascular cells (endothelial cells and pericytes) as well as non-vascular cells including neurons, macroglia and microglia. All vertebrate retinas are composed of three layers of nerve cell bodies and two layers of synapses. The outer nuclear layer contains cell bodies of the rods and cones, the inner nuclear layer contains cell bodies of the bipolar, horizontal and amacrine cells and the ganglion cell layer contains cell bodies of ganglion cells and displaced amacrine cells. Dividing these nerve cell layers are two neuropils where synaptic contacts occur.

The first area of neuropil is the outer plexiform layer (OPL) where connections between rod and cones, and vertically running bipolar cells and horizontally oriented horizontal cells occur. The second neuropil of the retina is the inner plexiform layer (IPL), and it functions as a relay station for the vertical-information-carrying nerve cells, the bipolar cells, to connect to ganglion cells. It is at the culmination of all this neural processing in the IPL that the message concerning the visual image is transmitted through ganglion cells to the brain along the optic nerve. A decrease in the IPL thickness is used as an indicator for retinal neurodegeneration (Kolb et al, 1995).

There are 1.2 million retinal ganglion cells (RGC) within each retina. The axons of those cells are unmyelinated. The axons acquire myelin as they leave the eye to form the optic nerve. (Prasad S, 2011) Optic neuropathies are degenerative diseases of the retina that result from ganglion cell degeneration, (Dana Blumberg, 2015). Optic neuropathies causes can be hereditary (Newman, 2004) as well as acquired (O'Neill, 2010). Glaucomatous optic neuropathy is special form of optic neuropathy with increased intraocular pressure as the major risk factor. It is characterized by progressive loss of retinal ganglion cells (RGCs) and their axons and leads to measurable structural and functional damage to the optic nerve, visual impairment, and blindness (Marianne L. Shahsuvaryan, 2013). Nonarteritic anterior ischemic optic neuropathy (NAION) is the most common form of ischemic optic neuropathy and the second most common optic neuropathy (Berry S, 2017).

Oxidative stress is an imbalance between the production and removal of reactive oxygen species (ROS), has been implicated in many types of nerve cell death in the central nervous system (CNS) and in the eye (Coyle J T, 1993). A final common pathway of oxidative stress-induced death was suggested in the RGCs cell death. Therapies that prevent oxidative stress in RGC can be neuroprotective regardless the initial cause of the oxidative stress and the underlying cause of the optic neuropathy (Pamela Maher, 2005). As mentioned earlier increased levels of reactive oxygen species (ROS) destroys the biological activity of nitric oxide (NO) and limit cGMP formation due to deregulated NO/sGC signaling in the retina (Schaefer et al. 2003).

Neuroprotective and regenerative agents are emerging therapeutics on the horizon to help combat optic neuropathies. The techniques and approaches hope to rejuvenate RGCs and repair the optic nerve structures, thereby providing a gain of function of the visual system for the glaucoma patients. Ocular hypertension has been proven to be an important risk factor involved in the onset and progression of glaucomatous optic neuropathy. However, the analysis of the clinical record of a sample of 592 subjects with glaucoma under intraocular pressure lowering medication showed that in the last visit 42.2% of them were blind in one eye and 16.4% were blind bilaterally. These data support the hypothesis that risk factors other than IOP intervene in the pathogenesis of the neuronal damage in glaucoma. More than 100 neuroprotective drug candidates have failed to demonstrate efficacy, acceptable safety, or patient benefit. Most of them, in fact, despite successful preclinical data, failed to pass most of the Phase 2 and virtually all the Phase 3 clinical trials. For instance, memantine, a non-competitive N-methyl-D-aspartate (NMDA) subtype of glutamate receptor antagonist (Nucci et al 2018). Similarly, several neuroprotection agents for ischemic stroke and various types of optic neuropathies have been evaluated extensively in experimental studies in animals and benefits claimed. However, translation of therapeutic strategies for neuroprotection from experimental research to humans has invariably been fraught with failure (Hayreh et al. 2019). Therefore, there is a high need for an oral treatment that can address the failure of the neuroprotective strategies.

Surprisingly we found that the compounds of the present invention protect the non-vascular neuronal element of the neurovascular unit as shown in experiment B-9 “Evaluation of the changes in rat retinal structure after Streptozotocin-induced DR model in rat (STZ rat model)” and experiment B-7 & B-8 “Evaluation of the changes in rat retinal structure after retinal ischemia reperfusion (I/R)”. In both experiments, the inner plexiform layer (IPL), with its functions as a relay station for the vertical-information-carrying nerve cells, the bipolar cells, to connect to ganglion cells was protected. With this unexpected finding it is made plausible that compounds of the invention protect against optic neuropathies and can prevent progression of retinal neurodegenerative diseases as glaucoma optic neuropathy, ischemic optic neuropathy, traumatic optic neuropathy, non-arteritic anterior ischemic optic neuropathy, optic neuropathy, leber's hereditary optic neuropathy, methanol associated optic neuropathy and age-related macular degeneration.

Two sGC modulators (sGC activator MGV354; and sGC stimulator IW-6463) were reported to be tested in treatment of glaucoma and CNS disorder. Both compounds differ from the current invention as MGV354 was topically applied while the IW-6463 was sGC stimulator.

MGV354 is a topically administered sGC activator (Ehara, 2018) that was reported to increase aqueous outflow through the trabecular meshwork and Schlemm canal by increasing production of cyclic guanosine monophosphate (cGMP) in these tissues in preclinical models (Ganesh Prasanna, 2018). However, this effect was not translatable to human eye (Rebecca Stacy, 2018). IW-6463 is an orally administrated CNS-penetrant sGC stimulator is tested for central nerve system diseases (E. S. Buys, 2018).

Cataract is defined as opacity within the clear lens inside the eye that reduces the amount of incoming light and results in deterioration of vision. Natural lens is a crystalline substance and a precise structure of water and protein to create a clear passage for light. Cataract is often described as being like looking through a waterfall or waxed paper. Senile cataract due to aging is more common than other types of cataract. Apart from aging, various risk factors of cataract like: Nutritional inadequacy, metabolic and inherited defects, ultraviolet radiation, and smoking have been implicated as significant risk factors in development of cataract. Evidently, direct in vivo and in vitro experimental studies suggest that diabetes is a cause of cataract. Uncontrolled DM results in hyperglycemia, which is associated in ocular tissues with non-enzymatic protein glycation, osmotic stress, and oxidative stress (Gupta V B, 2014).

Even though cataract surgery, the most common surgical ophthalmic procedure worldwide, is an effective cure, the elucidation of pathomechanisms to delay or prevent the development of cataract in diabetic patients remains a challenge (Pollreisz A and Schmidt-Erfurth U, 2010). Aldose-reductase inhibitors and antioxidants have been proven beneficial in the prevention or treatment of this sight-threatening condition in in vitro and in vivo experimental studies. (Robinson et al. 1996, Zhao et al 2000). Although a preclinical evidence of effect was present in animal models, there is a failure to translate this effect into human clinical observation (Meyer C H and Sekundo W, 2005). Both diabetes and cataract pose an enormous health and economic burden, particularly in developing countries, where diabetes treatment is insufficient and cataract surgery often inaccessible (Tabin et al 2008). Therefore, there is a high need for an oral treatment that allows for delay or prevent the development of cataract when surgery is not feasible or associated with high complication risk as in diabetic patient.

Accordingly, it is an object of the present invention to provide suitable compounds, compound combinations and pharmaceutical compositions for use in the treatment and/or prophylaxis of eye diseases, especially in the oral treatment and/or prophylaxis of eye diseases.

It is a further object of the present invention to provide suitable compounds, compound combinations and pharmaceutical compositions for use in the treatment and/or prophylaxis of eye diseases caused by neurovascular unit damage.

It is a further object of the present invention to provide suitable compounds, compound combinations and pharmaceutical compositions for use in the treatment and/or prophylaxis of eye diseases caused by neurovascular unit damage or retinal ganglion cell/photoreceptor neurodegeneration.

It is a further object of the present invention to provide suitable compounds, compound combinations and pharmaceutical compositions for use in the treatment and/or prophylaxis of eye diseases selected from the group consisting of non-proliferative diabetic retinopathy (NPDR), diabetic macular edema (DME), central retinal vein occlusion, branch retinal vein occlusion, retinal artery occlusion, retinopathy of prematurity, ocular ischemic syndrome, radiation retinopathy, anterior ischemic optic neuritis, anti-VEGF therapy driven ischemia, ocular neuropathies and choroidal ischemic diseases, for example diabetic choroidopathy.

It is a further preferred object of the present invention to provide suitable compounds, compound combinations and pharmaceutical compositions for use in the treatment and/or prophylaxis of non-proliferative diabetic retinopathy (NPDR) and diabetic macular edema (DME).

It is a further most preferred object of the present invention to provide suitable compounds, compound combinations and pharmaceutical compositions for use in the treatment and/or prophylaxis of non-proliferative diabetic retinopathy (NPDR).

Within the meaning of the present invention, the term “treating” or “treatment” as used in the present text is used conventionally, e.g., the management or care of a subject for the purpose of combating, alleviating, reducing, relieving, improving the condition of a disease or disorder, such as visual acuity, e.g. NPDR associated visual acuity and any associated condition.

Within the meaning of the present invention, the terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

As used herein, the term “activator of soluble Guanylyl Cyclase (sGC)” or “sGC activator” relates to an active compound that interacts with an oxidized or heme-free form of the sGC, to activate an oxidized or heme-free form of the sGC to catalyze the formation of cGMP (Schmidt et al. 2009).

As used herein, the term “activation” is to be understood as increasing the measured production of cGMP by at least 5% as compared to a control, e.g., a non-treated control, preferably by at least 10%, more preferably by at least 15%, even more preferably by at least 20%, even more preferably by at least 25%, even more preferably by at least 30% or by at least 40% or by at least 50%. Suitable controls are evident for the skilled person when considering the teaching of the present disclosure. Suitable assays to determine said activation are readily available to the skilled person from the pertinent literature. In one embodiment of the invention, experiment 12 “Stimulation and Activation of recombinant soluble guanylate cyclase (sGC) in vitro” is being used to determine said activation.

The term “eye disease” refers to a medical condition that prevents the physiological function of different eye components.

The term “neurovascular unit damage” refers to a medical condition that describes damage of neurovascular unit. In normal healthy retina there is functional coupling and interdependency of neurons, glial elements including Müller cells, and vascular cells, with associated immune cells such as microglia.

Diabetic retinopathy compromises the endothelial-mural cell interactions, vascular basement membrane damage, Müller cell gliosis, and immune cell activation. Together, these changes result in impairment of neurovascular coupling, with consequences including blood-retinal barrier breakdown and dysregulation of retinal blood flow, which are described as neurovascular unit damage (Duh et al. 2017).

The term “non-proliferative diabetic retinopathy” or “NPDR” refers to a medical condition that describes the retinal manifestations of diabetes mellitus before development of neovascularization. Clinically, the hallmark of the non-proliferative phase is microaneurysms and intraretinal abnormalities. Different stages of diabetic retinopathy can be differentiated and quantified using the diabetic retinopathy severity score (DRSS) as shown in Table 1 (ETDRS Report Number 12, 1991).

The NPDR conditions are levels between 35 and 53, with level 35 defined as “mild NPDR”, level 43 defined as “moderate NPDR”, level 47 defined as “moderately severe NPDR” and level 53 defined as “severe NPDR”. Detailed description of each level was provided by the ETDRS report number 12 (ETDRS Report Number 12, 1991).

TABLE 1

| Level | Description | Definition |
|---|---|---|
| 10 | DR absent | Microaneurysms and other |
| | | characteristics absent |
| 14-20 | DR questionable | HE, SE, or IRMA definite; |
| | | microaneurysms absent |
| 15 | DR questionable | Hemorrhage(s) definite; |
| | | microaneurysms absent |
| 20 | Microaneurysms | Microaneurysms definite, other |
| | only | characteristics absent |
| 35† | Mild NPDR | One or more of the following: |
| | | Venous loops ≥ D/1 |
| | | SE, IRMA, or VB = Q |
| | | Retinal haemorrhages present |
| | | HE ≥ D/1 |
| | | SE ≥ D/1 |
| 43 | Moderate NPDR | H/Ma = M/4-5 − S/1 or |
| | | IRMA = D/1-3 (not both) |
| 47 | Moderately | Both L43 characteristics and/or 1 |
| | severe NPDR | (only) of the following: |
| | | IRMA = D4-5 |
| | | H/Ma = S/2-3 |
| | | VB = D/1 |
| 53 | Severe NPDR* | One or more of the following: |
| | | ≥2 of the 3 L47 characteristics |
| | | H/Ma ≥ S/4-5 |
| | | IRMA ≥ M/1 |
| | | VB ≥ D/2-3 |
| 61 | Mild PDR | FPD or FPE present with NVD |
| | | and NVE absent; or NVE = D |
| 65 | Moderate PDR | Either of the following: |
| | | NVE ≥ M/1 or NVD = D and VH |
| | | or PRH = A or Q |
| | | VH or PRH = D and NVE < M/1 |
| | | and NVD absent |
| 71 | High-risk PDR | Any of the following: |
| | | VH or PRH ≥ M/1 |
| | | NVE ≥ M/1 and VH or PRH ≥ D/1 |
| | | NVD = 2 and VH or PRH ≥ D/1 |
| | | NVD ≥ M |
| 75 | High-risk PDR | NVD ≥ M and VH or PRH ≥ D/1 |
| 81 | Advanced PDR: | NVD = cannot grade, or NVD < D |
| | fundus partially | and NVE = cannot grade in 1 field |
| | obscured, center of | and absent in all others; and retinal |
| | macula attached | detachment at center of macula < D |
| 85 | Advanced PDR: | VH = VS in fields 1 and 2; or retinal |
| | posterior fundus | detachment at center of macula = D |
| | obscured, or center | |
| | of macula detached | |
| 90 | Cannot grade, | |
| | even sufficiently | |
| | for level 81 or 85 | |

Legend for Table 1: ETDRS, Early Treatment Diabetic Retinopathy Study; DR, diabetic retinopathy; FPD, fibrous proliferations disc; FPE, fibrous proliferations elsewhere; HE, hard exudates; H/Ma, haemorrhages/microaneurysms; IRMA, intraretinal microvascular abnormalities; NPDR, nonproliferative DR; NVD, new vessels disc (within one disc diameter of disc margin); NVE, new vessels elsewhere (>1 disc diameter from disc); PDR, proliferative DR; SE, soft exudates; VB, venous beading; VH, vitreous haemorrhage; PRH, preretinal haemorrhage.
*NPDR levels 35 and above all require presence of microaneurysms.

The term “diabetic macular edema” or “DME” refers to a medical condition that describes the retinal manifestations of diabetes mellitus where accumulation of fluid (oedema) is present in the retinal area serving central vision (macula).

The term “optic neuropathies” refers to a medical condition that describe degenerative diseases of the retina that result from ganglion cell degeneration.

The term “cataract” refers to a medical condition that describes the opacity within the clear lens inside the eye that reduces the amount of incoming light and results in deterioration of vision.

One embodiment of the invention is at least one sGC activator, preferably of formula I, (I-A), (I-B), (I-C), (I-D), (I-E), (I-F), (I-G), (I-H), (I-I), (I-J) or (I-K) for use in the treatment and/or prophylaxis of an eye disease, especially for use in the oral treatment and/or prophylaxis of an eye disease.

A further embodiment of the invention is at least one sGC activator, preferably of I, (I-A), (I-B), (I-C), (I-D), (I-E), (I-F), (I-G), (I-H), (I-I), (I-J) or (I-K) for in the treatment and/or prophylaxis of an eye disease associated with neurovascular unit damage.

A further embodiment of the invention is at least one sGC activator, preferably of I, (I-A), (I-B), (I-C), (I-D), (I-E), (I-F), (I-G), (I-H), (I-I), (I-J) or (I-K) for use in the treatment and/or prophylaxis of an eye disease associated with neurovascular unit damage or retinal ganglion cell/photoreceptor neurodegeneration.

A further embodiment of the invention is at least one sGC activator, preferably of formula I, (I-A), (I-B), (I-C), (I-D), (I-E), (I-F), (I-G), (I-H), (I-I), (I-J) or (I-K) for use in the treatment and/or prophylaxis of an ischemic eye disease.

A further embodiment of the invention is at least one sGC activator, preferably of formula I, (I-A), (I-B), (I-C), (I-D), (I-E), (I-F), (I-G), (I-H), (I-I), (I-J) or (I-K) for use in the treatment and/or prophylaxis of an eye disease selected from the group consisting of non-proliferative diabetic retinopathy (NPDR), diabetic macular edema (DME), central retinal vein occlusion, branch retinal vein occlusion, retinal artery occlusion, retinopathy of prematurity, ocular ischemic syndrome, radiation retinopathy, anterior ischemic optic neuritis, anti-VEGF therapy driven ischemia, ocular neuropathies and choroidal ischemic diseases, for example diabetic choroidopathy.

A further embodiment of the invention is at least one sGC activator, preferably of I, (I-A), (I-B), (I-C), (I-D), (I-E), (I-F), (I-G), (I-H), (I-I), (I-J) or (I-K) for use in the treatment and/or prophylaxis of an eye disease selected from the group consisting of non-proliferative diabetic retinopathy (NPDR) and diabetic macular edema (DME).

A further embodiment of the invention is at least one sGC activator, preferably of formula I, (I-A), (I-B), (I-C), (I-D), (I-E), (I-F), (I-G), (I-H), (I-I), (I-J) or (I-K) for use in the treatment and/or prophylaxis of an eye disease which is non-proliferative diabetic retinopathy (NPDR).

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is non-proliferative diabetic retinopathy (NPDR), whereas the diabetic retinopathy severity score (DRSS) is between 35 to 53.

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is non-proliferative diabetic retinopathy (NPDR), whereas the diabetic retinopathy severity score (DRSS) is between 43-53 (NPDR).

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is non-proliferative diabetic retinopathy (NPDR), whereas the diabetic retinopathy severity score (DRSS) is 35, 43, 47 or 53.

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is non-proliferative diabetic retinopathy, characterized in that the disease progression is stopped and the retinal function is restored to healthier status (reversal of disease progression).

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is non-proliferative diabetic retinopathy, whereas non-proliferative diabetic retinopathy is associated with ischemic macular edema.

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is non-proliferative diabetic retinopathy, whereas ischemic macular edema is caused by DR, branch retinal vein occlusion or radiation retinopathy.

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is an optic neuropathy.

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is an optic neuropathy, whereas the optic neuropathy is selected from the group consisting of glaucoma optic neuropathy, ischemic optic neuropathy, traumatic optic neuropathy, non-arteritic anterior ischemic optic neuropathy, optic neuropathy, leber's hereditary optic neuropathy, methanol associated optic neuropathy and age-related macular degeneration.

A further embodiment of the invention is at least one sGC activator, preferably of formula TI, (I-A), (I-B), (I-C), (I-D), (I-E), (I-F), (I-G), (I-H), (I-I), (I-J) or (I-K) for use in the treatment and/or prophylaxis of an eye disease which is glaucoma optic neuropathy.

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is glaucoma optic neuropathy caused by acute closed angle glaucoma.

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is associated with cataract formation.

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is associated with cataract formation, whereas the cataract formation cause is selected from a list consisting of age-related cataract, diabetes induced cataract (preferred), steroid induced cataract, traumatic cataract, congenital cataract.

A further embodiment of the invention is at least one sGC activator, preferably of formula I, I, (I-A), (I-B), (I-C), (I-D), (I-E), (I-F), (I-G), (I-H), (I-I), (I-J) or (I-K) for use in the treatment and/or prophylaxis of an eye disease which is associated with cataract formation, whereas the cataract formation cause is diabetes induced cataract secondary to type 1 or type 2 diabetes.

A further embodiment of the invention is at least one sGC activator, preferably of formula I, (I-A), (I-B), (I-C), (I-D), (I-E), (I-F), (I-G), (I-H), (I-I), (I-J) or (I-K) for use in the treatment and/or prophylaxis of an eye disease which is associated with cataract formation, whereas the cataract formation cause is diabetes induced cataract secondary to type 1 diabetes.

A further embodiment of the invention is at least one sGC activator for use in the treatment and/or prophylaxis of an eye disease mentioned above, preferably NPDR, whereas at least one sGC activator is a compound of formula (I)

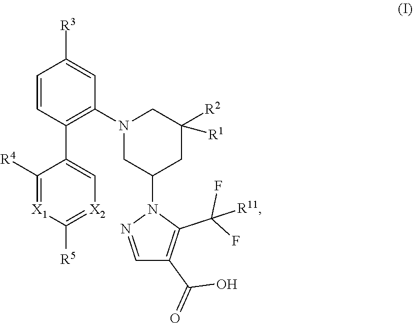

in which
R¹ represents hydrogen or halogen,
R² represents hydrogen or halogen,
R³ represents chloro or trifluoromethyl,
R⁴ represents hydrogen, C₁-C₄-alkyl,
R⁵ represents a group of the formula

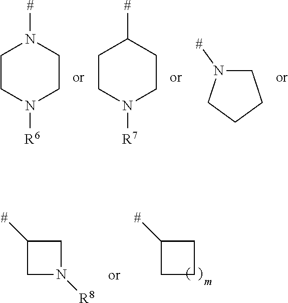

where #is the point of attachment to the aromatic or heteroaromatic 6 ring system; wherein m is 0-4
R⁶ represents
   C₁-C₆-alkyl, optionally substituted by one or more substituent independently selected from the group consisting of methyl, trifluoromethoxy, nitril, amido,
   C₂-C₆-halogenoalkyl, optionally substituted by 1 to 5 fluoro substituents,
   C₃-C₆-cycloalkyl,
   C₃-C₆-cycloalkyl-methyl, optionally substituted by 1 to 5 fluoro substituents or a trifluoromethyl group,
   C₁-C₆-alkylcarbonyl, optionally substituted by 1 to 3 fluoro substituents,
   C₃-C₆-cycloalkyl-carbonyl, optionally substituted by 1 to 3 fluoro substituents or
   (C₁-C₆)-alkoxy-carbonyl, optionally substituted with methoxy, trifluoromethoxy, C₃-C₆-cycloalkyl,
   (C₃-C₆)-cycloalkoxy-carbonyl,
   mono-(C₁-C₄)-alkylaminocarbonyl,
   (C₁-C₄)-alkylsulfonyl or
   oxetanyl,
   spiro[2.2]pentan-2-ylmethyl or [(3-fluoro-1-bicyclo[1.1.1]pentanyl)methyl,
R⁷ represents C₁-C₄-alkylcarbonyl, optionally substituted by a C₃-C₆-cycloalkyl group,
R⁸ represents C₂-C₄-alkyl, C₂-C₄-halogenoalkyl substituted by 1 to 6 fluoro substituents,
R¹¹ represents hydrogen or fluoro substituent
X₁ represents nitrogen or carbon or C—F
X₂ represents nitrogen or carbon
and the salts thereof, the solvates thereof and the solvates of the salts thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is corresponding to the following formula (I-A)

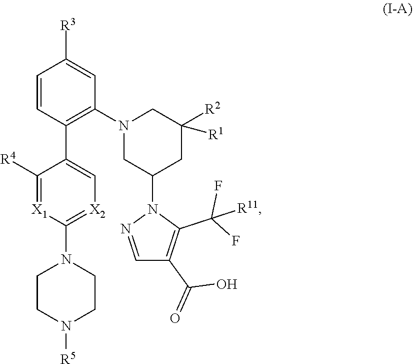

in which
R¹ represents hydrogen or halogen,
R² represents hydrogen or halogen,
R³ represents choro or tifluoromethyl,
R⁴ represents hydrogen or C₁-C₄-alkyl
R⁵ represents optionally substituted C n-C₆-alkyl
R¹¹ represents hydrogen or fluoro substituent
X₁ represents nitrogen or carbon
X₂ represents nitrogen or carbon
and the salts thereof, the solvates thereof and the solvates of the salts thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride corresponding to the following formula (I-B)

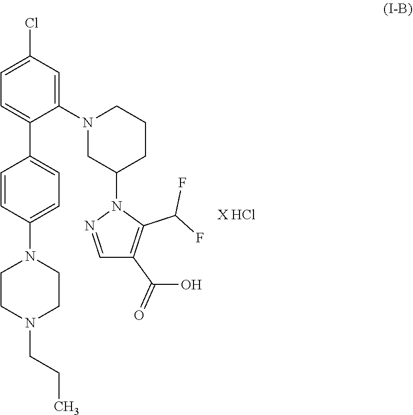

and salts, solvates and solvates of the salts thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride corresponding to the following formula (I-C)

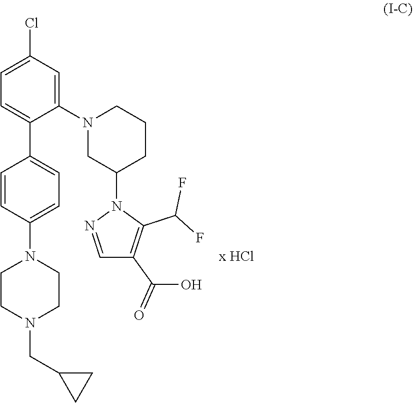 [SMILES: O=C(O)c1cnn(C2CCCN(c3cc(Cl)ccc3-c3ccc(N4CCN(CC5CC5)CC4)cc3)C2)c1C(F)F ; "x HCl"]

and salts, solvates and solvates of the salts thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-{1-[4-Chloro-4′-(4-isobutylpiperazin-1-yl)[biphenyl]-2-yl]piperidin-3-yl}-5-(difluoromethyl)-1H-pyrazole-4-carboxylic acid corresponding to the following formula (I-D)

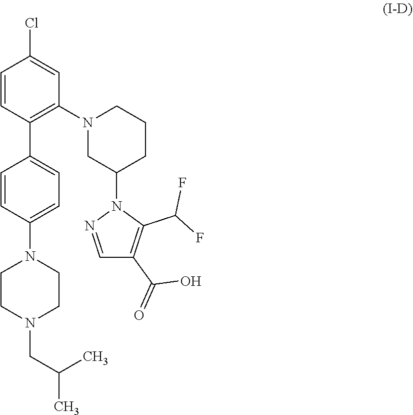 [SMILES: CC(C)CN1CCN(c2ccc(-c3ccc(Cl)cc3N3CCCC(n4ncc(C(=O)O)c4C(F)F)C3)cc2)CC1]

both enantiomers

and salts, solvates and solvates of the salts thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride corresponding to the following formula (I-E)

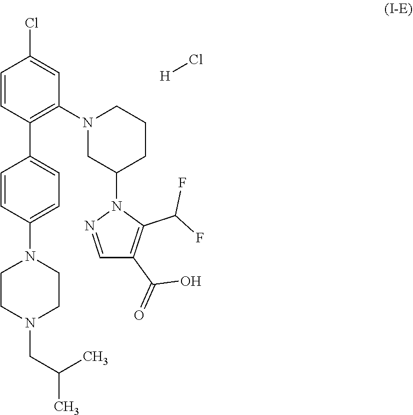 [SMILES: CC(C)CN1CCN(c2ccc(-c3ccc(Cl)cc3N3CCCC(n4ncc(C(=O)O)c4C(F)F)C3)cc2)CC1 ; Cl]

and solvates thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride corresponding to the following formula (I-E-R)

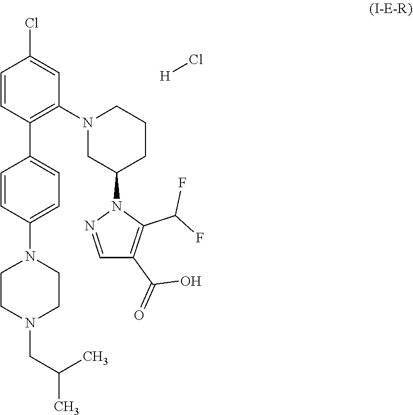 [SMILES: CC(C)CN1CCN(c2ccc(-c3ccc(Cl)cc3N3CCC[C@@H](n4ncc(C(=O)O)c4C(F)F)C3)cc2)CC1 ; Cl]

and solvates thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride hemihydrate corresponding to the following formula (I-E-R hemihydrate)

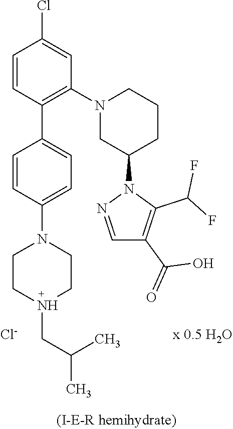

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid corresponding to the following formula (I-F)

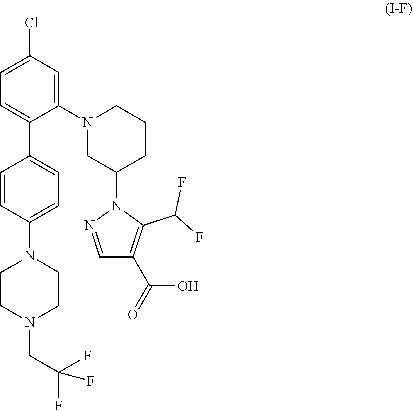 [SMILES: O=C(O)c1cnn(C2CCCN(c3cc(Cl)ccc3-c3ccc(N4CCN(CC(F)(F)F)CC4)cc3)C2)c1C(F)F]

and salts, solvates and solvates of the salts thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid corresponding to the following formula (I-H)

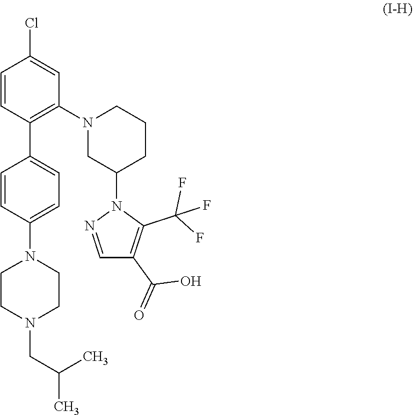 [SMILES: CC(C)CN1CCN(c2ccc(-c3ccc(Cl)cc3N3CCCC(n4ncc(C(=O)O)c4C(F)(F)F)C3)cc2)CC1]

and salts, solvates and solvates of the salts thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride corresponding to the following formula (I-I)

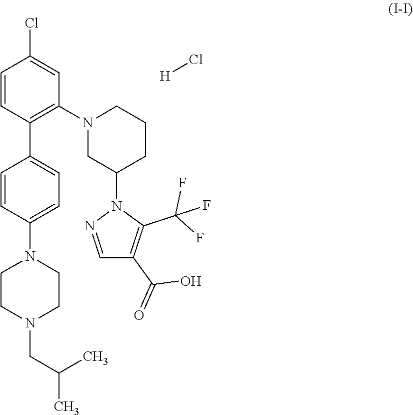 [SMILES: CC(C)CN1CCN(c2ccc(-c3ccc(Cl)cc3N3CCCC(n4ncc(C(=O)O)c4C(F)(F)F)C3)cc2)CC1 ; Cl]

and salts, solvates and solvates of the salts thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride corresponding to the following formula (I-J)

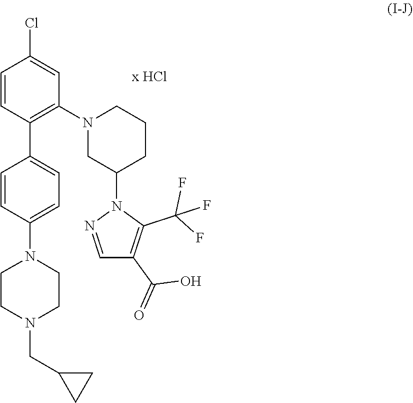 [SMILES: O=C(O)c1cnn(C2CCCN(c3cc(Cl)ccc3-c3ccc(N4CCN(CC5CC5)CC4)cc3)C2)c1C(F)(F)F ; "x HCl"]

and salts, solvates and solvates of the salts thereof.

A further embodiment of the invention is at least one sGC activator for use in the treatment and/or prophylaxis of an eye disease mentioned above, preferably NPDR, glaucoma optic neuropathy and/or an eye disease which is associated with cataract formation, whereas the at least one sGC activator is 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid corresponding to the following formula (I-K)

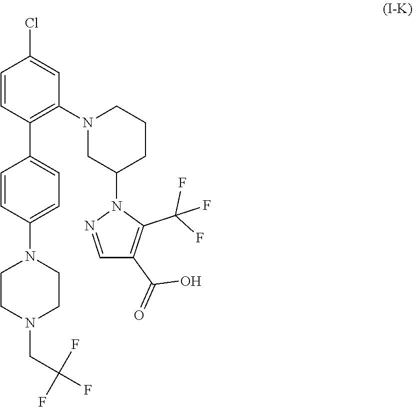 [SMILES: O=C(O)c1cnn(C2CCCN(c3cc(Cl)ccc3-c3ccc(N4CCN(CC(F)(F)F)CC4)cc3)C2)c1C(F)(F)F]

and salts, solvates and solvates of the salts thereof.

The compounds according to the invention are potent activators of soluble guanylate cyclase. They lead to vasorelaxation, inhibition of platelet aggregation and lowering of blood pressure and increase of coronary blood flow. These effects are mediated via direct haem-independent activation of soluble guanylate cyclase and an increase of intracellular cGMP.

In addition, the compounds according to the invention have advantageous pharmacokinetic properties, in particular with respect to their bioavailability and/or duration of action after intravenous or oral administration.

The compounds according to the invention have an unforeseeable useful pharmacological activity spectrum and good pharmacokinetic behavior, in particular a sufficient exposure of such a compound in the blood above the minimal effective concentration within a given dosing interval after oral administration. Such a profile results in an improved peak-to-trough ratio (quotient of maximum to minimum concentration) within a given dosing interval, which has the advantage that the compound can be administered less frequently and

The present invention furthermore provides the use of the compounds according to the invention for the treatment and/or prevention of disorders, in particular the disorders mentioned above.

The present invention furthermore provides the use of the compounds according to the invention for preparing a medicament for the treatment and/or prevention of disorders, in particular the disorders mentioned above.

The present invention furthermore provides a medicament comprising at least one of the compounds according to the invention for the treatment and/or prevention of disorders, in particular the disorders mentioned above.

The present invention furthermore provides the use of the compounds according to the invention in a method for the treatment and/or prevention of disorders, in particular the disorders mentioned above.

The present invention furthermore provides a method for the treatment and/or prevention of disorders, in particular the disorders mentioned above, using an effective amount of at least one of the compounds according to the invention.

They are therefore suitable for use as medicaments for the treatment and/or prophylaxis of diseases in humans and animals.

The present invention further provides medicaments comprising a compound according to the invention and one or more further active compounds, typically together with one or more inert, nontoxic, pharmaceutically suitable auxiliaries, and the use thereof for the aforementioned purposes.

The compounds, combinations, pharmaceutical compositions and medicaments according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Suitable for extraocular (topic) administration are administration forms which operate in accordance with the prior art, which release the active compound rapidly and/or in a modified or controlled manner and which contain the active compound in crystalline and/or amorphized and/or dissolved form such as, for example, eye drops, sprays and lotions (e.g. solutions, suspensions, vesicular/colloidal systems, emulsions, aerosols), powders for eye drops, sprays and lotions (e.g. ground active compound, mixtures, lyophilisates, precipitated active compound), semisolid eye preparations (e.g. hydrogels, in-situ hydrogels, creams and ointments), eye inserts (solid and semisolid preparations, e.g. bioadhesives, films/wafers, tablets, contact lenses).

Intraocular administration includes, for example, intravitreal, subretinal, subscleral, intrachoroidal, subconjunctival, retrobulbar and subtenon administration. Suitable for intraocular administration are administration forms which operate in accordance with the prior art, which release the active compound rapidly and/or in a modified or controlled manner and which contain the active compound in crystalline and/or amorphized and/or dissolved form such as, for example, preparations for injection and concentrates for preparations for injection (e.g. solutions, suspensions, vesicular/colloidal systems, emulsions), powders for preparations for injection (e.g. ground active compound, mixtures, lyophilisates, precipitated active compound), gels for preparations for injection (semisolid preparations, e.g. hydrogels, in-situ hydrogels) and implants (solid preparations, e.g. biodegradable and nonbiodegradable implants, implantable pumps).

Oral administration is preferred, especially in form of a tablet, most preferably in form of a tablet which release the compounds, combinations, pharmaceutical compositions or medicaments according to the invention in a modified manner.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients.

Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylnethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The compounds, combinations, pharmaceutical compositions and medicaments according to the invention can be converted to the administration forms mentioned. This can be done in a manner known per se, by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and flavour and/or odour correctants.

The present invention furthermore relates to a pharmaceutical composition which comprises at least one compound according to the invention, conventionally together with one or more pharmaceutically suitable excipient(s), and to their use according to the present invention.

An embodiment of the invention are pharmaceutical compositions comprising at least one compound of formula (I) according to the invention, preferably together with at least one inert, non-toxic, pharmaceutically suitable auxiliary, and the use of these pharmaceutical compositions for the above cited purposes.

In accordance with another aspect, the present invention covers pharmaceutical combinations, in particular medicaments, comprising at least one compound of general formula (I) of the present invention and at least one or more further active ingredients, in particular for the treatment and/or prophylaxis of ophthalmological diseases, preferably non-proliferative diabetic retinopathy (NPDR), diabetic macular edema (DME), retinal ganglion cell/photoreceptor neurodegeneration and cataract.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The inventive compounds can be employed alone or, if required, in combination with other active ingredients. The present invention further provides medicaments comprising at least one of the inventive compounds and one or more further active ingredients, especially for treatment and/or prophylaxis of the aforementioned disorders. Preferred examples of suitable active ingredient combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2, 5 and/or 9, especially PDE 5 inhibitors such as sildenafil, vardenafil, tadalafil, udenafil, desantafil, avanafil, mirodenafil, lodenafil or PF-00489791;
compounds which inhibit the breakdown of cyclic adenosine monophosphate (cAMP), for example inhibitors of phosphodiesterases (PDE) 3 and 4, especially cilostatzole, milrinone, roflumilast, apremilast, or crisaborole;
hypotensive active ingredients, by way of example and with preference from the group of the calcium antagonists, angiotensin AII antagonists, ACE inhibitors, NEP-inhibitors, vasopeptidase-inhibitors, endothelin antagonists, renin inhibitors, alpha-receptor blockers, beta-receptor blockers, mineralocorticoid receptor antagonists, rho-kinase-inhibitors and the diuretics;
antiarrhythmic agents, by way of example and with preference from the group of sodium channel blocker, beta-receptor blocker, potassium channel blocker, calcium antagonists, If-channel blocker, digitalis, parasympatholytics (vagoliytics), sympathomimetics and other antiarrhythmics as adenosin, adenosine receptor agonists as well as vernakalant;
positive-inotrop agents, by way of example cardiac glycoside (Dogoxin), beta-adrenergic and dopaminergic agonists, such as isoprenalin, adrenalin, noradrenalin, dopamin or dobutamin;
vasopressin-receptor-antagonists, by way of example and with preference from the group of conivaptan, tolvaptan, lixivaptan, mozavaptan, satavaptan, pecavaptan, SR-121463, RWJ 676070 or BAY 86-8050, as well as the compounds described in WO 2010/105770, WO2011/104322 and WO 2016/071212;
active ingredients which alter lipid metabolism, for example and with preference from the group of the thyroid receptor agonists, cholesterol synthesis inhibitors such as, by way of example and preferably, HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.
bronchodilatory agents, for example and with preference from the group of the beta-adrenergic rezeptor-agonists, such as, by way of example and preferably, albuterol, isoproterenol, metaproterenol, terbutalin, formoterol or salmeterol, or from the group of the anticholinergics, such as, by way of example and preferably, ipratropiumbromid;
anti-inflammatory agents, for example and with preference from the group of the glucocorticoids, such as, by way of example and preferably, prednison, prednisolon, methylprednisolon, triamcinolon, dexamethason, beclomethason, betamethason, flunisolid, budesonid or fluticason as well as the non-steroidal anti-inflammatory agents (NSAIDs), by way of example and preferably, acetyl salicylic acid (aspirin), ibuprofen and naproxen, 5-amino salicylic acid-derivates, leukotriene-antagonists, TNF-alpha-inhibitors and chemokin-receptor antagonists, such as CCR1, 2 and/or 5 inhibitors;
agents modulating the immune system, for example immunoglobulins;
agents that inhibit the signal transductions cascade, for example and with preference from the group of the kinase inhibitors, by way of example and preferably, from the group of the tyrosine kinase- and/or serine/threonine kinase inhibitors;
agents, that inhibit the degradation and modification of the extracellular matrix, for example and with preference from the group of the inhibitors of the matrix-metalloproteases (MMPs), by way of example and preferably, inhibitors of chymasee, stromelysine, collagenases, gelatinases and aggrecanases (with preference from the group of MMP-1, MMP-3, MMP-8, MMP-9, MMP-10, MMP-11 and MMP-13) as well as of the metallo-elastase (MMP-12) and neutrophil-elastase (HNE), as for example sivelestat or DX-890;
agents, that block the bindung of serotonin to its receptor, for example and with preference antagonists of the 5-HT2b-receptor;
organic nitrates and NO-donators, for example and with preference sodium nitroprussid, nitro-glycerine, isosorbid mononitrate, isosorbid dinitrate, molsidomine or SIN-1, as well as inhaled NO;
NO-independent, but heme-dependent stimulators of the soluble guanylate cyclase, for example and with preference the compounds described in WO 00/06568, WO 00/06569, WO 02/42301, WO 03/095451, WO 2011/147809, WO 2012/004258, WO 2012/028647 and WO 2012/059549;
NO-independent and heme-independent activators of the soluble guanylate cyclase, for example and with preference the compounds described in WO 01/19355, WO 01/19776, WO 01/19778, WO 01/19780, WO 02/070462 and WO 02/070510 beschriebenen Verbindungen;
agents, that stimulates the synthesis of cGMP, like for example sGC modulators, for example and with preference riociguat, cinaciguat, vericiguat or runcaciguat;
prostacyclin-analogs, for example and with preference iloprost, beraprost, treprostinil or epoprostenol;
agents, that inhibit soluble epoxidhydrolase (sEH), for example and with preference N,N′-Dicyclohexyl urea, 12-(3-Adamantan-1-yl-ureido)-dodecanic acid or 1-Adamantan-1-yl-3-{5-[2-(2-ethoxyethoxy)ethoxy]pentyl}-urea;
agents that interact with glucose metabolism, for example and with preference insuline, biguanide, thiazolidinedione, sulfonyl urea, acarbose, DPP4 inhibitors, GLP-1 analogs or SGLT-2 inhibitors, for example empagliflozin, dapagliflozin, canagliflozin, sotagliflozin;
natriuretic peptides, for example and with preference atrial natriuretic peptide (ANP), natriuretic peptide type B (BNP, Nesiritid) natriuretic peptide type C (CNP) or urodilatin;
activators of the cardiac myosin, for example and with preference omecamtiv mecarbil (CK-1827452);
calcium-sensitizers, for example and with preference levosimendan;
agents that affect the energy metabolism of the heart, for example and with preference etomoxir, dichloroacetat, ranolazine or trimetazidine, full or partial adenosine A1 receptor agonists such as GS-9667 (formerly known as CVT-3619), capadenoson, neladenoson and neladenoson bialanate;
agents that affect the heart rate, for example and with preference ivabradin;
cyclooxygenase inhibitors such as, for example, bromfenac and nepafenac;
inhibitors of the kallikrein-kinin system such as, for example, safotibant and ecallantide;
inhibitors of the sphingosine 1-phosphate signal paths such as, for example, sonepcizumab;
inhibitors of the complement-C5a receptor such as, for example, eculizumab;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors) such as, for example, tissue plasminogen activator (t-PA, for example Actilyse®), streptokinase, reteplase and urokinase or plasminogen-modulating substances causing increased formation of plasmin;
anticoagulatory substances (anticoagulants) such as, for example, heparin (UFH), low-molecular-weight heparins (LMW), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI) such as, for example, Pradaxa (dabigatran), atecegatran (AZD-0837), DP-4088, SSR-182289A, argatroban, bivalirudin and tanogitran (BIBT-986 and prodrug BIBT-1011) and hirudin;
direct factor Xa inhibitors such as, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux;
inhibitors of coagulation factor XI and XIa such as, for example, FXI ASO-LICA, fesomersen, BAY 121-3790, MAA868, BMS986177, EP-7041 and AB-022;
substances which inhibit the aggregation of platelets (platelet aggregation inhibitors, thrombocyte aggregation inhibitors), such as, for example, acetylsalicylic acid (such as, for example, aspirin), P2Y12 antagonists such as, for example, ticlopidine (Ticlid), clopidogrel (Plavix), prasugrel, ticagrelor, cangrelor and elinogrel, and PAR-1 antagonists such as, for example, vorapaxar, and PAR-4 antagonists;
platelet adhesion inhibitors such as GPVI and/or GPIb antagonists such as, for example, Revacept or caplacizumab;
fibrinogen receptor antagonists (glycoprotein-IIb/IIIa antagonists) such as, for example, abciximab, eptifibatide, tirofiban, lamifiban, lefradafiban and fradafiban;
recombinant human activated protein C such as, for example, Xigris or recombinant thrombomodulin.

Antithrombotic agents are preferably understood to mean compounds from the group of the platelet aggregation inhibitors, the anticoagulants or the profibrinolytic substances.

In a preferred embodiment of the invention, the inventive compounds are administered in combination with a platelet aggregation inhibitor, by way of example and with preference aspirin, clopidogrel, prasugrel, ticagrelor, ticlopidin or dipyridamole.

In a preferred embodiment of the invention, the inventive compounds are administered in combination with a thrombin inhibitor, by way of example and with preference ximelagatran, dabigatran, melagatran, bivalirudin or clexane.

In a preferred embodiment of the invention, the inventive compounds are administered in combination with a GPIIb/IIIa antagonist such as, by way of example and with preference, tirofiban or abciximab.

In a preferred embodiment of the invention, the inventive compounds are administered in combination with a factor Xa inhibitor, by way of example and with preference rivaroxaban (BAY 59-7939), DU-176b, apixaban, betrixaban, otamixaban, fidexaban, razaxaban, letaxaban, eribaxaban, fondaparinux, idraparinux, PMD-3112, darexaban (YM-150), KFA-1982, EMD-503982, MCM-17, MLN-1021, DX 9065a, DPC 906, JTV 803, SSR-126512 or SSR-128428.

In a preferred embodiment of the invention, the inventive compounds are administered in combination with a factor XI or factor XIa inhibitor, by way of example and with preference FXI ASO-LICA, fesomersen, BAY 121-3790, MAA868, BMS986177, EP-7041 or AB-022.

In a preferred embodiment of the invention, the inventive compounds are administered in combination with heparin or with a low molecular weight (LMW) heparin derivative.

In a preferred embodiment of the invention, the inventive compounds are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, rho-kinase inhibitors and the diuretics.

In a preferred embodiment of the invention, the inventive compounds are administered in combination with a calcium antagonist, by way of example and with preference nifedipine, amlodipine, verapamil or diltiazem.

In a preferred embodiment of the invention, the inventive compounds are administered in combination with an alpha-1-receptor blocker, by way of example and with preference prazosin.

In a preferred embodiment of the invention, the inventive compounds are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the inventive compounds are administered in combination with an angiotensin AII antagonist, by way of example and with preference losartan, candesartan, valsartan, telmisartan or embusartan or a dual angiotensin AII antagonist/neprilysin-inhibitor, by way of example and with preference LCZ696 (valsartan/sacubitril).

In a preferred embodiment of the invention, the inventive compounds are administered in combination with an ACE inhibitor, by way of example and with preference enalapril, captopril, lisinopril, ramipril, delapril, fosinopril, quinopril, perindopril or trandopril.

In a preferred embodiment of the invention, the inventive compounds are administered in combination with an endothelin antagonist, by way of example and with preference bosentan, darusentan, ambrisentan or sitaxsentan.

In a preferred embodiment of the invention, the inventive compounds are administered in combination with a renin inhibitor, by way of example and with preference aliskiren, SPP-600 or SPP-800.

In a preferred embodiment of the invention, the inventive compounds are administered in combination with a mineralocorticoid receptor antagonist, by way of example and with preference spironolactone, AZD9977, finerenone or eplerenone.

In a preferred embodiment of the invention, the inventive compounds are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the inventive compounds are administered in combination with a CETP inhibitor, by way of example and with preference dalcetrapib, anacetrapib, torcetrapib (CP-529 414), JJT-705 or CETP vaccine (Avant).

In a preferred embodiment of the invention, the inventive compounds are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the inventive compounds are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the inventive compounds are administered in combination with a squalene synthesis inhibitor, by way of example and with preference BMS-188494 or TAK-475.

In a preferred embodiment of the invention, the inventive compounds are administered in combination with an ACAT inhibitor, by way of example and with preference avasimibe, melinamide, pactimibe, eflucimibe or SMP-797.

In a preferred embodiment of the invention, the inventive compounds are administered in combination with an MTP inhibitor, by way of example and with preference implitapide, BMS-201038, R-103757 or JTT-130.

In a preferred embodiment of the invention, the inventive compounds are administered in combination with a PPAR-gamma agonist, by way of example and with preference pioglitazone or rosiglitazone.

In a preferred embodiment of the invention, the inventive compounds are administered in combination with a PPAR-delta agonist, by way of example and with preference GW 501516 or BAY 68-5042.

In a preferred embodiment of the invention, the inventive compounds are administered in combination with a cholesterol absorption inhibitor, by way of example and with preference ezetimibe, tiqueside or pamaqueside.

In a preferred embodiment of the invention, the inventive compounds are administered in combination with a lipase inhibitor, a preferred example being orlistat.

In a preferred embodiment of the invention, the inventive compounds are administered in combination with a polymeric bile acid adsorbent, by way of example and with preference cholestyramine, colestipol, colesolvam, CholestaGel or colestimide.

In a preferred embodiment of the invention, the inventive compounds are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the inventive compounds are administered in combination with a lipoprotein(a) antagonist, by way of example and with preference, gemcabene calcium (CI-1027) or nicotinic acid.

In a preferred embodiment of the invention, the inventive compounds are administered in combination with a lipoprotein(a) antagonist, by way of example and with preference, gemcabene calcium (CI-1027) or nicotinic acid.

In a preferred embodiment of the invention, the inventive compounds are administered in combination with sGC modulators, by way of example and with preference, riociguat, cinaciguat or vericiguat.

In a preferred embodiment of the invention, the inventive compounds are administered in combination with an agent affecting the glucose metabolism, by way of example and with preference, insuline, a sulfonyl urea, acarbose, DPP4 inhibitors, GLP-1 analogs or SGLT-1 inhibitors empagliflozin, dapagliflozin, canagliflozin, sotagliflozin.

In a preferred embodiment of the invention, the compounds according to the invention are administered in combination with a TGFbeta antagonist, by way of example and with preference pirfenidone or fresolimumab.

In a preferred embodiment of the invention, the compounds according to the invention are administered in combination with a CCR2 antagonist, by way of example and with preference CCX-140.

In a preferred embodiment of the invention, the compounds according to the invention are administered in combination with a TNFalpha antagonist, by way of example and with preference adalimumab.

In a preferred embodiment of the invention, the compounds according to the invention are administered in combination with a galectin-3 inhibitor, by way of example and with preference GCS-100.

In a preferred embodiment of the invention, the compounds according to the invention are administered in combination with a Nrf-2 inhibitor, by way of example and with preference bardoxolone

In a preferred embodiment of the invention, the compounds according to the invention are administered in combination with a BMP-7 agonist, by way of example and with preference THR-184.

In a preferred embodiment of the invention, the compounds according to the invention are administered in combination with a NOX1/4 inhibitor, by way of example and with preference GKT-137831.

In a preferred embodiment of the invention, the compounds according to the invention are administered in combination with a medicament which affects the vitamin D metabolism, by way of example and with preference calcitriol, alfacalcidol, doxercalciferol, maxacalcitol, paricalcitol, cholecalciferol or paracalcitol.

In a preferred embodiment of the invention, the compounds according to the invention are administered in combination with a cytostatic agent, by way of example and with preference cyclophosphamide.

In a preferred embodiment of the invention, the compounds according to the invention are administered in combination with an immunosuppressive agent, by way of example and with preference ciclosporin.

In a preferred embodiment of the invention, the compounds according to the invention are administered in combination with a phosphate binder, by way of example and with preference colestilan, sevelamer hydrochloride and sevelamer carbonate, Lanthanum and lanthanum carbonate.

In a preferred embodiment of the invention, the compounds according to the invention are administered in combination with renal proximal tubule sodium-phosphate co-transporter, by way of example and with preference, niacin or nicotinamide.

In a preferred embodiment of the invention, the compounds according to the invention are administered in combination with a calcimimetic for therapy of hyperparathyroidism.

In a preferred embodiment of the invention, the compounds according to the invention are administered in combination with agents for iron deficit therapy, by way of example and with preference iron products.

In a preferred embodiment of the invention, the compounds according to the invention are administered in combination with agents for the therapy of hyperurikaemia, by way of example and with preference allopurinol or rasburicase.

In a preferred embodiment of the invention, the compounds according to the invention are administered in combination with glycoprotein hormone for the therapy of anaemia, by way of example and with preference erythropoietin, daprodustat, molidustat, roxadustat, vadadustat, desidustat.

In a preferred embodiment of the invention, the compounds according to the invention are administered in combination with biologics for immune therapy, by way of example and with preference abatacept, rituximab, eculizumab or belimumab.

In a preferred embodiment of the invention, the compounds according to the invention are administered in combination with vasopressin antagonists (group of the vaptanes) for the treatment of heart failure, by way of example and with preference tolvaptan, conivaptan, lixivaptan, mozavaptan, satavaptan, pecavaptan or relcovaptan.

In a preferred embodiment of the invention, the compounds according to the invention are administered in combination with Jak inhibitors, by way of example and with preference ruxolitinib, tofacitinib, baricitinib, CYT387, GSK2586184, lestaurtinib, pacritinib (SB1518) or TG101348.

In a preferred embodiment of the invention, the compounds according to the invention are administered in combination with prostacyclin analogs for therapy of microthrombi.

In a preferred embodiment of the invention, the compounds according to the invention are administered in combination with an alkali therapy, by way of example and with preference sodium bicarbonate.

In a preferred embodiment of the invention, the compounds according to the invention are administered in combination with an mTOR inhibitor, by way of example and with preference everolimus or rapamycin.

In a preferred embodiment of the invention, the compounds according to the invention are administered in combination with an NHE3 inhibitor, by way of example and with preference AZD1722 or tenapanor.

In a preferred embodiment of the invention, the compounds according to the invention are administered in combination with an eNOS modulator, by way of example and with preference sapropterin.

In a preferred embodiment of the invention, the compounds according to the invention are administered in combination with a CTGF inhibitor, by way of example and with preference FG-3019.

The total amount of the active ingredient to be administered will generally range from about 0.001 mg/kg to about 200 mg/kg body weight per day, and preferably from about 0.01 mg/kg to about 50 mg/kg body weight per day, and more preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Nevertheless, it may optionally be necessary to deviate from the stated amounts, namely depending on body weight, route of administration, individual response to the active substance, type of preparation and time point or interval when application takes place. Thus, in some cases it may be sufficient to use less than the aforementioned minimum amount, whereas in other cases the stated upper limit must be exceeded. When applying larger amounts, it may be advisable to distribute these in several individual doses throughout the day.

According to a further embodiment, the compounds of formula (I) according to the invention are administered orally once or twice or three times a day. According to a further embodiment, the compounds of formula (I) according to the invention are administered orally once or twice a day. According to a further embodiment, the compounds of formula (I) according to the invention are administered orally once a day.

For the oral administration, a rapid release or a modified release dosage form may be used.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

The present invention further provides pharmaceutical compositions which comprise at least one of the above mentioned sGC activators, typically together with one or more inert, nontoxic, pharmaceutically suitable excipients, and for the use thereof for the aforementioned purposes. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

Another embodiment of the invention is a pharmaceutical composition comprising at least one of the above mentioned sGC activators in combination with one or more inert non-toxic pharmaceutically suitable excipients for use in the treatment and/or prophylaxis of ophthalmologic diseases selected from the group consisting of diabetic retinopathy, non-proliferative diabetic retinopathy (NPDR) and diabetic macular edema (DME) glaucoma optic neuropathy and/or an eye disease which is associated with cataract formation.

Another embodiment of the invention is a pharmaceutical composition comprising at least one of the above mentioned sGC activators in combination with one or more inert non-toxic pharmaceutically suitable excipients for use in the treatment and/or prophylaxis of ophthalmologic diseases selected from the group consisting of diabetic retinopathy, non-proliferative diabetic retinopathy (NPDR) and diabetic macular edema (DME) glaucoma optic neuropathy and/or an eye disease which is associated with cataract formation.

Another embodiment of the invention is a pharmaceutical composition comprising at least one of the above mentioned sGC activators and at least one compound selected from the group consisting of inhibitors of phosphodiesterases (PDE) 1, 2 and/or 5, for use in the treatment and/or prophylaxis of ophthalmologic diseases selected from the group consisting diabetic retinopathy, non-proliferative diabetic retinopathy (NPDR) and diabetic macular edema (DME) glaucoma optic neuropathy and/or an eye disease which is associated with cataract formation.

Another embodiment of the invention is a pharmaceutical composition comprising at least one of the above mentioned sGC activators and at least one compound selected from the group consisting of inhibitors of phosphodiesterases (PDE) 1, 2 and/or 5, for use in the treatment and/or prophylaxis of ophthalmologic diseases selected from the group consisting of diabetic retinopathy, non-proliferative diabetic retinopathy (NPDR) and diabetic macular edema (DME) glaucoma optic neuropathy and/or an eye disease which is associated with cataract formation.

A further embodiment of the invention is a combination for use in the treatment and/or prophylaxis of an eye disease selected from the group consisting of non-proliferative diabetic retinopathy (NPDR), diabetic macular edema (DME), central retinal vein occlusion, branch retinal vein occlusion, retinal artery occlusion, retinopathy of prematurity, ocular ischemic syndrome, radiation retinopathy, anterior ischemic optic neuritis, anti-VEGF therapy driven ischemia, ocular neuropathies and choroidal ischemic diseases, for example diabetic choroidopathy, preferably NPDR, comprising at least one sGC activator and at least one compound selected from the group consisting of inhibitors of phosphodiesterases 1, 2 and/or 5, calcium, vitamin D and metabolites of vitamin D, bisphosphonates, selected from etidronate, clodronate, tiludronate, teriparatide, pamidronate, neridronate, olpadronate, alendronate, ibandronate, risedronate, and zoledronate, strontium ranelate, active ingredients suitable for hormone replacement therapy in osteoporosis, selected from estrogen and a combination of estrogen and progesterone, selective estrogen receptor modulators (SERMs), parathyroid hormone and analogs of parathyroid hormone, modulators of receptor activator of nuclear factor kappa-B ligand (RANKL), sclerostin inhibitors, and TGF-β inhibitors.

A further embodiment of the invention is the before mentioned combination for use in the treatment and/or prophylaxis of an eye disease mentioned above, preferably NPDR, whereas the at least one sGC activator is selected from a list consisting of 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E).

A further embodiment of the invention is the before mentioned combination for use in the treatment and/or prophylaxis of an eye disease mentioned above, preferably NPDR, glaucoma optic neuropathy and/or an eye disease which is associated with cataract formation whereas the at least one sGC activator is selected from a list consisting of 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[i-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E).

A further embodiment of the invention is one of the before mentioned combination for use in the treatment and/or prophylaxis of an eye disease mentioned above, whereas the at least one inhibitor of phosphodiesterase 5 is selected from the group consisting of sildenafil, vardenafil, tadalafil and avanafil.

A further embodiment of the invention is the before mentioned combination for use in the treatment and/or prophylaxis of an eye disease mentioned above, preferably NPDR, glaucoma optic neuropathy and/or an eye disease which is associated with cataract formation whereas the at least one sGC activator is selected from a list consisting of 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E).

and whereas the at least one inhibitor of phosphodiesterase 5 is selected from the group consisting of sildenafil, vardenafil, tadalafil and avanafil.

A further embodiment of the invention is the before mentioned combination for use in the treatment and/or prophylaxis of an eye disease mentioned above, preferably NPDR, glaucoma optic neuropathy and/or an eye disease which is associated with cataract formation whereas the at least one sGC activator is selected from a list consisting of 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E) and wherein the mineralocorticoid-receptor antagonist is selected from the group consisting of spironolactone, eplerenone or finerenone.

A further embodiment of the invention is a pharmaceutical composition for use in the treatment and/or prophylaxis of an eye disease mentioned above, preferably NPDR and/or DME, more preferably NPDR, comprising at least one sGC activator, preferably of 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E) in combination with one or more inert non-toxic pharmaceutically suitable excipients.

A further embodiment of the invention is a pharmaceutical composition for use in the treatment and/or prophylaxis of an eye disease mentioned above, preferably NPDR and/or DME, more preferably NPDR, or diabetic macular edema (DME), glaucoma optic neuropathy and/or an eye disease which is associated with cataract formation comprising at least one sGC activator, preferably of 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E) in combination with one or more inert non-toxic pharmaceutically suitable excipients.

A further embodiment of the invention is the before mentioned pharmaceutical composition for use in the treatment and/or prophylaxis of an eye disease mentioned above, preferably NPDR and/or DME, diabetic macular edema (DME), glaucoma optic neuropathy and/or an eye disease which is associated with cataract formation, more preferably NPDR, whereas the at least one sGC activator is selected from a list consisting of 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E).

A further embodiment of the invention is a pharmaceutical composition for use in the treatment and/or prophylaxis of an eye disease mentioned above, preferably NPDR and/or DME, diabetic macular edema (DME), glaucoma optic neuropathy and/or an eye disease which is associated with cataract formation more preferably NPDR, comprising at least one of the combinations mentioned above in combination with one or more inert non-toxic pharmaceutically suitable excipients.

Method for the treatment and/or prevention of an eye disease mentioned above, preferably NPDR and/or DME, diabetic macular edema (DME), glaucoma optic neuropathy and/or an eye disease which is associated with cataract formation more preferably NPDR, in humans and animals by administration of an effective amount of at least one sGC activator mentioned above, preferably 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E) or a pharmaceutical composition mentioned above.

In general, it has been found to be advantageous in the case of parenteral administration to administer amounts of about 0.001 to 1 mg/kg/day, preferably about 0.01 to 0.5 mg/kg/day, of body weight to achieve effective results. In the case of oral administration, the oral administration form contains 0.1 mg to 500 mg, preferably 1 mg to 120 mg, most preferable 2.5 mg to 50 mg or 2.5 mg to 60 mg of at least one compound according to the invention.

In a preferred embodiment the oral administration form contains 0.1 mg to 500 mg, preferably 1 mg to 120 mg, most preferable 2.5 mg to 50 mg or 2.5 mg to 60 mg of compound of formula 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E).

In a further preferred embodiment the oral administration form contains 0.1 mg to 500 mg, preferably 1 mg to 120 mg, most preferable 2.5 mg to 50 mg or 2.5 mg to 60 mg or 4 mg to 45 mg or 4 to 90 mg or 4 to 180 mg of compound of formula 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (compound of formula I-B), and/or 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (compound of formula I-C), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D), and/or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), and/or 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (compound of formula I-F), and/or (compound of formula I-G), and/or 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (compound of formula I-H), and/or 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (compound of formula I-I), and/or 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (compound of formula I-J), and/or 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (compound of formula I-K), preferably 1-{1-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D) or 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E), very preferably 1-{1(3R)-[4-Chloro-4′-(4-isobutylpiperazin-1-yl)[biphenyl]-2-yl]piperidin-3-yl}-5-(difluoromethyl)-1H-pyrazole-4-carboxylic acid (compound of formula I-D-R) or 1-{1(3R)-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (compound of formula I-E-R).

Suitable dosages for oral administration forms are for example 1 mg, 2 mg, 2.5 mg, 3 mg, 4 mg, 5 mg, 6 mg, 7 mg, 7.5 mg, 8 mg, 9 mg, 10 mg, 12.5 mg, 15 mg, 17.5 mg, 20 mg, 25 mg, 30 mg, 35 mg, 40 mg, 45 mg, 50 mg, 60 mg, 70 mg, 75 mg, 80 mg, 90 mg, 100 mg, 120 mg, 125 mg, 150 mg, 175 mg or 200 mg, preferably 4 mg, 5 mg, 6 mg, 7 mg, 7.5 mg, 8 mg, 9 mg, 10 mg, 12.5 mg, 15 mg, 17.5 mg, 20 mg, 25 mg, 30 mg, 35 mg, 40 mg, 45 mg.

It may nevertheless be necessary where appropriate to deviate from the stated amounts, specifically as a function of the body weight, route of administration, individual response to the active compound, nature of the preparation and time or interval over which administration takes place. For instance, in some cases, less than the aforementioned minimum amount may be sufficient, while in other cases the upper limit mentioned must be exceeded. In the case of administration of relatively large amounts, it may be advisable to divide these into several individual doses over the course of the day.

### SPECIFIC EMBODIMENTS

1. sGC activator of formula (I) for use in the oral treatment and/or prophylaxis of an eye disease

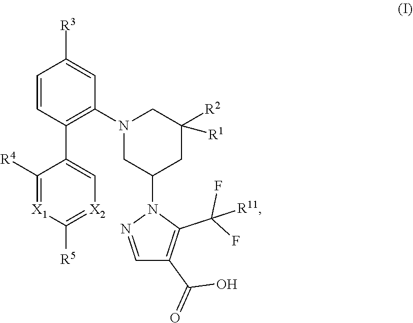

in which
R¹ represents hydrogen or halogen,
R² represents hydrogen or halogen,
R³ represents chloro or trifluoromethyl,
R⁴ represents hydrogen, C₁-C₄-alkyl,
R⁵ represents a group of the formula

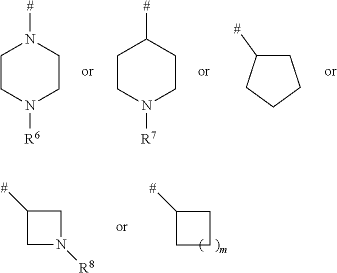

where #is the point of attachment to the aromatic or heteroaromatic 6 ring system; wherein m is 0-4
R⁶ represents
   C₁-C₆-alkyl, optionally substituted by one or more substituent independently selected from the group consisting of methyl, trifluoromethoxy, nitril, amido,
   C₂-C₆-halogenoalkyl, optionally substituted by 1 to 5 fluoro substituents,
   C₃-C₆-cycloalkyl,
   C₃-C₆-cycloalkyl-methyl, optionally substituted by 1 to 5 fluoro substituents or a trifluoromethyl group,
   C₁-C₆-alkylcarbonyl, optionally substituted by 1 to 3 fluoro substituents,
   C₃-C₆-cycloalkyl-carbonyl, optionally substituted by 1 to 3 fluoro substituents or
   (C₁-C₆)-alkoxy-carbonyl, optionally substituted with methoxy, trifluoromethoxy, C₃-C₆-cycloalkyl,
   (C₃-C₆)-cycloalkoxy-carbonyl,
   mono-(C₁-C₄)-alkylaminocarbonyl,
   (C₁-C₄)-alkylsulfonyl or
   oxetanyl,
   spiro[2.2]pentan-2-ylmethyl or [(3-fluoro-1-bicyclo[1.1.1]pentanyl)methyl,
R⁷ represents C₁-C₄-alkylcarbonyl, optionally substituted by a C₃-C₆-cycloalkyl group,
R⁸ represents C₂-C₄-alkyl, C₂-C₄-halogenoalkyl substituted by 1 to 6 fluoro substituents,
R¹¹ represents hydrogen or fluoro substituent
X₁ represents nitrogen or carbon or C—F
X₂ represents nitrogen or carbon
and salts, solvates and solvates of the salts thereof.
2. sGC activator for use according to Claim 1, whereas the sGC activator is corresponding to the following formula (I-A)

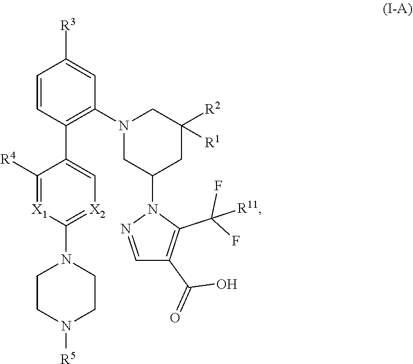

in which
R¹ represents hydrogen or halogen,
R² represents hydrogen or halogen,
R³ represents chloro or trifluoromethyl,
R⁴ represents hydrogen or C₁-C₄-alkyl
R⁵ represents optionally substituted C₁-C₆-alkyl
R¹¹ represents hydrogen or fluoro substituent
X₁ represents nitrogen or carbon
X₂ represents nitrogen or carbon
and the salts thereof, the solvates thereof and the solvates of the salts thereof.
3. sGC activator for use according to Claim 1, whereas the sGC activator is selected from the group consisting of

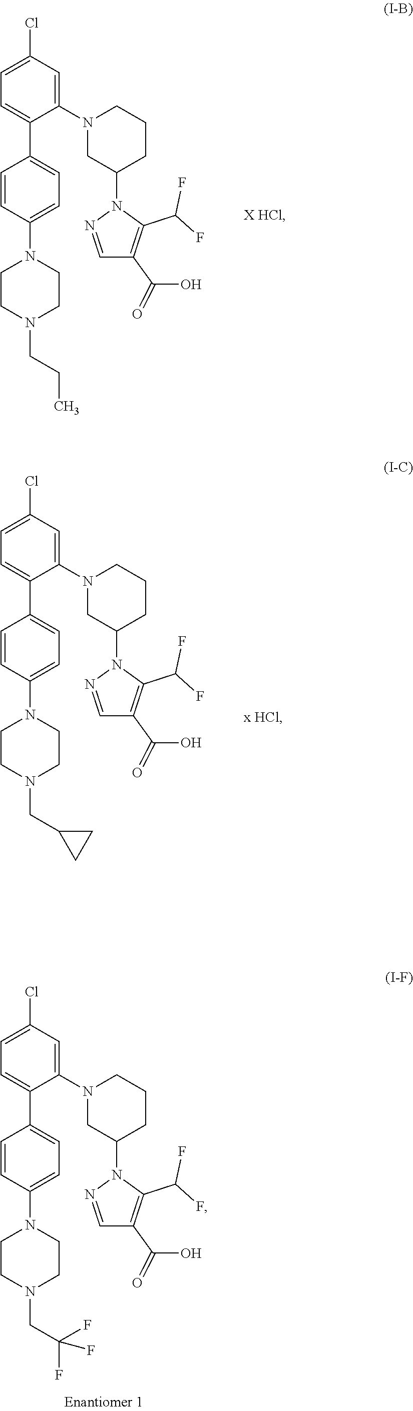 [SMILES: CCCN1CCN(c2ccc(-c3ccc(Cl)cc3N3CCCC(n4ncc(C(=O)O)c4C(F)F)C3)cc2)CC1 ; O=C(O)c1cnn(C2CCCN(c3cc(Cl)ccc3-c3ccc(N4CCN(CC5CC5)CC4)cc3)C2)c1C(F)F ; O=C(O)c1cnn(C2CCCN(c3cc(Cl)ccc3-c3ccc(N4CCN(CC(F)(F)F)CC4)cc3)C2)c1C(F)F]

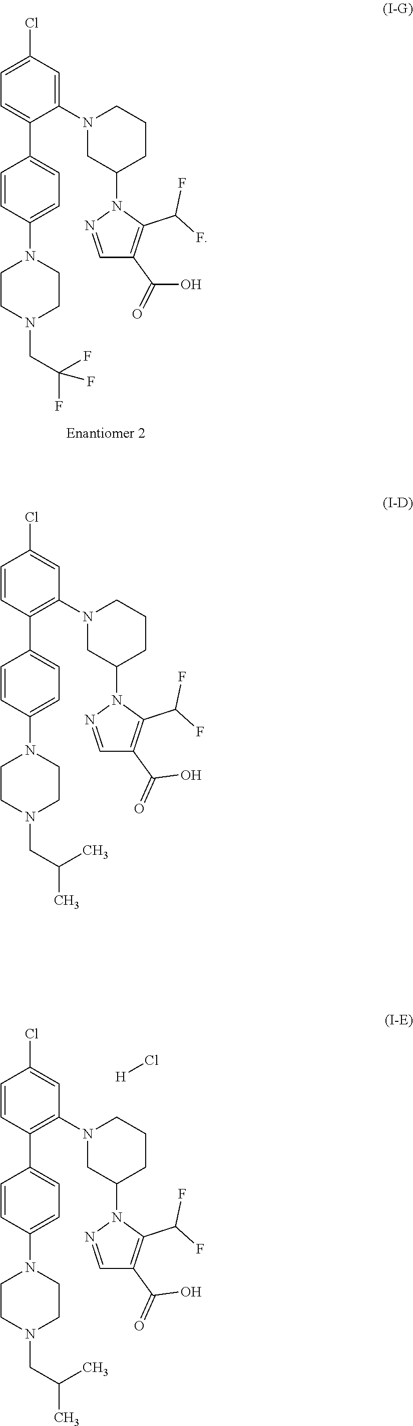

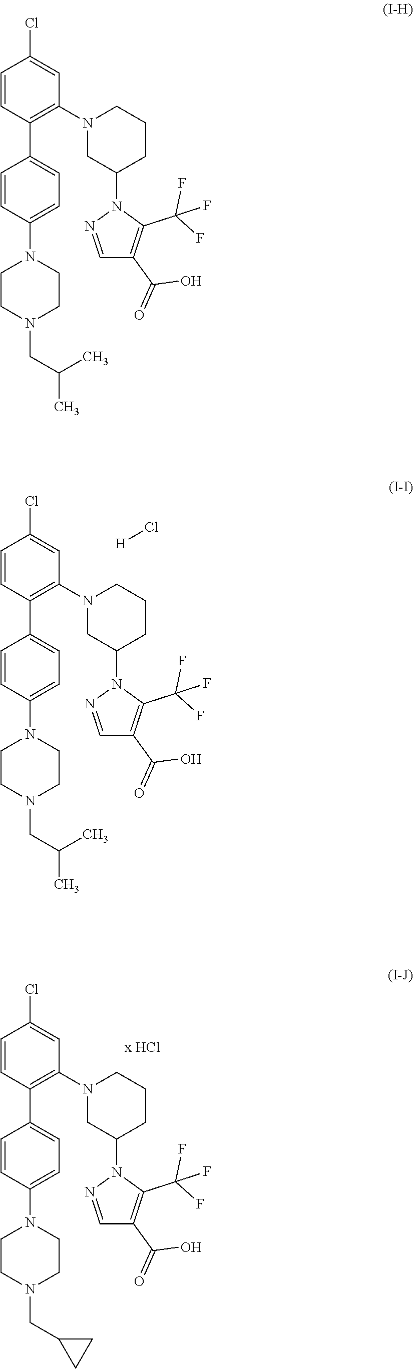

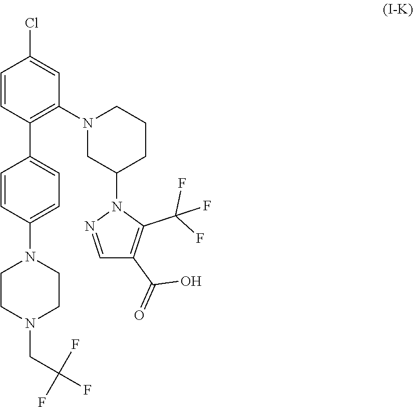 [SMILES: O=C(O)c1cnn(C2CCCN(c3cc(Cl)ccc3-c3ccc(N4CCN(CC(F)(F)F)CC4)cc3)C2)c1C(F)(F)F]

and salts, solvates and solvates of the salts thereof.
4. sGC activator for use according to Claim 1, whereas the sGC activator is selected from the group consisting of

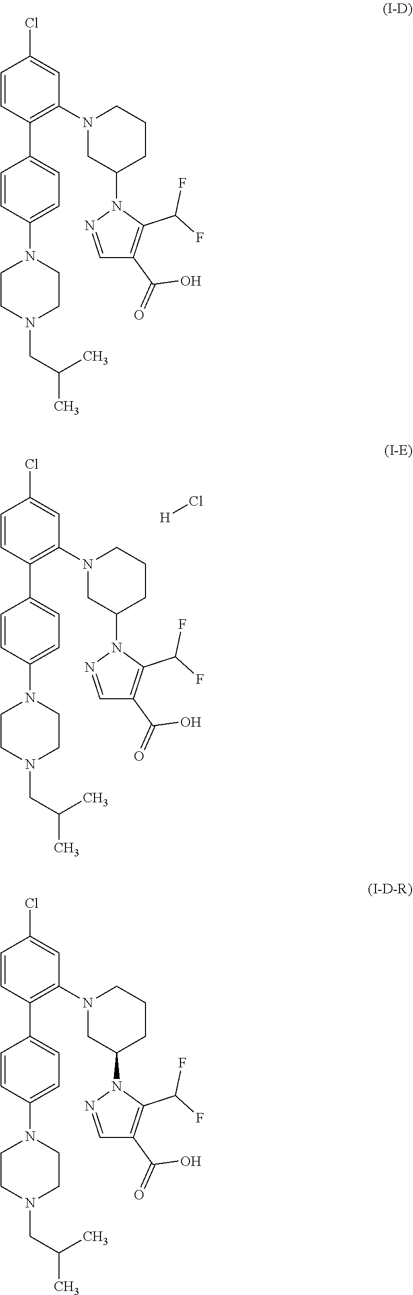 [SMILES: CC(C)CN1CCN(c2ccc(-c3ccc(Cl)cc3N3CCCC(n4ncc(C(=O)O)c4C(F)F)C3)cc2)CC1 ; CC(C)CN1CCN(c2ccc(-c3ccc(Cl)cc3N3CCCC(n4ncc(C(=O)O)c4C(F)F)C3)cc2)CC1 ; Cl ; CC(C)CN1CCN(c2ccc(-c3ccc(Cl)cc3N3CCC[C@@H](n4ncc(C(=O)O)c4C(F)F)C3)cc2)CC1]

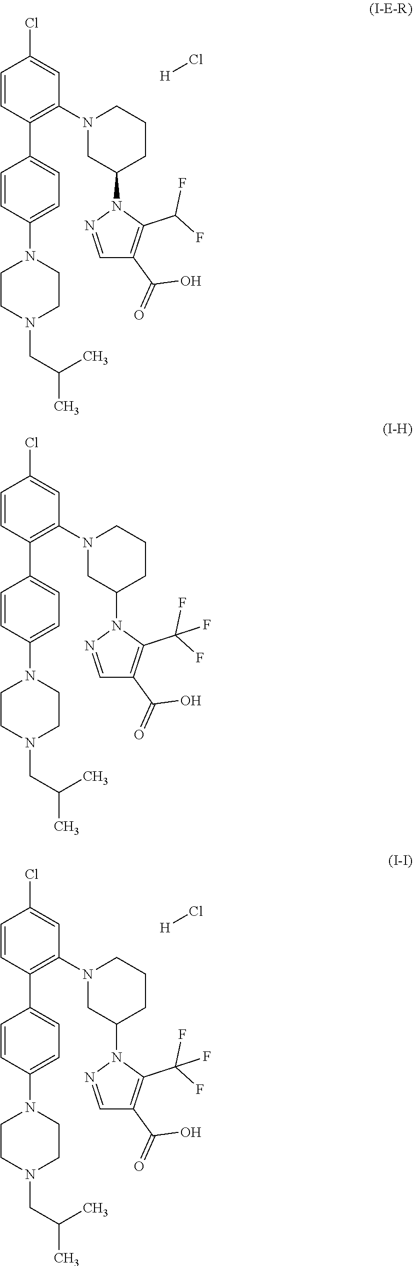

and salts, solvates and solvates of the salts thereof.
5. sGC activator for use according to Claim 1, whereas the sGC activator is selected from the group consisting of

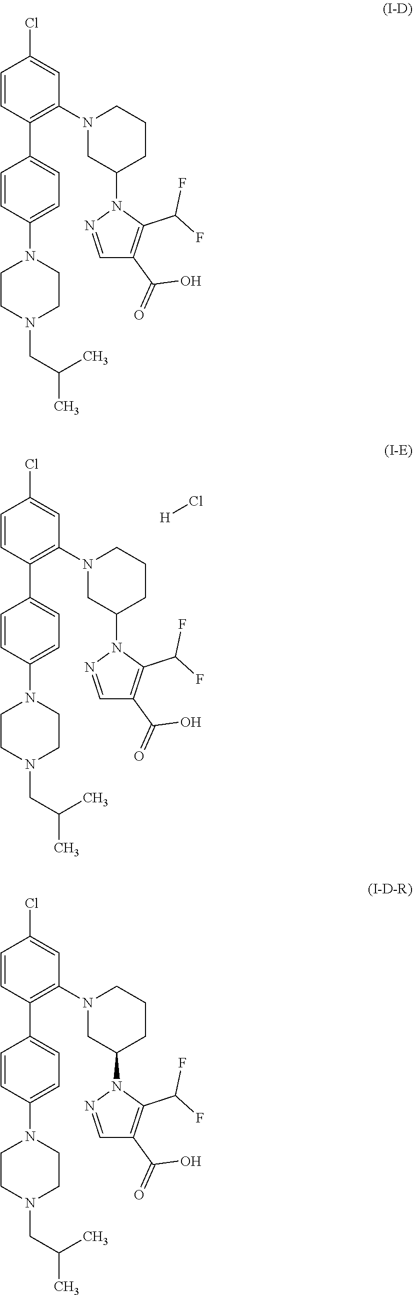 [SMILES: CC(C)CN1CCN(c2ccc(-c3ccc(Cl)cc3N3CCCC(n4ncc(C(=O)O)c4C(F)F)C3)cc2)CC1 ; CC(C)CN1CCN(c2ccc(-c3ccc(Cl)cc3N3CCCC(n4ncc(C(=O)O)c4C(F)F)C3)cc2)CC1 ; Cl ; CC(C)CN1CCN(c2ccc(-c3ccc(Cl)cc3N3CCC[C@@H](n4ncc(C(=O)O)c4C(F)F)C3)cc2)CC1]

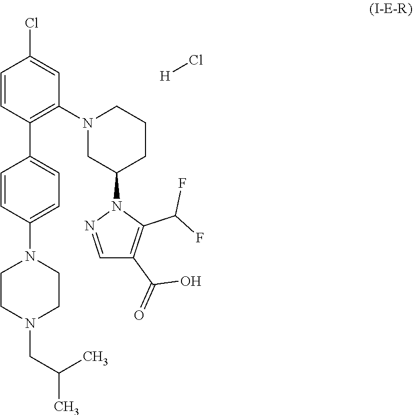 [SMILES: CC(C)CN1CCN(c2ccc(-c3ccc(Cl)cc3N3CCC[C@@H](n4ncc(C(=O)O)c4C(F)F)C3)cc2)CC1 ; Cl]

and salts, solvates and solvates of the salts thereof.
6. sGC activator for use according to Claim 1, whereas the sGC activator is ((I-D)

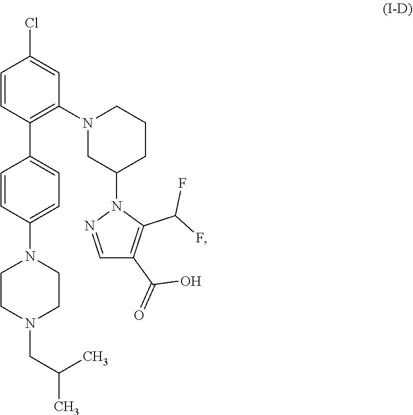 [SMILES: CC(C)CN1CCN(c2ccc(-c3ccc(Cl)cc3N3CCCC(n4ncc(C(=O)O)c4C(F)F)C3)cc2)CC1]

and salts, solvates and solvates of the salts thereof.
7. sGC activator for use according to Claim 1, whereas the sGC activator is ((I-D-R)

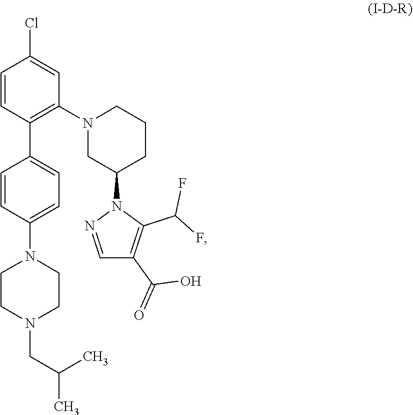 [SMILES: CC(C)CN1CCN(c2ccc(-c3ccc(Cl)cc3N3CCC[C@@H](n4ncc(C(=O)O)c4C(F)F)C3)cc2)CC1]

and salts, solvates and solvates of the salts thereof.
8. sGC activator for use according to Claim 1, whereas the sGC activator is corresponding to the following formula (I-H)

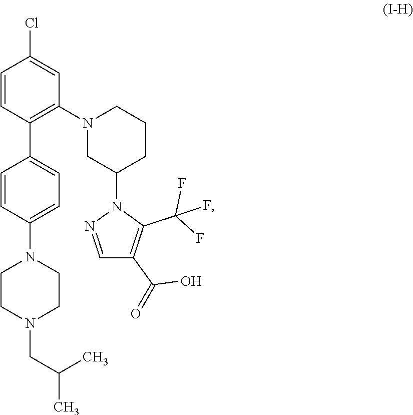 [SMILES: CC(C)CN1CCN(c2ccc(-c3ccc(Cl)cc3N3CCCC(n4ncc(C(=O)O)c4C(F)(F)F)C3)cc2)CC1]

and salts, solvates and solvates of the salts thereof.
9. sGC activator for use according to Claim 1, whereas the sGC activator is corresponding to the following formula (I-E)

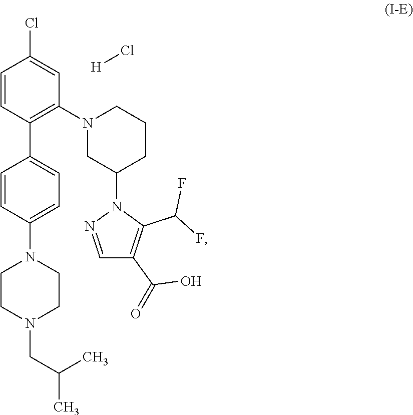

and solvates thereof.
10. sGC activator for use according to Claim 1, whereas the sGC activator is corresponding to the following formula (I-E-R)

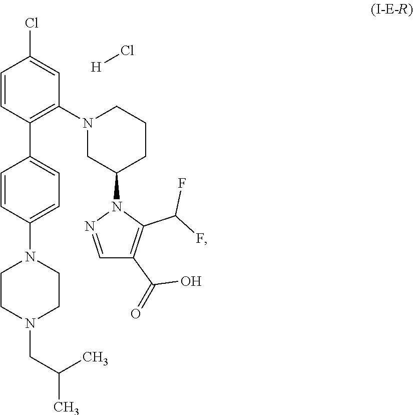

and solvates thereof.
11. sGC activator for use according to Claim 1, whereas the sGC activator is corresponding to the following formula (I-E-R hemihydrate)

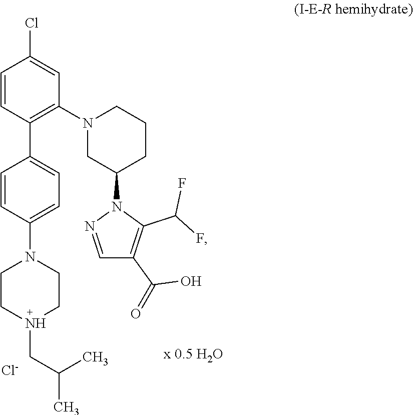 [SMILES: CC(C)CN1CCN(c2ccc(-c3ccc(Cl)cc3N3CCC[C@@H](n4ncc(C(=O)O)c4C(F)F)C3)cc2)CC1 ; [Cl-]]

12. sGC activator for use according to any of claims 1 to 11, whereas the eye disease is associated with neurovascular unit damage, lens opacity (cataract) or retinal ganglion cell/photoreceptor neurodegeneration.
13. sGC activator for use according to any of Claims 1 to 12, whereas the eye disease is selected from a list consisting of non-proliferative diabetic retinopathy, diabetic macular edema, central retinal vein occlusion, branch retinal vein occlusion, retinal artery occlusion, retinopathy of prematurity, ocular ischemic syndrome, radiation retinopathy, anterior ischemic optic neuritis, anti-VEGF therapy driven ischemia, ocular neuropathies and choroidal ischemic diseases.
14. sGC activator for use according to any of Claims 1 to 13, whereas the eye disease is selected from a list consisting of non-proliferative diabetic retinopathy, optic neuropathies and cataract.
15. sGC activator for use according to any of Claims 1 to 14, whereas the eye disease is non-proliferative diabetic retinopathy.
16. sGC activator for use in non-proliferative diabetic retinopathy according to Claim 15, whereas the diabetic retinopathy severity score (DRSS) is between 35 to 53.
17. sGC activator for use in non-proliferative diabetic retinopathy according to Claim 15, whereas the diabetic retinopathy severity score (DRSS) is between 43 to 53.
18. sGC activator for use in non-proliferative diabetic retinopathy according to Claim 15, characterized in that the disease progression is stopped and the retinal function is restored to healthier status (reversal of disease progression).
19. sGC activator for use in non-proliferative diabetic retinopathy according to Claim 15, whereas non-proliferative diabetic retinopathy is complicated by ischemic macular edema.
20. sGC activator for use according to Claim 19, whereas ischemic macular edema is caused by DR, branch retinal vein occlusion or radiation retinopathy.
21. sGC activator for use according to any of Claims 1 to 11, whereas the eye disease is selected from a list of optic neuropathies consisting of glaucomatous optic neuropathy, ischemic optic neuropathy, traumatic optic neuropathy, non-arteritic anterior ischemic optic neuropathy, optic neuropathy, leber's hereditary optic neuropathy, methanol associated optic neuropathy and age-related macular degeneration.
22. sGC activator for use according to Claim 21, wherein the optic neuropathy is glaucoma optic neuropathy.
23. sGC activator for use according to Claim 21, whereas the glaucomatous optic neuropathy is caused by acute closed angle glaucoma.
24. sGC activator for use according to Claims 1 to 11, whereas the eye disease is associated with cataract formation.
25. sGC activator for use according to Claim 24, whereas the cataract formation cause is selected from a list consisting of age-related cataract, diabetes induced cataract, steroid induced cataract, traumatic cataract, congenital cataract.
26. sGC activator for use according to any of Claim 24, whereas the cataract formation cause is diabetes induced cataract secondary to type 1 or type 2 diabetes.
27. sGC activator for use according to any of Claim 24, whereas the cataract formation cause is diabetes induced cataract secondary to type 1 diabetes.
28. Combination for use according to any of Claims 1 to 27 comprising at least one sGC activator according to any of Claims 1 to 11 and at least one compound selected from the group consisting of inhibitors of phosphodiesterases 1, 2 and/or 5, calcium, vitamin D and metabolites of vitamin D, bisphosphonates, selected from etidronate, clodronate, tiludronate, teriparatide, pamidronate, neridronate, olpadronate, alendronate, ibandronate, risedronate, and zoledronate, strontium ranelate, active ingredients suitable for hormone replacement therapy in osteoporosis, selected from estrogen and a combination of estrogen and progesterone, selective estrogen receptor modulators, parathyroid hormone and analogs of parathyroid hormone, modulators of receptor activator of nuclear factor kappa-B ligand, sclerostin inhibitors, and TGF-β inhibitors.
29. Combination for use according to Claim 28, wherein the at least one inhibitor of phosphodiesterase 5 is selected from the group consisting of sildenafil, vardenafil, tadalafil and avanafil.
30. Combination for use according to any of Claims 1 to 27 comprising at least one sGC activator according to any of Claims 1 to 11 and at least one mineralocorticoid-receptor antagonist selected from the group consisting of spironolactone, eplerenone or finerenone.
31. Pharmaceutical composition for use according to any of Claims 1 to 27 comprising at least one sGC activator according to any of Claims 1 to 11 and one or more inert non-toxic pharmaceutically suitable excipients.
32. Pharmaceutical composition for use according to any of Claims 1 to 27 comprising at least one sGC activator according to any of Claims 1 to 11 and one or more inert non-toxic pharmaceutically suitable excipients wherein the formulation is in form of an osmotic release system.
33. Pharmaceutical composition for use according to any of Claims 1 to 27 comprising a sGC activator according to any of Claims 1 to 11 and one or more inert non-toxic pharmaceutically suitable excipients, characterized in that the sGC activator is selected form the group consisting of compound of formula I, (I-A), (I-B), (I-C), (I-D), (I-D-R), (I-E), (I-E-R), (I-F), (I-G), (I-H), (I-I), (I-J), (I-K), preferably ((I-D), (I-D-R) or (I-E), (I-E-R) or (I-H) or (I-I) and that the sGC activator is present in an amount of 0.1 mg to 500 mg, preferably 1 mg to 120 mg, most preferable 2.5 mg to 50 mg or 2.5 mg to 60 mg.
34. Pharmaceutical composition for use in the oral treatment and/or prophylaxis of an eye disease, wherein the eye diseases is NPDR, comprising a sGC activator according to according to any of Claims 1 to 11 and one or more inert non-toxic pharmaceutically suitable excipients, characterized in that the sGC activator is selected form the group consisting of compound of formula ((I-D), (I-D-R), (I-E), (I-E-R) or (I-H) or (I-I) and that the sGC activator is present in an amount of 0.1 mg to 500 mg, preferably 1 mg to 120 mg, most preferable 2.5 mg to 50 mg or 2.5 mg to 60 mg.
35. Pharmaceutical composition for use in the oral treatment and/or prophylaxis of an eye disease, wherein the eye diseases is NPDR, comprising a sGC activator according to according to any of Claims 1 to 11 and one or more inert non-toxic pharmaceutically suitable excipients, characterized in that the sGC activator is selected form the group consisting of compound of formula ((I-D), (I-D-R), (I-E) or (I-E-R) and that the sGC activator is present in an amount of 0.1 mg to 500 mg, preferably 1 mg to 120 mg, most preferable 2.5 mg to 50 mg or 2.5 mg to 60 mg.
36. Pharmaceutical composition for use in the oral treatment and/or prophylaxis of an eye disease, wherein the eye diseases is NPDR, comprising a sGC activator according to according to any of Claims 1 to 11 and one or more inert non-toxic pharmaceutically suitable excipients, characterized in that the sGC activator is selected form the group consisting of compound of formula ((I-D), (I-D-R), (I-E) or (I-E-R) and that the sGC activator is present in an amount of 0.1 mg to 500 mg, preferably 1 mg to 120 mg, most preferable 2.5 mg to 50 mg or 2.5 mg to 60 mg, also most preferably 4 mg to 45 mg or 4 to 90 mg or 4 to 180 mg.
37. Pharmaceutical composition for use according to any of Claims 1 to 27 comprising a combination according to Claims 28 or 29 and one or more inert non-toxic pharmaceutically suitable excipients.
38. Pharmaceutical composition for use according to any of Claims 1 to 27 comprising a combination according to Claims 28 or 29 and one or more inert non-toxic pharmaceutically suitable excipients, characterized in that the sGC activator is selected form the group consisting of compound of formula formula I, (I-A), (I-B), (I-C), (I-D), (I-D-R), (I-E), (I-E-R), (I-F), (I-G), (I-H), (I-I), (I-J), (I-K), preferably ((I-D), (I-D-R) or (I-E), (I-E-R) or (I-H) or (I-I) and that the sGC activator is present in an amount of 0.1 mg to 500 mg, preferably 1 mg to 120 mg, most preferable 2.5 mg to 50 mg or 2.5 mg to 60 mg.
39. Pharmaceutical composition for use according to any of Claims 1 to 27 comprising a combination according to Claims 28 or 29 and one or more inert non-toxic pharmaceutically suitable excipients, characterized in that the sGC activator is selected form the group consisting of compound of formula ((I-D), (I-D-R) or (I-E), (I-E-R) and that the sGC activator is present in an amount of 0.1 mg to 500 mg, preferably 1 mg to 120 mg, most preferable 2.5 mg to 50 mg or 2.5 mg to 60 mg, also most preferably 4 mg to 45 mg or 4 to 90 mg or 4 to 180 mg.
40. Method for the treatment and/or prevention of an eye disease selected from a list consisting of non-proliferative diabetic retinopathy and diabetic macular edema in humans and animals by administration of an effective amount of at least one sGC activator according to any of Claims 1 to 11 or a pharmaceutical composition as defined in any of Claims 31 to 39.
41. Method for the treatment and/or prevention of an eye disease selected from a list consisting of non-proliferative diabetic retinopathy and diabetic macular edema in humans and animals by administration of an effective amount of at least one sGC activator selected from the group consisting of a compound of formula ((I-D), (I-D-R) or (I-E), (I-E-R) or a pharmaceutical composition as defined in any of Claims 31 to 39.
42. Method for the oral treatment and/or prevention of an eye disease selected from a list consisting of non-proliferative diabetic retinopathy, optic neuropathies and cataract in humans and animals by administration of an effective amount of at least one sGC activator according to any of Claims 1 to 11 or a pharmaceutical composition as defined in any of Claims 31 to 39.
43. Method for the oral treatment and/or prevention of an eye disease selected from a list consisting of non-proliferative diabetic retinopathy, glaucoma optic neuropathy, whereas the eye disease is associated with cataract formation and diabetic macular edema in humans and animals by administration of an effective amount of at least one sGC activator according to any of Claims 1 to 10 or a pharmaceutical composition as defined in any of Claims 31 to 39.

### EXPERIMENTAL SECTION

TABLE 1
Abbreviations

| | |
|---|---|
| Abbreviation | Meaning |

The following table lists the abbreviations used herein.

| | |
|---|---|
| BH₃ · THF | Borane-tetrahydrofuran |
| BINAP | 2,2′-Bis(diphenylphosphino)-1,1′-binaphthyl |
| br | broad (¹H-NMR signal) |
| CI | chemical ionisation |
| d | doublet (¹H-NMR signal) |
| d | day(s) |
| DAD | diode array detector |
| dd | double-doublet |
| DMF | N,N-dimethylformamide |
| DMSO | dimethylsulfoxide |
| ESI | electrospray (ES) ionisation |
| EtOAc | Ethyl acetate |
| h | hour(s) |
| HATU | 1-[Bis(dimethylamino)methylene]-1H-1,2,3- |
| | triazolo[4,5-b]pyridinium 3-oxide |
| | hexafluorophosphate, CAS 148893-10-1 |
| HPLC | high performance liquid chromatography |
| LC-MS | liquid chromatography mass spectrometry |
| m | multiplet (¹H-NMR signal) |
| M | molar |
| min | minute(s) |
| MS | mass spectrometry |
| MTBE | methyl-tert-butylether |
| NaBH₄ | Sodium borohydride, sodium tetrahydroborate |
| NaHCO₃ | Sodium hydrogen carbonate |
| Na₂SO₄ | Sodium sulphate |
| NMR | nuclear magnetic resonance spectroscopy: chemical |
| | shifts (δ) are given in ppm. The chemical shifts |
| | were corrected by setting the DMSO signal to 2.50 ppm |
| | unless otherwise stated. |
| PDA | Photo Diode Array |
| Pd₂dba₃ | Tris(dibenzylideneacetone)dipalladium (0), |
| | CAS 51364-51-3 |
| Pd(PPh₃)₄ | Tetrakis(triphenylphosphane)palladium(0), |
| | CAS 14221-01-3 |
| quant. | quantitative |
| rac | racemic |
| Rₜ, Rt | retention time (as measured either with HPLC or |
| | UPLC) in minutes |
| RuPhos Pd G3 | (2-Dicyclohexylphosphino-2′,6′-diisopropoxy-1,1′- |
| | biphenyl)[2-(2′-amino-1,1′-biphenyl)]palladium(II) |
| | methanesulfonate, CAS 1445085-77-7 |
| s | singlet (¹H-NMR signal) |
| SFC | Supercritical Fluid Chromatography |
| SQD | Single-Quadrupole-Detector |
| t | triplet (¹H-NMR signal) |
| td | triple-doublet (¹H-NMR signal) |
| TFA | trifluoroacetic acid |
| THF | tetrahydrofuran |
| UPLC | ultra performance liquid chromatography |
| X-Phos | 2-Dicyclohexylphosphino-2′,4′,6′-triisopropylbiphenyl, |
| | CAS 564483-18-7 |

Other abbreviations not specified herein have their meanings customary to the skilled person.

The various aspects of the invention described in this application are illustrated by the following examples which are not meant to limit the invention in any way. All publications mentioned herein are incorporated by reference in their entirety.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

NMR peak forms are stated as they appear in the spectra, possible higher order effects have not been considered.

The ¹H-NMR data of selected compounds are listed in the form of ¹H-NMR peaklists. For each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensity), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of target compounds (also the subject of the invention), and/or peaks of impurities. The peaks of stereoisomers, and/or peaks of impurities are typically displayed with a lower intensity compared to the peaks of the target compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the target compounds by known methods (MestReC, ACD simulation, or by use of empirically evaluated expectation values), can isolate the peaks of target compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures). In the peak picking routine, as described in the Research Disclosure Database Number 605005, the parameter “MinimumHeight” can be adjusted between 1% and 4%. Depending on the chemical structure and/or depending on the concentration of the measured compound it may be reasonable to set the parameter “MinimumHeight”<1%.

In NMR spectra of mixtures of stereoisomers, numbers mentioned with “/” indicate that the stereoisomers show separate signals for the respective hydrogen atom, i.e. “ . . . / . . . (2s, 1H)” means that one hydrogen atom is represented by 2 singlets, each singlet from one or more different stereoisomer(s).

IUPAC names of the following intermediates and example compounds were generated using the ACD/Name software (batch version 14.00; Advanced Chemistry Development, Inc.) or the naming tool implemented in the BIOVIA Draw software (version 4.2 SPI; Dassault Systemes SE).

Analytical LC-MS methods

Method 1

MS instrument type: SHIMADZU LCMS-2020, Column: Kinetex EVO C18 30*2.1 mm, 5 um, mobile phase A: 0.0375% TFA in water (v/v), B: 0.01875% TFA in Acetonitrile (v/v), gradient: 0.0 min 0% B→0.8 min 95% B→1.2 min 95% B→1.21 min 5% B→1.55 min 5% B, flow rate: 1.5 mL/min, oven temperature: 50° C.; UV detection: 220 nm & 254 n.

Method 2

HPLC instrument type: SHIMADZU LCMS-2020, Column: Kinetex EVO C18 50*4.6 mm, 5 um, mobile phase A: 0.0375% TFA in water (v/v), B: 0.01875% TFA in Acetonitrile (v/v), gradient: 0.0 min 10% B→2.4 min 80% B→3.7 min 80% B→3.71 min 10% B→4.0 min 10% B, flow rate: 1.5 mL/min, oven temperature: 50° C.; UV detection: 220 nm & 215 nm & 254 n.

Method 3 (LC-MS)

Instrument MS: Thermo Scientific FT-MS; Instrument type UHPLC+: Thermo Scientific UltiMate 3000; Column: Waters, HSST3, 2.1×75 mm, C18 1.8 μm; Eluent A: 11 water+0.01% formic acid; Eluent B: 11 Acetonitrile+0.01% formic acid; Gradient: 0.0 min 10% B→2.5 min 95% B→3.5 min 95% B; oven: 50° C.; flow rate: 0.90 ml/min; UV-Detection: 210 nm/Optimum Integration Path 210-300 nm.

Method 4 (LC-MS)

Instrument: Waters ACQUITY SQD UPLC System; Column: Waters Acquity UPLC HSS T3 1.8 μm 50×1 mm; Eluent A: 11 water+0.25 ml formic acid, Eluent B: 11 Acetonitrile+0.25 ml formic acid; Gradient: 0.0 min 90% A→1.2 min 5% A→2.0 min 5% A; oven: 50° C.; flow rate: 0.40 ml/min; UV-Detection: 210 nm.

Method 5 (LC-MS)

Instrument: Waters ACQUITY SQD UPLC System; Column: Waters Acquity UPLC HSS T3 1.8 μm 50×1 mm; Eluent A: 11 water+0.25 ml formic acid, Eluent B: 11 Acetonitrile+0.25 ml formic acid; Gradient: 0.0 min 95% A→6.0 min 5% A→7.5 min 5% A; oven: 50° C.; flow rate: 0.35 ml/min; UV-Detection: 210 nm.

Method 6 (LC-MS)

Instrument: Agilent MS Quad 6150; HPLC: Agilent 1290; Column: Waters Acquity UPLC HSS T3 1.8 μm 50×2.1 mm; Eluent A: 1 l water+0.25 ml formic acid, Eluent B: 1 l Acetonitrile+0.25 ml formic acid; Gradient: 0.0 min 90% A→0.3 min 90% A→1.7 min 5% A→3.0 min 5% A oven: 50° C.; flow rate: 1.20 ml/min; UV-Detection: 205-305 nm.

Method 7 (LC-MS)

Instrument: Waters Single Quad MS System; Instrument Waters UPLC Acquity; Column: Waters BEH C18 1.7μ 50×2.1 mm; Eluent A: 1 l water+1.0 mL (25% aqueous Ammonia)/L, Eluent B: 1 l Acetonitrile; Gradient: 0.0 min 92% A→0.1 min 92% A→1.8 min 5% A→3.5 min 5% A; oven: 50° C.; flow rate: 0.45 mL/min; UV-Detection: 210 n.

Method 8 (LC-MS)

System MS: Waters TOF instrument; System UPLC: Waters Acquity I-CLASS; Column: Waters Acquity UPLC HSS T3 1.8 μm 50×1 mm; Eluent A: 1 l Water+0.100 ml 99% ige Formic acid, Eluent B: 1 l Acetonitrile+0.100 ml 99% ige Formic acid; Gradient: 0.0 min 90% A→1.2 min 5% A→2.0 min 5% A Oven: 50° C.; Flow: 0.40 ml/min; UV-Detection: 210 n.

Method 9 (LC-MS):

System MS: Waters TOF instrument; System UPLC: Waters Acquity I-CLASS; Column: Waters Acquity UPLC HSS T3 1.8 μm 50×1 mm; Eluent A: 1 l Water+0.100 ml 99% ige Formic acid, Eluent B: 1 l Acetonitrile+0.100 ml 99% ige Formic acid; Gradient: 0.0 min 95% A→6.0 min 5% A→7.5 min 5% A Oven: 50° C.; Flow: 0.35 ml/min; UV-Detection: 210 n.

Preparative HPLC Methods

Instrument: Waters Prep LC/MS System, column: Phenomenex Kinetex C18 5 μm 100×30 mm, UV-detection 200-400 n, room temperature, At-Column Injection (complete injection), eluent A: water, eluent B: acetonitrile, eluent C: 2% formic acid in water, eluent D: acetonitrile/water (80 vol. %/20 vol. %); flow: 80 ml/min, gradient profil: 0 to 2 min: eluent A 47 ml/min, eluent B 23 ml/min; 2 to 10 min: eluent A from 47 ml/min to 23 ml/min, eluent B from 23 ml/min to 47 ml/min; 10 to 12 min eluent A 0 ml/min and eluent B 70 ml/min; eluent C and eluent D have a constant flow of 5 ml/min each over the whole running time.

Microwave: Reactions employing microwave irradiation may be run with a Biotage Initator® microwave oven optionally equipped with a robotic unit. The reported reaction times employing microwave heating are intended to be understood as fixed reaction times after reaching the indicated reaction temperature.

When compounds according to the invention are purified by preparative HPLC by the above-described methods in which the eluents contain additives, for example trifluoroacetic acid, formic acid or ammonia, the compounds according to the invention may be obtained in salt form, for example as trifluoroacetate, formate or ammonium salt, if the compounds according to the invention contain a sufficiently basic or acidic functionality. Such a salt can be converted to the corresponding free base or acid by various methods known to the person skilled in the art.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process.

Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

Enantiomer 1 is an enantiomer which eluted first out of the column.

Enantiomer 2 is an enantiomer which eluted second out of the column.

Diastereomeric mixture 1 defines a compound where its starting material is defined as Enantiomer 1 and is reacted with a building block containing at least one chiral center and where the configuration is not defined

Diastereomeric mixture 2 defines a compound where its starting material is defined as Enantiomer 2 and is reacted with a building block containing at least one chiral center and where the configuration is not defined

Diastereomer 1 and Diastereomer 2 defines the two compounds resulting from the chiral separation of the diastereomeric mixture 1 described above.

Diastereomer 3 and Diastereomer 4 defines the two compounds resulting from the chiral separation of the diastereomeric mixture 2 described above.

Stereoisomer 1 defines a compound where its starting material is defined as Enantiomer 1 and is reacted with a building block containing at least one chiral center and where the configuration is defined Stereoisomer 2 defines a compound where its starting material is defined as Enantiomer 2 and is reacted with a building block containing at least one chiral center and where the configuration is defined

### Starting Compounds and Intermediates

### Intermediate 1A

### Example 1A

### Tert-butyl 3-{2-[(benzyloxy)carbonyl]hydrazino}piperidine-1-carboxylate (Racemate)

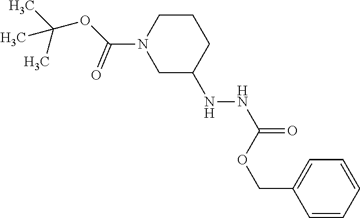 [SMILES: CC(C)(C)OC(=O)N1CCCC(NNC(=O)OCc2ccccc2)C1]

To a solution of tert-butyl 3-oxopiperidine-1-carboxylate [CAS No. 989-36-7] (300 g, 1.51 mol) in tetrahydrofuran (1.50 L) and Methanol (300 mL) was added benzyl hydrazinecarboxylate [CAS No. 5331-43-1] (250 g, 1.51 mol) at 25° C., then, the mixture was stirred at 25° C. for 1 h. Afterwards NaBH₄ (114 g, 3.01 mol) was added in portions to the mixture at 25° C. and stirred at 25° C. for 2 h. The reaction mixture was cooled to 10° C., and sat. NH₄Cl was added dropwise to pH˜6. The mixture was extracted with EtOAc (300 mL*2) and concentrated in vacuo. The residue was dissolved in MTBE (300 mL) and petroleum ether (300 mL) was added. The mixture was filtrated off and the precipitate was washed with petroleum ether (100 mL) affording the title compound (400 g, 1.14 mol, 76.0% yield) as a white solid.

LC-MS: (Method 1) Rₜ=0.832 min, MS (M−100+1=250.4).

### Example 2A

### Tert-butyl 3-hydrazinopiperidine-1-carboxylate acetic acid adduct (Racemate)

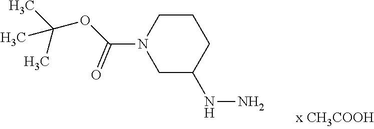

To a solution of tert-butyl 3-{2-[(benzyloxy)carbonyl]hydrazino}piperidine-1-carboxylate (prepared in analogy to Example 1A, 1.20 kg, 3.43 mol) in ethanol (11.0 L) was added acetic acid (415 g, 6.91 mol, 395 mL) and Pd/C (120 g, 20% purity) under H₂ (15 Psi). The mixture was stirred at 25° C. for 12 h. The mixture was filtrated and the precipitate was washed with ethanol (11.0 L) to give a solution of the title compound in ethanol (945 g, acetic acid salt) as a black liquid, the filtrate was used for the next step without purification.

¹H-NMR (400 MHz, CDCl₃) δ [ppm]: 7.52 (s, 5H), 3.59 (d, J=6.0 Hz, 12H), 3.30-3.24 (m, 2H), 2.75-2.71 (m, 2H), 1.38-1.34 (m, 1H), 1.20-1.18 (m, 1H), 1.10 (s, 9H)

LC-MS: (Method 1) Rₜ=0.263 min, MS (M−56+1=160.2)

### Example 3A

### Ethyl 2-(ethoxymethylidene)-4,4-difluoro-3-oxobutanoate

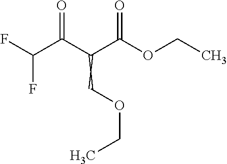

A solution of ethyl 4,4-difluoro-3-oxobutanoate [CAS No. 352-24-9] (120 g, 722 mmol) and (diethoxymethoxy)ethane (240 ml, 1.4 mol) in acetic acid anhydride (200 ml, 2.2 mol) was stirred overnight at 140° C. and evaporated to dryness affording 155 g (quant.) of the titlte compound which was used in the next step without further purification.

¹H-NMR (600 MHz, CDCl₃) δ [ppm]: 1.306 (6.05), 1.318 (16.00), 1.330 (14.48), 1.341 (4.56), 1.428 (5.99), 1.436 (5.01), 1.440 (12.20), 1.448 (9.25), 1.451 (6.31), 1.460 (4.48), 2.095 (1.59), 2.225 (1.56), 4.247 (1.97), 4.260 (5.79), 4.271 (5.85), 4.277 (1.55), 4.283 (2.00), 4.289 (4.40), 4.301 (4.37), 4.308 (2.03), 4.313 (1.64), 4.320 (5.74), 4.332 (5.78), 4.340 (1.60), 4.344 (2.01), 4.351 (4.21), 4.364 (4.20), 4.375 (1.37), 6.262 (1.79), 6.339 (1.35), 6.352 (3.56), 6.429 (2.63), 6.442 (1.72), 6.519 (1.28), 7.867 (5.48), 7.880 (7.31).

### Example 4A

### Tert-butyl 3-[5-(difluoromethyl)-4-(ethoxycarbonyl)-1H-pyrazol-1-yl]piperidine-1-carboxylate

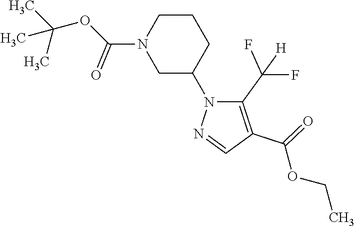 [SMILES: CCOC(=O)c1cnn(C2CCCN(C(=O)OC(C)(C)C)C2)c1C(F)F]

To a mixture of tert-butyl 3-hydrazinopiperidine-1-carboxylate acetic acid (Example 2A, 945 g, 3.43 mol) in ethanol (20.0 L) was added ethyl 2-(ethoxymethylene)-4,4-difluoro-3-oxobutanoate (prepared in analogy to Example 3A, 840 g, 3.78 mol). The mixture was stirred at 25° C. for 12 h. The reaction mixture was concentrated. The residue was poured into saturated NaHCO₃ aqueous solution (10.0 L), and extracted with Ethyl acetate (10.0 L*2). The combined organic layer was washed with brine (10.0 L), dried over Na₂SO₄, filtered and concentrated. The residue was purified by column chromatography on silica gel eluted with Petroleum ether:Ethyl acetate (50:1-25:1-10:1, R_{f}=0.3) affording 530 g (41.4% yield) of the title compound.

¹H-NMR (400 MHz, CDCl₃) δ [ppm]: 7.84 (s, 1H), 7.51 (t, J=12.8 Hz, 1H), 4.47-4.41 (m, 1H), 4.30-4.10 (m, 4H), 3.19-3.13 (m, 1H), 2.69 (s, 1H), 2.15-2.10 (m, 2H), 1.83-1.78 (m, 1H), 1.60-1.55 (m, 1H), 1.40 (s, 9H), 1.32-1.29 (m, 3H)

LC-MS (Method 1) Rₜ=0.992 min, MS (M−56+1=318.0).

### Example 5A

### Ethyl 5-(difluoromethyl)-1-(piperidin-3-yl)-1H-pyrazole-4-carboxylate (Racemate)

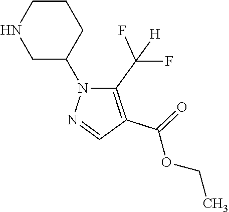 [SMILES: CCOC(=O)c1cnn(C2CCCNC2)c1C(F)F]

Tert-butyl 3-[5-(difluoromethyl)-4-(ethoxycarbonyl)-1H-pyrazol-1-yl]piperidine-1-carboxylate (prepared in analogy to Example 4A, 593 g, 1.59 mol) was added to a solution of hydrogen chloride in dioxane (4 M, 2.50 L), the mixture was stirred at 25° C. for 12 h. The mixture was evaporated and the residue was dissolved in 1.00 L water and extracted with MTBE 500 mL. The aqueous phase was separated and adjusted pH to 8-9 with NaHCO₃. The aqueous phase was extracted with dichloromethane (1.00 L×2), and the combined organic phases were washed with brine (1.00 L), dried over Na₂SO₄ and concentrated to give 350 g (80.6% yield) of the title compound.

¹H-NMR (400 MHz, CDCl₃) δ [ppm]: 7.87 (s, 1H), 7.54 (t, J=12.8 Hz, 1H), 4.55-4.54 (m, 1H), 4.34-4.28 (m, 2H), 3.25-3.03 (m, 3H), 2.71-2.65 (m, 1H), 2.19-1.86 (m, 4H), 1.63-1.60 (m, 1H), 1.35 (t, J=7.2 Hz, 3H)

LC-MS: (Method 1) Rₜ=0.644 min, MS (M+1)=274.6

In analogy to Example 5A, ethyl 5-(difluoromethyl)-1-(piperidin-3-yl)-1H-pyrazole-4-carboxylate (Racemate) was prepared using different protecting groups. The two enantiomers were separated by SFC [sample preparation: 20 g were dissolved in 500 ml methanol; injection volume: 15 ml; column: Daicel AZ SCF 20 μm, 400×50 mm; eluent: carbone dioxide/methanol/aqueous ammonia (1%) 80:19:1 to 60:39:1; flow rate: 400 ml/min; temperature: 40° C.; UV detection: 220 nm]. After separation, 8.1 g of enantiomer 1 (Example 6A), which eluted first, and 8.0 g of enantiomer 2 (Example 7A), which eluted later, were isolated.

### Example 6A

### Ethyl 5-(difluoromethyl)-1-(piperidin-3-yl)-1H-pyrazole-4-carboxylate (Enantiomer 1)

For separation conditions see Example 5A.

Analytical SFC: Rₜ=0.980 min, e.e. =100% [Column Chiralpak IC-3: 50×4.6 mm; eluent: CO₂/[methanol+0.2% diethyl amine]: 90:10 flow rate: 3.0 ml/min; temperature: 25° C.; UV detection: 220 nm]. ¹H-NMR (400 MHz, DMSO-d6) δ[ppm]: 8.00 (s, 1H), 7.75-7.44 (m, 1H), 4.50-4.36 (m, 1H), 4.33-4.18 (m, 2H), 3.10-2.95 (m, 1H), 2.91-2.76 (m, 2H), 2.48-2.33 (m, 2H), 2.08-1.94 (m, 2H), 1.81-1.66 (m, 1H), 1.62-1.40 (m, 1H), 1.37-1.21 (m, 3H).

### Example 7A

### Ethyl 5-(difluoromethyl)-1-(piperidin-3-yl)-1H-pyrazole-4-carboxylate (Enantiomer 2)

For separation conditions see Example 5A.

Analytical SFC: Rₜ=1.227 min, e.e. =97% [Column Chiralpak IC-3: 50×4.6 mm; eluent: CO2/[methanol+0.2% diethyl amine]: 90:10 flow rate: 3.0 ml/min; temperature: 25° C.; UV detection: 220 nm]. ¹H-NMR (400 MHz, DMSO-d6) δ[ppm]: 8.01 (s, 1H), 7.75-7.43 (m, 1H), 4.50-4.37 (m, 1H), 4.27 (q, 2H), 3.09-2.97 (m, 1H), 2.94-2.81 (m, 2H), 2.47-2.34 (m, 2H), 2.06-1.92 (m, 2H), 1.79-1.66 (m, 1H), 1.60-1.41 (m, 1H), 1.29 (t, 3H).

### Example 8A

### 2-Bromo-4-chloro-1-[(4-methoxyphenyl)methoxy]benzene

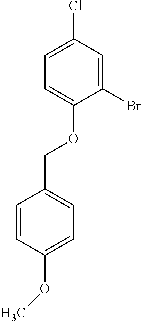 [SMILES: COc1ccc(COc2ccc(Cl)cc2Br)cc1]

A solution of 2-bromo-4-chlorophenol [CAS No. 695-96-5] (10.0 g, 48.2 mmol) in acetone (75 ml) was treated with potassium carbonate (13.3 g, 96.4 mmol) and potassium iodide (12.0 g, 72.3 mmol) and 1-(chloromethyl)-4-methoxybenzene (7.55 g, 48.2 mmol). The resulting mixture was stirred ˜19 hours at 70° C. The reaction mixture was diluted with water and extracted twice with ethyl acetate. The combined organic layers were dried over sodium sulphate and evaporated. The residue was purified by flash chromatography (silica gel, cyclohexane/ethyl acetate gradient) affording 13.8 g (86% yield) of the title compound.

LC-MS (Method 3): Rₜ=2.48 min; MS (ESIneg): m/z=324 [M−H]⁻

¹H-NMR (600 MHz, DMSO-d6) δ[ppm]: 3.349 (10.98), 5.124 (16.00), 6.949 (0.87), 6.954 (8.36), 6.957 (2.68), 6.965 (2.83), 6.968 (8.92), 6.973 (1.00), 7.218 (5.23), 7.233 (6.21), 7.380 (0.90), 7.384 (7.80), 7.399 (7.44), 7.402 (4.47), 7.406 (3.89), 7.417 (3.04), 7.421 (3.07), 7.697 (6.51), 7.702 (6.34).

### Example 9A

### Ethyl 1-[1-{5-chloro-2-[(4-methoxyphenyl)methoxy]phenyl}piperidin-3-yl]-5-(difluoromethyl)-1H-pyrazole-4-carboxylate (Enantiomer 1)

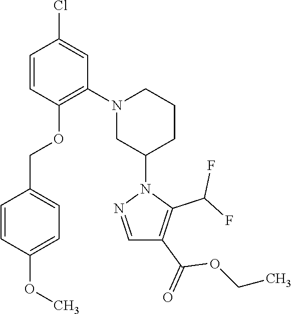 [SMILES: CCOC(=O)c1cnn(C2CCCN(c3cc(Cl)ccc3OCc3ccc(OC)cc3)C2)c1C(F)F]

Under argon, a solution of 2-bromo-4-chloro-1-[(4-methoxyphenyl)methoxy]benzene (prepared in analogy to Example 8A, 10.0 g, 30.5 mmol) and ethyl 5-(difluoromethyl)-1-[piperidin-3-yl]-1H-pyrazole-4-carboxylate (prepared in analogy to Example 6A, Enantiomer 1, 8.34 g, 30.5 mmol) in 1,4-dioxane (100 ml) was treated with caesium carbonate (29.8 g, 91.6 mmol), Pd₂dba₃ (2.80 g, 3.05 mmol) and rac-BINAP (3.80 g, 6.10 mmol) and the resulting mixture was stirred overnight at 100° C. The reaction mixture was combined with a 500 mg test reaction, filtered over celite, rinsed with ethyl acetate and evaporated. The residue was retaken in water and extracted three times with ethyl acetate. The combined organic layers were washed with a saturated solution of sodium chloride, dried over sodium sulphate and evaporated. The residue was purified by flash chromatography (silica gel, cyclohexane/ethyl acetate gradient) affording 10.1 g (60% yield) of the title compound.

LC-MS (Method 4): Rₜ=1.44 min; MS (ESIpos): m/z=520 [M+H]⁺

### Example 10A

### Ethyl 1-[1-(5-chloro-2-hydroxyphenyl)piperidin-3-yl]-5-(difluoromethyl)-1H-pyrazole-4-carboxylate (Enantiomer 1)

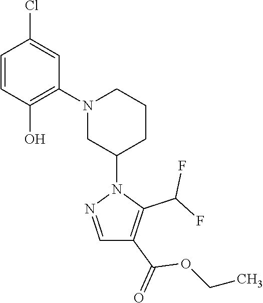 [SMILES: CCOC(=O)c1cnn(C2CCCN(c3cc(Cl)ccc3O)C2)c1C(F)F]

A solution of ethyl 1-[1-{5-chloro-2-[(4-methoxyphenyl)methoxy]phenyl}piperidin-3-yl]-5-(difluoromethyl)-1H-pyrazole-4-carboxylate (Example 9A, Enantiomer 1, 10.1 g, 19.4 mmol) in dichloromethane (200 ml) was treated with trifluoroacetic acid and stirred over night at room temperature. The reaction mixture was evaporated. The residue was retaken in ethyl acetate and washed once with water, once with a saturated solution of sodium hydrogencarbonate and finally once with a saturated solution of sodium chloride. The organic phase was dried over sodium sulphate and evaporated. The residue was purified by flash chromatography (silica gel, cyclohexane/ethyl acetate gradient) affording 7.17 g (83% purity, 77% yield) of the title compound.

LC-MS (Method 8): Rₜ=1.26 min; MS (ESIpos): m/z=400 [M+H]⁺

### Example 11A

### Ethyl 1-[1-{5-chloro-2-[(trifluoromethanesulfonyl)oxy]phenyl}piperidin-3-yl]-5-(difluoromethyl)-1H-pyrazole-4-carboxylate (Enantiomer 1)

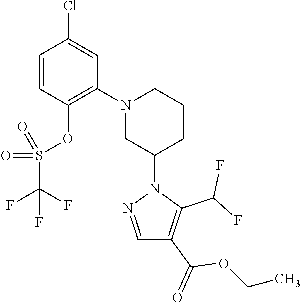 [SMILES: CCOC(=O)c1cnn(C2CCCN(c3cc(Cl)ccc3OS(=O)(=O)C(F)(F)F)C2)c1C(F)F]

Under argon, a solution of ethyl 1-[1-(5-chloro-2-hydroxyphenyl)piperidin-3-yl]-5-(difluoromethyl)-1H-pyrazole-4-carboxylate (Example 10A, Enantiomer 1, 7.17 g, 83% purity, 14.9 mmol) in dichloromethane (160 ml) was treated with triethylamine (5.2 ml, 37 mmol) and cooled to 0° C. Trifluoromethanesulfonic anhydride was added dropwise and the resulting mixture was stirred 45 minutes at 0° C. The reaction mixture was diluted with dichloromethane (150 ml) and washed three times with water. The organic phase was dried over sodium sulphate and evaporated. The residue was purified by flash chromatography (silica gel, cyclohexane/ethyl acetate gradient) affording 7.89 g (quant.) of the title compound.

LC-MS (Method 4): Rₜ=1.47 min; MS (ESIpos): m/z=532 [M+H]⁺

### Example 12A

### Ethyl 1-[1-{5-chloro-2-[(4-methoxyphenyl)methoxy]phenyl}piperidin-3-yl]-5-(difluoromethyl)-1H-pyrazole-4-carboxylate (Enantiomer 2)

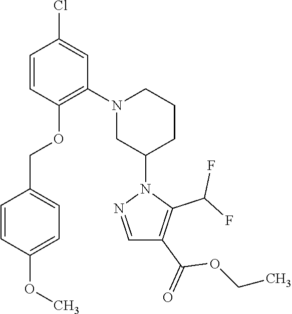 [SMILES: CCOC(=O)c1cnn(C2CCCN(c3cc(Cl)ccc3OCc3ccc(OC)cc3)C2)c1C(F)F]

Under argon, a solution of ethyl 5-(difluoromethyl)-1-[piperidin-3-yl]-1H-pyrazole-4-carboxylate (prepared in analogy to Example 7A, Enantiomer 2, 43.6 g, 160 mmol) and 2-bromo-4-chloro-1-[(4-methoxyphenyl)methoxy]benzene (prepared in analogy to Example 8A, 52.3 g, 160 mmol) in 1,4-dioxane (680 ml) was treated with Pd₂(dba)₃ (14.6 g, 16.0 mmol), rac-BINAP (19.9 g, 31.9 mmol) and freshly ground caesium carbonate (156 g, 479 mmol) and stirred 18 hours at 100° C. The reaction mixture was diluted with ethyl acetate and a 10% solution of sodium chloride, filtered over Celite and rinsed with ethyl acetate. The aqueous phase of the filtrate was extracted with ethyl acetate. The combined organic layers were washed with a 10% solution of sodium chloride, dried over sodium sulphate and evaporated. The residue was purified flash chromatography over silica gel (dichloromethane/petrol ether 4:1) affording 42 g (82% yield) of the title compound.

LC-MS (Method 3): Rₜ=2.78 min; MS (ESIpos): m/z=520 [M+H]⁺

¹H-NMR (400 MHz, DMSO-d6) δ[ppm]: 1.272 (3.65), 1.290 (7.68), 1.307 (3.76), 1.686 (0.44), 1.717 (0.54), 1.852 (0.73), 1.885 (0.50), 1.989 (0.47), 2.019 (0.56), 2.058 (0.99), 2.084 (0.61), 2.587 (0.51), 2.616 (0.89), 2.642 (0.45), 3.030 (0.76), 3.057 (1.51), 3.084 (0.83), 3.447 (0.72), 3.474 (0.69), 3.613 (0.74), 3.640 (0.67), 3.737 (16.00), 4.251 (1.13), 4.269 (3.48), 4.287 (3.45), 4.304 (1.12), 4.624 (0.40), 4.639 (0.48), 4.650 (0.76), 4.661 (0.51), 5.035 (6.45), 6.872 (3.47), 6.893 (5.67), 6.947 (0.98), 6.952 (0.85), 6.968 (1.72), 6.974 (1.67), 7.017 (2.84), 7.039 (1.57), 7.305 (3.66), 7.326 (3.43), 7.340 (0.56), 7.380 (0.41), 7.439 (0.93), 7.463 (0.64), 7.476 (0.48), 7.569 (1.65), 7.699 (0.76), 8.044 (3.66).

### Example 13A

### Ethyl 1-[1-(5-chloro-2-hydroxyphenyl)piperidin-3-yl]-5-(difluoromethyl)-1H-pyrazole-4-carboxylate (Enantiomer 2)

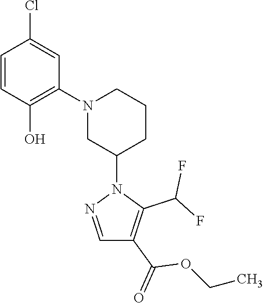 [SMILES: CCOC(=O)c1cnn(C2CCCN(c3cc(Cl)ccc3O)C2)c1C(F)F]

A solution of ethyl 1-[1-{5-chloro-2-[(4-methoxyphenyl)methoxy]phenyl}piperidin-3-yl]-5-(difluoromethyl)-1H-pyrazole-4-carboxylate (prepared in analogy to Example 12A, Enantiomer 2, 67.5 g, 130 mmol) in dichloromethane (1.0 l) was treated with trifluoroacetic acid (100 ml, 1.3 mol) and stirred overnight at room temperature. The reaction mixture was diluted with water (750 ml) and carefully treated with a 10% solution of sodium carbonate (450 ml) until no more carbon dioxide was generated. The organic phase was dried over sodium sulphate and evaporated affording 52 g (90% yield) of the title compound which was used in the next step without further purification.

LC-MS (Method 3): Rₜ=2.42 min; MS (ESIpos): m/z=400 [M+H]⁺

### Example 14A

### Ethyl 1-[1-{5-chloro-2-[(trifluoromethanesulfonyl)oxy]phenyl}piperidin-3-yl]-5-(difluoromethyl)-1H-pyrazole-4-carboxylate (Enantiomer 2)

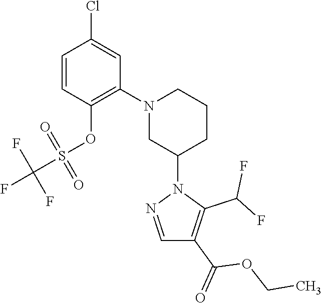 [SMILES: CCOC(=O)c1cnn(C2CCCN(c3cc(Cl)ccc3OS(=O)(=O)C(F)(F)F)C2)c1C(F)F]

A solution of ethyl 1-[1-(5-chloro-2-hydroxyphenyl)piperidin-3-yl]-5-(difluoromethyl)-1H-pyrazole-4-carboxylate (Example 13A, Enantiomer 2, 52.0 g, 117 mmol) and triethylamine (49 ml, 350 mmol) in dichloromethane (330 ml) was cooled to −50° C. Trifluoromethanesulfonic acid (28 ml, 160 mmol) was added dropwise and the resulting mixture was stirred 1 hour at −50° C. The reaction mixture was then diluted with dichloromethane (330 ml) and water (370 ml). The aqueous phase was extracted with dichloromethane (330 ml). The combined organic layers were washed with (370 ml), dried over sodium sulphate and evaporated. The resulting mixture was purified by flash chromatography (silica gel, dichloromethane/petrol ether 6:4) affording 60 g (96% yield) of the title compound.

LC-MS (Method 3): Rₜ=2.74 min; MS (ESIpos): m/z=532 [M+H]⁺

¹H-NMR (600 MHz, DMSO-d6) δ[ppm]: −0.021 (0.65), 1.082 (0.51), 1.270 (7.69), 1.282 (16.00), 1.294 (7.63), 1.772 (0.48), 1.780 (0.51), 1.787 (0.63), 1.793 (0.66), 1.801 (0.62), 1.808 (0.60), 1.910 (1.25), 1.914 (0.99), 1.927 (0.67), 1.932 (0.89), 2.068 (0.72), 2.075 (1.03), 2.086 (2.45), 2.091 (2.40), 2.100 (1.41), 2.792 (0.71), 2.796 (0.83), 2.812 (1.48), 2.816 (1.50), 2.832 (0.83), 2.836 (0.72), 3.142 (1.17), 3.161 (1.04), 3.201 (1.21), 3.219 (2.80), 3.237 (1.83), 3.278 (1.37), 3.285 (1.56), 4.251 (2.26), 4.263 (7.09), 4.275 (7.06), 4.287 (2.20), 4.755 (0.50), 4.765 (0.90), 4.773 (0.89), 4.781 (0.90), 4.791 (0.49), 5.734 (2.17), 7.261 (2.19), 7.265 (2.27), 7.275 (2.69), 7.279 (2.82), 7.391 (4.65), 7.406 (3.75), 7.431 (4.73), 7.435 (4.51), 7.492 (1.26), 7.579 (2.61), 7.666 (1.07), 8.026 (6.37).

### Example 15A

### Tert-butyl 4-(4′-chloro-2′-{3-[5-(difluoromethyl)-4-(ethoxycarbonyl)-1H-pyrazol-1-yl]piperidin-1-yl}[1,1′-biphenyl]-4-yl)piperazine-1-carboxylate (Enantiomer 2)

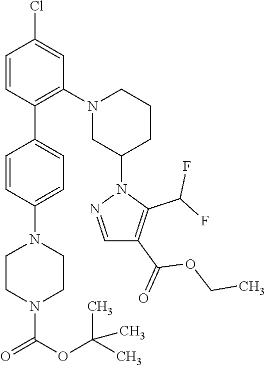 [SMILES: CCOC(=O)c1cnn(C2CCCN(c3cc(Cl)ccc3-c3ccc(N4CCN(C(=O)OC(C)(C)C)CC4)cc3)C2)c1C(F)F]

Under argon, a solution of ethyl 1-[1-{5-chloro-2-[(trifluoromethanesulfonyl)oxy]phenyl}piperidin-3-yl]-5-(difluoromethyl)-1H-pyrazole-4-carboxylate (Example 14A, Enantiomer 2, 57.0 g, 107 mmol) and tert-butyl 4-[4-(4,4,5,5-tetramethyl-1,3,2-dioxaborolan-2-yl)phenyl]piperazine-1-carboxylate [CAS No. 470478-90-1] (49.9 g, 129 mmol) in toluene (600 ml) and ethanol (600 ml) was treated with an aqueous solution of sodium carbonate (160 ml, 2.0 M, 320 mmol) and Tetrakis(triphenylphosphine)palladium(0) (6.19 g, 5.36 mmol). The resulting mixture was stirred 4 hours at 100° C. The reaction mixture was cooled to room temperature, filtered over Celite, washed with ethyl acetate and evaporated. The residue was purified by flash chromatography (silica gel, petrol ether/ethyl acetate 9:1 to 8:2) affording 62 g (89% yield) of the title compound.

LC-MS (Method 3): Rₜ=3.15 min; MS (ESIpos): m/z=644 [M+H]⁺

### Example 16A

### Ethyl 1-{1-[4-chloro-4′-(piperazin-1-yl)[1,1′-biphenyl]-2-yl]piperidin-3-yl}-5-(difluoromethyl)-1H-pyrazole-4-carboxylate hydrochloride (Enantiomer 2)

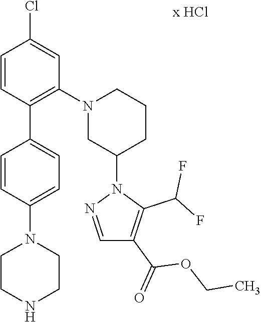 [SMILES: CCOC(=O)c1cnn(C2CCCN(c3cc(Cl)ccc3-c3ccc(N4CCNCC4)cc3)C2)c1C(F)F]

A solution of tert-butyl 4-(4′-chloro-2′-{(3-[5-(difluoromethyl)-4-(ethoxycarbonyl)-1H-pyrazol-1-yl]piperidin-1-yl}[1,1′-biphenyl]-4-yl)piperazine-1-carboxylate (Example 15A, Enantiomer 2, 60.0 g, 93.1 mmol) in dichloromethane (250 ml) was treated with a solution of hydrogen chloride in dioxane (230 ml, 4.0 M, 930 mmol). The resulting mixture was stirred 3 hours at room temperature and evaporated. The residue was co-evaporated twice with diethyl ether (250 ml×2), stirred 4 days in diisopropyl ether. The suspension was filtered, the solid was washed twice with diisopropyl ether affording 57 g (quant.) of the title compound.

LC-MS (Method 3): Rₜ=1.78 min; MS (ESIpos): m/z=544 [M+H]⁺

¹H-NMR (400 MHz, DMSO-d6) δ[ppm]: 1.029 (13.49), 1.044 (13.77), 1.262 (7.53), 1.280 (16.00), 1.297 (7.81), 1.496 (0.79), 1.506 (0.62), 1.527 (0.91), 1.559 (0.40), 1.716 (1.24), 1.749 (0.95), 1.888 (0.84), 1.897 (0.78), 1.918 (0.98), 1.926 (0.93), 1.966 (1.38), 1.995 (0.69), 2.580 (1.54), 2.606 (0.83), 2.992 (1.21), 3.018 (2.69), 3.044 (2.33), 3.063 (1.24), 3.435 (5.96), 3.448 (7.25), 3.460 (5.00), 3.570 (5.78), 3.586 (0.87), 3.601 (1.12), 3.616 (0.85), 4.227 (5.38), 4.238 (6.62), 4.256 (9.26), 4.273 (7.97), 4.291 (2.70), 4.444 (0.41), 4.455 (0.77), 4.470 (0.89), 4.481 (1.31), 4.491 (0.92), 4.507 (0.68), 7.045 (6.02), 7.067 (6.86), 7.074 (5.10), 7.079 (5.42), 7.099 (2.25), 7.104 (1.49), 7.120 (3.55), 7.125 (3.10), 7.164 (6.27), 7.185 (3.37), 7.383 (1.62), 7.483 (6.90), 7.505 (6.40), 7.513 (3.75), 7.643 (1.34), 8.005 (5.77), 9.399 (1.97).

### Example 17A

### Ethyl 1-[1-{4-chloro-4′-[4-(2-methylpropyl)piperazin-1-yl][1,1′-biphenyl]-2-yl}piperidin-3-yl]-5-(difluoromethyl)-1H-pyrazole-4-carboxylate (Enantiomer 2)

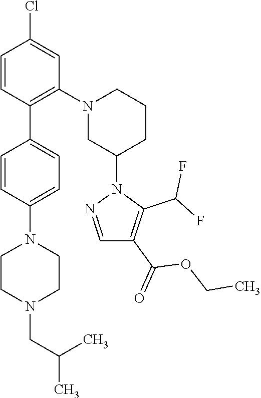 [SMILES: CCOC(=O)c1cnn(C2CCCN(c3cc(Cl)ccc3-c3ccc(N4CCN(CC(C)C)CC4)cc3)C2)c1C(F)F]

A solution of ethyl 1-{1-[4-chloro-4′-(piperazin-1-yl)[1,1′-biphenyl]-2-yl]piperidin-3-yl}-5-(difluoromethyl)-1H-pyrazole-4-carboxylate hydrogen chloride (Example 16A, Enantiomer 2, 52.0 g, 84.3 mmol) in THF was treated with N,N-diisopropylethylamine (59 ml, 340 mmol) and 2-methylpropanal [CAS No. 78-84-2] (38 ml, 420 mmol) and stirred 1 hour at room temperature. Sodium triacetoxyborohydride (71.5 g, 337 mmol) was then added and the resulting mixture was stirred 18 hours at room temperature. The reaction mixture was diluted with an aqueous solution of sodium hydrogen carbonate (10%) and ethyl acetate. The aqueous layer was extracted twice with ethyl acetate. The combined organic layers were washed with an aqueous solution of sodium chloride, dried over sodium sulphate and evaporated. The residue was purified by flash chromatography (silica gel, petrol ether/ethyl acetate 8:2) affording 47 g (93% yield) of the title compound.

LC-MS (Method 9): Rₜ=3.42 min; MS (ESIpos): m/z=600 [M+H]⁺

### Example 18A

### 1-(2-Methylpropyl)-4-[4-(4,4,5,5-tetramethyl-1,3,2-dioxaborolan-2-yl)phenyl]piperazine

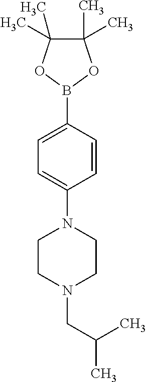 [SMILES: CC(C)CN1CCN(c2ccc(B3OC(C)(C)C(C)(C)O3)cc2)CC1]

1-[4-(4,4,5,5-tetramethyl-1,3,2-dioxaborolan-2-yl)phenyl]piperazine (350 mg, 1.21 mmol) was placed in 7.4 ml THF and N,N-diisopropylethylamine (320 μl, 1.8 mmol) was added. Then 2-methylpropanal (440 μl, 4.9 mmol) was added and the mixture was stirred for 10 min. Then sodium triacetoxyborohydride (772 mg, 3.64 mmol) was added and the mixture was stirred at 55° C. for 4 h. The reaction mixture was cooled to room temperature, saturated aqueous sodium bicarbonate solution was added and the mixture was extracted three times with ethyl acetate. The combined organic phases were washed once with saturated, aqueous sodium chloride solution, dried over sodium sulphate, filtered and evaporated. 342 mg of the target compound (79% of theory, purity 97%) were obtained.

LC-MS (Method 3): Rₜ=1.23 min; MS (ESIpos): m/z=345 [M+H]⁺

¹H-NMR (600 MHz, DMSO-d6) δ[ppm]: 0.058 (0.55), 0.927 (4.09), 0.938 (4.13), 1.316 (16.00), 2.121 (0.98), 2.133 (0.89), 2.492 (0.99), 2.508 (0.99), 2.559 (2.25), 2.599 (2.62), 3.241 (1.07), 3.249 (1.38), 3.257 (0.98), 6.935 (1.05), 6.949 (1.07), 7.552 (1.15), 7.566 (1.07).

### Example 19A

### 1-Propyl-4-[4-(4,4,5,5-tetramethyl-1,3,2-dioxaborolan-2-yl)phenyl]piperazine

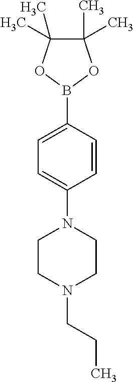

1-[4-(4,4,5,5-tetramethyl-1,3,2-dioxaborolan-2-yl)phenyl]piperazine (300 mg, 1.04 mmol) was placed in 6.4 ml of THF and N,N-diisopropylethylamine (270 ul, 1.6 mmol) was added. Propanal (242 mg, 4.16 mmol) was then added and the mixture was stirred for 10 min. Then sodium triacetoxyborohydride (662 mg, 3.12 mmol) was added and the mixture was stirred at 55° C. for 1.5 h. The reaction mixture was cooled to room temperature, saturated aqueous sodium bicarbonate solution was added and the mixture was extracted three times with ethyl acetate. The combined organic phases were washed once with saturated aqueous sodium chloride solution, dried over sodium sulfate, filtered and evaporated. The mixture was purified by means of silica gel chromatography (dichloromethane/methanol 100/1, then isocratic dichloromethane/methanol: 50/1). 186 mg of the target compound (53% of theory) were obtained.

LC-MS (Method 6): Rₜ=0.97 min; MS (ESIpos): m/z=331 [M+H]⁺

¹H-NMR (500 MHz, DMSO-d6) δ[ppm]: 0.856 (1.10), 0.871 (2.41), 0.886 (1.18), 1.070 (6.41), 1.258 (16.00), 1.457 (0.59), 1.472 (0.58), 2.250 (0.49), 2.265 (0.64), 2.279 (0.45), 2.453 (0.86), 2.462 (1.15), 2.472 (0.89), 3.181 (0.94), 3.192 (1.13), 3.201 (0.86), 3.916 (1.09), 6.877 (1.01), 6.894 (1.02), 7.490 (1.17), 7.507 (1.04).

### Example 20A

### Ethyl 1-{1-[4-chloro-4′-(4-propylpiperazin-1-yl)[1,1′-biphenyl]-2-yl]piperidin-3-yl}-5-(difluoromethyl)-1H-pyrazole-4-carboxylate trifluoroacetic acid adduct (Enantiomer 2)

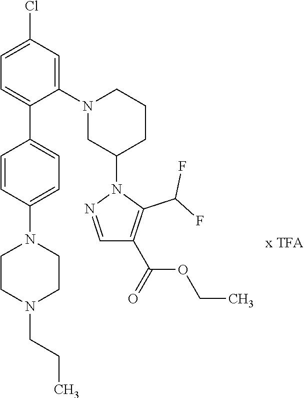 [SMILES: CCCN1CCN(c2ccc(-c3ccc(Cl)cc3N3CCCC(n4ncc(C(=O)OCC)c4C(F)F)C3)cc2)CC1]

Ethyl 1-[1-{5-chloro-2-[(trifluoromethanesulfonyl)oxy]phenyl}piperidin-3-yl]-5-(difluoromethyl)-1H-pyrazole-4-carboxylate (Enantiomer 2, 90.0 mg, 169 μmol) and 1-propyl-4-[4-(4,4,5,5-tetramethyl-1,3,2-dioxaborolan-2-yl)phenyl]piperazine (67.1 mg, 203 μmol) were placed under argon in toluene/ethanol (940 μl/940 μl), 2 M sodium carbonate solution (250 l) and tetrakis(triphenylphosphine)palladium(0) (9.78 mg, 8.46 μmol) were added and the mixture was stirred at 100° C. overnight. Tetrakis(triphenylphosphine)palladium(0) (9.78 mg, 8.46 μmol) was added to the mixture, flushed with argon and stirred at 100° C. for 3 h. The reaction mixture was diluted with ethyl acetate and water. The aqueous phase was acidified with 1M hydrochloric acid. The phases were separated and the aqueous phase was extracted twice with ethyl acetate. The combined organic phases were dried over sodium sulfate, filtered and evaporated. The residue was purified by preparative HPLC (RP18 column, mobile phase: acetonitrile/water gradient with the addition of 0.1% trifluoroacetic acid). 43 mg of the target compound were obtained (36% of theory).

LC-MS (Method 4): Rₜ=2.07 min; MS (ESIpos): m/z=586 [M+H]⁺

### Example 21A

### Ethyl 1-{1-[4-chloro-4′-(piperazin-1-yl)[1,1′-biphenyl]-2-yl]piperidin-3-yl}-5-(difluoromethyl)-1H-pyrazole-4-carboxylate hydrochloride (Enantiomer 2)

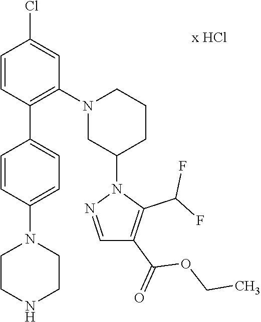 [SMILES: CCOC(=O)c1cnn(C2CCCN(c3cc(Cl)ccc3-c3ccc(N4CCNCC4)cc3)C2)c1C(F)F]

A solution of tert-butyl 4-(4′-chloro-2′-{(3-[5-(difluoromethyl)-4-(ethoxycarbonyl)-1H-pyrazol-1-yl]piperidin-1-yl}[1,1′-biphenyl]-4-yl)piperazine-1-carboxylate (Enantiomer 2, 60.0 g, 93.1 mmol) in dichloromethane (250 ml) was treated with a solution of hydrogen chloride in dioxane (230 ml, 4.0 M, 930 mmol). The resulting mixture was stirred 3 hours at room temperature and evaporated. The residue was co-evaporated twice with diethyl ether (250 ml×2), stirred 4 days in diisopropyl ether. The suspension was filtered, the solid was washed twice with diisopropyl ether affording 57 g (quant.) of the title compound.

LC-MS (Method 4): Rₜ=1.78 min; MS (ESIpos): m/z=544 [M+H]⁺

¹H-NMR (400 MHz, DMSO-d6) δ[ppm]: 1.029 (13.49), 1.044 (13.77), 1.262 (7.53), 1.280 (16.00), 1.297 (7.81), 1.496 (0.79), 1.506 (0.62), 1.527 (0.91), 1.559 (0.40), 1.716 (1.24), 1.749 (0.95), 1.888 (0.84), 1.897 (0.78), 1.918 (0.98), 1.926 (0.93), 1.966 (1.38), 1.995 (0.69), 2.580 (1.54), 2.606 (0.83), 2.992 (1.21), 3.018 (2.69), 3.044 (2.33), 3.063 (1.24), 3.435 (5.96), 3.448 (7.25), 3.460 (5.00), 3.570 (5.78), 3.586 (0.87), 3.601 (1.12), 3.616 (0.85), 4.227 (5.38), 4.238 (6.62), 4.256 (9.26), 4.273 (7.97), 4.291 (2.70), 4.444 (0.41), 4.455 (0.77), 4.470 (0.89), 4.481 (1.31), 4.491 (0.92), 4.507 (0.68), 7.045 (6.02), 7.067 (6.86), 7.074 (5.10), 7.079 (5.42), 7.099 (2.25), 7.104 (1.49), 7.120 (3.55), 7.125 (3.10), 7.164 (6.27), 7.185 (3.37), 7.383 (1.62), 7.483 (6.90), 7.505 (6.40), 7.513 (3.75), 7.643 (1.34), 8.005 (5.77), 9.399 (1.97).

### Example 22A

### 1-{1-[4-Chloro-4′-(piperazin-1-yl)[1,1′-biphenyl]-2-yl]piperidin-3-yl}-5-(difluoromethyl)-1H-pyrazole-4-carboxylic acid (Enantiomer 2)

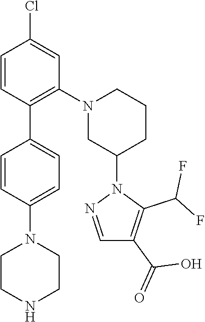 [SMILES: O=C(O)c1cnn(C2CCCN(c3cc(Cl)ccc3-c3ccc(N4CCNCC4)cc3)C2)c1C(F)F]

An aqueous solution of lithium hydroxide (4.0 ml, 1.0 M, 4.0 mmol) was added to a solution of ethyl 1-{1-[4-chloro-4′-(piperazin-1-yl)[1,1′-biphenyl]-2-yl]piperidin-3-yl}-5-(difluoromethyl)-1H-pyrazole-4-carboxylate hydrochloride (Enantiomer 2, 281 mg, 82% purity, 396 μmol) in a THF/methanol mixture 10:1 (8.8 ml). The resulting mixture was stirred 2 hours at room temperature. The reaction mixture was acidified with an aqueous solution of hydrogen chloride (2 N) and evaporated. The residue was purified by preparative HPLC (RP18 column, eluent: Acetonitrile/water gradient) affording 175 mg (86% yield) of the title compound.

LC-MS (Method 3): Rₜ=0.83 min; MS (ESIpos): m/z=516 [M+H]⁺

### Example 23A

### Tert-butyl 4-(2′-bromo-4′-chloro[1,1′-biphenyl]-4-yl)piperazine-1-carboxylate

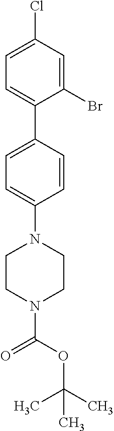 [SMILES: CC(C)(C)OC(=O)N1CCN(c2ccc(-c3ccc(Cl)cc3Br)cc2)CC1]

Under argon, a suspension of 2-bromo-4-chloro-1-iodobenzene (518 mg, 1.63 mmol), {4-[4-(tert-butoxycarbonyl)piperazin-1-yl]phenyl}boronic acid (500 mg, 1.63 mmol), Pd(PPh₃)₄ (94.4 mg, 81.7 μmol) was treated with an aqueous solution of sodium carbonate (2.4 ml, 2.0 M, 4.9 mmol) and heated overnight at 85° C. The reaction mixture was cooled to room temperature, diluted with water and extracted three times with ethyl acetate. The combined organic layers were dried over magnesium sulfate and evaporated. The residue was purified by flash chromatography (silica gel, cyclohexane/ethyl acetate gradient) affording 390 mg (52% yield) of the title compound.

LC-MS (Method 4): Rₜ=2.82 min; MS (ESIpos): m/z=451 [M+H]⁺

¹H-NMR (400 MHz, DMSO-d6) δ[ppm]: −0.008 (0.43), 0.008 (0.47), 1.419 (1.16), 1.428 (16.00), 3.166 (0.88), 3.179 (1.25), 3.192 (1.05), 3.458 (0.93), 3.471 (1.13), 3.483 (0.78), 7.006 (1.04), 7.028 (1.24), 7.258 (1.42), 7.280 (1.16), 7.355 (0.92), 7.376 (1.19), 7.488 (0.68), 7.493 (0.69), 7.508 (0.50), 7.514 (0.52), 7.820 (1.11), 7.826 (1.09).

### Example 24A

### 1-(2′-Bromo-4′-chloro[1,1′-biphenyl]-4-yl)piperazine hydrochloride

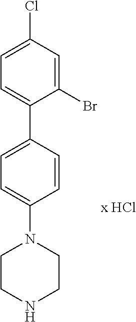 [SMILES: Clc1ccc(-c2ccc(N3CCNCC3)cc2)c(Br)c1]

A solution of tert-butyl 4-(2′-bromo-4′-chloro[1,1′-biphenyl]-4-yl)piperazine-1-carboxylate (prepared in analogy to Example 46A, 664 mg, 1.47 mmol) in dichloromethane (8.0 ml) was treated with a solution of hydrogen chloride in dioxane (3.7 ml, 4.0 M, 15 mmol), stirred 2.5 hours and evaporated. The residue was triturated in diethyl ether. The solid was filtered off affording 602 mg (quant.) of the title compound.

LC-MS (Method 4): Rₜ=1.46 min; MS (ESIpos): m/z=351 [M+H]⁺

¹H-NMR (400 MHz, DMSO-d6) δ[ppm]: 0.146 (0.66), 1.596 (1.27), 2.329 (0.80), 2.671 (0.82), 3.360 (1.28), 3.437 (12.88), 3.451 (14.96), 3.463 (10.26), 3.568 (1.96), 4.670 (3.35), 5.756 (2.57), 7.002 (0.87), 7.055 (12.43), 7.077 (14.87), 7.294 (16.00), 7.315 (12.89), 7.362 (11.21), 7.382 (14.09), 7.502 (7.29), 7.507 (7.43), 7.522 (5.26), 7.528 (5.74), 7.835 (12.20), 7.840 (11.66), 9.202 (2.59).

### Example 25A

### 1-(2′-Bromo-4′-chloro[1,1′-biphenyl]-4-yl)-4-(2,2,2-trifluoroethyl)piperazine

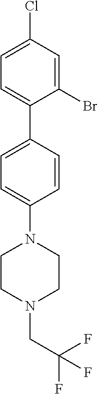 [SMILES: FC(F)(F)CN1CCN(c2ccc(-c3ccc(Cl)cc3Br)cc2)CC1]

Under argon, a solution of 1-(2′-bromo-4′-chloro[1,1′-biphenyl]-4-yl)piperazine hydrochloride (600 mg, 1.55 mmol) in DMF (7.8 ml) was treated with N,N-diisopropylethylamine (1.6 ml, 9.3 mmol) and 2,2,2-trifluoroethyl trifluoromethanesulfonate (670 μl, 4.6 mmol) and stirred overnight at room temperature. The reaction mixture was diluted with water and extracted three times with ethyl acetate. The combined organic layers were dried over magnesium sulfate and evaporated. The residue was purified by flash chromatography (silica gel, cyclohexane/ethyl acetate gradient) affording 536 mg (80% yield) of the title compound.

LC-MS (Method 3): Rₜ=1.42 min; MS (ESIpos): m/z=433 [M+H]⁺ (isotope 1) m/z=435 [M+H]⁺ (isotope 2)

¹H-NMR (400 MHz, DMSO-d6) δ[ppm]: −0.007 (1.98), 0.008 (2.22), 2.740 (0.98), 2.758 (11.29), 2.770 (14.79), 2.782 (12.18), 3.115 (0.79), 3.128 (0.89), 3.140 (0.72), 3.205 (12.89), 3.212 (13.88), 3.218 (16.00), 3.230 (12.50), 3.236 (11.79), 3.262 (9.39), 3.288 (3.13), 6.914 (0.51), 6.935 (0.60), 6.990 (11.06), 7.011 (13.08), 7.204 (0.54), 7.246 (13.70), 7.267 (11.56), 7.354 (8.19), 7.375 (10.92), 7.485 (5.59), 7.490 (5.77), 7.506 (4.15), 7.511 (4.36), 7.817 (7.70), 7.823 (7.57).

### Example 26A

### Ethyl 1-[1-{4-chloro-4′-[4-(2,2,2-trifluoroethyl)piperazin-1-yl][1,1′-biphenyl]-2-yl}piperidin-3-yl]-5-(difluoromethyl)-1H-pyrazole-4-carboxylate (Enantiomer 1)

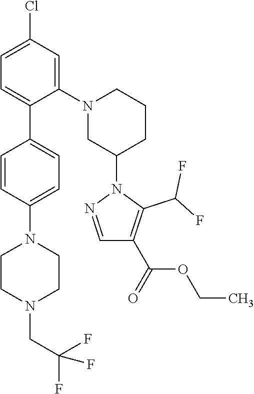 [SMILES: CCOC(=O)c1cnn(C2CCCN(c3cc(Cl)ccc3-c3ccc(N4CCN(CC(F)(F)F)CC4)cc3)C2)c1C(F)F]

Under argon, a solution of 1-(2′-bromo-4′-chloro[1,1′-biphenyl]-4-yl)-4-(2,2,2-trifluoroethyl)piperazine (150 mg, 346 μmol) and ethyl 5-(difluoromethyl)-1-[piperidin-3-yl]-1H-pyrazole-4-carboxylate (Enantiomer 1, 94.5 mg, 346 μmol) in toluene (3.0 ml) was treated with caesium carbonate (282 mg, 865 μmol) and RuPhos Pd G3 (57.9 mg, 69.2 μmol) and stirred 16 hours at 100° C. The reaction mixture was cooled to room temperature, diluted with ethyl acetate, filtered over celite and evaporated. The residue was purified by preparative HPLC (RP18 column, eluent: Acetonitrile/water+0.5% formic acid gradient) affording 62.7 mg (29% yield) of the title compound.

LC-MS (Method 3): Rₜ=1.58 min; MS (ESIpos): m/z=626 [M+H]⁺

### Example 27A

### Ethyl 1-[1-{4-chloro-4′-[4-(2,2,2-trifluoroethyl)piperazin-1-yl][1,1′-biphenyl]-2-yl}piperidin-3-yl]-5-(difluoromethyl)-1H-pyrazole-4-carboxylate (Enantiomer 2)

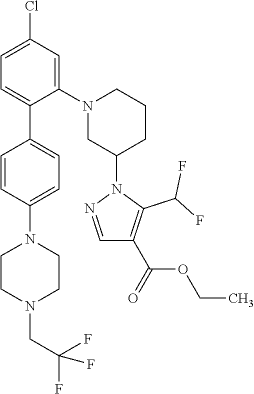 [SMILES: CCOC(=O)c1cnn(C2CCCN(c3cc(Cl)ccc3-c3ccc(N4CCN(CC(F)(F)F)CC4)cc3)C2)c1C(F)F]

Under argon, a solution of 1-(2′-bromo-4′-chloro[1,1′-biphenyl]-4-yl)-4-(2,2,2-trifluoroethyl)piperazine (150 mg, 346 μmol) and ethyl 5-(difluoromethyl)-1-[piperidin-3-yl]-1H-pyrazole-4-carboxylate (Enantiomer 2, 94.5 mg, 346 μmol) in toluene (3.0 ml) was treated with caesium carbonate (282 mg, 865 μmol) and RuPhos Pd G3 (57.9 mg, 69.2 μmol) and stirred 16 hours at 100° C. The reaction mixture was cooled to room temperature, diluted with ethyl acetate, filtered over celite and evaporated. The residue was purified by preparative HPLC (RP18 column, eluent: Acetonitrile/water+0.5% formic acid gradient) affording 66.6 mg (30% yield) of the title compound.

LC-MS (Method 3): Rₜ=1.58 min; MS (ESIpos): m/z=626 [M+H]⁺

### Example 28A

### Tert-butyl 3-[4-(ethoxycarbonyl)-5-(trifluoromethyl)-1H-pyrazol-1-yl]piperidine-1-carboxylate

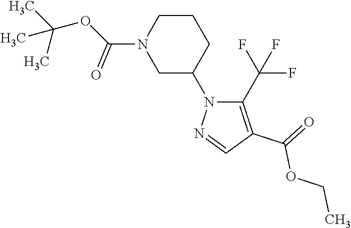 [SMILES: CCOC(=O)c1cnn(C2CCCN(C(=O)OC(C)(C)C)C2)c1C(F)(F)F]

Tert-butyl 3-hydrazinopiperidine-1-carboxylate acetic acid (945 g, 3.43 mol) in ethanol (20 L) was treated with ethyl 2-(ethoxymethylene)-4,4,4-trifluoro-3-oxobutanoate (907 g, 3.78 mol). The resulting mixture was stirred 16 hours at 25° C., diluted with a saturated solution of sodium hydrogencarbonate (2.0 L) and concentrated to ˜5.0 L. The resulting mixture was diluted with water (5.0 L) and extracted with ethyl acetate (5.0 L). The organic phase was washed with a saturated solution of sodium chloride (5.0 L) and evaporated. The residue was purified by flash chromatography (silica gel, petroleum ether/ethyl acetate, 10:1) affording 548 g (41% yield) of the title compound.

¹H-NMR (400 MHz, CDCl₃) δ [ppm]: 7.90 (s, 1H), 4.33-3.09 (m, 5H), 3.26-3.12 (m, 1H), 2.89-2.61 (m, 1H), 2.35-2.05 (m, 2H), 1.98-1.78 (m, 1H), 1.71-1.51 (m, 1H), 1.50-1.37 (m, 9H), 1.32 (m, 3H)

### Example 29A

### Ethyl 1-(piperidin-3-yl)-5-(trifluoromethyl)-1H-pyrazole-4-carboxylate (Racemate)

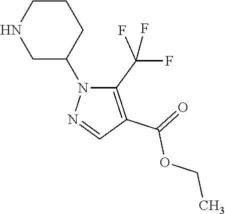 [SMILES: CCOC(=O)c1cnn(C2CCCNC2)c1C(F)(F)F]

Tert-butyl 3-[4-(ethoxycarbonyl)-5-(trifluoromethyl)-1H-pyrazol-1-yl]piperidine-1-carboxylate (548 g, 1.40 mol) was treated with a solution of hydrogen chloride in dioxane (4 M, 2.38 L), stirred 2 hours at 25° C. and evaporated. The residue was retaken in 1.0 L water and extracted with MTBE (500 mL×1). The aqueous phase was separated and adjusted to pH to 8-9 with a saturated solution of sodium hydrogencarbonate. The aqueous phase was extracted with dichloromethane (1.0 L×2), and the combined organic layers were washed with a saturated solution of sodium chloride (1 L), dried over sodium sulfate and evaporated affording 325 g (80% yield) of the title compound.

LC-MS: (Method 1) Rₜ=0.955 min, MS (M+1)=299.2.

The two enantiomers were separated by SFC [325 g, column: Phenomenex-Cellulose-2 (250 mm*50 mm, 10 μm); eluent: CO₂/(methanol+0.1% aqueous ammonia); 75:25, 4.5 min; 1400 min] affording 103.0 g of enantiomer 1 (Example 5A) and 110.1 g of enantiomer 2 (Example 6A).

### Example 30A

### Ethyl 1-(piperidin-3-yl)-5-(trifluoromethyl)-1H-pyrazole-4-carboxylate (Enantiomer 1)

For separation conditions see Example 29A.

Analytical SFC: Rₜ=1.345 min, e.e. =99% [Column Cellulose 2-3: 50×4.6 mm; eluent: CO2/[methanol+0.5% diethyl amine]: 95:5 to 60:40 flow rate: 3.0 ml/min; temperature: 35° C.; UV detection: 220 nm, back pressure 100 bar].

LCMS (Method 2), Rₜ=0.906 min, MS (M+1)=292.1.

¹H-NMR (400 MHz, CDCl₃) δ [ppm]: 7.89 (s, 1H), 4.50-4.47 (m, 1H), 4.31-4.25 (m, 2H), 3.24-3.05 (m, 4H), 2.70-2.67 (m, 1H), 2.10-2.02 (m, 2H), 1.92-1.79 (m, 1H), 1.74-1.56 (m, 1H), 1.31 (t, J=7.2 Hz, 3H).

### Example 31A

### Ethyl 1-(piperidin-3-yl)-5-(trifluoromethyl)-1H-pyrazole-4-carboxylate (Enantiomer 2)

For separation conditions see Example 29A.

Analytical SFC: Rₜ=1.071 min, e.e. =99% [Column Cellulose 2-3: 50×4.6 mm; eluent: CO2/[methanol+0.5% diethyl amine]: 95:5 to 60:40 flow rate: 3.0 ml/min; temperature: 35° C.; UV detection: 220 nm, back pressure 100 bar].

LCMS (Method 2), Rₜ=0.906 min, MS (M+1)=292.1.

¹H-NMR (400 MHz, CDCl₃) δ [ppm]: 7.91 (s, 1H), 4.58-4.41 (m, 1H), 4.35-4.23 (m, 2H), 3.70-3.56 (m, 1H), 3.31-3.12 (m, 2H), 3.11-3.02 (m, 1H), 2.75-2.62 (m, 1H), 2.15-2.02 (m, 2H), 1.92-1.79 (m, 1H), 1.74-1.56 (m, 1H), 1.33 (t, J=7.2 Hz, 3H).

### Example 32A

### Ethyl 1-[1-{5-chloro-2-[(4-methoxyphenyl)methoxy]phenyl}piperidin-3-yl]-5-(trifluoromethyl)-1H-pyrazole-4-carboxylate (Enantiomer 1)

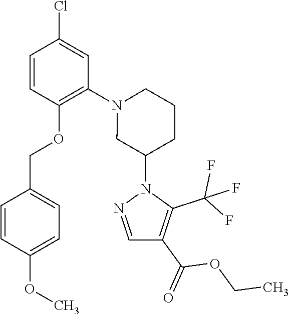 [SMILES: CCOC(=O)c1cnn(C2CCCN(c3cc(Cl)ccc3OCc3ccc(OC)cc3)C2)c1C(F)(F)F]

Under argon, a solutionf ethyl 1-[piperidin-3-yl]-5-(trifluoromethyl)-1H-pyrazole-4-carboxylate (Enantiomer 1, 75.0 g, 257 mmol) and 2-bromo-4-chloro-1-[(4-methoxyphenyl)methoxy]benzene (84.4 g, 257 mmol) in 1,4-dioxane (1.1 l) was treated with Pd₂dba₃ (23.6 g, 25.7 mmol), rac-BINAP (32.1 g, 51.5 mmol) and cesium carbonate (252 g, 772 mmol). The resulting mixture was stirred 3 days at 100° C. and cooled to room temperature. The reaction mixture was diluted with an aqueous solution of sodium chloride (10%) and ethyl acetate, filtered over celite and rinsed with ethyl acetate. The aqueous phase of the filtrate was separated and extracted with ethyl acetate. The combined organic layers were washed with an aqueous solution of sodium chloride (10%), dried over sodium sulfate and evaporated. The residue was purified by flash chromatography (silica gel, dichloromethane/petrol ether gradient) affording 119 g (71% yield) of the title compound.

LC-MS (Method 3): Rₜ=2.81 min; MS (ESIpos): m/z=538 [M+H]⁺

### Example 33A

### Ethyl 1-[1-(5-chloro-2-hydroxyphenyl)piperidin-3-yl]-5-(trifluoromethyl)-1H-pyrazole-4-carboxylate (Enantiomer 1)

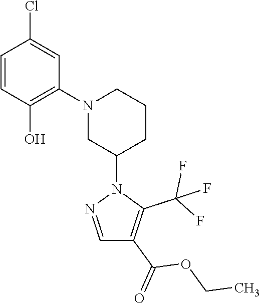 [SMILES: CCOC(=O)c1cnn(C2CCCN(c3cc(Cl)ccc3O)C2)c1C(F)(F)F]

A solution of ethyl 1-[1-{5-chloro-2-[(4-methoxyphenyl)methoxy]phenyl}piperidin-3-yl]-5-(trifluoromethyl)-1H-pyrazole-4-carboxylate (Enantiomer 1, 119 g, 221 mmol) in dichloromethane (1.8 l) was treated with trifluoroacetic acid (170 ml, 2.2 mol) and the resulting mixture was stirred 3 days at room temperature. Thre reaction mixture was quenched carefully with an aqueous solution of sodium hydrogenocarbonate (10%) until pH=8. The phases were separated. The organic layer was evaporated and the residue was purified by flash chromatography (silica gel, dichloromethane/petrol ether gradient) affording 85 g (90% purity, 92% yield) of the title compound.

LC-MS (Method 3): Rₜ=2.47 min; MS (ESIpos): m/z=418 [M+H]⁺

### Example 34A

### Ethyl 1-[1-{5-chloro-2-[(trifluoromethanesulfonyl)oxy]phenyl}piperidin-3-yl]-5-(trifluoromethyl)-1H-pyrazole-4-carboxylate (Enantiomer 1)

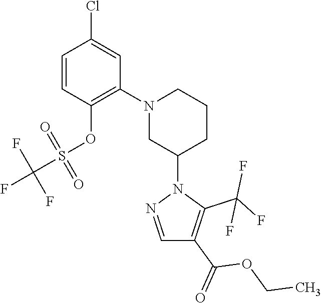 [SMILES: CCOC(=O)c1cnn(C2CCCN(c3cc(Cl)ccc3OS(=O)(=O)C(F)(F)F)C2)c1C(F)(F)F]

Under argon, a solution of ethyl 1-[1-(5-chloro-2-hydroxyphenyl)piperidin-3-yl]-5-(trifluoromethyl)-1H-pyrazole-4-carboxylate (Enantiomer 1, 85.0 g, 90% purity, 184 mmol) in dichloromethane (520 ml) was cooled to −50° C. and treated with triethylamine (77 ml, 550 mmol). Trifluoromethanesulfonic anhydride (43 ml, 260 mmol) was added dropwise to the reaction mixture and the resulting solution was stirred 1 hour at −50° C. The reaction mixture was diluted with dichloromethane (520 ml) and ice-cooled water (590 ml). The aqueous layer was extracted with dichloromethane (520 ml). The combined organice layers were washed once with ice-cooled water (590 ml), dried over sodium sulfate and evaporated. The residue was purified by flash chromatography (silica gel, dichloromethane/petrol ether gradient) affording 94 g (93% yield) of the title compound.

LC-MS (Method 3): Rₜ=2.79 min; MS (ESIpos): m/z=550 [M+H]⁺

¹H-NMR (400 MHz, DMSO-d6) δ[ppm]: 1.259 (7.63), 1.271 (16.00), 1.282 (7.94), 1.771 (0.45), 1.779 (0.80), 1.786 (0.61), 1.793 (0.61), 1.800 (0.94), 1.807 (0.61), 1.821 (0.45), 1.932 (1.22), 1.955 (0.96), 2.099 (0.77), 2.106 (0.73), 2.120 (1.04), 2.126 (1.33), 2.138 (1.91), 2.820 (0.73), 2.825 (0.87), 2.841 (1.56), 2.845 (1.61), 2.861 (0.93), 2.865 (0.82), 3.140 (1.18), 3.159 (1.09), 3.186 (1.39), 3.204 (2.87), 3.222 (1.78), 3.318 (1.51), 3.324 (1.60), 3.336 (1.08), 3.342 (1.04), 4.247 (2.31), 4.259 (7.26), 4.270 (7.27), 4.282 (2.41), 4.669 (0.70), 4.679 (0.84), 4.686 (1.34), 4.694 (0.96), 4.704 (0.72), 4.711 (0.42), 7.286 (2.29), 7.290 (2.44), 7.300 (2.89), 7.304 (3.11), 7.415 (5.01), 7.430 (4.13), 7.457 (5.11), 7.461 (5.05), 8.123 (6.61).

### Example 35A

### Tert-butyl 4-(4′-chloro-2′-{3-[4-(ethoxycarbonyl)-5-(trifluoromethyl)-1H-pyrazol-1-yl]piperidin-1-yl}[1,1′-biphenyl]-4-yl)piperazine-1-carboxylate (Enantiomer 1)

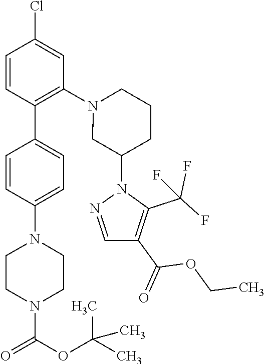 [SMILES: CCOC(=O)c1cnn(C2CCCN(c3cc(Cl)ccc3-c3ccc(N4CCN(C(=O)OC(C)(C)C)CC4)cc3)C2)c1C(F)(F)F]

Under argon, a solution of ethyl 1-[1-{5-chloro-2-[(trifluoromethanesulfonyl)oxy]phenyl}piperidin-3-yl]-5-(trifluoromethyl)-1H-pyrazole-4-carboxylate (Enantiomer 1, 92.1 g, 167 mmol) and tert-butyl 4-[4-(4,4,5,5-tetramethyl-1,3,2-dioxaborolan-2-yl)phenyl]piperazine-1-carboxylate (78.0 g, 201 mmol) in toluene (840 ml) and ethanol (840 ml) was treatd with an aqueous solution of sodium carbonate (250 ml, 2.0 M, 500 mmol) and Pd(PPh₃)₄ (9.68 g, 8.37 mmol) and the resulting mixture was stirred overnight at 100° C. The reaction mixture was cooled to room temperature, filtered over celite, rinsed with ethyl acetate and evaporated. The residue was purified by flash chromatography (silica gel, petrol ether/ethyl acetate gradient) affording 94 g (85% yield) of the title compound.

LC-MS (Method 3): Rₜ=3.19 min; MS (ESIpos): m/z=662 [M+H]⁺

¹H-NMR (400 MHz, DMSO-d6) δ[ppm]: −0.008 (0.66), 0.008 (0.84), 1.038 (0.55), 1.088 (0.76), 1.232 (1.60), 1.250 (3.49), 1.268 (1.69), 1.419 (0.77), 1.431 (16.00), 1.989 (0.77), 2.957 (0.43), 3.127 (0.91), 3.140 (1.32), 3.152 (1.09), 3.457 (0.99), 3.470 (1.25), 3.481 (0.88), 4.211 (0.45), 4.228 (1.43), 4.246 (1.38), 4.264 (0.43), 6.985 (1.10), 7.007 (1.20), 7.068 (0.79), 7.073 (1.06), 7.089 (0.49), 7.109 (0.80), 7.114 (0.69), 7.146 (1.34), 7.166 (0.65), 7.433 (1.33), 7.455 (1.19), 8.062 (1.56).

### Example 36A

### Ethyl 1-{1-[4-chloro-4′-(piperazin-1-yl)[1,1′-biphenyl]-2-yl]piperidin-3-yl}-5-(trifluoromethyl)-1H-pyrazole-4-carboxylate hydrochloride (Enantiomer 1)

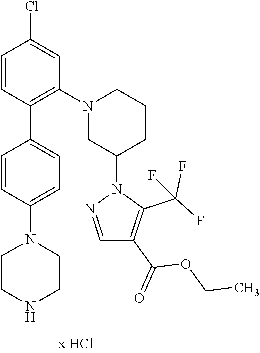 [SMILES: CCOC(=O)c1cnn(C2CCCN(c3cc(Cl)ccc3-c3ccc(N4CCNCC4)cc3)C2)c1C(F)(F)F]

A solution of tert-butyl 4-(4′-chloro-2′-{3-[4-(ethoxycarbonyl)-5-(trifluoromethyl)-1H-pyrazol-1-yl]piperidin-1-yl}[1,1′-biphenyl]-4-yl)piperazine-1-carboxylate (Enantiomer 1, 93.0 g, 140 mmol) in dichloromethane (290 ml) was treated with a solution of hydrogen chloride in dioxane (350 ml, 4.0 M, 1.4 mol) and stirred 3 hours at room temperature. The reaction mixture was evaporated and the residue co-evaporated with MTBE affording 95 g (quant.) of the title compound which was used in the next step without further purification.

LC-MS (Method 3): Rₜ=1.97 min; MS (ESIpos): m/z=562 [M+H]⁺

### Example 37A Ethyl 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylate (Enantiomer 1)

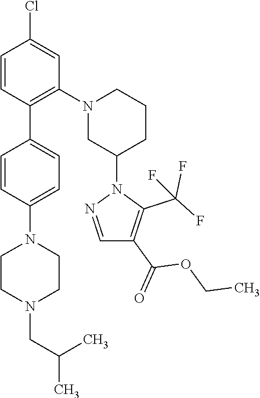 [SMILES: CCOC(=O)c1cnn(C2CCCN(c3cc(Cl)ccc3-c3ccc(N4CCN(CC(C)C)CC4)cc3)C2)c1C(F)(F)F]

A solution of ethyl 1-{1-[4-chloro-4′-(piperazin-1-yl)[1,1′-biphenyl]-2-yl]piperidin-3-yl}-5-(trifluoromethyl)-1H-pyrazole-4-carboxylate hydrochloride (Enantiomer 1, 95.0 g, 150 mmol) in THF (1.8 l) was treated with N,N-diisopropylethylamine (100 ml, 600 mmol) and 2-methylpropanal [CAS No. 78-84-2] (53.9 g, 748 mmol) and stirred 1 hour at room temperature. Sodium triacetoxyborohydride (127 g, 598 mmol]) was added and the resulting mixture was stirred 18 hours at room temperature. The reaction mixture was diluted with an aqueous solution of sodium hydrogen carbonate (10%) and extracted three times with ethyl acetate. The combined organic layers were washed with a saturated solution of sodium chloride, dried over sodium sulphate and evaporated. The residue was purified by flash chromatography (silica gel, petrol ether/ethyl acetate gradient) affording 78 g (84% yield) of the title compound.

LC-MS (Method 3): Rₜ=2.03 min; MS (ESIpos): m/z=618 [M+H]⁺

¹H-NMR (600 MHz, DMSO-d6) δ[ppm]: 0.827 (0.53), 0.839 (0.55), 0.867 (0.66), 0.871 (0.81), 0.883 (15.73), 0.894 (16.00), 1.041 (0.92), 1.090 (1.35), 1.241 (4.62), 1.252 (9.48), 1.264 (4.77), 1.554 (0.58), 1.575 (0.64), 1.753 (0.80), 1.775 (0.81), 1.786 (0.63), 1.798 (0.90), 1.809 (1.08), 1.820 (0.88), 1.831 (0.48), 1.889 (0.64), 1.895 (0.58), 1.909 (0.66), 1.916 (0.63), 1.988 (0.94), 1.998 (0.79), 2.015 (0.59), 2.085 (4.57), 2.097 (4.16), 2.467 (3.65), 2.476 (5.06), 2.483 (4.05), 2.595 (0.61), 2.612 (1.08), 2.615 (1.10), 2.631 (0.59), 2.937 (0.86), 2.955 (1.65), 2.972 (0.98), 3.073 (0.84), 3.093 (0.80), 3.156 (3.65), 3.164 (4.72), 3.172 (3.66), 3.212 (0.97), 3.227 (0.83), 4.221 (1.39), 4.233 (4.19), 4.245 (4.15), 4.256 (1.41), 4.362 (0.49), 4.379 (0.85), 4.397 (0.49), 6.949 (3.70), 6.963 (3.93), 7.060 (2.66), 7.063 (3.31), 7.082 (1.52), 7.085 (1.15), 7.095 (2.12), 7.099 (1.92), 7.141 (3.61), 7.154 (2.29), 7.411 (4.15), 7.426 (3.95), 8.049 (4.51).

### Example 38A

### 1-(Cyclopropylmethyl)-4-[4-(4,4,5,5-tetramethyl-1,3,2-dioxaborolan-2-yl)phenyl]piperazine

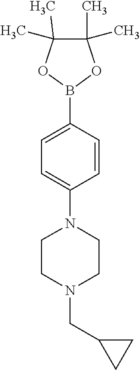 [SMILES: CC1(C)OB(c2ccc(N3CCN(CC4CC4)CC3)cc2)OC1(C)C]

1-[4-(4,4,5,5-tetramethyl-1,3,2-dioxaborolan-2-yl)phenyl]piperazine (380 mg, 1.32 mmol) was dissolved in 8 ml THF and N,N-diisopropylethylamine (340 μl, 2.0 mmol) was added. Then cyclopropanecarbaldehyde (370 mg, 5.27 mmol) was added and the mixture was stirred for 10 min. Then sodium triacetoxyborohydride (838 mg, 3.96 mmol) was added and the mixture was stirred at 55° C. for 4 h. The reaction mixture was cooled to room temperature, saturated aqueous sodium bicarbonate solution was added and the mixture was extracted three times with ethyl acetate. The combined organic phases were washed once with saturated aqueous sodium chloride solution, dried over sodium sulfate, filtered and evaporated. 519 mg of the target compound (98% of theory, purity 85%) were obtained.

LC-MS (Method 3): Rₜ=1.18 min; MS (ESIpos): m/z=343 [M+H]⁺

¹H-NMR (600 MHz, DMSO-d6) δ[ppm]: 0.089 (0.56), 0.096 (0.58), 0.471 (0.53), 0.483 (0.55), 1.158 (0.59), 1.175 (0.52), 1.259 (16.00), 1.989 (1.00), 3.210 (0.89), 3.216 (0.92), 3.226 (0.55), 6.885 (0.93), 6.900 (0.95), 7.494 (1.10), 7.509 (1.02).

### Example 39A

### Ethyl 1-[1-{4-chloro-4′-[4-(cyclopropylmethyl)piperazin-1-yl][1,1′-biphenyl]-2-yl}piperidin-3-yl]-5-(trifluoromethyl)-1H-pyrazole-4-carboxylate (Enantiomer 1)

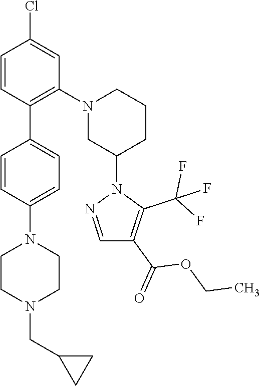 [SMILES: CCOC(=O)c1cnn(C2CCCN(c3cc(Cl)ccc3-c3ccc(N4CCN(CC5CC5)CC4)cc3)C2)c1C(F)(F)F]

Ethyl 1-[1-{5-chloro-2-[(trifluoromethanesulfonyl)oxy]phenyl}piperidin-3-yl]-5-(trifluoromethyl)-1H-pyrazole-4-carboxylate (Enantiomer 1, 150 mg, 273 μmol) and 1-(cyclopropylmethyl)-4-[4-(4,4,5,5-tetramethyl-1,3,2-dioxaborolan-2-yl)phenyl]piperazine (112 mg, purity 85%, 278 μmol) were dissolved under argon in toluene/ethanol (1.5/1.5 ml). Tetrakis(triphenylphosphine)palladium(0) (15.8 mg, 13.6 μmol) and 2 M sodium carbonate solution (410 μl, 820 μmol) were added and stirred at 100° C. for 2 h. The reaction mixture was diluted with ethyl acetate and water. The phases were separated and the aqueous phase was extracted three times with ethyl acetate. The organic phase was then dried over sodium sulfate, filtered and evaporated. The residue was dissolved in acetonitrile and a few drops of water and purified by means of prep HPLC (RP18 column, acetonitrile/water gradient with addition of 0.1% TFA). 191 mg of the target compound as TFA adduct (81% of theory) were obtained.

LC-MS (Method 3): Rₜ=2.09 min; MS (ESIpos): m/z=616 [M+H]⁺

### Example 40A

### 1-{1-[4-Chloro-4′-(piperazin-1-yl)[1,1′-biphenyl]-2-yl]piperidin-3-yl}-5-(trifluoromethyl)-1H-pyrazole-4-carboxylic acid (Enantiomer 1)

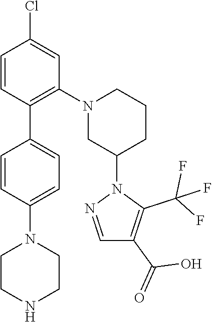 [SMILES: O=C(O)c1cnn(C2CCCN(c3cc(Cl)ccc3-c3ccc(N4CCNCC4)cc3)C2)c1C(F)(F)F]

A solution of ethyl 1-{1-[4-chloro-4′-(piperazin-1-yl)[1,1′-biphenyl]-2-yl]piperidin-3-yl}-5-(trifluoromethyl)-1H-pyrazole-4-carboxylate hydrochloride (Enantiomer 1, 290 mg, 516 μmol) in a THF/methanol mixture (10:1) (11 ml) was treated with an aqueous solution of lithium hydroxide (5.2 ml, 1.0 M, 5.2 mmol) and stirred 2.5 hours at room temperature. The reaction mixture was acidified with an aqueous solution of hydrogen chloride (2N) and evaporated. The residue was purified by preparative HPLC (RP18 column, eluent: Acetonitrile/water gradient) affording 316 mg (73% yield) of the title compound.

LC-MS (Method 3): Rₜ=1.62 min; MS (ESIpos): m/z=534 [M+H]⁺

### Example 41A

### 2,2,2-trifluoroethyl 1-(1-{4-chloro-4′-[4-(2,2,2-trifluoroethyl)piperazin-1-yl][biphenyl]-2-yl}piperidin-3-yl)-5-(trifluoromethyl)-1H-pyrazole-4-carboxylate (Enantiomer 1)

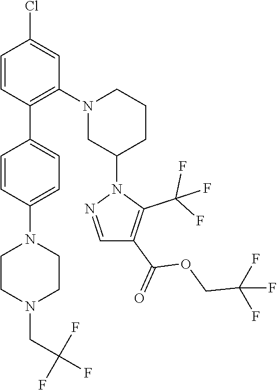 [SMILES: O=C(OCC(F)(F)F)c1cnn(C2CCCN(c3cc(Cl)ccc3-c3ccc(N4CCN(CC(F)(F)F)CC4)cc3)C2)c1C(F)(F)F]

Under argon, a solution of 1-{1-[4-chloro-4′-(piperazin-1-yl)[1,1′-biphenyl]-2-yl]piperidin-3-yl}-5-(trifluoromethyl)-1H-pyrazole-4-carboxylic acid (Enantiomer 1, 100 mg, 187 μmol) in DMF (1.7 ml) was treated with N,N-diisopropylethylamine (100 μl, 580 μmol) and 2,2,2-trifluoroethyl trifluoromethanesulfonate (81 μl, 560 μmol). The resulting mixture was stirred 2 hours at room temperature, acidified with formic acid and purified by preparative HPLC (RP18 column, eluent: Acetonitrile/water+0.1% formic acid gradient) affording 88 mg (67% yield) of the title compound.

LC-MS (Method 3): Rₜ=3.01 min; MS (ESIpos): m/z=698 [M+H]⁺

### Experimental Section—Example Compounds

### Example 1

### 1-[1-{4-Chloro-4′-[4-(2-methylpropyl)piperazin-1-yl][1,1′-biphenyl]-2-yl}piperidin-3-yl]-5-(difluoromethyl)-1H-pyrazole-4-carboxylic acid hydrochloride (Enantiomer 1)

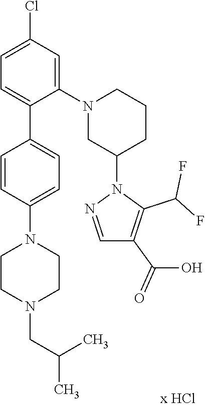 [SMILES: CC(C)CN1CCN(c2ccc(-c3ccc(Cl)cc3N3CCCC(n4ncc(C(=O)O)c4C(F)F)C3)cc2)CC1]

Ethyl 1-[1-{5-chloro-2-[(trifluoromethanesulfonyl)oxy]phenyl}piperidin-3-yl]-5-(difluoromethyl)-1H-pyrazole-4-carboxylate (prepared in analogy to Example 11A, Enantiomer 1, 80.0 mg, 147 μmol) and 1-(2-methylpropyl)-4-[4-(4,4,5,5-tetramethyl-1,3,2-dioxaborolan-2-yl)phenyl]piperazine (Example 18A 62.8 mg, 97% purity, 177 μmol) were placed under argon in toluene/ethanol (820/820 μl). 2 M sodium carbonate solution (220 μl, 2.0 M, 440 μmol) and tetrakis(triphenylphosphine)palladium(0) (8.52 mg, 7.37 μmol) were added and the mixture was stirred at 100° C. overnight. The reaction mixture was diluted with ethyl acetate and 1 M hydrochloric acid was added. The aqueous phase was extracted three times with ethyl acetate. The organic phase was dried with sodium sulfate, filtered off and evaporated. The crude mixture was dissolved with THF/ethanol (2.0/0.2 ml), 1 M lithium hydroxide solution (1.5 ml, 1.5 mmol) was added and the mixture was stirred at room temperature overnight. A 1 M lithium hydroxide solution (740 μl, 740 μmol) was added again. After about 6 h the reaction mixture was evaporated at 50° C. The residue was dissolved in acetonitrile/water/0.25 ml trifluoroacetic acid and purified by preparative HPLC (RP18 column, acetonitrile/water gradient with the addition of 0.1% trifluoroacetic acid). The crude product was purified by means of thick layer chromatography (dichloromethane/methanol/formic acid: 10/1/0.1). The silica gel mixture was stirred with dichloromethane/1 M hydrochloric acid in dioxane (10/1) in ethanol, filtered off and carefully evaporated at 30° C. and lyophilized. 34 mg of the target compound (36% of theory, purity 95%) were obtained.

LC-MS (Method 6): Rₜ=1.23 min; MS (ESIpos): m/z=572 [M−HCl+H]⁺

¹H-NMR (600 MHz, DMSO-d6) δ[ppm]: 1.004 (15.87), 1.015 (16.00), 1.500 (0.51), 1.521 (0.57), 1.728 (0.73), 1.750 (0.61), 1.897 (0.57), 1.917 (0.62), 1.975 (0.79), 2.122 (0.42), 2.133 (0.84), 2.144 (1.02), 2.156 (0.79), 2.571 (0.47), 2.587 (0.91), 2.610 (0.52), 3.004 (0.84), 3.022 (2.01), 3.026 (2.20), 3.038 (3.72), 3.048 (2.50), 3.065 (0.75), 3.154 (2.66), 3.161 (2.75), 3.169 (2.36), 3.177 (1.88), 3.224 (0.84), 3.237 (0.70), 3.589 (1.41), 3.602 (1.80), 3.825 (1.02), 3.841 (0.78), 3.866 (1.05), 3.882 (0.75), 4.223 (2.57), 4.445 (0.68), 4.463 (0.97), 4.481 (0.57), 7.045 (0.55), 7.055 (3.63), 7.070 (3.72), 7.084 (2.72), 7.087 (3.09), 7.110 (1.47), 7.113 (1.11), 7.123 (2.19), 7.127 (2.02), 7.163 (3.67), 7.177 (2.19), 7.215 (0.46), 7.428 (0.83), 7.495 (4.24), 7.510 (4.02), 7.515 (2.07), 7.602 (0.82), 7.959 (4.79), 9.484 (0.54).

### Example 2

### 1-[1-{4-Chloro-4′-[4-(2-methylpropyl)piperazin-1-yl][1,1′-biphenyl]-2-yl}piperidin-3-yl]-5-(difluoromethyl)-1H-pyrazole-4-carboxylic acid (Enantiomer 2)

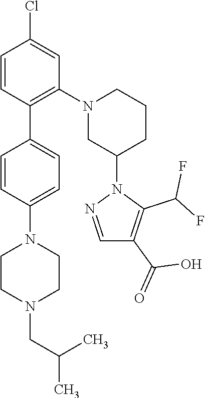 [SMILES: CC(C)CN1CCN(c2ccc(-c3ccc(Cl)cc3N3CCCC(n4ncc(C(=O)O)c4C(F)F)C3)cc2)CC1]

Method A

A solution of ethyl 1-[1-{4-chloro-4′-[4-(2-methylpropyl)piperazin-1-yl][1,1′-biphenyl]-2-yl}piperidin-3-yl]-5-(difluoromethyl)-1H-pyrazole-4-carboxylate (prepared in analogy to Example 17A, Enantiomer 2, 50.8 g, 84.6 mmol) in a THF/methanol mixture 9:1 (1.0 l) was treated with an aqueous solution of lithium hydroxide (850 ml, 1.0 M, 850 mmol) and stirred overnight at room temperature. The reaction mixture was concentrated, diluted with dichloromethane (1.5 l) and adjusted to pH=2 with an aqueous solution of hydrogen chloride (2N). The resulting suspension was stirred 45 minutes at room temperature. The solid was filtered, washed with water and dried under vacuum affording 43 g (90% yield) of the title compound.

LC-MS (Method 7): Rₜ=1.27 min; MS (ESIpos): m/z=572 [M+H]⁺

¹H-NMR (600 MHz, DMSO-d6) δ[ppm]: 1.002 (15.68), 1.013 (16.00), 1.080 (0.57), 1.092 (1.18), 1.103 (0.63), 1.498 (0.74), 1.519 (0.83), 1.719 (1.03), 1.741 (0.88), 1.902 (0.78), 1.908 (0.74), 1.922 (0.88), 1.928 (0.83), 1.943 (0.45), 1.978 (1.13), 1.994 (0.74), 2.102 (0.71), 2.112 (0.85), 2.123 (0.70), 2.571 (1.40), 2.591 (0.77), 2.882 (1.10), 3.018 (1.27), 3.035 (3.01), 3.053 (2.14), 3.239 (2.40), 3.254 (2.32), 3.368 (1.13), 3.379 (1.40), 3.391 (1.33), 3.403 (0.92), 3.493 (0.76), 4.463 (0.65), 4.482 (1.12), 4.500 (0.62), 7.033 (4.22), 7.048 (4.45), 7.074 (3.47), 7.077 (4.04), 7.100 (1.85), 7.103 (1.52), 7.113 (2.53), 7.117 (2.34), 7.162 (4.18), 7.175 (2.71), 7.439 (1.03), 7.481 (4.88), 7.495 (4.57), 7.526 (2.04), 7.613 (0.91), 7.952 (5.28).

Method B

1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (prepared in analogy to Example 3, Enantiomer 2, 31.2 mg, 51.3 μmol) were dissolved in 17 ml of dichloromethane and 1 ml of methanol. The solution was shaken once with 1.5 ml of saturated, aqueous sodium bicarbonate solution. The phases were separated. 5 ml of dichloromethane and 3 ml of methanol were added to the organic phase. The organic phase was then dried over sodium sulfate, filtered, evaporated and purified by preparative HPLC (RP18 column, acetonitrile/water gradient, neutral without acid addition). Product fractions were combined and lyophilized. 22 mg of the target compound (74% of theory) were obtained.

LC-MS (Method 3): Rₜ=1.73 min; MS (ESIpos): m/z=572 [M+H]⁺

¹H-NMR (600 MHz, DMSO-d6) δ[ppm]: 0.887 (15.60), 0.898 (16.00), 1.493 (0.64), 1.514 (0.70), 1.695 (0.89), 1.718 (0.74), 1.799 (0.48), 1.811 (0.88), 1.822 (1.12), 1.833 (0.92), 1.844 (0.48), 1.890 (0.68), 1.910 (0.74), 1.977 (0.93), 1.995 (0.62), 2.118 (3.91), 2.130 (3.66), 2.516 (5.14), 3.017 (1.09), 3.035 (2.76), 3.053 (1.94), 3.181 (5.03), 3.185 (5.02), 3.267 (1.53), 4.473 (0.55), 4.491 (0.96), 4.509 (0.54), 6.963 (3.96), 6.977 (4.06), 7.048 (3.13), 7.051 (3.31), 7.081 (1.60), 7.084 (1.26), 7.095 (2.21), 7.098 (1.89), 7.152 (3.52), 7.165 (2.42), 7.434 (4.45), 7.448 (4.50), 7.533 (1.51), 7.621 (0.67), 7.930 (4.14).

### Example 3

### 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (Enantiomer 2)

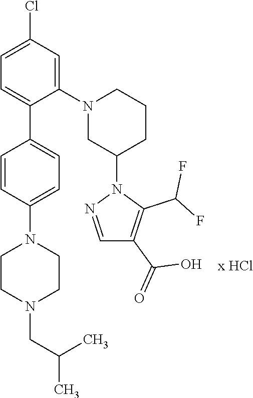 [SMILES: CC(C)CN1CCN(c2ccc(-c3ccc(Cl)cc3N3CCCC(n4ncc(C(=O)O)c4C(F)F)C3)cc2)CC1]

Method A

A suspension of 1-[1-{4-chloro-4′-[4-(2-methylpropyl)piperazin-1-yl][1,1′-biphenyl]-2-yl}piperidin-3-yl]-5-(difluoromethyl)-1H-pyrazole-4-carboxylic acid (prepared in analogy to Example 2, Enantiomer 2, 43.5 g, 76.0 mmol) in diethyl ether (870 ml) was treated with a solution of hydrogen chloride in diethyl ether (84 ml, 1.0 M, 84 mmol). The resulting mixture was stirred overnight at room temperature and evaporated affording 46.1 g (quant.) of the title compound.

LC-MS (Method 3): Rₜ=1.72 min; MS (ESIpos): m/z=572 [M+H]⁺

¹H-NMR (600 MHz, DMSO-d6) δ[ppm]: 1.026 (15.64), 1.037 (16.00), 1.497 (0.56), 1.519 (0.61), 1.722 (0.78), 1.743 (0.65), 1.903 (0.59), 1.910 (0.53), 1.924 (0.66), 1.930 (0.61), 1.978 (0.82), 1.994 (0.50), 2.142 (0.45), 2.154 (0.91), 2.165 (1.11), 2.176 (0.89), 2.187 (0.45), 2.557 (0.64), 2.577 (1.02), 2.594 (0.55), 2.992 (1.81), 3.002 (2.77), 3.012 (1.87), 3.018 (1.15), 3.036 (2.40), 3.054 (1.60), 3.133 (1.12), 3.148 (1.19), 3.168 (0.53), 3.237 (0.88), 3.250 (0.76), 3.338 (0.81), 3.360 (1.42), 3.379 (0.88), 3.580 (1.61), 3.791 (0.89), 3.819 (1.25), 3.844 (0.81), 4.463 (0.89), 4.474 (0.97), 4.481 (1.26), 4.488 (0.99), 4.499 (0.88), 7.051 (3.56), 7.065 (3.77), 7.077 (2.72), 7.080 (3.14), 7.103 (1.42), 7.106 (1.13), 7.116 (2.00), 7.120 (1.84), 7.165 (3.40), 7.178 (2.22), 7.443 (0.84), 7.489 (4.04), 7.504 (3.79), 7.531 (1.66), 7.618 (0.72), 7.954 (4.33), 10.519 (0.49).

Method B

Ethyl 1-[1-{5-chloro-2-[(trifluoromethanesulfonyl)oxy]phenyl}piperidin-3-yl]-5-(difluoromethyl)-1H-pyrazole-4-carboxylate (prepared in analogy to Example 14A, Enantiomer 2, 80.0 mg, 150 μmol) and 1-(2-methylpropyl)-4-[4-(4,4,5,5-tetramethyl-1,3,2-dioxaborolan-2-yl)phenyl]piperazine (Example 18A 64.1 mg, 97% purity, 180 μmol) were dissolved under argon in toluene/ethanol (0.83/0.83 ml). Tetrakis(triphenylphosphine)palladium(0) (8.69 mg, 7.52 μmol) and 2 M sodium carbonate solution (226 μl, 452 μmol) were added and the mixture was stirred at 100° C. overnight. The reaction mixture was diluted with ethyl acetate and water. The aqueous phase was acidified with 1 M hydrochloric acid. The phases were separated and the aqueous phase was extracted twice with ethyl acetate. The combined organic phases were dried over sodium sulfate, filtered and evaporated. The crude product was dissolved in THF/ethanol (3.9/0.39 ml), 1 M aqueous lithium hydroxide solution (1.5 ml, 1.5 mmol) was added and the mixture was stirred overnight at room temperature. The mixture was evaporated, the residue was dissolved in acetonitrile/TFA/water and purified using preparative HPLC (RP18 column, acetonitrile/water gradient with the addition of 0.1% TFA). The product fractions were combined and evaporated. The residue was mixed with 0.1 M hydrochloric acid in dioxane, carefully evaporated at 30° C. (twice) and then lyophilized. 53 mg of the target compound (55% of theory, purity 95%) were obtained.

LC-MS (Method 4): Rₜ=0.91 min; MS (ESIpos): m/z=572 [M−HCl+H]⁺

¹H-NMR (400 MHz, DMSO-d6) δ[ppm]: 1.004 (15.46), 1.020 (16.00), 1.491 (0.44), 1.522 (0.50), 1.722 (0.68), 1.753 (0.55), 1.890 (0.47), 1.920 (0.55), 1.967 (0.84), 2.129 (0.76), 2.146 (0.96), 2.163 (0.76), 2.582 (0.91), 2.613 (0.48), 2.999 (0.86), 3.010 (1.71), 3.025 (3.88), 3.041 (2.30), 3.131 (0.88), 3.161 (1.25), 3.177 (2.08), 3.213 (1.75), 3.242 (1.16), 3.467 (1.06), 3.496 (0.84), 3.503 (0.60), 3.519 (0.54), 3.525 (0.50), 3.549 (0.75), 3.555 (0.84), 3.572 (1.57), 3.582 (1.48), 3.589 (1.38), 3.601 (2.78), 3.608 (1.89), 3.633 (0.44), 3.640 (0.41), 3.811 (0.94), 3.847 (1.32), 3.878 (0.71), 4.329 (0.49), 4.439 (0.46), 4.466 (0.73), 4.477 (0.52), 4.839 (0.49), 7.047 (3.30), 7.070 (3.64), 7.082 (2.61), 7.087 (3.29), 7.104 (1.46), 7.109 (0.86), 7.124 (2.34), 7.129 (2.03), 7.160 (3.99), 7.181 (1.96), 7.388 (0.88), 7.490 (4.02), 7.512 (3.81), 7.519 (2.20), 7.650 (0.72), 7.959 (3.78), 9.708 (0.41).

[α]_{D}=−73.05°, c=0.465 g/100 cm³, trichloromethane.

Enantiomer 2 has an absolute configuration of R as shown in example 3A below.

### 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride

### Example 3A

### 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride hemihydrate

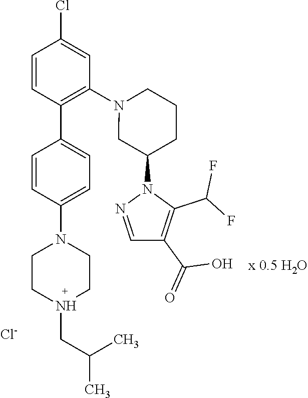 [SMILES: [Cl-] ; CC(C)CN1CCN(c2ccc(-c3ccc(Cl)cc3N3CCC[C@@H](n4ncc(C(=O)O)c4C(F)F)C3)cc2)CC1]

100 mg 1-{1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride (Enantiomer 2) (example 3) were solved at 60° C. in 3.5 ml 2-propanol, wherein the 2-propanol was dosed portionwise in 100 μl-portions at 60° C. until a clear solution was obtained. Afterwards the vessel was closed with a septum and placed into a slowly cooling sand bath from 60° C. to room temperature over the weekend->small amounts of solids were detected. Thereafter the septum was provided with a canula, in order to slowly let the solvent evaporate. After 4 weeks crystals were collected and inspected under a microscope.

Single Crystal X-Ray Structure Analysis:

The Crystal structure determination was carried out using a Bruker diffractometer (QS-no.: 02506) equipped with an Apex II-CCD area detector, an IμS-microsource with CuKa radiation, mirrors as monochromator and a Cryostream low temperature device (T=110 K). Fullsphere data collection, omega and phi scans. Programs used: Data collection and reduction Apex II v2014.11.0 (Bruker AXS, 2014), absorption correction/scaling SADABS. Crystal structure solution was achieved using direct methods as implemented in SHELXTL Version 6.14 (Bruker AXS, 2003) and visualized using XP program. Missing atoms were subsequently located from difference Fourier synthesis and added to the atom list. Least-squares refinement on F2 using all measured intensities was carried out using the program SHELXTL Version 6.14 (Bruker AXS, 2003). All non hydrogen atoms were refined including anisotropic displacement parameters.

| | Correct | Inverted |
|---|---|---|
| Chirality Check* | structure | structure |
| Flack Parameter (standard deviation) | 0.094 (0.009) | 0.906 (0.009) |
| wR2-value (with Flack Parameter) | 0.2357 | 0.2522 |
| Chirality | R(C22) | S(C22) |

*H. D. Flack, Acta Cryst., 1983, A39, 876-881
H. D. Flack, G. Bernardinelli, J. Appl. Cryst., 2000, 33, 1143-1148
S. Parsons, H. D. Flack, T. Wagner, Acta Cryst., 2013, B69, 249-259.

TABLE 1
Crystal data and structure refinement for example 3A

| Identification code | example 3A | |
|---|---|---|
| Empirical formula | C60 H76 Cl4 F4 N10 O5 | |
| Formula weight | 1235.10 | |
| Temperature | 110 K | |
| Wavelength | 1.54178 Å | |
| Crystal system | Trigonal | |
| Space group | P3₂21 | |
| Unit cell dimensions | a = 9.8693(5) Å | α = 90°. |
| | b = 9.8693(5) Å | β = 90°. |
| | c = 54.159(3) Å | γ = 120°. |

| | |
|---|---|
| Volume | 4568.5(5) Å³ |
| Z | 3 |
| Density (calculated) | 1.347 Mg/m³ |
| Absorption coefficient | 2.341 mm⁻¹ |
| F(000) | 1950 |
| Crystal size | 0.14 × 0.10 × 0.06 mm³ |
| Theta range for data collection | 4.899 to 63.664°. |
| Index ranges | −11 ≤ h ≤ 10, −10 ≤ k ≤ |
| | 11, −62 ≤ l ≤ 61 |
| Reflections collected | 27868 |
| Independent reflections | 4640 [R(int) = 0.0378] |
| Completeness to | 95.9% |
| theta = 63.664° | |
| Absorption correction | Semi-empirical from equivalents |
| Max. and min. transmission | 0.87 and 0.74 |
| Refinement method | Full-matrix least-squares on F² |
| Data/restraints/parameters | 4640/11/593 |
| Goodness-of-fit on F² | 1.047 |
| Final R indices [I > 2sigma(I)] | R1 = 0.0848, wR2 = 0.2336 |
| R indices (all data) | R1 = 0.0864, wR2 = 0.2357 |
| Absolute structure parameter | 0.094(9) |
| Extinction coefficient | n/a |
| Largest diff. peak and hole | 0.601 and −0.650 e·Å⁻³ |

TABLE 2
Bond lengths [Å] and angles [°] for example 3A.

| Cl(2)—C(3) | 1.767 | (13) | N(1)—C(10) | 1.416 | (9) |
|---|---|---|---|---|---|
| Cl(2′)—C(3′) | 1.772 | (13) | N(1)—C(16) | 1.434 | (12) |
| F(1)—C(30) | 1.341 | (7) | N(1)—C(13) | 1.470 | (10) |
| F(2)—C(30) | 1.339 | (7) | N(2)—C(14) | 1.497 | (9) |
| F(1′)—C(30′) | 1.339 | (7) | N(2)—C(15) | 1.498 | (9) |
| F(2′)—C(30′) | 1.38 | (2) | N(2)—C(17) | 1.512 | (8) |
| O(1)—C(29) | 1.22 | (2) | N(2)—H(2C) | 1.0000 | |
| O(2)—C(29) | 1.30 | (2) | N(3)—C(25) | 1.46 | (2) |
| O(2)—H(2A) | 0.8400 | | N(3)—C(21) | 1.46 | (5) |
| O(1′)—C(29′) | 1.17 | (2) | N(3)—C(1) | 1.47 | (3) |
| O(2′)—C(29′) | 1.36 | (2) | N(4)—C(26) | 1.30 | (3) |
| O(2′)—H(2B) | 0.8400 | | N(4)—N(5) | 1.32 | (3) |
| N(4)—C(22) | 1.47 | (2) | C(12)—H(12A) | 0.9500 | |
| N(5)—C(28) | 1.37 | (2) | C(13)—C(14) | 1.524 | (10) |
| N(3′)—C(1′) | 1.38 | (3) | C(13)—H(13A) | 0.9900 | |
| N(3′)—C(21′) | 1.44 | (4) | C(13)—H(13B) | 0.9900 | |
| N(3′)—C(25′) | 1.46 | (2) | C(14)—H(14A) | 0.9900 | |
| N(4′)—N(5′) | 1.38 | (3) | C(14)—H(14B) | 0.9900 | |
| N(4′)—C(26′) | 1.42 | (3) | C(15)—C(16) | 1.519 | (10) |
| N(4′)—C(22′) | 1.46 | (2) | C(15)—H(15A) | 0.9900 | |
| N(5′)—C(28′) | 1.32 | (2) | C(15)—H(15B) | 0.9900 | |
| C(1)—C(6) | 1.35 | (3) | C(16)—H(16A) | 0.9900 | |
| C(1)—C(2) | 1.42 | (4) | C(16)—H(16B) | 0.9900 | |
| C(2)—C(3) | 1.37 | (3) | C(17)—C(18) | 1.499 | (10) |
| C(2)—H(2D) | 0.9500 | | C(17)—H(17A) | 0.9900 | |
| C(3)—C(4) | 1.33 | (2) | C(17)—H(17B) | 0.9900 | |
| C(4)—C(5) | 1.390 | (19) | C(18)—C(20) | 1.509 | (11) |
| C(4)—H(4A) | 0.9500 | | C(18)—C(19) | 1.538 | (10) |
| C(5)—C(6) | 1.41 | (2) | C(18)—H(18A) | 1.0000 | |
| C(5)—H(5A) | 0.9500 | | C(19)—H(19A) | 0.9800 | |
| C(6)—C(7) | 1.506 | (17) | C(19)—H(19B) | 0.9800 | |
| C(7)—C(8) | 1.36 | (2) | C(19)—H(19C) | 0.9800 | |
| C(7)—C(12) | 1.382 | (19) | C(20)—H(20A) | 0.9800 | |
| C(7)—C(6′) | 1.58 | (2) | C(20)—H(20B) | 0.9800 | |
| C(8)—C(9) | 1.378 | (13) | C(20)—H(20C) | 0.9800 | |
| C(8)—H(8A) | 0.9500 | | C(21)—C(22) | 1.541 | (7) |
| C(9)—C(10) | 1.390 | (15) | C(21)—H(21A) | 0.9900 | |
| C(9)—H(9A) | 0.9500 | | C(21)—H(21B) | 0.9900 | |
| C(10)—C(11) | 1.390 | (16) | C(22)—C(23) | 1.56 | (2) |
| C(11)—C(12) | 1.391 | (11) | C(22)—H(22A) | 1.0000 | |
| C(11)—H(11A) | 0.9500 | | C(23)—C(24) | 1.52 | (3) |
| C(23)—H(23A) | 0.9900 | | C(21′)—C(22′) | 1.59 | (2) |
| C(23)—H(23B) | 0.9900 | | C(21′)—H(21C) | 0.9900 | |
| C(24)—C(25) | 1.52 | (2) | C(21′)—H(21D) | 0.9900 | |
| C(24)—H(24A) | 0.9900 | | C(22′)—C(23′) | 1.52 | (2) |
| C(24)—H(24B) | 0.9900 | | C(22′)—H(22B) | 1.0000 | |
| C(25)—H(25A) | 0.9900 | | C(23′)—C(24′) | 1.52 | (2) |
| C(25)—H(25B) | 0.9900 | | C(23′)—H(23C) | 0.9900 | |
| C(26)—C(27) | 1.42 | (2) | C(23′)—H(23D) | 0.9900 | |
| C(26)—C(30) | 1.500 | (7) | C(24′)—C(25′) | 1.55 | (2) |
| C(27)—C(28) | 1.34 | (3) | C(24′)—H(24C) | 0.9900 | |
| C(27)—C(29) | 1.50 | (3) | C(24′)—H(24D) | 0.9900 | |
| C(28)—H(28A) | 0.9500 | | C(25′)—H(25C) | 0.9900 | |
| C(30)—H(30A) | 1.0000 | | C(25′)—H(25D) | 0.9900 | |
| C(1′)—C(2′) | 1.39 | (3) | C(26′)—C(27′) | 1.35 | (3) |
| C(1′)—C(6′) | 1.42 | (2) | C(26′)—C(30′) | 1.46 | (3) |
| C(2′)—C(3′) | 1.39 | (3) | C(27′)—C(28′) | 1.41 | (2) |
| C(2′)—H(2E) | 0.9500 | | C(27′)—C(29′) | 1.50 | (3) |
| C(3′)—C(4′) | 1.36 | (2) | C(28′)—H(28B) | 0.9500 | |
| C(4′)—C(5′) | 1.392 | (19) | C(30′)—H(30B) | 1.0000 | |
| C(4′)—H(4B) | 0.9500 | | O(1W)—H(1W) | 0.9010 | |
| C(5′)—C(6′) | 1.40 | (2) | O(1W)—H(1W)#1 | 0.9010 | |
| C(5′)—H(5B) | 0.9500 | | C(14)—N(2)—C(17) | 108.8 | (5) |
| C(29)—O(2)—H(2A) | 109.5 | | C(15)—N(2)—C(17) | 113.0 | (5) |
| C(29′)—O(2′)—H(2B) | 109.5 | | C(14)—N(2)—H(2C) | 108.6 | |
| C(10)—N(1)—C(16) | 117.9 | (8) | C(15)—N(2)—H(2C) | 108.6 | |
| C(10)—N(1)—C(13) | 113.5 | (6) | C(17)—N(2)—H(2C) | 108.6 | |
| C(16)—N(1)—C(13) | 109.6 | (5) | C(25)—N(3)—C(21) | 107 | (2) |
| C(14)—N(2)—C(15) | 109.2 | (5) | C(1)—C(6)—C(7) | 112.0 | (17) |
| C(25)—N(3)—C(1) | 116.5 | (18) | C(5)—C(6)—C(7) | 128.4 | (16) |
| C(21)—N(3)—C(1) | 112.2 | (18) | C(8)—C(7)—C(12) | 115.2 | (8) |
| C(26)—N(4)—N(5) | 113 | (2) | C(8)—C(7)—C(6) | 109.3 | (13) |
| C(26)—N(4)—C(22) | 127 | (2) | C(12)—C(7)—C(6) | 135.5 | (15) |
| N(5)—N(4)—C(22) | 120 | (2) | C(8)—C(7)—C(6′) | 136.3 | (13) |
| N(4)—N(5)—C(28) | 104 | (2) | C(12)—C(7)—C(6′) | 108.4 | (14) |
| C(1′)—N(3′)—C(21′) | 112.1 | (19) | C(7)—C(8)—C(9) | 124.1 | (12) |
| C(1′)—N(3′)—C(25′) | 117.2 | (19) | C(7)—C(8)—H(8A) | 118.0 | |
| C(21′)—N(3′)—C(25′) | 119.2 | (19) | C(9)—C(8)—H(8A) | 118.0 | |
| N(5′)—N(4′)—C(26′) | 109 | (2) | C(8)—C(9)—C(10) | 120.2 | (13) |
| N(5′)—N(4′)—C(22′) | 118.1 | (15) | C(8)—C(9)—H(9A) | 119.9 | |
| C(26′)—N(4′)—C(22′) | 128 | (2) | C(10)—C(9)—H(9A) | 119.9 | |
| C(28′)—N(5′)—N(4′) | 106.9 | (15) | C(9)—C(10)—C(11) | 117.3 | (8) |
| C(6)—C(1)—C(2) | 119 | (2) | C(9)—C(10)—N(1) | 121.7 | (10) |
| C(6)—C(1)—N(3) | 120.5 | (18) | C(11)—C(10)—N(1) | 120.9 | (9) |
| C(2)—C(1)—N(3) | 120 | (2) | C(10)—C(11)—C(12) | 120.2 | (11) |
| C(3)—C(2)—C(1) | 118.4 | (19) | C(10)—C(11)—H(11A) | 119.9 | |
| C(3)—C(2)—H(2D) | 120.8 | | C(12)—C(11)—H(11A) | 119.9 | |
| C(1)—C(2)—H(2D) | 120.8 | | C(7)—C(12)—C(11) | 123.0 | (13) |
| C(4)—C(3)—C(2) | 123.8 | (15) | C(7)—C(12)—H(12A) | 118.5 | |
| C(4)—C(3)—Cl(2) | 120.9 | (12) | C(11)—C(12)—H(12A) | 118.5 | |
| C(2)—C(3)—Cl(2) | 115.1 | (14) | N(1)—C(13)—C(14) | 110.8 | (6) |
| C(3)—C(4)—C(5) | 117.5 | (14) | N(1)—C(13)—H(13A) | 109.5 | |
| C(3)—C(4)—H(4A) | 121.3 | | C(14)—C(13)—H(13A) | 109.5 | |
| C(5)—C(4)—H(4A) | 121.3 | | N(1)—C(13)—H(13B) | 109.5 | |
| C(4)—C(5)—C(6) | 121.0 | (15) | C(14)—C(13)—H(13B) | 109.5 | |
| C(4)—C(5)—H(5A) | 119.5 | | H(13A)—C(13)—H(13B) | 108.1 | |
| C(6)—C(5)—H(5A) | 119.5 | | N(2)—C(14)—C(13) | 110.7 | (6) |
| C(1)—C(6)—C(5) | 119.5 | (15) | C(18)—C(19)—H(19A) | 109.5 | |
| N(2)—C(14)—H(14A) | 109.5 | | C(18)—C(19)—H(19B) | 109.5 | |
| C(13)—C(14)—H(14A) | 109.5 | | H(19A)—C(19)—H(19B) | 109.5 | |
| N(2)—C(14)—H(14B) | 109.5 | | C(18)—C(19)—H(19C) | 109.5 | |
| C(13)—C(14)—H(14B) | 109.5 | | H(19A)—C(19)—H(19C) | 109.5 | |
| H(14A)—C(14)—H(14B) | 108.1 | | H(19B)—C(19)—H(19C) | 109.5 | |
| N(2)—C(15)—C(16) | 110.4 | (6) | C(18)—C(20)—H(20A) | 109.5 | |
| N(2)—C(15)—H(15A) | 109.6 | | C(18)—C(20)—H(20B) | 109.5 | |
| C(16)—C(15)—H(15A) | 109.6 | | H(20A)—C(20)—H(20B) | 109.5 | |
| N(2)—C(15)—H(15B) | 109.6 | | C(18)—C(20)—H(20C) | 109.5 | |
| C(16)—C(15)—H(15B) | 109.6 | | H(20A)—C(20)—H(20C) | 109.5 | |
| H(15A)—C(15)—H(15B) | 108.1 | | H(20B)—C(20)—H(20C) | 109.5 | |
| N(1)—C(16)—C(15) | 112.1 | (7) | N(3)—C(21)—C(22) | 106 | (3) |
| N(1)—C(16)—H(16A) | 109.2 | | N(3)—C(21)—H(21A) | 110.4 | |
| C(15)—C(16)—H(16A) | 109.2 | | C(22)—C(21)—H(21A) | 110.4 | |
| N(1)—C(16)—H(16B) | 109.2 | | N(3)—C(21)—H(21B) | 110.4 | |
| C(15)—C(16)—H(16B) | 109.2 | | C(22)—C(21)—H(21B) | 110.4 | |
| H(16A)—C(16)—H(16B) | 107.9 | | H(21A)—C(21)—H(21B) | 108.6 | |
| C(18)—C(17)—N(2) | 115.7 | (5) | N(4)—C(22)—C(21) | 110 | (2) |
| C(18)—C(17)—H(17A) | 108.4 | | N(4)—C(22)—C(23) | 106.8 | (16) |
| N(2)—C(17)—H(17A) | 108.4 | | C(21)—C(22)—C(23) | 105 | (2) |
| C(18)—C(17)—H(17B) | 108.4 | | N(4)—C(22)—H(22A) | 111.7 | |
| N(2)—C(17)—H(17B) | 108.4 | | C(21)—C(22)—H(22A) | 111.7 | |
| H(17A)—C(17)—H(17B) | 107.4 | | C(23)—C(22)—H(22A) | 111.7 | |
| C(17)—C(18)—C(20) | 114.1 | (6) | C(24)—C(23)—C(22) | 108.9 | (13) |
| C(17)—C(18)—C(19) | 108.2 | (6) | C(24)—C(23)—H(23A) | 109.9 | |
| C(20)—C(18)—C(19) | 110.6 | (6) | C(22)—C(23)—H(23A) | 109.9 | |
| C(17)—C(18)—H(18A) | 107.9 | | C(24)—C(23)—H(23B) | 109.9 | |
| C(20)—C(18)—H(18A) | 107.9 | | C(22)—C(23)—H(23B) | 109.9 | |
| C(19)—C(18)—H(18A) | 107.9 | | F(1)—C(30)—H(30A) | 109.9 | |
| H(23A)—C(23)—H(23B) | 108.3 | | C(26)—C(30)—H(30A) | 109.9 | |
| C(23)—C(24)—C(25) | 112.6 | (13) | N(3′)—C(1′)—C(2′) | 119.2 | (17) |
| C(23)—C(24)—H(24A) | 109.1 | | N(3′)—C(1′)—C(6′) | 120.3 | (18) |
| C(25)—C(24)—H(24A) | 109.1 | | C(2′)—C(1′)—C(6′) | 120 | (2) |
| C(23)—C(24)—H(24B) | 109.1 | | C(1′)—C(2′)—C(3′) | 118.4 | (18) |
| C(25)—C(24)—H(24B) | 109.1 | | C(1′)—C(2′)—H(2E) | 120.8 | |
| H(24A)—C(24)—H(24B) | 107.8 | | C(3′)—C(2′)—H(2E) | 120.8 | |
| N(3)—C(25)—C(24) | 107.3 | (15) | C(4′)—C(3′)—C(2′) | 125.1 | (15) |
| N(3)—C(25)—H(25A) | 110.3 | | C(4′)—C(3′)—Cl(2′) | 118.0 | (12) |
| C(24)—C(25)—H(25A) | 110.3 | | C(2′)—C(3′)—Cl(2′) | 116.8 | (12) |
| N(3)—C(25)—H(25B) | 110.3 | | C(3′)—C(4′)—C(5′) | 114.4 | (13) |
| C(24)—C(25)—H(25B) | 110.3 | | C(3′)—C(4′)—H(4B) | 122.8 | |
| H(25A)—C(25)—H(25B) | 108.5 | | C(5′)—C(4′)—H(4B) | 122.8 | |
| N(4)—C(26)—C(27) | 107.8 | (18) | C(4′)—C(5′)—C(6′) | 125.3 | (14) |
| N(4)—C(26)—C(30) | 124 | (2) | C(4′)—C(5′)—H(5B) | 117.3 | |
| C(27)—C(26)—C(30) | 127.8 | (16) | C(6′)—C(5′)—H(5B) | 117.3 | |
| C(28)—C(27)—C(26) | 102.7 | (18) | C(5′)—C(6′)—C(1′) | 116.2 | (16) |
| C(28)—C(27)—C(29) | 133 | (2) | C(5′)—C(6′)—C(7) | 109.8 | (15) |
| C(26)—C(27)—C(29) | 124.0 | (19) | C(1′)—C(6′)—C(7) | 131.7 | (15) |
| C(27)—C(28)—N(5) | 112.9 | (19) | N(3′)—C(21′)—C(22′) | 109 | (2) |
| C(27)—C(28)—H(28A) | 123.6 | | N(3′)—C(21′)—H(21C) | 109.9 | |
| N(5)—C(28)—H(28A) | 123.6 | | C(22′)—C(21′)—H(21C) | 109.9 | |
| O(1)—C(29)—O(2) | 123 | (2) | N(3′)—C(21′)—H(21D) | 109.9 | |
| O(1)—C(29)—C(27) | 125.0 | (19) | C(22′)—C(21′)—H(21D) | 109.9 | |
| O(2)—C(29)—C(27) | 112 | (2) | H(21C)—C(21′)—H(21D) | 108.3 | |
| F(2)—C(30)—F(1) | 104.4 | (13) | N(4′)—C(22′)—C(23′) | 108.7 | (16) |
| F(2)—C(30)—C(26) | 112.1 | (18) | N(4′)—C(22′)—C(21′) | 111.0 | (16) |
| F(1)—C(30)—C(26) | 110.6 | (17) | C(23′)—C(22′)—C(21′) | 117.6 | (19) |
| F(2)—C(30)—H(30A) | 109.9 | | C(27′)—C(26′)—N(4′) | 105.4 | (19) |
| N(4′)—C(22′)—H(22B) | 106.3 | | C(27′)—C(26′)—C(30′) | 134.7 | (19) |
| C(23′)—C(22′)—H(22B) | 106.3 | | N(4′)—C(26′)—C(30′) | 120 | (3) |
| C(21′)—C(22′)—H(22B) | 106.3 | | C(26′)—C(27′)—C(28′) | 108.0 | (15) |
| C(22′)—C(23′)—C(24′) | 107.4 | (15) | C(26′)—C(27′)—C(29′) | 128.4 | (19) |
| C(22′)—C(23′)—H(23C) | 110.2 | | C(28′)—C(27′)—C(29′) | 123.1 | (17) |
| C(24′)—C(23′)—H(23C) | 110.2 | | N(5′)—C(28′)—C(27′) | 110.3 | (16) |
| C(22′)—C(23′)—H(23D) | 110.2 | | N(5′)—C(28′)—H(28B) | 124.8 | |
| C(24′)—C(23′)—H(23D) | 110.2 | | C(27′)—C(28′)—H(28B) | 124.8 | |
| H(23C)—C(23′)—H(23D) | 108.5 | | O(1′)—C(29′)—O(2′) | 126.1 | (19) |
| C(23′)—C(24′)—C(25′) | 114.0 | (14) | O(1′)—C(29′)—C(27′) | 124.4 | (16) |

| | | | | |
|---|---|---|---|---|
| C(23′)—C(24′)—H(24C) | 108.8 | O(2′)—C(29′)—C(27′) | 109.4 | (19) |
| C(25′)—C(24′)—H(24C) | 108.8 | F(1′)—C(30′)—F(2′) | 107.3 | (18) |
| C(23′)—C(24′)—H(24D) | 108.8 | F(1′)—C(30′)—C(26′) | 111.2 | (19) |
| C(25′)—C(24′)—H(24D) | 108.8 | F(2′)—C(30′)—C(26′) | 112.0 | (17) |

| H(24C)—C(24′)—H(24D) | 107.7 | | F(1′)—C(30′)—H(30B) | 108.7 |
|---|---|---|---|---|
| N(3′)—C(25′)—C(24′) | 106.9 | (15) | F(2′)—C(30′)—H(30B) | 108.7 |

| N(3′)—C(25′)—H(25C) | 110.3 | C(26′)—C(30′)—H(30B) | 108.7 |
|---|---|---|---|
| C(24′)—C(25′)—H(25C) | 110.3 | H(1W)—O(1W)—H(1W)#1 | 107.2 |
| N(3′)—C(25′)—H(25D) | 110.3 | | |
| C(24′)—C(25′)—H(25D) | 110.3 | | |
| H(25C)—C(25′)—H(25D) | 108.6 | | |

Symmetry transformations used to generate equivalent atoms: #1 y − 1, x + 1, −z + 1

TABLE 3
Torsion angles [°] for example 3A

| | | | | | |
|---|---|---|---|---|---|
| C(26)—N(4)—N(5)—C(28) | 4 | (2) | C(25)—N(3)—C(1)—C(2) | −25 | (3) |
| C(22)—N(4)—N(5)—C(28) | −173.4 | (17) | C(21)—N(3)—C(1)—C(2) | 99 | (3) |
| C(26′)—N(4′)—N(5′)—C(28′) | 0 | (2) | C(6)—C(1)—C(2)—C(3) | 9 | (3) |
| C(22′)—N(4′)—N(5′)—C(28′) | −157.8 | (16) | N(3)—C(1)—C(2)—C(3) | −177.2 | (18) |
| C(25)—N(3)—C(1)—C(6) | 148.8 | (17) | C(1)—C(2)—C(3)—C(4) | −7 | (3) |
| C(21)—N(3)—C(1)—C(6) | −87 | (3) | C(1)—C(2)—C(3)—C1(2) | 178.6 | (14) |
| C(2)—C(3)—C(4)—C(5) | 5 | (3) | C(10)—C(11)—C(12)—C(7) | 0.0 | (14) |
| Cl(2)—C(3)—C(4)—C(5) | 178.8 | (12) | C(10)—N(1)—C(13)—C(14) | 167.0 | (7) |
| C(3)—C(4)—C(5)—C(6) | −4 | (2) | C(16)—N(1)—C(13)—C(14) | −58.8 | (8) |
| C(2)—C(1)—C(6)—C(5) | −8 | (3) | C(15)—N(2)—C(14)—C(13) | −55.6 | (7) |
| N(3)—C(1)—C(6)—C(5) | 178.0 | (16) | C(17)—N(2)—C(14)—C(13) | −179.3 | (6) |
| C(2)—C(1)—C(6)—C(7) | 169.6 | (16) | N(1)—C(13)—C(14)—N(2) | 57.9 | (8) |
| N(3)—C(1)—C(6)—C(7) | −5 | (2) | C(14)—N(2)—C(15)—C(16) | 55.1 | (8) |
| C(4)—C(5)—C(6)—C(1) | 6 | (2) | C(17)—N(2)—C(15)—C(16) | 176.4 | (6) |
| C(4)—C(5)—C(6)—C(7) | −171.3 | (14) | C(10)—N(1)—C(16)—C(15) | −168.9 | (6) |
| C(1)—C(6)—C(7)—C(8) | 148.5 | (14) | C(13)—N(1)—C(16)—C(15) | 59.3 | (7) |
| C(5)—C(6)—C(7)—C(8) | −34.4 | (18) | N(2)—C(15)—C(16)—N(1) | −58.4 | (8) |
| C(1)—C(6)—C(7)—C(12) | −33.3 | (19) | C(14)—N(2)—C(17)—C(18) | 178.4 | (6) |
| C(5)—C(6)—C(7)—C(12) | 143.8 | (15) | C(15)—N(2)—C(17)—C(18) | 56.9 | (8) |
| C(12)—C(7)—C(8)—C(9) | 2.5 | (13) | N(2)—C(17)—C(18)—C(20) | 58.0 | (8) |
| C(6)—C(7)—C(8)—C(9) | −178.9 | (9) | N(2)—C(17)—C(18)—C(19) | −178.5 | (6) |
| C(6′)—C(7)—C(8)—C(9) | 178.8 | (11) | C(25)—N(3)—C(21)—C(22) | −75 | (3) |
| C(7)—C(8)—C(9)—C(10) | −1.1 | (13) | C(1)—N(3)—C(21)—C(22) | 156 | (2) |
| C(8)—C(9)—C(10)—C(11) | −1.0 | (11) | C(26)—N(4)—C(22)—C(21) | 131 | (3) |
| C(8)—C(9)—C(10)—N(1) | −179.4 | (7) | N(5)—N(4)—C(22)—C(21) | −52 | (3) |
| C(16)—N(1)—C(10)—C(9) | −176.9 | (7) | C(26)—N(4)—C(22)—C(23) | −116 | (2) |
| C(13)—N(1)—C(10)—C(9) | −46.9 | (9) | N(5)—N(4)—C(22)—C(23) | 61 | (2) |
| C(16)—N(1)—C(10)—C(11) | 4.8 | (10) | N(3)—C(21)—C(22)—N(4) | −177 | (2) |
| C(13)—N(1)—C(10)—C(11) | 134.9 | (8) | N(3)—C(21)—C(22)—C(23) | 68 | (3) |
| C(9)—C(10)—C(11)—C(12) | 1.5 | (12) | N(4)—C(22)—C(23)—C(24) | −173.8 | (14) |
| N(1)—C(10)—C(11)—C(12) | 179.9 | (7) | C(21)—C(22)—C(23)—C(24) | −57 | (2) |
| C(8)—C(7)—C(12)—C(11) | −1.9 | (13) | C(22)—C(23)—C(24)—C(25) | 53.5 | (18) |
| C(6)—C(7)—C(12)—C(11) | 179.9 | (11) | C(21)—N(3)—C(25)—C(24) | 67 | (2) |
| C(6′)—C(7)—C(12)—C(11) | −179.2 | (10) | C(1)—N(3)—C(25)—C(24) | −166.6 | (17) |
| C(23)—C(24)—C(25)—N(3) | −56.8 | (19) | Cl(2′)—C(3′)—C(4′)—C(5′) | 179.0 | (12) |
| N(5)—N(4)—C(26)—C(27) | −3 | (2) | C(3′)—C(4′)—C(5′)—C(6′) | 1 | (2) |
| C(22)—N(4)—C(26)—C(27) | 174.2 | (19) | C(4′)—C(5′)—C(6′)—C(1′) | 4 | (3) |
| N(5)—N(4)—C(26)—C(30) | 179.8 | (15) | C(4′)—C(5′)—C(6′)—C(7) | 168.4 | (15) |
| C(22)—N(4)—C(26)—C(30) | −3 | (3) | N(3′)—C(1′)—C(6′)—C(5′) | 177.6 | (19) |
| N(4)—C(26)—C(27)—C(28) | 1 | (2) | C(2′)—C(1′)—C(6′)—C(5′) | −5 | (3) |
| C(30)—C(26)—C(27)—C(28) | 177.7 | (15) | N(3′)—C(1′)—C(6′)—C(7) | 17 | (3) |
| N(4)—C(26)—C(27)—C(29) | −175.0 | (16) | C(2′)—C(1′)—C(6′)—C(7) | −166.3 | (19) |
| C(30)—C(26)—C(27)—C(29) | 2 | (3) | C(8)—C(7)—C(6′)—C(5′) | −39.2 | (19) |
| C(26)—C(27)—C(28)—N(5) | 2 | (2) | C(12)—C(7)—C(6′)—C(5′) | 137.2 | (12) |
| C(29)—C(27)—C(28)—N(5) | 176.9 | (18) | C(8)—C(7)—C(6′)—C(1′) | 122.5 | (19) |
| N(4)—N(5)—C(28)—C(27) | −3.6 | (19) | C(12)—C(7)—C(6′)—C(1′) | −61 | (2) |
| C(28)—C(27)—C(29)—O(1) | 146.4 | (19) | C(1′)—N(3′)—C(21′)—C(22′) | 168.4 | (18) |
| C(26)—C(27)—C(29)—O(1) | −39 | (3) | C(25′)—N(3′)—C(21′)—C(22′) | −49 | (3) |
| C(28)—C(27)—C(29)—O(2) | −31 | (3) | N(5′)—N(4′)—C(22′)—C(23′) | 65 | (2) |
| C(26)—C(27)—C(29)—O(2) | 143 | (2) | C(26′)—N(4′)—C(22′)—C(23′) | −88 | (3) |
| N(4)—C(26)—C(30)—F(2) | 53 | (2) | N(5′)—N(4′)—C(22′)—C(21′) | −66 | (3) |
| C(27)—C(26)—C(30)—F(2) | −124 | (2) | C(26′)—N(4′)—C(22′)—C(21′) | 141 | (2) |
| N(4)—C(26)—C(30)—F(1) | −63 | (2) | N(3′)—C(21′)—C(22′)—N(4′) | 169.0 | (19) |
| C(27)—C(26)—C(30)—F(1) | 120 | (2) | N(3′)—C(21′)—C(22′)—C(23′) | 43 | (3) |
| C(21′)—N(3′)—C(1′)—C(2′) | 112 | (2) | N(4′)—C(22′)—C(23′)—C(24′) | −173.4 | (15) |
| C(25′)—N(3′)—C(1′)—C(2′) | −31 | (3) | C(21′)—C(22′)—C(23′)—C(24′) | −46 | (2) |
| C(21′)—N(3′)—C(1′)—C(6′) | −71 | (2) | C(22′)—C(23′)—C(24′)—C(25′) | 55 | (2) |
| C(25′)—N(3′)—C(1′)—C(6′) | 146.4 | (17) | C(1′)—N(3′)—C(25′)—C(24′) | −161.9 | (18) |
| N(3′)—C(1′)—C(2′)—C(3′) | 180.0 | (19) | C(21′)—N(3′)—C(25′)—C(24′) | 58 | (2) |
| C(6′)—C(1′)—C(2′)—C(3′) | 3 | (3) | C(23′)—C(24′)—C(25′)—N(3′) | −59 | (2) |
| C(1′)—C(2′)—C(3′)—C(4′) | 2 | (3) | N(5′)—N(4′)—C(26′)—C(27′) | −1 | (2) |
| C(1′)—C(2′)—C(3′)—Cl(2′) | 179.1 | (15) | C(22′)—N(4′)—C(26′)—C(27′) | 154 | (2) |
| C(2′)—C(3′)—C(4′)—C(5′) | −4 | (3) | N(5′)—N(4′)—C(26′)—C(30′) | −176.3 | (16) |
| C(22′)—N(4′)—C(26′)—C(30′) | −21 | (3) | C(26′)—C(27′)—C(29′)—O(1′) | 162.9 | (18) |
| N(4′)—C(26′)—C(27′)—C(28′) | 1.2 | (19) | C(28′)—C(27′)—C(29′)—O(1′) | −8 | (3) |
| C(30′)—C(26′)—C(27′)—C(28′) | 175.6 | (19) | C(26′)—C(27′)—C(29′)—O(2′) | −21 | (2) |
| N(4′)—C(26′)—C(27′)—C(29′) | −171.1 | (16) | C(28′)—C(27′)—C(29′)—O(2′) | 167.6 | (16) |
| C(30′)—C(26′)—C(27′)—C(29′) | 3 | (3) | C(27′)—C(26′)—C(30′)—F(1′) | 132 | (2) |
| N(4′)—N(5′)—C(28′)—C(27′) | 1 | (2) | N(4′)—C(26′)—C(30′)—F(1′) | −54 | (2) |
| C(26′)—C(27′)—C(28′)—N(5′) | −1 | (2) | C(27′)—C(26′)—C(30′)—F(2′) | −108 | (2) |
| C(29′)—C(27′)—C(28′)—N(5′) | 171.6 | (15) | N(4′)—C(26′)—C(30′)—F(2′) | 66 | (2) |

Symmetry transformation used to generate equivalent atoms: #1 y − 1, x + 1, −z + 1

TABLE 4
Hydrogen bonds for example 3A [Å and °].

| D—H | d(D—H) | d(H . . . A) | <DHA | d(D . . . A) | A | |
|---|---|---|---|---|---|---|
| O2\hat{ }a—H2A\hat{ }a | 0.840 | 2.268 | 171.52 | 3.102 | Cl1 | [x + 1, |
| | | | | | | y − 1, z] |
| O2′\hat{ }b—H2B\hat{ }b | 0.840 | 2.219 | 158.79 | 3.018 | Cl1 | [x + 1, |
| | | | | | | y − 1, z] |
| N2—H2C | 1.000 | 2.158 | 162.74 | 3.128 | Cl1 | [y, |
| | | | | | | x, −z + 1] |

| | | | | | |
|---|---|---|---|---|---|
| O1W—H1W | 0.901 | 2.448 | 164.20 | 3.324 | Cl1 |

FIG. 6: Ortep-Plot (50%) with labeling scheme (without disorder), example 3A

FIG. 7: Independent molecules in the asymmetric unit (with disorder), example 3A

FIG. 8: Configuration of C22, example 3A

### Example 4

### 1-{1-[4-Chloro-4′-(4-propylpiperazin-1-yl)[biphenyl]-2-yl]piperidin-3-yl}-5-(difluoromethyl)-1H-pyrazole-4-carboxylic acid hydrochloride (Enantiomer 2)

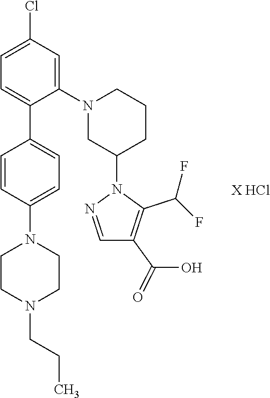

Ethyl 1-{1-[4-chloro-4′-(4-propylpiperazin-1-yl)[1,1′-biphenyl]-2-yl]piperidin-3-yl}-5-(difluoromethyl)-1H-pyrazole-4-carboxylate (Enantiomer 2, 97.0 mg, 139 μmol) was dissolved in THF/ethanol (1.9/0.19 ml). 1 M aqueous lithium hydroxide solution (1.4 ml, 1.4 mmol) was added and the mixture was stirred overnight at room temperature. The mixture was evaporated, then acidified and purified using preparative HPLC (RP18 column, acetonitrile/water gradient with the addition of 0.1% TFA). The product fractions were combined and evaporated. Then the residue was mixed with 0.1 M hydrochloric acid in dioxane, carefully evaporated at 30° C. (twice) and then lyophilized. 68 mg of the target compound (82% of theory) were obtained.

LC-MS (Method 4): Rₜ=1.72 min; MS (ESIpos): m/z=558 [M−HCl+H]⁺

¹H-NMR (600 MHz, DMSO-d6) δ[ppm]: 0.927 (7.59), 0.940 (16.00), 0.952 (7.89), 1.474 (0.44), 1.496 (1.25), 1.517 (1.37), 1.538 (0.56), 1.719 (1.74), 1.741 (1.49), 1.753 (0.97), 1.766 (2.44), 1.779 (3.67), 1.786 (2.49), 1.793 (3.59), 1.806 (2.32), 1.818 (0.65), 1.880 (0.45), 1.886 (0.49), 1.900 (1.35), 1.906 (1.24), 1.921 (1.45), 1.927 (1.37), 1.942 (0.70), 1.977 (1.87), 1.992 (1.13), 2.572 (2.30), 2.592 (1.22), 3.016 (2.00), 3.034 (5.57), 3.052 (5.43), 3.058 (5.00), 3.077 (3.34), 3.086 (2.24), 3.115 (2.60), 3.130 (2.85), 3.150 (1.37), 3.215 (1.95), 3.548 (4.23), 3.569 (5.69), 3.827 (2.12), 3.851 (3.24), 3.876 (1.87), 4.023 (0.52), 4.329 (0.46), 4.459 (1.32), 4.470 (1.47), 4.477 (2.13), 4.484 (1.51), 4.495 (1.26), 4.842 (0.83), 7.053 (8.27), 7.068 (8.76), 7.076 (6.37), 7.079 (6.96), 7.102 (3.18), 7.105 (2.52), 7.115 (4.43), 7.119 (3.97), 7.165 (7.58), 7.179 (4.92), 7.443 (1.92), 7.486 (9.24), 7.500 (8.36), 7.530 (3.79), 7.618 (1.70), 7.952 (9.70), 11.078 (0.84).

### Example 5

### 1-(1-{4-Chloro-4′-[4-(cyclopropylmethyl)piperazin-1-yl][biphenyl]-2-yl}piperidin-3-yl)-5-(difluoromethyl)-1H-pyrazole-4-carboxylic acid hydrochloride (Enantiomer 2)

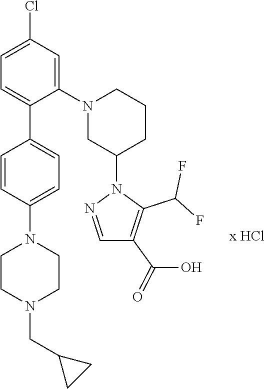 [SMILES: O=C(O)c1cnn(C2CCCN(c3cc(Cl)ccc3-c3ccc(N4CCN(CC5CC5)CC4)cc3)C2)c1C(F)F]

A solution of 1-{1-[4-chloro-4′-(piperazin-1-yl)[1,1′-biphenyl]-2-yl]piperidin-3-yl}-5-(difluoromethyl)-1H-pyrazole-4-carboxylic acid (Enantiomer 2, 175 mg, 339 μmol) in acetonitrile (3.1 ml) was treated with cyclopropanecarboxaldehyde (180 μl, 2.4 mmol) and sodium triacetoxyborohydride (216 mg, 1.02 mmol) and stirred overnight at room temperature. The reaction mixture was diluted with water and purified by preparative HPLC (RP18 column, eluent: Acetonitrile/water gradient) and evaporated. The residue was strirred in an aqeous solution of hydrogen chloride and lyophilized affording 193 mg (94% yield) of the title compound.

LC-MS (Method 4): Rₜ=1.71 min; MS (ESIpos): m/z=570 [M−HCl+H]⁺

¹H-NMR (400 MHz, DMSO-d6) δ[ppm]: 0.008 (3.75), 0.404 (1.78), 0.416 (7.42), 0.428 (7.54), 0.441 (2.23), 0.667 (5.71), 0.687 (6.02), 1.163 (1.94), 1.492 (1.30), 1.524 (1.42), 1.721 (2.07), 1.754 (1.58), 1.889 (1.40), 1.918 (1.60), 1.969 (2.29), 2.329 (0.68), 2.368 (0.70), 2.584 (2.75), 2.613 (1.40), 2.672 (0.72), 2.712 (0.80), 2.999 (2.19), 3.026 (4.93), 3.053 (7.20), 3.069 (7.86), 3.161 (7.98), 3.216 (2.87), 3.242 (2.05), 3.629 (14.26), 3.644 (16.00), 3.864 (4.63), 3.893 (4.81), 3.919 (3.11), 4.440 (1.32), 4.468 (2.19), 4.494 (1.18), 7.054 (9.40), 7.076 (11.45), 7.081 (9.08), 7.086 (9.54), 7.103 (3.93), 7.124 (6.28), 7.128 (5.61), 7.163 (11.39), 7.183 (5.71), 7.394 (2.53), 7.490 (11.05), 7.512 (10.19), 7.525 (5.19), 7.655 (2.09), 7.958 (11.99), 10.563 (0.64).

### Example 6

### 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (Enantiomer 1)

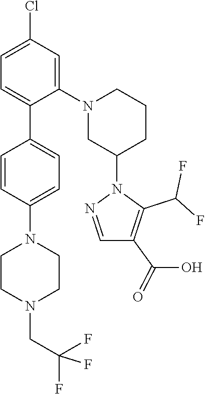 [SMILES: O=C(O)c1cnn(C2CCCN(c3cc(Cl)ccc3-c3ccc(N4CCN(CC(F)(F)F)CC4)cc3)C2)c1C(F)F]

An aqueous solution of lithium hydroxide (1.0 ml, 1.0 M, 1.0 mmol) was added to a solution of ethyl 1-[1-[5-chloro-2-[4-[4-(2,2,2-trifluoroethyl)piperazin-1-yl]phenyl]phenyl]-3-piperidyl]-5-(difluoromethyl)pyrazole-4-carboxylate (Enantiomer 1, 62.7 mg, 100 μmol) in a THF/Methanol mixture (10:1, 2.2 mL). The reaction mixture was stirred overnight at room temperature. An ageuouse solution of hydrogen chloride (6N) was then added and the resulting mixture was extracted with dichloromethane. The combined organic layers were evaporated affording 57.9 mg (90% purity, 93% yield) of the title compound.

LC-MS (Method 4): Rₜ=2.68 min; MS (ESIpos): m/z=598 [M+H]⁺

¹H-NMR (400 MHz, DMSO-d6) δ[ppm]: 0.886 (0.89), 1.117 (0.75), 1.128 (1.08), 1.170 (2.21), 1.183 (0.78), 1.237 (0.99), 1.271 (0.42), 1.356 (1.93), 1.489 (1.60), 1.520 (1.83), 1.700 (2.49), 1.743 (5.12), 1.752 (5.38), 1.760 (12.17), 1.769 (5.24), 1.776 (4.32), 1.794 (0.56), 1.864 (0.59), 1.885 (1.74), 1.916 (2.02), 1.978 (2.75), 2.329 (0.70), 2.367 (0.92), 2.578 (2.44), 2.671 (0.96), 2.711 (1.13), 2.810 (13.27), 3.005 (2.68), 3.032 (7.52), 3.058 (5.17), 3.262 (4.51), 3.283 (4.75), 3.310 (4.28), 3.585 (8.11), 3.601 (15.55), 3.618 (10.01), 4.459 (1.48), 4.486 (2.61), 4.513 (1.41), 5.754 (12.15), 7.002 (6.93), 7.024 (7.45), 7.059 (8.88), 7.064 (10.57), 7.086 (4.82), 7.091 (3.41), 7.107 (7.47), 7.112 (6.51), 7.155 (12.55), 7.175 (6.93), 7.387 (3.10), 7.452 (12.64), 7.473 (11.21), 7.518 (5.80), 7.649 (2.54), 7.954 (16.00).

### Example 7

### 1-[1-[5-Chloro-2-[4-[4-(2,2,2-trifluoroethyl)piperazin-1-yl]phenyl]phenyl]-3-piperidyl]-5-(difluoromethyl)pyrazole-4-carboxylic acid (Enantiomer 2)

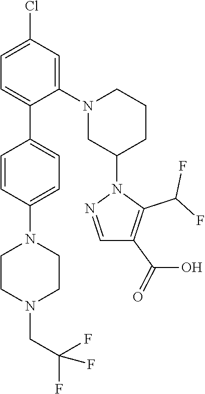 [SMILES: O=C(O)c1cnn(C2CCCN(c3cc(Cl)ccc3-c3ccc(N4CCN(CC(F)(F)F)CC4)cc3)C2)c1C(F)F]

An aqueous solution of lithium hydroxide (1.1 ml, 1.0 M, 1.1 mmol) was added to a solution of ethyl 1-[1-[5-chloro-2-[4-[4-(2,2,2-trifluoroethyl)piperazin-1-yl]phenyl]phenyl]-3-piperidyl]-5-(difluoromethyl)pyrazole-4-carboxylate (Enantiomer 2, 66.6 mg, 106 μmol) in a THF/Methanol mixture (10:1, 2.2 mL). The reaction mixture was stirred overnight at RT. An aqueous solution of hydrogen chloride (6N) was then added and the resulting mixture was extracted with dichloromethane. The combined organic layers were evaporated affording 54.7 mg (90% purity, 77% yield) of the title compound.

LC-MS (Method 4): Rₜ=2.67 min; MS (ESIpos): m/z=598 [M+H]⁺

¹H-NMR (400 MHz, DMSO-d6) δ[ppm]: 0.886 (0.65), 0.948 (0.41), 1.092 (0.48), 1.107 (0.60), 1.116 (1.02), 1.128 (0.84), 1.169 (2.79), 1.183 (1.08), 1.236 (0.93), 1.271 (0.60), 1.356 (1.02), 1.489 (1.77), 1.521 (2.07), 1.700 (2.77), 1.743 (5.62), 1.751 (5.95), 1.760 (12.78), 1.768 (5.69), 1.776 (4.73), 1.794 (0.61), 1.864 (0.69), 1.885 (1.93), 1.916 (2.20), 1.978 (3.01), 1.988 (2.81), 2.328 (0.80), 2.367 (0.80), 2.580 (2.29), 2.670 (1.00), 2.711 (0.89), 2.823 (13.80), 3.004 (2.83), 3.031 (7.94), 3.057 (5.49), 3.260 (4.30), 3.301 (4.33), 3.325 (4.15), 3.585 (5.71), 3.601 (12.71), 3.618 (6.10), 3.731 (4.95), 4.021 (0.52), 4.038 (0.47), 4.457 (1.62), 4.484 (2.81), 4.511 (1.47), 5.754 (11.96), 7.013 (6.70), 7.034 (7.14), 7.061 (9.75), 7.066 (11.52), 7.088 (5.10), 7.093 (3.57), 7.108 (7.87), 7.113 (6.81), 7.156 (13.10), 7.176 (7.16), 7.387 (3.31), 7.456 (13.47), 7.477 (11.85), 7.518 (6.20), 7.649 (2.72), 7.954 (16.00).

### Example 8

### 1-[1-{4-Chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (Enantiomer 1)

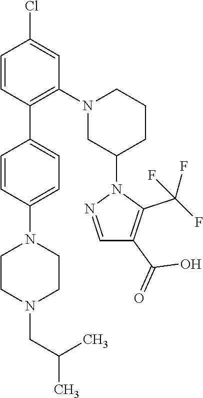 [SMILES: CC(C)CN1CCN(c2ccc(-c3ccc(Cl)cc3N3CCCC(n4ncc(C(=O)O)c4C(F)(F)F)C3)cc2)CC1]

An aqueous solution of lithium hydroxide (1.2 l, 1.0 M, 1.2 mol) was added to a solution of ethyl 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylate (Enantiomer 1, 77.0 g, 125 mmol) in a THF/methanol mixture (9:1) (1.5 l). The resulting mixture was stirred overnight at room temperature and acidified to pH˜2 with an aqueous solution of hydrogen chloride (2N). The reaction mixture was diluted with dichloromethane. The organic layer was washed with water and evaporated affording 74 g (quant.) of the title compound which was used in the next step without further purification.

LC-MS (Method 3): Rₜ=1.74 min; MS (ESIpos): m/z=590 [M+H]⁺

¹H-NMR (600 MHz, DMSO-d6) δ[ppm]: 0.830 (0.48), 0.841 (0.49), 1.009 (16.00), 1.020 (16.00), 1.045 (0.89), 1.094 (1.29), 1.187 (0.45), 1.363 (0.58), 1.528 (0.64), 1.549 (0.68), 1.750 (2.54), 1.755 (2.82), 1.760 (5.81), 1.766 (2.92), 1.771 (2.27), 1.919 (0.75), 1.926 (0.61), 1.940 (0.72), 1.946 (0.67), 2.003 (0.92), 2.019 (0.59), 2.105 (0.70), 2.117 (0.84), 2.128 (0.67), 2.579 (0.64), 2.863 (1.29), 2.981 (1.01), 2.998 (1.83), 3.016 (1.09), 3.051 (1.01), 3.069 (0.93), 3.216 (1.08), 3.238 (1.70), 3.256 (1.35), 3.573 (0.51), 3.594 (2.25), 3.604 (4.74), 3.615 (1.96), 4.383 (0.57), 4.394 (0.64), 4.400 (0.96), 4.407 (0.64), 4.418 (0.51), 7.033 (4.04), 7.048 (4.15), 7.082 (3.12), 7.085 (3.80), 7.099 (1.74), 7.102 (1.19), 7.113 (2.42), 7.116 (2.08), 7.155 (4.12), 7.168 (2.53), 7.473 (4.69), 7.488 (4.22), 8.020 (5.33).

### Example 9

### 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (Enantiomer 1)

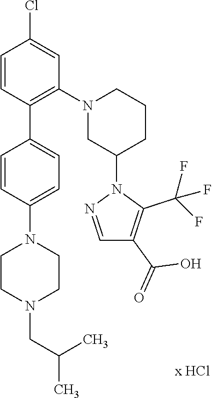 [SMILES: CC(C)CN1CCN(c2ccc(-c3ccc(Cl)cc3N3CCCC(n4ncc(C(=O)O)c4C(F)(F)F)C3)cc2)CC1]

Method A

A solution of 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid (Enantiomer 1, 78.0 g, 132 mmol) in diethyl ether (1.5 l) was treated with a solution of hydrogen chloride in diethyl ether (150 ml, 150 mmol). The resulting mixture was stirred overnight at room temperature and evaporated affording 82 g (quant.) of the title compound.

LC-MS (Method 3): Rₜ=1.77 min; MS (ESIpos): m/z=590 [M−HCl+H]⁺

¹H-NMR (400 MHz, DMSO-d6) δ[ppm]: −0.839 (0.40), 1.013 (0.67), 1.029 (15.68), 1.039 (16.00), 1.057 (0.67), 1.081 (1.77), 1.092 (3.56), 1.104 (1.68), 1.360 (0.54), 1.520 (0.54), 1.540 (0.60), 1.741 (0.73), 1.750 (0.52), 1.761 (0.83), 1.921 (0.56), 1.927 (0.52), 1.941 (0.61), 1.947 (0.58), 2.004 (0.77), 2.020 (0.52), 2.147 (0.46), 2.158 (0.88), 2.169 (1.10), 2.180 (0.89), 2.192 (0.47), 2.578 (0.57), 2.984 (0.91), 2.995 (1.90), 3.004 (3.37), 3.016 (1.95), 3.044 (0.88), 3.062 (0.77), 3.119 (0.77), 3.125 (0.78), 3.135 (1.02), 3.145 (0.85), 3.151 (0.86), 3.244 (0.80), 3.258 (0.71), 3.361 (1.04), 3.368 (0.87), 3.380 (3.28), 3.391 (1.89), 3.403 (1.41), 3.570 (1.41), 3.589 (1.33), 3.603 (0.52), 3.785 (0.79), 3.814 (0.99), 3.838 (0.75), 4.383 (0.46), 4.394 (0.53), 4.400 (0.79), 4.407 (0.55), 4.418 (0.46), 7.055 (3.42), 7.070 (3.66), 7.085 (2.44), 7.089 (3.02), 7.105 (1.54), 7.108 (1.13), 7.118 (2.11), 7.121 (1.92), 7.157 (3.63), 7.171 (2.19), 7.485 (3.98), 7.500 (3.68), 8.023 (4.23), 10.650 (0.49).

Method B

Ethyl 1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylate (Enantiomer 1, 149 mg, 241 μmol) was dissolved in THF/ethanol (6.3/0.63 ml). 1 M aqueous lithium hydroxide solution (2.4 ml, 2.4 mmol) was added and the mixture was stirred overnight at room temperature. The mixture was evaporated, then acidified and purified using preparative HPLC (RP18 column, acetonitrile/water gradient with the addition of 0.1% TFA). The product fractions were combined and evaporated. Then the residue was dissolved in acetonitrile, mixed with 0.1 M hydrochloric acid in dioxane, carefully evaporated at 30° C. (thrice) and then lyophilized. 130 mg of the target compound (85% of theory) were obtained.

LC-MS (Method 3): Rₜ=1.81 min; MS (ESIpos): m/z=590 [M−HCl+H]⁺

¹H-NMR (600 MHz, DMSO-d6) δ[ppm]: 1.014 (15.69), 1.025 (16.00), 1.522 (0.64), 1.543 (0.70), 1.747 (0.89), 1.769 (0.75), 1.916 (0.67), 1.935 (0.74), 2.003 (0.95), 2.020 (0.62), 2.133 (0.48), 2.144 (0.96), 2.155 (1.17), 2.166 (0.94), 2.177 (0.49), 2.588 (0.65), 2.605 (1.20), 2.624 (0.66), 2.968 (0.93), 2.986 (1.83), 3.006 (2.63), 3.017 (3.24), 3.027 (1.95), 3.052 (1.01), 3.070 (0.94), 3.115 (0.97), 3.133 (1.36), 3.148 (1.12), 3.230 (1.08), 3.251 (1.98), 3.273 (2.05), 3.292 (1.01), 3.578 (1.97), 3.597 (1.82), 3.800 (1.79), 3.823 (2.21), 3.841 (2.82), 4.367 (0.59), 4.385 (1.02), 4.403 (0.56), 7.051 (3.88), 7.065 (4.03), 7.092 (3.43), 7.110 (1.41), 7.123 (2.32), 7.155 (3.64), 7.169 (1.99), 7.486 (4.35), 7.501 (3.97), 8.028 (4.91), 10.135 (0.55).

### Example 10

### 1-[1-[5-Chloro-2-[4-[4-(cyclopropylmethyl)piperazin-1-yl]phenyl]phenyl]-3-piperidyl]-5-(trifluoromethyl)pyrazole-4-carboxylic acid hydrochloride (Enantiomer 1)

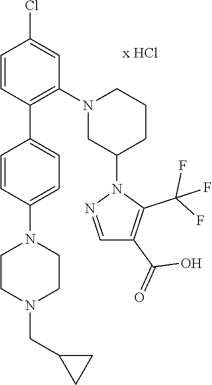 [SMILES: O=C(O)c1cnn(C2CCCN(c3cc(Cl)ccc3-c3ccc(N4CCN(CC5CC5)CC4)cc3)C2)c1C(F)(F)F]

Ethyl 1-[1-{4-chloro-4′-[4-(cyclopropylmethyl)piperazin-1-yl][1,1′-biphenyl]-2-yl}piperidin-3-yl]-5-(trifluoromethyl)-1H-pyrazole-4-carboxylate trifluoroacetic acid (Enantiomer 1, 161 mg, 221 μmol) was dissolved in THF/ethanol (6.8/0.68 ml). 1 M aqueous lithium hydroxide solution (2.6 ml, 2.6 mmol) was added and the mixture was stirred overnight at room temperature. The mixture was evaporated, then acidified and purified using preparative HPLC (RP18 column, acetonitrile/water gradient with the addition of 0.1% TFA). The product fractions were combined and evaporated. Then the residue was dissolved in acetonitrile, mixed with 0.1 M hydrochloric acid in dioxane, carefully evaporated at 30° C. (thrice) and then lyophilized. 134 mg of the target compound (97% of theory) were obtained.

LC-MS (Method 3): Rₜ=1.82 min; MS (ESIpos): m/z=588 [M−HCl+H]⁺

¹H-NMR (600 MHz, DMSO-d6) δ[ppm]: 0.426 (11.09), 0.434 (11.21), 0.443 (2.91), 0.665 (9.10), 0.678 (9.20), 0.687 (2.36), 1.161 (1.39), 1.169 (2.49), 1.173 (2.48), 1.181 (3.33), 1.193 (2.23), 1.522 (2.23), 1.543 (2.37), 1.565 (0.99), 1.746 (3.16), 1.767 (2.56), 1.903 (0.93), 1.917 (2.39), 1.937 (2.58), 1.957 (1.13), 2.003 (3.29), 2.021 (2.11), 2.585 (2.13), 2.602 (3.93), 2.621 (2.20), 2.972 (3.14), 2.990 (5.91), 3.008 (3.45), 3.054 (9.53), 3.065 (12.33), 3.074 (8.23), 3.131 (3.14), 3.149 (4.65), 3.164 (4.39), 3.184 (7.56), 3.205 (7.92), 3.228 (5.44), 3.248 (3.11), 3.649 (6.16), 3.857 (3.86), 3.878 (3.65), 3.892 (4.10), 3.912 (3.42), 4.373 (2.27), 4.391 (3.67), 4.408 (2.14), 4.718 (2.01), 7.060 (13.16), 7.075 (13.71), 7.092 (11.58), 7.109 (4.98), 7.123 (7.67), 7.158 (11.82), 7.172 (6.72), 7.488 (14.54), 7.502 (13.17), 8.029 (16.00), 10.907 (2.16).

### Example 11

### 1-[1-[5-Chloro-2-[4-[4-(2,2,2-trifluoroethyl)piperazin-1-yl]phenyl]phenyl]-3-piperidyl]-5-(trifluoromethyl)pyrazole-4-carboxylic acid (Enantiomer 1)

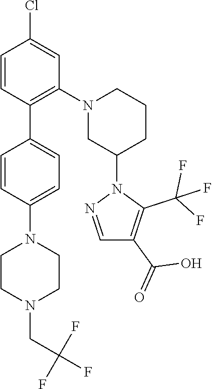 [SMILES: O=C(O)c1cnn(C2CCCN(c3cc(Cl)ccc3-c3ccc(N4CCN(CC(F)(F)F)CC4)cc3)C2)c1C(F)(F)F]

An aqueous solution of lithium hydroxide (1.3 ml, 1.0 M, 1.3 mmol) was added to a solution of 2,2,2-trifluoroethyl 1-(1-{4-chloro-4′-[4-(2,2,2-trifluoroethyl)piperazin-1-yl][biphenyl]-2-yl}piperidin-3-yl)-5-(trifluoromethyl)-1H-pyrazole-4-carboxylate (Enantiomer 1, 88.0 mg, 126 μmol) in a THF/methanol mixture (10/1, 2.5 ml). The resulting mixture was stirred 2 hours at room temperature. The reaction mixture was acidified with an aqueous solution of hydrogen chloride (2 N), evaporated and purified by preparative HPLC (RP18 column, eluent: Acetonitrile/water gradient) affording 73.0 mg (93% yield) of the title compound.

LC-MS (Method 3): Rₜ=2.71 min; MS (ESIpos): m/z=616 [M+H]⁺

¹H-NMR (600 MHz, DMSO-d6) δ[ppm]: 1.233 (0.40), 1.356 (7.08), 1.522 (0.63), 1.543 (1.76), 1.563 (1.88), 1.585 (0.83), 1.747 (2.39), 1.768 (2.00), 1.879 (0.71), 1.892 (1.90), 1.898 (1.75), 1.913 (1.92), 1.919 (1.86), 1.934 (0.90), 1.995 (2.40), 2.012 (1.64), 2.183 (1.02), 2.386 (0.47), 2.425 (0.46), 2.588 (1.81), 2.608 (3.38), 2.624 (1.77), 2.654 (0.50), 2.763 (11.02), 2.771 (16.00), 2.779 (11.98), 2.937 (2.72), 2.955 (5.21), 2.973 (3.04), 3.067 (2.64), 3.086 (2.38), 3.175 (10.75), 3.182 (13.42), 3.185 (13.56), 3.192 (10.16), 3.212 (3.33), 3.224 (5.64), 3.241 (8.96), 3.258 (8.54), 3.275 (3.26), 4.353 (1.52), 4.370 (2.62), 4.388 (1.43), 6.871 (0.68), 6.971 (12.63), 6.986 (12.93), 7.067 (9.07), 7.071 (10.95), 7.090 (5.19), 7.094 (3.70), 7.104 (7.44), 7.107 (6.37), 7.147 (12.95), 7.160 (7.73), 7.429 (14.45), 7.443 (12.97), 8.005 (13.58), 13.136 (0.44).

### Comparative Example 174 (WO2012/058132)

### 1-{1-[4-Chloro-4′-(4-cyclopropylmethylpiperazin-1-yl)[biphenyl]-2-yl]pyridin-3-yl}-5-(trifluoromethyl)-1H-pyrazole-4-carboxylic acid

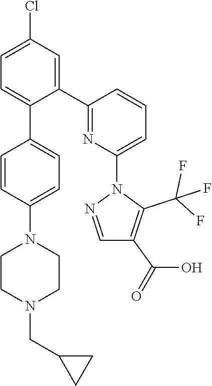 [SMILES: O=C(O)c1cnn(-c2cccc(-c3cc(Cl)ccc3-c3ccc(N4CCN(CC5CC5)CC4)cc3)n2)c1C(F)(F)F]

The compound was synthesized according to the procedures disclosed in WO 2012/058132 (experimental part, pages 58 to 84).

### B. Assessment of Pharmacological Efficacy and Pharmacokinetic Profile

The following abbreviations are used:
ATP adenosine triphosphate
Brij35 polyoxyethylene(23) lauryl ether
BSA bovine serum albumin:
DTT dithiothreitol
TEA triethanolamine

Biological Investigations

The example testing experiments described herein serve to illustrate the present invention and the invention is not limited to the examples given.

The following assays can be used to illustrate the commercial utility of the compounds according to the present invention.

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values calculated utilizing data sets obtained from testing of one or more synthetic batch.

The in vitro activity of the compounds of the present invention can be demonstrated in the following assays.

The pharmacological action of the compounds of the invention can be demonstrated in the following assays:

B-1. Effect on a Recombinant Guanylate Cyclase Reporter Cell Line

The cellular activity of the compounds according to the invention was determined using a recombinant guanylate cyclase reporter cell line, as described in F. Wunder et al., Anal. Biochem. 339, 104-112 (2005).

Representative MEC values (MEC=minimum effective concentration) and EC₅₀ values (half maximal effective concentration) for the compounds of the invention are shown in the table below (in some cases as mean values from individual determinations):

TABLE 2

| Example | MEC [nM] | EC₅₀ [nM] |
|---|---|---|
| 1 | 2.3 | 9.2 |
| 2 | 1.0 | 8.6 |
| 3 | 0.6 | 2.7 |
| 4 | 0.3 | 3.2 |
| 5 | <0.3 | 3.6 |
| 6 | 1.6 | 19.3 |
| 7 | 1.6 | 13.7 |
| 8 | 6.5 | 40 |
| 9 | 2.2 | 11.0 |
| 10 | 2.0 | 10.3 |
| 11 | 0.6 | 5.2 |

B-2. Determination of Pharmacokinetic Parameters Following Intravenous and Oral Administration

The pharmacokinetic parameters of the compounds according to the invention were determined in male Wistar rats and and/or in female beagles and/or in cynomolgus monkeys and/or in male CD-1 mice. Intravenous administration in the case of mice and rats was carried out by means of a species-specific plasma/DMSO formulation, and in the case of dogs and monkeys by means of a water/PEG400/ethanol formulation. In all species, oral administration of the dissolved substance was performed via gavage, based on a water/PEG400/ethanol formulation.

An internal standard (which may also be a chemically unrelated substance) was added to the samples of the compounds of the invention, calibration samples and qualifiers, and there followed protein precipitation by means of acetonitrile in excess. Addition of a buffer solution matched to the LC conditions, and subsequent vortexing, was followed by centrifugation at 1000 g. The supernatant was analysed by LC-MS/MS using C18 reversed-phase columns and variable mobile phase mixtures. The substances were quantified via the peak heights or areas from extracted ion chromatograms of specific selected ion monitoring experiments.

The plasma concentration/time plots determined were used to calculate the pharmacokinetic parameters such as AUC, Cₘₐₓ, t_{1/2} (terminal half-life), F (bioavailability), MRT (mean residence time) and CL (clearance), by means of a validated pharmacokinetic calculation program.

Since the substance quantification was performed in plasma, it was necessary to determine the blood/plasma distribution of the substance in order to be able to adjust the pharmacokinetic parameters correspondingly.

For this purpose, a defined amount of substance was incubated in K3 EDTA whole blood of the species in question in a rocking roller mixer for 20 min. After centrifugation at 1000 g, the plasma concentration was measured (by means of LC-MS/MS; see above) and determined by calculating the ratio of the C_{blood}/Cₚₗₐₛₘₐ value.

Table 3 shows data of representative compounds of the present invention following intravenous administration in rats:

TABLE 3

| | AUCnorm | CLplasma | t½ | MRT |
|---|---|---|---|---|
| Example | [kg · h/L] | [L/h/kg] | [h] | [h] |
| 1 | 1.77 | 0.56 | 1.64 | 2.24 |
| 2 | 7.08 | 0.14 | 3.13 | 3.44 |
| 4 | 1.40 | 0.72 | 2.09 | 2.62 |
| 5 | 1.23 | 0.81 | 2.43 | 2.88 |
| 6 | 2.29 | 0.44 | 5.13 | 4.16 |
| 7 | 7.23 | 0.14 | 3.83 | 4.41 |
| 9 | 5.48 | 0.18 | 4.10 | 5.83 |
| 10 | 1.37 | 0.73 | 1.89 | 2.39 |
| 174 | 0.77 | 1.30 | 2.33 | 2.78 |
| (WO2012/058132) | | | | |

Table 4 shows data of representative compounds of the present invention following oral administration (p.o.) in rats:

TABLE 4

| | AUCnorm | t½ | MRT | F |
|---|---|---|---|---|
| Example | [kg · h/L] | [h] | [h] | [%] |
| 1 | 0.57 | 3.24 | 6.28 | 31.4 |
| 2 | 3.77 | 3.96 | 6.23 | 53.3 |
| 4 | 0.84 | 3.02 | 5.91 | 59.9 |
| 5 | 0.70 | 3.76 | 7.53 | 58.2 |
| 6 | 2.11 | 7.37 | 14.2 | 91.7 |
| 7 | 4.04 | 3.63 | 7.32 | 56.1 |
| 9 | 4.50 | 4.96 | 8.51 | 82.1 |
| 10 | 0.52 | 3.41 | 4.04 | 37.4 |
| 174 | 0.63 | 3.60 | 8.40 | 81.8 |
| (WO2012/058132) | | | | |

Table 5 shows data of representative compounds of the present invention following intravenous administration (i.v.) in dogs:

TABLE 5

| | AUCnorm | CLplasma | t½ | MRT |
|---|---|---|---|---|
| Example | [kg · h/L] | [L/h/kg] | [h] | [h] |
| 2 | 81.7 | 0.01 | 17.7 | 25.6 |
| 9 | 37.4 | 0.03 | 7.86 | 10.3 |
| 174 | 5.00 | 0.20 | 10.8 | 7.23 |
| (WO2012/058132) | | | | |

Table 6 shows data of representative compounds of the present invention following oral administration (p.o.) in dogs:

TABLE 6

| | AUCnorm | t½ | MRT | F |
|---|---|---|---|---|
| Example | [kg · h/L] | [h] | [h] | [%] |
| 2 | 67.7 | 14.0 | 21.3 | 82.8 |
| 9 | 31.7 | 9.28 | 13.7 | 84.8 |
| 174 | 2.08 | 7.05 | 6.10 | 41.6 |
| (WO2012/058132) | | | | |

The compounds according to the present invention show low plasma clearance (CLplasma) in all tested species, e.g. examples 2 and 9 show lower CLₚₗₐₛₘₐ (up to 10 times) and therefore much higher exposure (AUCnorm) in comparison to the compounds disclosed in the prior art, e.g. example 174 (WO2012/058132) in rats as well as in dogs (see tables 3 and 5). Examples 2 and 9 also show long half-life and mean residence time (MRT) in all tested species after p.o. (per oral) application (see tables 4 and 6). Due to the lower plasma clearance (CLplasma) of examples 2 and 9 and the resulting higher exposure (AUCₙₒᵣₘ) with good bioavailability after p.o. application in all tested species, examples 2 and 9 show superior pharmacokinetic properties versus the compounds disclosed in the prior art, e.g. example 174 (WO2012/058132).

B-3. Metabolic Study

To determine the metabolic profile of the inventive compounds, they were incubated with recombinant human cytochrome P450 (CYP) enzymes, liver microsomes or primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds of the invention were incubated with a concentration of about 0.1-10 μM. To this end, stock solutions of the compounds of the invention having a concentration of 0.01-1 mM in acetonitrile were prepared, and then pipetted with a 1:100 dilution into the incubation mixture. The liver microsomes and recombinant enzymes were incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation mixtures were stopped with acetonitrile (final concentration about 30%) and the protein was centrifuged off at about 15 000×g. The samples thus stopped were either analyzed directly or stored at −20° C. until analysis.

The analysis was carried out by high-performance liquid chromatography with ultraviolet and mass spectrometry detection (HPLC-UV-MS/MS). To this end, the supernatants of the incubation samples were chromatographed with suitable C18 reversed-phase columns and variable mobile phase mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic reduction of the compound of the invention in the incubation mixtures.

B-4. Caco-2 Permeability Test

The permeability of a test substance was determined with the aid of the Caco-2 cell line, an established in vitro model for permeability prediction at the gastrointestinal barrier (Artursson, P. and Karlsson, J. (1991). Correlation between oral drug absorption in humans and apparent drug permeability coefficients in human intestinal epithelial (Caco-2) cells. Biochem. Biophys. 175 (3), 880-885). The Caco-2 cells (ACC No. 169, DSMZ, Deutsche Sammlung von Mikroorganismen und Zellkulturen, Braunschweig, Germany) were sown in 24-well plates having an insert and cultivated for 14 to 16 days. For the permeability studies, the test substance was dissolved in DMSO and diluted to the final test concentration with transport buffer (Hanks Buffered Salt Solution, Gibco/Invitrogen, with 19.9 mM glucose and 9.8 mM HEPES). In order to determine the apical to basolateral permeability (PₐₚₚA-B) of the test substance, the solution comprising the test substance was applied to the apical side of the Caco-2 cell monolayer, and transport buffer to the basolateral side. In order to determine the basolateral to apical permeability (PₐₚₚB-A) of the test substance, the solution comprising the test substance was applied to the basolateral side of the Caco-2 cell monolayer, and transport buffer to the apical side. At the start of the experiment, samples were taken from the respective donor compartment in order to ensure the mass balance. After an incubation time of two hours at 37° C., samples were taken from the two compartments. The samples were analyzed by means of LC-MS/MS and the apparent permeability coefficients (Pₐₚₚ) were calculated. For each cell monolayer, the permeability of Lucifer Yellow was determined to ensure cell layer integrity. In each test run, the permeability of atenolol (marker for low permeability) and sulfasalazine (marker for active excretion) was also determined as quality control.

B-5. Solubility Determination of Substances in Buffer pH 6.5

2-4 mg of the test compound were dissolved in DMSO to reach a concentration of 50 g/L (solution A, 515 μg/l). To 10 μl of this solution 960 μl PBS buffer pH 6.5 were added; the mixture was shaken for 24 h at rt in a 96 well plate. An aliquot was centrifuged at 42000 rpm for 30 min. The supernatant was diluted with ACN/water (8:2) 1:10 and 1:1000 resp. This diluted samples were analyzed by LC-MSMS.

Calibration: 10 μl of solution A were diluted with 823 μl DMSO (final concentration: 600 μg/ml), which was further diluted with ACN/water 8:2 by a factor of 100 (solution B).

The calibration curve was obtained from solution B by further diluting with ACN/water 8:2 with target concentrations of 1.2-12-60-600 ng/ml and injecting these four solutions for MS measurement.

MS Method Optimization:

Solution B was utilized for MS method optimization.

PBS-Puffer: 6.18 g sodium chloride and 3.96 g sodium dihydrogen phosphate were dissolved in 1 L aqua dist., the pH was adjusted to 6.5 with 1N sodium hydroxide.

LC-MSMS Optimization:

The following configurations were used for optimization

AB Sciex TRIPLE QUAD 4500, Agilent 1260 Infinity (G1312B), degasser (G4225A), column oven (G1316C or G1316A), CTC Analytics PAL injection system HTS-xt or HTC-xt.

Eluent A: 0.5 ml formic acid (50% ig)/L water, Eluent B: 0.5 ml formic acid (50% ig)/L acetonitrile

| time [min] | flow [μl/min] | % B |
|---|---|---|
| 0.00 | 200 | 70 |
| 0.08 | 200 | 70 |
| 0.09 | 25 | 70 |
| 0.60 | 25 | 70 |
| 0.65 | 200 | 70 |
| 1.10 | 200 | 70 |

Autosampler: without auto inject ahead setting

column: stainless steel capillary

oven temperature: 22° C.

flow rate: flow gradient

injected volume: 2 μl

Water Quattro Micro MS, Agilent 1100 (G1312A), degasser (G1322A), column oven (G1316A), CTC Analytics PAL injection system HTS, eluents as above

| time [min] | flow [μl/min] | % B |
|---|---|---|
| 0.00 | 250 | 70 |
| 1.50 | 250 | 70 |

Autosampler: with auto inject ahead setting

column: stainless steel capillary

oven temperature: 22° C.

flow rate: flow gradient

injected volume: 5 μl
MS method: Flow Injection Analysis (FIA) for optimization (“MS-OPTI”); Ionization mode ABSciex-MS: ESI-pos/neg, Waters-MS: ESI-pos

HPLC Method for MSMS Quantification:

Eluent A, B as above

ABSciex-MS

| time [min] | % A | % B |
|---|---|---|
| 0 | 90 | 10 |
| 0.5 | 5 | 95 |
| 0.84 | 5 | 95 |
| 0.85 | 90 | 10 |
| 1.22 | 90 | 10 |

Autosampler: without auto inject ahead setting

column: Waters OASIS HLB, 2, 1×20 mm, 25 g

column temperature: 30° C.

flow rate: 2.5 ml

injected volume: 2 μl

Splitter (before MS) 1:20

Waters-MS

| time [min] | % A | % B |
|---|---|---|
| 0 | 90 | 10 |
| 0.5 | 5 | 95 |
| 0.84 | 5 | 95 |
| 0.85 | 90 | 10 |
| 1.5 | 90 | 10 |

Autosampler: with auto inject ahead setting

column: Waters OASIS HLB, 2, 1×20 mm, 25 g

column temperature: 30° C.

flow rate: 2.5 ml

injected volume: 5 μl

Splitter (before MS) 1:20

MS method: Multiple Reaction Monitoring (MRM)

B-6. Determination of Solubility from Solid

For each solvent, an Eppendorf plastic vial was charged with 0.5-1 mg of the test compound (exact weight), 2-3 μlass pearls (diameter 3 mm) and 1.0 ml of the respective solvent. The vial was closed and shaken at RT for 24 h (1400 rpm; Thermomixer, Eppendorf). Thereafter, 230 μl each of the solution/suspension was transferred into one or more centrifuge vials (Beckman Coulter) and were centrifuged at 42000 rpm for 30 min (Beckman Coulter Optima L90). At least 100 μl of the supernatant were withdrawn and further diluted with DMSO in two dilution strength: 1:5 and 1:50 (the latter obtained from the 1:5 dilution step by subsequent DMSO addition). This liquid handling was done either manually or with the help of a pipetting robot (Lissy, Zinsser Analytic).

For HPLC quantification, calibration solutions of the test compound in DMSO were prepared. Starting from an initial concentration of 600 μg/ml, three calibration solutions were prepared: 100 μg/ml, 20 μg/ml and 2.5 μg/ml (manually or via Lissy).

Both calibration solutions and the supernatant were analyzed by HPLC/UV-detection at an appropriate wave length. The solubility was determined using the linear calibration curve.

HPLC Systems:

Hewlett Packard/Agilent HPLC systems, G1311A+G1316A+G1315B as well as G1312A+G1316A+G1315A

injector system: CTC-Analytik HTC PAL

or with a Agilent UPLC System (G7117C, G7116B, G7167B and G7120)

oven temperature: 30° C., detection: 210 and/or 254 nm, injected volume: 20 μl

eluent A: 0.1% TFA in water, eluent B: 0.1% TFA in acetonitrile

column: ZORBAX Extend-C18, 3.0×50 mm, 3.5 μm

Gradient:

| time [min] | A [%] | B [%] | Flow rate: [ml/min] |
|---|---|---|---|
| 0.0 | 98 | 2 | 1.5 |
| 0.2 | 98 | 2 | 1.5 |
| 3.3 | 10 | 90 | 1.5 |
| 4.0 | 10 | 90 | 1.5 |
| 4.1 | 98 | 2 | 2.5 |
| 4.7 | 98 | 2 | 2.5 |
| 5.0 | 98 | 2 | 1.5 |

B-7 Evaluation of Acute Changes in Rat Retinal Structure after Retinal Ischemia Reperfusion (I/R) Prophylactic Settings

Six male Wistar Unilever rats were used per experimental group. On induction day, rats were anesthetized with an intraperitoneal injection of Rompun® and Ketavet® before the pupils of the right eyes (oculus dextrus, OD) were dilated with Alcain eye drops and in addition treated with Vigamox® eye drops. The left eye (oculus sinister, OS) is covered with Bepanthen® eye cream. Under deep anesthesia, the retina and the optic nerve were examined by optical coherence tomography (OCT) as a baseline measurement. 15 minutes before induction, group 2 received an intravenous (IV) bolus of compound (e.g. example 3, formula I-E-R) (i.v. 3 mg/kg in rat plasma). Then, the anterior chamber was punctured with a 30 G needle. Through a tube, 0.9% NaCl solution was pumped into the anterior chamber with a pressure 120 mm Hg. The pressure is regulated with a blood pressure cuff. The intraocular pressure (IOP) was elevated for 45 min. The procedure was successful as the eyeballs discolored because of vascular obstruction. After 45 min, the needle was removed, eye cream was put on the right eye and the animal could wake up. Compound (e.g. example 3, formula I-E-R) or its vehicle (Transcutol/Cremophor EL/H2O (10%/20%/70%) were orally applied, once daily (QD). The application volume was 5 mL/kg. Treatment was initiated 2 days before induction day and continued 6 days after induction. In addition, at the day of induction (day 3), 15 minutes before the induction, group 2 received compound in rat plasma IV treatment.

On day 7 following induction, an OCT and ERG examinations were done. At day 7 post induction, under deep anesthesia, the eyes were collected for histopathology and preserved in Davidson's solution. The eye sections were stained with hematoxylin and eosin staining.

Functional read-out of retinal electrical signal in response to light stimulation “b-wave amplitude (μv)” represents inner retinal function. The retinal function ERG (Electroretinography) was tested at day 7 post induction according to the method disclosed in McCulloch et al., 2015. IR animals at day 7 had significantly lower b wave amplitude compared to age matched normal animals (Non IR), which reflects the development of the retinal ischemic damage phenotype. Animals which received compound (e.g. example 3, formula I-E-R) therapy (IR+ compound (e.g. example 3, formula I-E-R)) had significant higher b wave amplitude compared to vehicle treated animals (IR+ Vehicle) and compared to untreated induced animals (IR only) as shown in FIG. 1A & 1B.

Examination of the retina at day 7 post ischemic induction reveals a marked distortion of different retinal layers specially the RPE Photoreceptor layer in IR only group. Animal treated with the compound (e.g. example 3, formula I-E-R) showed a preserved retinal structure in both OCT and histological examination. This was reflected as a preserved retinal function as measure by ERG in compound (e.g. example 3, formula I-E-R) treated animals compared to control (FIG. 1B).

The compounds according to the invention, e.g. example 3 protect(s) the retina from acute ischemic damage and preserves retinal function and morphology.

B-8 Evaluation of Sub-Chronic Changes in Rat Retinal Structure after Retinal Ischemia Reperfusion (I/R) Therapeutic and Prophylactic Settings

Six male Wistar Unilever rats were used per experimental group. On induction day, rats were anesthetized with an intraperitoneal injection of Rompun® and Ketavet® before the pupils of the right eyes were dilated with Alcain eye drops and in addition treated with Vigamox® eye drops. The left eye is covered with Bepanthen® eye cream. Under deep anesthesia, the retina and the optic nerve were examined by optical coherence tomography (OCT) as a baseline measurement. The induction was done as the anterior chamber was punctured with a 30 G needle. Through a tube, 0.9% NaCl solution was pumped into the anterior chamber with a pressure 120 mm Hg. The pressure is regulated with a blood pressure cuff. The intraocular pressure (IOP) was elevated for 45 min. The procedure was successful as the eyeballs discolored because of vascular obstruction. After 45 min, the needle was removed, eye cream was put on the right eye and the animal could wake up. Compound (e.g. example 3, formula I-E-R) or its vehicle were orally applied, once daily (QD). The application volume was 5 mL/kg. In prophylactic setting, animals started the treatment 2 days before induction and received an intravenous (IV) bolus of compound (e.g. example 3, formula I-E-R) (i.v. 3 mg/kg in rat plasma) 15 minutes before induction. Treatment continued then after for 21 days. In the therapeutic setting, animals received an intravenous (IV) bolus of compound (e.g. example 3, formula I-E-R) (i.v. 3 mg/kg in rat plasma) 15 minutes after induction. Treatment continued then after for 21 days; once daily (3 mg/kg, PO QD). At day 7 and day 21, the retina and the optic nerve were examined by optical coherence tomography (OCT). The retinal function was evaluated by the ERG (Electroretinography) at day 7 and day 21 post induction according to the method disclosed in McCulloch et al., 2015. Functional read-out of retinal electrical signal in response to light stimulation “b-wave amplitude (μv)” represents inner retinal function. Then the eyes were collected for histopathology and preserved in Davidson's solution. The eye sections were stained with hematoxylin and eosin staining. The total retinal thickness and the inner plexiform layer thickness was measured at 1000 μm distances from the optic nerve using (Microscope Software ZEN, Zeiss, Germany).

Neuroprotection: The inner plexiform layer (IPL) functions as a relay station for the vertical-information-carrying nerve cells, the bipolar cells, to connect the photoreceptor cells to the ganglion cells. The IPL layer thickness was measured in histological sections stained with (H&E).

Animals exposed to retinal ischemia (IR only) showed a progressive reduction of the retinal thickness (retinal degeneration) compared to baseline. Compound (e.g. example 3, formula I-E-R) treated animals showed a significantly higher retinal thickness compared to IR only animals at 3 weeks. This was reflected as significant difference in retinal function measured by ERG between IR and compound (e.g. example 3, formula I-E-R) treated animals (FIG. 2).

At day 7 and day 21, the retina and the optic nerve were examined by optical coherence tomography (OCT). The mean total retinal thickness was maintained at 1- and 3-weeks post induction compared to JR only animal that experienced a progressive degeneration of the retina consistent with the histopathological finding, FIG. 3.

The IPL thickness was reduced in vehicle treated animals and preserved in compound (e.g. example 3, formula I-E-R) treated animals in both prophylactic (FIG. 4A) and therapeutic settings (FIG. 4B). Retinas of IR only animals showed photoreceptors structural changes with accumulation of homogenous structures in the sub-photoreceptor space. In animals treated received compound (e.g. example 3, formula I-E-R) therapy, the retinal structure was preserved, and the photoreceptors integrity was intact.

The compounds according to the invention, e.g. example 3 protect(s) the retinal structure and maintains retinal function in both prophylactic and therapeutic settings.

As shown in FIG. 9, the photoreceptors (see arrow in FIG. 9) were degenerated in IR animals (left panel) and protected in compound treated animals (middle and right panel). This was reflected as significant difference in retinal function as measured by ERG between IR and compound treated animals.

B-9 Streptozotocin-Induced DR Model in Rat (STZ Rat Model)

135 male 6-week-old male SD rats (200 to 250 g) were randomized to become diabetic or non-diabetic. Following an overnight fast, SD rats were assigned to become diabetic by receiving a single intraperitoneal injection of streptozotocin (55 mg per kg; Sigma-Aldrich, St. Louis, USA) diluted in 0.1M citrate buffer, pH 4.5. Rats were weighed and their blood glucose levels measured (Accu-check Advantage II Blood Glucose Monitor, Roche Diagnostics, USA). Only rats with blood glucose levels greater than 250 mg/dL were considered diabetic (Li et al. 2002). Insulin was administered three times per week to reduce mortality and promote weight gain (2 to 4 units s.c. Humulin NPH, Eli Lilly and Co., Indianapolis, Ind., USA). The pathological events that occur in the early stages of DR were evaluated. Functional read-out of retinal electrical signal in response to light stimulation “b-wave amplitude (μv)” represents inner retinal function. The retinal function ERG (Electroretinography) was tested 2 months after STZ injection according to the method disclosed in McCulloch et al., 2015 and animals were randomized into subgroups with same severity. The animals received treatment after randomization for 2 months and were terminated then after. Treatment was applied by oral gavage, once daily (QD) with a range of doses, including e.g. 5 mg/kg, 15 mg/kg compound (e.g. example 3, formula I-E-R) (STZ+compound (e.g. example 3, formula I-E-R) or vehicle (STZ+vehicle (Transcutol/Cremophor EL/H2O (10%/20%/70%))). Diabetic animals at 2 months had significantly lower b wave amplitude compared to age matched normal animals (Non STZ), which reflects the development of DR disease phenotype. Animals which received compound (e.g. 15 mg/kg of example 3, formula I-E-R) therapy (STZ+compound (e.g. example 3, formula I-E-R) had significant higher b wave amplitude compared to vehicle treated animals as shown in FIG. 5.

While diabetic animals under vehicle therapy (STZ+vehicle) continue to progress to a more severe form of the disease (i.e. lower levels of b wave values), surprisingly diabetic animals under compound (e.g. example 3, formula I-E-R) therapy (STZ+compound (e.g. example 3, formula I-E-R) prevented disease progression and were significantly better than vehicle treated animals in a chronic diabetic retinopathy model.

Treatment is also applied by oral gavage, once daily (QD) with a range of doses, including 0.5 mg/kg, 1.5 mg/kg, 5 mg/kg, 15 mg/kg compound (e.g. example 3, formula I-E-R) (STZ+compound (e.g. example 3, formula I-E-R) or vehicle (STZ+vehicle (Transcutol/Cremophor EL/H2O (10%/20%/70%))).

B-10 Stimulation and Activation of Recombinant Soluble Guanylate Cyclase (sGC) In Vitro

Investigations on the modulation of recombinant soluble guanylate cyclase (sGC) by the compounds according to the invention with and without sodium nitroprusside, and with and without the heme-dependent sGC inhibitor 1H-1,2,4-oxadiazolo[4,3a]quinoxalin-1-one (ODQ), are carried out by the method described in Hoenickaet al., 1999. The heme-free guanylate cyclase is obtained by adding Tween 20 to the sample buffer (0.5% in the final concentration).

As described in WO 2012/139888, combination of sGC activators and 2-(N,N-diethylamino)diazenolate 2-oxide (DEA/NO), an NO donor, show no synergistic effect, i.e. the effect of DEA/NO is not potentiated as is expected with an sGC modulator acting via a heme-dependent mechanism. In addition, the effect of the sGC activator according to the invention is not blocked by 1H-1,2,4-oxadiazolo[4,3a]quinoxalin-1-one (ODQ), a heme-dependent inhibitor of soluble guanylate cyclase, but is in fact increased.

Thus, this test is suitable to distinguish between the heme-dependent sGC Stimulators and the heme-independent sGC Activators.

### FIGURES

The patent or application file contains at least one drawing executed in color. Copies of this patent or patent application publication with color drawing(s) will be provided by the Office upon request and payment of the necessary fee.

While the invention is illustrated and described in detail in the drawing and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Any reference signs should not be construed as limiting the scope.

FIG. 1A Compound (example 3, formula I-E-R) reduces ischemia induced retinal dysfunction measured by b wave amplitude in SD rats at one week post induction.

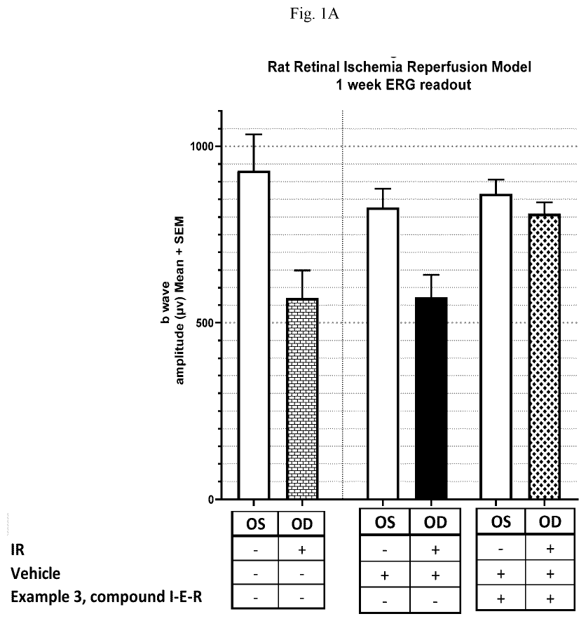

FIG. 1B Animal with the compound (example 3, formula I-E-R) showed a preserved retinal structure in both OCT and histological examination.

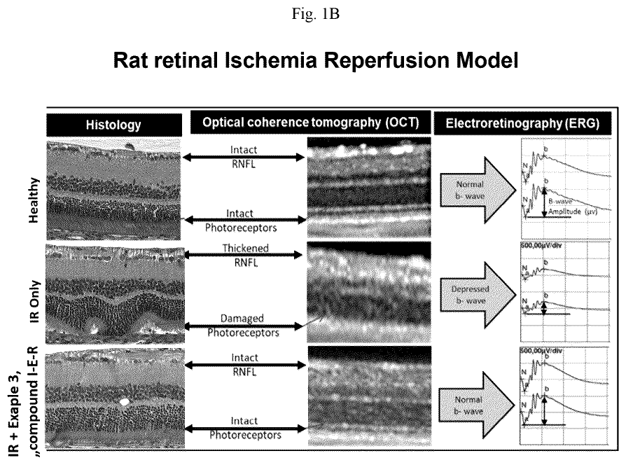

FIG. 2 Compound (example 3, formula I-E-R) reduces ischemia induced retinal dysfunction measured by b wave amplitude in SD rats at one week and 3 weeks post induction.

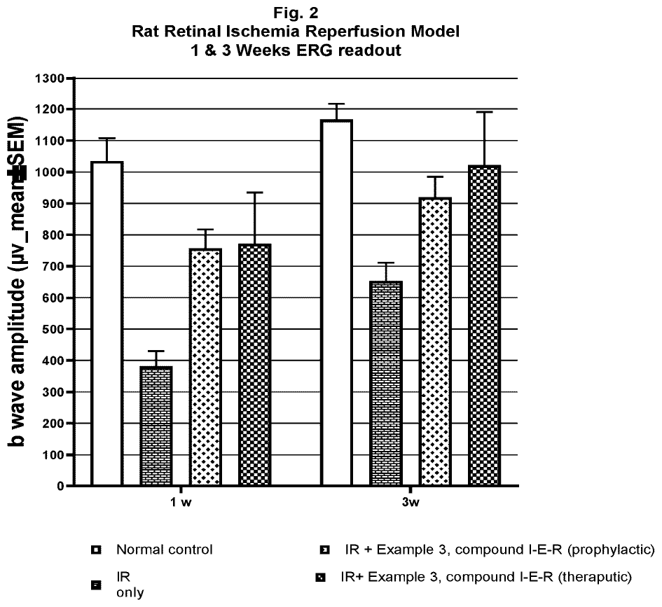

FIG. 3 Compound of (example 3, formula I-E-R) therapy protect retinal structure as measured by optical coherence tomography (OCT) at 1- and 3-weeks post induction compared to IR only animal.

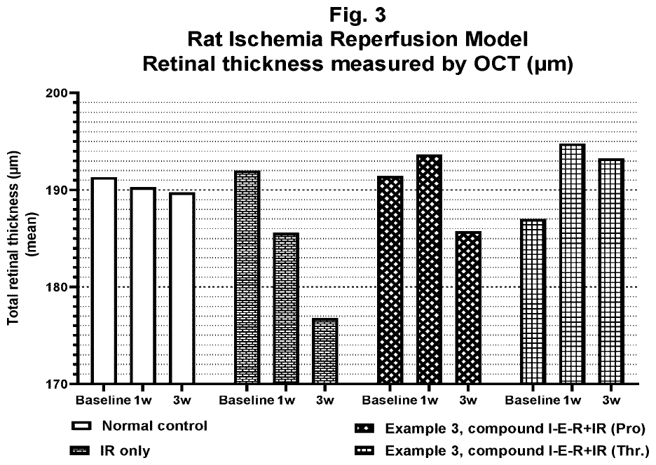

FIG. 4A shows changes of the rat retina in a rat IR model following treatment with formula I-E-R of example 3 in a prophylactic setting.

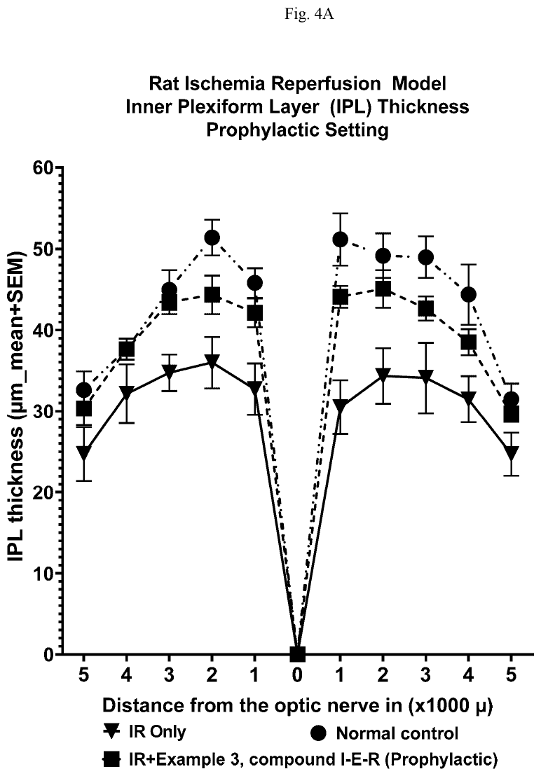

FIG. 4B shows changes of the rat retina in a rat IR model following treatment with formula I-E-R of example 3 in a therapeutic setting.

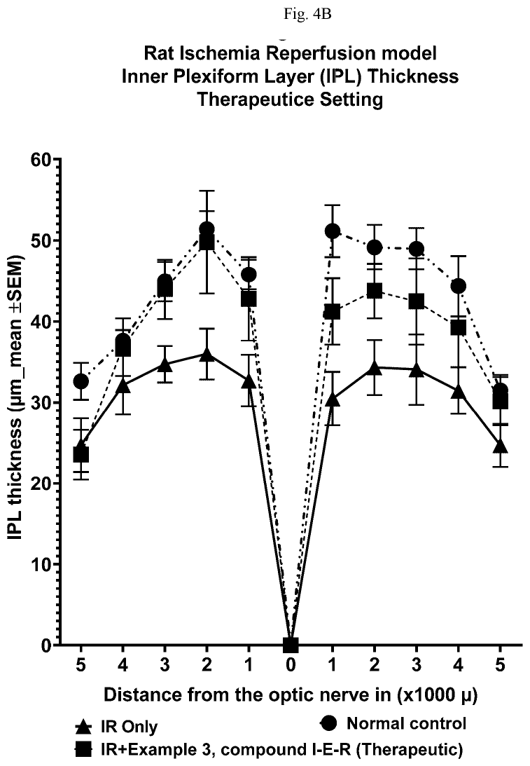

FIG. 5 Compound (example 3 of formula I-E-R) reduces diabetic induced retinal dysfunction and diabetic retinopathy progression measured by b wave amplitude in STZ rats.

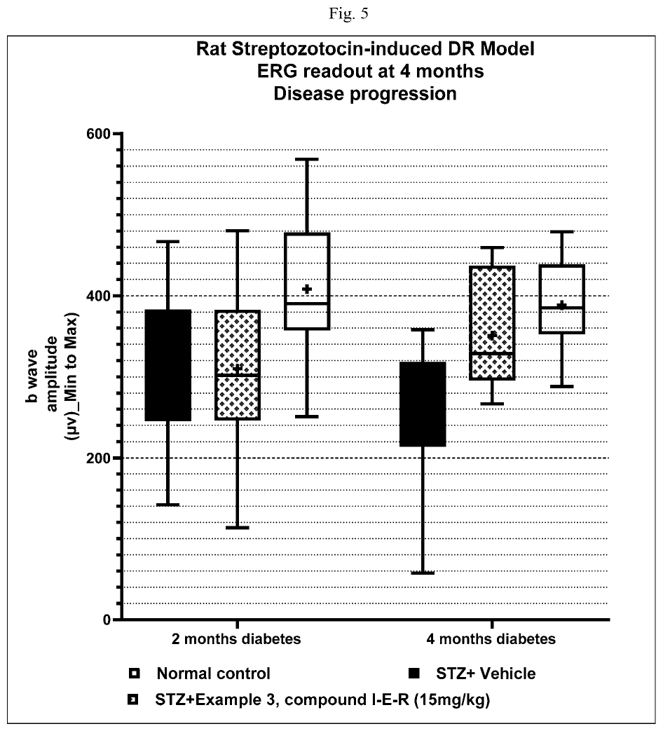

FIG. 6 shows an Ortep-Plot (50%) with labeling scheme (without disorder), as defined in example 3A.

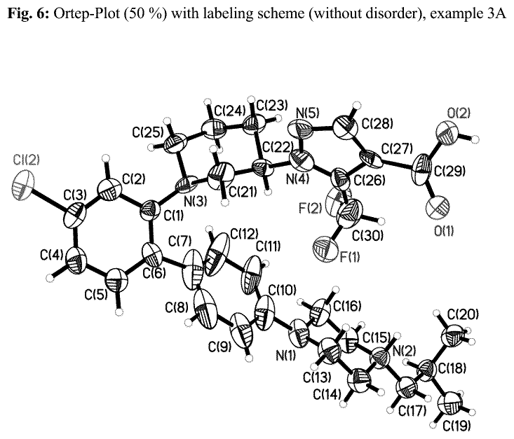

FIG. 7 shows independent molecules in the asymmetric unit (with disorder), as defined in example 3A.

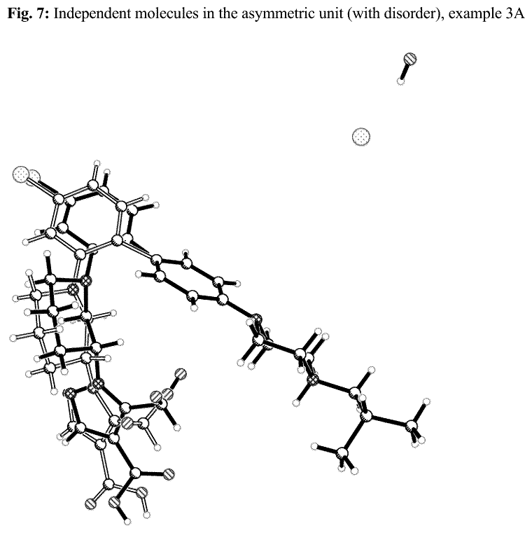

FIG. 8 shows the configuration of C₂₂, as defined in example 3A.

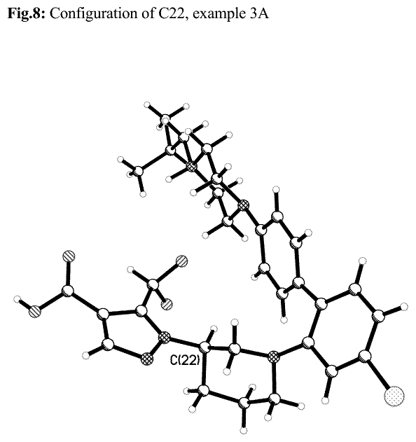

FIG. 9 shows differences in retinal function for compound-treated animals compared to untreated animals.

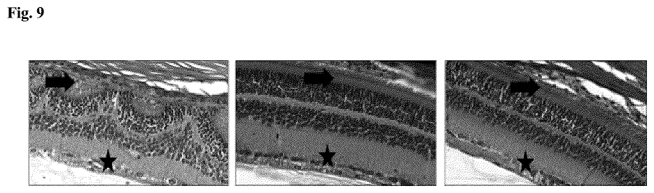

### REFERENCES

Berry S, L. W. (2017). Nonarteritic anterior ischemic optic neuropathy: cause, effect, and management. 9, 23-28.
Bolinger et al. Int J Mol Sci. 2016 September; 17(9): 1498
Chatziralli, Diabetes Ther. 2018 February; 9(1):431-434
Coyle J T, P. P. (1993). Oxidative stress, glutamate, and neurodegenerative disorders. 262, 689-695.
Dana Blumberg, A. S. (2015). Progress in Brain Research (Vol. 221). (C. N. Giacinto Bagetta, Ed.) Elsevier.
De Groef et al. 2016 Exp Eye Res. 2016 145:235-247
Douglas et al. Vis Neurosci. 2005 September-October; 22(5):677-84
Duh et al., JCI Insight. 2017 Jul. 20; 2(14)
E. S. Buys, D. Z. (2018). Discovery and development of next generation sGC stimulators with diverse multidimensional pharmacology and broad therapeutic potential. 78, 72-80.
Ehara, T. A. (2018). The Discovery of (S)-1-(6-(3-((4-(1-(Cyclopropanecarbonyl)piperidin-4-yl)-2-methylphenyl)amino)-2,3-dihydro-1H-inden-4-yl)pyridin-2-yl)-5-methyl-1H-pyrazole-4-carboxylic Acid, a Soluble Guanylate Cyclase Activator Specifically Designed for Topical Ocu. 61(6), 2552-2570.
ETDRS Report Number 12, Ophthalmology. 1991 May; 98(5 Suppl):823-33
Evgenov O V et al, Nat. Rev. Drug Discovery 2006, 5, 755-768
Ganesh Prasanna, L. F. (2018). A Novel Selective Soluble Guanylate Cyclase Activator, MGV354, Lowers Intraocular Pressure in Preclinical Models, Following Topical Ocular Dosing. 59, 1704-1716.
Gardner T W and Davila J R. Graefes Arch Clin Exp Ophthalmol., 2017 January; 255(1):1-6
Giannakaki-Zimmermann et al., Transl Vis Sci Technol. 2016 August; 5(4): 11
Gross et al., JAMA Ophthalmol. 2018 Oct. 1; 136(10):1138-1148
Gupta V B, R. M. (2014). Etiopathogenesis of cataract: an appraisal. Indian J Ophthalmol., 103-110.
Hayreh S S. Controversies on neuroprotection therapy in non-arteritic anterior ischaemic optic neuropathy. Br J Ophthalmol. 2020; 104(2):153-156. doi:10.1136/bjophthalmol-2019-314656
Hoenicka et al., J. Mol. Med. 1999, 77, 14-23
Huang et al., Toxicol Appl Pharmacol 2015 289:525-533
Kolb et al., 1995, The Organization of the Retina and Visual System
Li et al., Exp Eye Res. 2002 May; 74(5):615-25
Li Q et al., Invest Ophthalmol Vis Sci. 2010, 51(10):5240-6
Marianne L. Shahsuvaryan. (2013). Glaucomatous Optic Neuropathy Management: the Role of Neuroprotective Agents. 2, 2, 41-46.
McCulloch D. L. et al. Doc Ophthalmol 2015, 130: 1
Metea M R and Newman E A, Exp Physiol 2007, 92:635-640
Meyer C H, Sekundo W. Nutritional supplementation to prevent cataract formation. Dev Ophthalmol. 2005; 38:103-119. doi:10.1159/000082771
Newman, N. B. (2004). Hereditary optic neuropathies. 18, 1144-1160.
Nucci, C., Martucci, A., Giannini, C. et al. Neuroprotective agents in the management of glaucoma. Eye 32, 938-945 (2018). https://doi.org/10.1038/s41433-018-0050-2
O'Neill, E. D.-M. (2010). The optic nerve head in acquired optic neuropathies. 6(221-236).
Pamela Maher, A. H. (2005). The Molecular Basis of Oxidative Stress-Induced Cell Death in an Immortalized Retinal Ganglion Cell Line. 46, 749-757.
Pollreisz A, Schmidt-Erfurth U. Diabetic cataract-pathogenesis, epidemiology and treatment. J Ophthalmol. 2010; 2010:608751. doi:10.1155/2010/608751
Prasad S, G. S. (2011). Handbook of Clinical Neurology (Vol. vol 102 (3rd series)). (L. R. Kennard C, Ed.) Oxford: Elsevier.
Prusky et al. Invest Ophthalmol Vis Sci. 2004 December; 45(12):4611-6
Rebecca Stacy, K. H.-H. (2018). A Randomized, Controlled Phase I/II Study to Evaluate the Safety and Efficacy of MGV354 for Ocular Hypertension or Glaucoma,. 192, 113-123.
W G Robinson, N M Laver, J L Jacot, J P Glover, M D Basso, P Blouin, T C Hohman; Diabetic-like retinopathy ameliorated with the aldose reductase inhibitor WAY-121,509. Invest. Ophthalmol. Vis. Sci. 1996; 37(6):1149-1156. doi: https://doi.org/.
Schaefer S et al., Antioxid Redox Signal. 2003 August; 5(4):457-65
Schmidt et al., Handb Exp Pharmacol. (191):309-39
Sivaprasad et al. LARITY Study Group. Lancet. 2017 Jun. 3; 389(10085):2193-2203.
Solomon S D, Chew E, Duh E J et al., Diabetes Care. 2017 March; 40(3):412-418
Stasch J P et al., Br. J. Pharmacol. 2002, 136(5):773-783
Stasch J P et al., J. Clin. Invest. 2006, 116(9):2552-61
G. Tabin, M. Chen, and L. Espandar, “Cataract surgery for the developing world,” Current Opinion in Ophthalmology, vol. 19, no. 1, pp. 55-59, 2008.
Van Hove et al., International Journal of Molecular Sciences 2016 17(11) pii: E1825
Zhao et al. Drugs Context. 2018; 7: 212532
Zhao, W., Devamanoharan, P. S., Henein, M., Ali, A. H. and Varma, S. D. (2000), Diabetes-induced biochemical changes in rat lens: attenuation of cataractogenesis by pyruvate. Diabetes, Obesity and Metabolism, 2: 165-174. doi:10.1046/j.1463-1326.2000.00079.x

### C. Working Examples of Pharmaceutical Compositions

The compounds of the invention can be converted to pharmaceutical preparations as follows:

Tablet:

Composition:

100 mg of the compound according to the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

Production:

The mixture of compound of the invention, lactose and starch is granulated with a 5% solution (w/w) of the PVP in water. The granules are dried and then mixed with the magnesium stearate for 5 minutes. This mixture is compressed using a conventional tableting press (see above for format of the tablet). The guide value used for the pressing is a pressing force of 15 kN.

Suspension for Oral Administration:

Composition:

1000 mg of the compound of the invention, 1000 mg of ethanol (96%), 400 mg of Rhodigel® (xanthan gum from FMC, Pennsylvania, USA) and 99 g of water. 10 ml of oral suspension correspond to a single dose of 100 mg of the compound of the invention.

Production:

The Rhodigel is suspended in ethanol; the compound of the invention is added to the suspension. The water is added while stirring. The mixture is stirred for about 6 h until the swelling of the Rhodigel is complete.

Solution for Oral Administration:

Composition:

500 mg of the compound of the invention, 2.5 g of polysorbate and 97 g of polyethylene glycol 400. 20 g of oral solution correspond to a single dose of 100 mg of the compound of the invention.

Production:

The compound of the invention is suspended in the mixture of polyethylene glycol and polysorbate with stirring. The stirring process is continued until the compound according to the invention has completely dissolved.

Example Solution for Oral Administration:

Compound (e.g. example 3, formula I-E-R) is solubilized in vehicle comprising a mixture of Transcutol/Cremophor EL/H₂O (10%/20%/70%).

i.v. Solution:

The compound according to the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

1. A method for the oral treatment and/or prophylaxis of an eye disease comprising administering an effective amount of an sGC activator of formula (I) 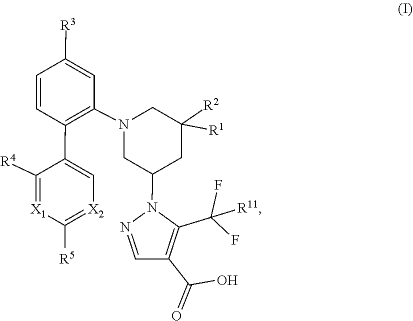
in which
R¹ represents hydrogen or halogen,
R² represents hydrogen or halogen,
R³ represents chloro or trifluoromethyl,
R⁴ represents hydrogen, C₁-C₄-alkyl,
R⁵ represents a group of the formula 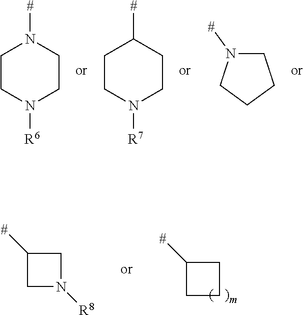
where #is the point of attachment to the aromatic or heteroaromatic 6 ring system; wherein m is 0-4
R⁶ represents
C₁-C₆-alkyl, optionally substituted by one or more substituent independently selected from the group consisting of methyl, trifluoromethoxy, nitril, amido,
C₂-C₆-halogenoalkyl, optionally substituted by 1 to 5 fluoro substituents,
C₃-C₆-cycloalkyl,
C₃-C₆-cycloalkyl-methyl, optionally substituted by 1 to 5 fluoro substituents or a trifluoromethyl group,
C₁-C₆-alkylcarbonyl, optionally substituted by 1 to 3 fluoro substituents,
C₃-C₆-cycloalkyl-carbonyl, optionally substituted by 1 to 3 fluoro substituents or
(C₁-C₆)-alkoxy-carbonyl, optionally substituted with methoxy, trifluoromethoxy, C₃-C₆-cycloalkyl,
(C₃-C₆)-cycloalkoxy-carbonyl,
mono-(C₁-C₄)-alkylaminocarbonyl,
(C₁-C₄)-alkylsulfonyl or
oxetanyl,
spiro[2.2]pentan-2-ylmethyl or [(3-fluoro-1-bicyclo[1.1.1]pentanyl)methyl,
R⁷ represents C₁-C₄-alkylcarbonyl, optionally substituted by a C₃-C₆-cycloalkyl group,
R⁸ represents C₂-C₄-alkyl, C₂-C₄-halogenoalkyl substituted by 1 to 6 fluoro substituents,
R¹¹ represents hydrogen or fluoro substituent
X₁ represents nitrogen or carbon or C—F
X₂ represents nitrogen or carbon
and the salts thereof, the solvates thereof and the solvates of the salts thereof, to a patient in need of said method.

2. The method according to claim 1, wherein the sGC activator is an SGC activator of formula (I-A) 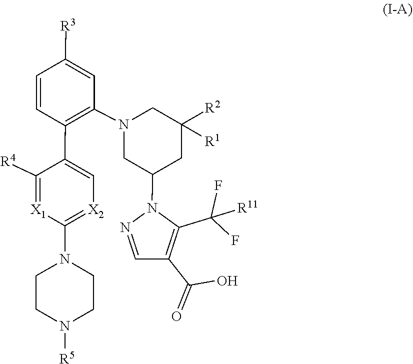
in which
R¹ represents hydrogen or halogen,
R² represents hydrogen or halogen,
R³ represents chloro or trifluoromethyl,
R⁴ represents hydrogen or C₁-C₄-alkyl
R⁵ represents optionally substituted C₁-C₆-alkyl
R¹¹ represents hydrogen or fluoro substituent
X₁ represents nitrogen or carbon
X₂ represents nitrogen or carbon
and the salts thereof, the solvates thereof and the solvates of the salts thereof.

3. The method according to claim 1, wherein the sGC activator is selected from the group consisting of 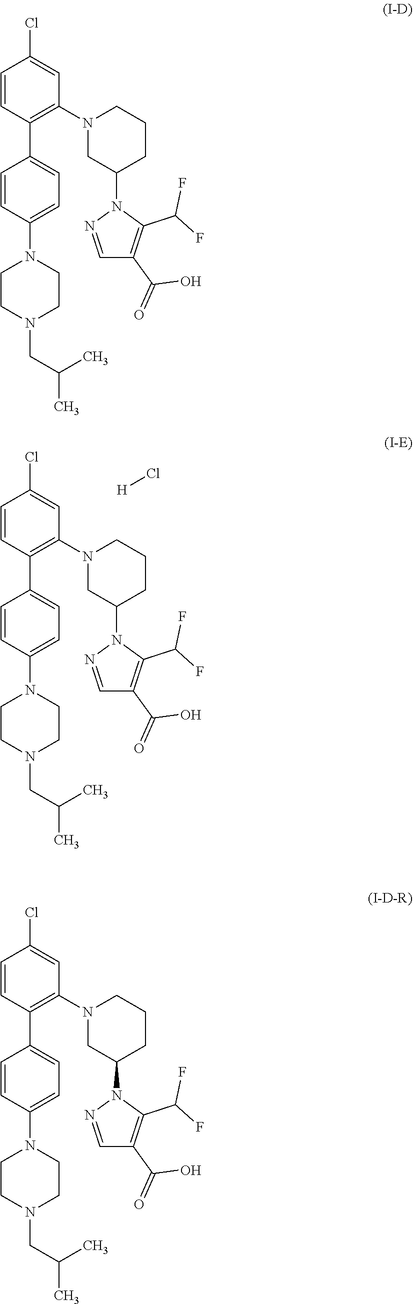 [SMILES: CC(C)CN1CCN(c2ccc(-c3ccc(Cl)cc3N3CCCC(n4ncc(C(=O)O)c4C(F)F)C3)cc2)CC1 ; CC(C)CN1CCN(c2ccc(-c3ccc(Cl)cc3N3CCCC(n4ncc(C(=O)O)c4C(F)F)C3)cc2)CC1 ; Cl ; CC(C)CN1CCN(c2ccc(-c3ccc(Cl)cc3N3CCC[C@@H](n4ncc(C(=O)O)c4C(F)F)C3)cc2)CC1] 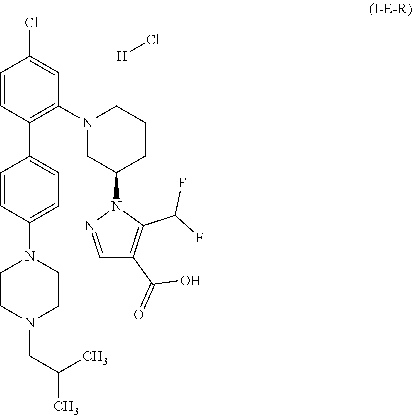 [SMILES: CC(C)CN1CCN(c2ccc(-c3ccc(Cl)cc3N3CCC[C@@H](n4ncc(C(=O)O)c4C(F)F)C3)cc2)CC1 ; Cl]
or one of the salts thereof, solvates thereof or solvates of the salts thereof.

4. The method according to claim 1, wherein the sGC activator is an SGC activator of formula (I-D) 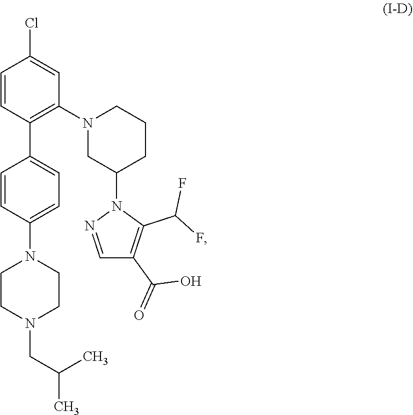 [SMILES: CC(C)CN1CCN(c2ccc(-c3ccc(Cl)cc3N3CCCC(n4ncc(C(=O)O)c4C(F)F)C3)cc2)CC1]
and salts, solvates and solvates of the salts thereof.

5. The method according to claim 1, wherein the sGC activator is an SGC activator of formula (I-E) 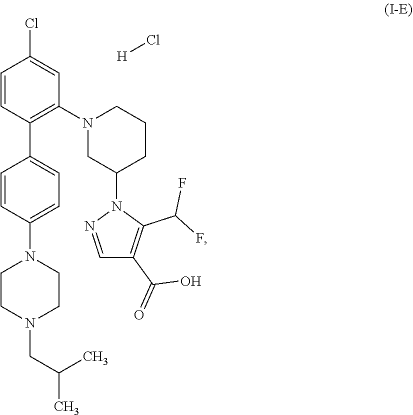 [SMILES: CC(C)CN1CCN(c2ccc(-c3ccc(Cl)cc3N3CCCC(n4ncc(C(=O)O)c4C(F)F)C3)cc2)CC1 ; Cl]
and salts, solvates and solvates of the salts thereof.

6. The method according to claim 1, wherein the eye disease is associated with neurovascular unit damage or retinal ganglion cell/photoreceptor neurodegeneration.

7. The method according to claim 1, wherein the eye disease is selected from the group consisting of non-proliferative diabetic retinopathy, diabetic macular edema, central retinal vein occlusion, branch retinal vein occlusion, retinal artery occlusion, retinopathy of prematurity, ocular ischemic syndrome, radiation retinopathy, anterior ischemic optic neuritis, anti-VEGF therapy driven ischemia, ocular neuropathies and choroidal ischemic diseases.

8. The method according to claim 1, wherein the eye disease is selected from the group consisting of non-proliferative diabetic retinopathy, optic neuropathies and cataract.

9. The method according to claim 1, wherein the eye disease is non-proliferative diabetic retinopathy.

10. The method according to claim 1, wherein the eye disease is selected from the group of optic neuropathies consisting of glaucomatous optic neuropathy, ischemic optic neuropathy, traumatic optic neuropathy, non-arteritic anterior ischemic optic neuropathy, optic neuropathy, leber's hereditary optic neuropathy, methanol associated optic neuropathy and age-related macular degeneration.

11. The method according to claim 10, wherein the optic neuropathy is glaucomatous optic neuropathy.

12. A method for the oral treatment and/or prophylaxis of an eye disease comprising administering an effective amount of a combination to a patient in need thereof, the combination comprising at least one sGC activator according to claim 1 and at least one compound selected from the group consisting of inhibitors of phosphodiesterases 1, 2 and/or 5, calcium, vitamin D and metabolites of vitamin D, bisphosphonates, selected from etidronate, clodronate, tiludronate, teriparatide, pamidronate, neridronate, olpadronate, alendronate, ibandronate, risedronate, and zoledronate, strontium ranelate, active ingredients suitable for hormone replacement therapy in osteoporosis, selected from estrogen and a combination of estrogen and progesterone, selective estrogen receptor modulators, parathyroid hormone and analogs of parathyroid hormone, modulators of receptor activator of nuclear factor kappa-B ligand, sclerostin inhibitors, and TGF-0 inhibitors.

13. A method for the oral treatment and/or prophylaxis of an eye disease comprising administering a pharmaceutical composition to a patient in need thereof, the pharmaceutical composition comprising an effective amount of at least one sGC activator according to claim 1 and one or more inert non-toxic pharmaceutically suitable excipients.

14. A method for the oral treatment and/or prophylaxis of an eye disease comprising administering an effective amount of a pharmaceutical composition to a patient in need thereof, the pharmaceutical composition comprising a combination comprising (1) at least one sGC activator according to claim 1; (2) at least one compound selected from the group consisting of inhibitors of phosphodiesterases 1, 2 and/or 5, calcium, vitamin D and metabolites of vitamin D, bisphosphonates, selected from etidronate, clodronate, tiludronate, teriparatide, pamidronate, neridronate, olpadronate, alendronate, ibandronate, risedronate, and zoledronate, strontium ranelate, active ingredients suitable for hormone replacement therapy in osteoporosis, selected from estrogen and a combination of estrogen and progesterone, selective estrogen receptor modulators, parathyroid hormone and analogs of parathyroid hormone, modulators of receptor activator of nuclear factor kappa-B ligand, sclerostin inhibitors, and TGF-β inhibitors; and (3) one or more inert non-toxic pharmaceutically suitable excipients.

15. A method for the oral treatment and/or prevention of an eye disease selected from the group consisting of non-proliferative diabetic retinopathy, optic neuropathies and cataract in humans and animals comprising administering an effective amount of at least one sGC activator according to claim 1 to a human or animal in need thereof.
